SEQUENCE LISTING (417 sequences)

SEQ ID NO: 1 - protein, 21 aa, synthetic construct
MALPVTALLLPLALLLHAARP

SEQ ID NO: 2 - protein, 45 aa, synthetic construct
TTTPAPRPPTPAPTIASQPLSLRPEACRPAAGGAVHTRGLDFACD

SEQ ID NO: 3 - protein, 230 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKM

SEQ ID NO: 4 - protein, 282 aa, synthetic construct
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 5 - protein, 10 aa, synthetic construct
GGGGSGGGGS

SEQ ID NO: 6 - protein, 24 aa, synthetic construct
IYIWAPLAGTCGVLLLSLVITLYC

SEQ ID NO: 7 - protein, 42 aa, synthetic construct
KRGRKKLLYIFKQPFMRPVQTTQEEDGCSCRFPEEEEGGCEL

SEQ ID NO: 8 - protein, 48 aa, synthetic construct
QRRKYRSNKGESPVEPAEPCRYSCPREEEGSTIPIQEDYRKPEPACSP

SEQ ID NO: 9 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 1184 bp, synthetic construct
  source 1..1184: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 12 - DNA, 63 bp, synthetic construct
  source 1..63: mol_type = other DNA
atggccctgcctgtgacagccctgctgctgcctctggctctgctgctgcatgccgctagaccc

SEQ ID NO: 13 - DNA, 135 bp, synthetic construct
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 14 - DNA, 690 bp, synthetic construct
  source 1..690: mol_type = other DNA
gagagcaagtacggccctccctgccccccttgccctgcccccgagttcctgggcggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttcaatagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaaccaggtgtccctgacctgcctggtgaagggcttctaccccagcgacatcgccgtggagtgggagagcaacggccagcccgagaacaactacaagaccaccccccctgtgctggacagcgacggcagcttcttcctgtacagccggctgaccgtggacaagagccggtggcaggagggcaacgtctttagctgctccgtgatgcacgaggccctgcacaaccactacacccagaagagcctgagcctgtccctgggcaagatg

SEQ ID NO: 15 - DNA, 847 bp, synthetic construct
  source 1..847: mol_type = other DNA
aggtggcccgaaagtcccaaggcccaggcatctagtgttcctactgcacagccccaggcagaaggcagcctagccaaagctactactgcacctgccactacgcgcaatactggccgtggcggggaggagaagaaaaaggagaaagagaaagaagaacaggaagagagggagaccaagacccctgaatgtccatcccatacccagccgctgggcgtctatctcttgactcccgcagtacaggacttgtggcttagagataaggccacctttacatgtttcgtcgtgggctctgacctgaaggatgcccatttgacttgggaggttgccggaaaggtacccacagggggggttgaggaagggttgctggagcgccattccaatggctctcagagccagcactcaagactcacccttccgagatccctgtggaacgccgggacctctgtcacatgtactctaaatcatcctagcctgcccccacagcgtctgatggcccttagagagccagccgcccaggcaccagttaagcttagcctgaatctgctcgccagtagtgatcccccagaggccgccagctggctcttatgcgaagtgtccggctttagcccgcccaacatcttgctcatgtggctggaggaccagcgagaagtgaacaccagcggcttcgctccagcccggcccccaccccagccgggttctaccacattctgggcctggagtgtcttaagggtcccagcaccacctagcccccagccagccacatacacctgtgttgtgtcccatgaagatagcaggaccctgctaaatgcttctaggagtctggaggtttcctacgtgactgaccatt

SEQ ID NO: 16 - DNA, 30 bp, synthetic construct
  source 1..30: mol_type = other DNA
ggtggcggaggttctggaggtggaggttcc

SEQ ID NO: 17 - DNA, 72 bp, synthetic construct
  source 1..72: mol_type = other DNA
atctacatctgggcgcccttggccgggacttgtggggtccttctcctgtcactggttatcaccctttactgc

SEQ ID NO: 18 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 19 - DNA, 123 bp, synthetic construct
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 20 - DNA, 336 bp, synthetic construct
  source 1..336: mol_type = other DNA
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 21 - DNA, 336 bp, synthetic construct
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 22-24 - skipped (3 entries)

SEQ ID NO: 25 - protein, 5 aa, synthetic construct
GGGGS

SEQ ID NO: 26 - protein, 30 aa, synthetic construct
  VARIANT 1..30: note = This sequence may encompass 1-6 GGGGS repeating units
GGGGSGGGGSGGGGSGGGGSGGGGSGGGGS

SEQ ID NO: 27 - protein, 20 aa, synthetic construct
GGGGSGGGGSGGGGSGGGGS

SEQ ID NO: 28 - protein, 15 aa, synthetic construct
GGGGSGGGGSGGGGS

SEQ ID NO: 29 - protein, 4 aa, synthetic construct
GGGS

SEQ ID NO: 30 - RNA, 5000 nt, synthetic construct
  source 1..5000: mol_type = other RNA
  misc_difference 1..5000: note = This sequence may encompass 50-5000 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 31 - DNA, 100 bp, synthetic construct
  source 1..100: mol_type = other DNA
tttttttttttttttttttttttttttttttttttttttttttttttttttttttttttttttttttttttttttttttttttttttttttttttttttt

SEQ ID NO: 32 - DNA, 5000 bp, synthetic construct
  source 1..5000: mol_type = other DNA
  misc_difference 1..5000: note = This sequence may encompass 50-5000 nucleotides
tttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttt

SEQ ID NO: 33 - RNA, 5000 nt, synthetic construct; identical to SEQ ID NO: 30
  source 1..5000: mol_type = other RNA
  misc_difference 1..5000: note = This sequence may encompass 100-5000 nucleotides

SEQ ID NO: 34 - RNA, 400 nt, synthetic construct
  source 1..400: mol_type = other RNA
  misc_difference 1..400: note = This sequence may encompass 100-400 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 35 - RNA, 2000 nt, synthetic construct
  source 1..2000: mol_type = other RNA
  misc_difference 1..2000: note = This sequence may encompass 50-2000 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 36 - protein, 41 aa, synthetic construct
RSKRSRLLHSDYMNMTPRRPGPTRKHYQPYAPPRDFAAYRS

SEQ ID NO: 37 - DNA, 123 bp, synthetic construct; identical to SEQ ID NO: 19
  source 1..123: mol_type = other DNA

SEQ ID NO: 38 - protein, 35 aa, synthetic construct
TKKKYSSSVHDPNGEYMFMRAVNTAKKSRLTDVTL

SEQ ID NO: 39 - DNA, 105 bp, synthetic construct
  source 1..105: mol_type = other DNA
acaaaaaagaagtattcatccagtgtgcacgaccctaacggtgaatacatgttcatgagagcagtgaacacagccaaaaaatccagactcacagatgtgacccta

SEQ ID NO: 40 - DNA, 30 bp, synthetic construct
  source 1..30: mol_type = other DNA
ggtggcggaggttctggaggtgggggttcc

SEQ ID NO: 41 - protein, 4 aa, synthetic construct
GGGS

SEQ ID NO: 42 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

SEQ ID NO: 43 - protein, 18 aa, synthetic construct
GSTSGSGKPGSGEGSTKG

SEQ ID NO: 44-189 - skipped (146 entries)

SEQ ID NO: 190 - DNA, 521 bp, unidentified
  source 1..521: mol_type = unassigned DNA
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccgcctcgtccttcgcagcggccccccgggtgttcccatcgccgcttctaggcccactgcgacgcttgcctgcacttcttacacgctctgggtcccagccgcggcgacgcaaagggccttggtgcgggtctcgtcggcgcagggacgcgtttgggtcccgacggaaccttttccgcgttggggttggggcaccataagct

SEQ ID NO: 191 - DNA, 221 bp, synthetic construct
  source 1..221: mol_type = other DNA
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacg

SEQ ID NO: 192 - DNA, 324 bp, synthetic construct
  source 1..324: mol_type = other DNA
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccg

SEQ ID NO: 193 - DNA, 422 bp, synthetic construct
  source 1..422: mol_type = other DNA
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccgcctcgtccttcgcagcggccccccgggtgttcccatcgccgcttctaggcccactgcgacgcttgcctgcacttcttacacgctctgggtcccagccg

SEQ ID NO: 194-197 - skipped (4 entries)

SEQ ID NO: 198 - DNA, 118 bp, synthetic construct
  source 1..118: mol_type = other DNA
acccctctctccagccactaagccagttgctccctcggctgacggctgcacgcgaggcctccgaacgtcttacgccttgtggcgcgcccgtccttgtcccgggtgtgatggcggggtg

SEQ ID NO: 199 - DNA, 63 bp, synthetic construct
  source 1..63: mol_type = other DNA
atggccctccctgtcaccgccctgctgcttccgctggctcttctgctccacgccgctcggccc

SEQ ID NO: 200-224 - skipped (25 entries)

SEQ ID NO: 225 - protein, 465 aa, synthetic construct
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 226-249 - skipped (24 entries)

SEQ ID NO: 250 - protein, 120 aa, synthetic construct
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 251 - protein, 107 aa, synthetic construct
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 252-274 - skipped (23 entries)

SEQ ID NO: 275 - protein, 132 aa, synthetic construct
DVPDYASLGGPSSPKKKRKVSRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETSY

SEQ ID NO: 276 - protein, 108 aa, synthetic construct
VQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETS

SEQ ID NO: 277 - protein, 93 aa, synthetic construct [sequence omitted]

SEQ ID NO: 278 - protein, 95 aa, synthetic construct
ILWHEMWHEGLIEASRLYFGERNVKGMFEVLEPLHAMMERGPQTLKETSFNQAYGRDLMEAQEWCRKYMKSGNVKDLTQAWDLYYHVFRRISKTS

SEQ ID NO: 279 - protein, 95 aa, synthetic construct
ILWHEMWHEGLLEASRLYFGERNVKGMFEVLEPLHAMMERGPQTLKETSFNQAYGRDLMEAQEWCRKYMKSGNVKDLTQAWDLYYHVFRRISKTS

SEQ ID NO: 280 - protein, 95 aa, synthetic construct
ILWHEMWHEGLEEASRLYFGERNVKGMFEVLEPLHAMMERGPQTLKETSFNQAYGRDLMEAQEWCRKYMKSGNVKDLLQAWDLYYHVFRRISKTS

SEQ ID NO: 281 - protein, 95 aa, synthetic construct
  VARIANT 12: note = Any amino acid
  VARIANT 78: note = Any amino acid
ILWHEMWHEGLXEASRLYFGERNVKGMFEVLEPLHAMMERGPQTLKETSFNQAYGRDLMEAQEWCRKYMKSGNVKDLXQAWDLYYHVFRRISKTS

SEQ ID NO: 282 - protein, 95 aa, synthetic construct
ILWHEMWHEGLIEASRLYFGERNVKGMFEVLEPLHAMMERGPQTLKETSFNQAYGRDLMEAQEWCRKYMKSGNVKDLLQAWDLYYHVFRRISKTS

SEQ ID NO: 283 - protein, 95 aa, synthetic construct
ILWHEMWHEGLLEASRLYFGERNVKGMFEVLEPLHAMMERGPQTLKETSFNQAYGRDLMEAQEWCRKYMKSGNVKDLLQAWDLYYHVFRRISKTS

SEQ ID NO: 284 - protein, 1132 aa, Homo sapiens [sequence omitted]

SEQ ID NO: 285-291 - skipped (7 entries)

SEQ ID NO: 292 - protein, 465 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 293 - protein, 242 aa, synthetic construct
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 294 - protein, 465 aa, synthetic construct
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 295 - protein, 5 aa, synthetic construct
DYGVS

SEQ ID NO: 296 - protein, 16 aa, synthetic construct
VIWGSETTYYNSALKS

SEQ ID NO: 297 - protein, 12 aa, synthetic construct
HYYYGGSYAMDY

SEQ ID NO: 298 - protein, 11 aa, synthetic construct
RASQDISKYLN

SEQ ID NO: 299 - protein, 7 aa, synthetic construct
HTSRLHS

SEQ ID NO: 300 - protein, 9 aa, synthetic construct
QQGNTLPYT

SEQ ID NO: 301 - protein, 486 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 302 - DNA, 1458 bp, synthetic construct
  source 1..1458: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 303 - protein, 242 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 304 - protein, 16 aa, synthetic construct
VIWGSETTYYQSSLKS

SEQ ID NO: 305 - DNA, 813 bp, synthetic construct
  source 1..813: mol_type = other DNA
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagccaccaccatcatcaccatcaccat

SEQ ID NO: 306 - protein, 486 aa, synthetic construct
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 307 - DNA, 1458 bp, synthetic construct
  source 1..1458: mol_type = other DNA
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 308 - skipped

SEQ ID NO: 309 - protein, 284 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 310-330 - skipped (21 entries)

SEQ ID NO: 331 - protein, 120 aa, synthetic construct
QVQLQESGPGLVKPSETLSLTCTVSGVSLPDYGVSWIRQPPGKCLEWIGVIWGSETTYYQSSLKSRVTISKDNSKNQVSLKLSSVTAADTAVYYCAKHYYYGGSYAMDYWGQGTLVTVSS

SEQ ID NO: 332 - protein, 107 aa, synthetic construct
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGCGTKLEIK

SEQ ID NO: 333-348 - skipped (16 entries)

SEQ ID NO: 349 - protein, 486 aa, synthetic construct
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 350-353 - skipped (4 entries)

SEQ ID NO: 354 - DNA, 1461 bp, synthetic construct
  source 1..1461: mol_type = other DNA
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 355 - DNA, 1458 bp, synthetic construct
  source 1..1458: mol_type = other DNA
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 356 - DNA, 1398 bp, synthetic construct
  source 1..1398: mol_type = other DNA
gaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 357-416 - skipped (60 entries)

SEQ ID NO: 417 - DNA, 1395 bp, synthetic construct
  source 1..1395: mol_type = other DNA
gaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

(19) United States
(12) Patent Application Publication
(10) Pub. No.: US 2026/0295043 A1
(43) Pub. Date: Oct. 1, 2026

(54) TREATMENT OF AUTOIMMUNE DISORDERS USING CHIMERIC ANTIGEN RECEPTOR THERAPY

(71) Applicant: Novartis AG, Basel (CH)

(72) Inventors: Bambang Adiwijaya, Lexington, MA (US); Thomas Calzascia, Binningen (CH); Peter Gergely, Oberwil (CH); David Scott Pearson, Allschwil (CH); Martin Stangel, Basel (CH)

(21) Appl. No.: 19/704,783

(22) Filed: Jun. 11, 2026

Related U.S. Application Data
(63) Continuation of application No. 19/111,481, filed on Sep. 14, 2023.
(60) Provisional application No. 63/375,776, filed on Sep. 15, 2022.
(60) Provisional application No. 63/507,141, filed on Jun. 9, 2023.

(51) Int. Cl. A61K 40/31; A61K 31/7088; A61K 35/17; A61K 40/11; A61K 40/13; A61K 40/15; A61K 40/42; A61K 40/50; A61K 45/06; A61P 37/02; C12N 5/0781; C12N 5/0783

(52) U.S. Cl. CPC A61K 40/31; A61K 31/7088; A61K 35/17; A61K 40/11; A61K 40/13; A61K 40/15; A61K 40/4211; A61K 40/50; A61K 45/06; A61P 37/02; C12N 5/0635; C12N 5/0636; A61K 2239/10; C12N 2501/2315; C12N 2501/515

(57) ABSTRACT

The invention provides methods of making immune effector cells (for example, T cells, NK cells) that express a chimeric antigen receptor (CAR), and compositions generated by such methods, and therapeutic uses thereof for treating autoimmune diseases or disorders.

### CROSS REFERENCE TO RELATED APPLICATIONS

This application is a continuation of U.S. application Ser. No. 19/111,481, filed Mar. 13, 2025, which is a U.S. National Phase Application under 35 U.S.C. § 371 of International Application No. PCT/EP2023/075316, filed Sep. 14, 2023, which claims the benefit of U.S. Provisional Application No. 63/375,776, filed Sep. 15, 2022, and U.S. Provisional Application No. 63/507,141, filed Jun. 9, 2023. The contents of the aforementioned applications are hereby incorporated by reference in their entirety.

### SEQUENCE LISTING

The instant application contains a Sequence Listing which has been submitted electronically in XML format compliant with WIPO Standard ST.26 and is hereby incorporated by reference in its entirety. Said XML copy, created on Jun. 10, 2026, is named N2067-720320FT_SL.xml and is 182,359 bytes in size.

### FIELD OF THE INVENTION

The present invention relates generally to methods of making immune effector cells (for example, T cells or NK cells) engineered to express a Chimeric Antigen Receptor (CAR), compositions comprising the same, and therapeutic uses thereof for treating autoimmune diseases or disorders.

### BACKGROUND OF THE INVENTION

Current therapies for severe autoimmune diseases such as systemic lupus erythematosus (SLE) includes conventional immunomodulatory and anti-inflammatory agents such as antimalarials, glucocorticoids, and immunosuppressives (e.g. methotrexate, azathioprine, mycophenolate and cyclophosphamide) and biologics (such as, belimumab and very recently, anifrolumab as well as rituximab commonly used in the severe stage of the disease). Severe refractory SLE (srSLE) patients, with or without renal involvement, after having failed immunosuppressive and biological therapies, have very limited treatment options. Autologous stem cell transplantation (ASCT) may be performed; however, it remains experimental and is associated with significant toxicities including mortality.

Thus, there exists an unmet need for new treatments for severe autoimmune diseases, including srSLE.

### SUMMARY OF THE INVENTION

The present disclosure pertains to methods of making immune effector cells (for example, T cells or NK cells) engineered to express a CAR, and compositions generated using such methods. Also disclosed are methods of using such compositions for treating a disease, for example, an autoimmune disease or disorder, e.g., lupus (e.g., systemic lupus erythematosus (SLE), e.g., severe refractory systemic lupus erythematosus (srSLE), or lupus nephritis), systemic sclerosis (e.g., rapidly progressing systemic sclerosis (SSc) with significant lung involvement (e.g. as for autoHSCT)), idiopathic inflammatory myopathies (e.g., polymyositis, dermatomyositis, anti-synthetase syndrome, immune-mediated necrotizing myopathy, inclusion body myositis, overlap myositis, cancer associated myositis, e.g. anti-synthetase syndrome with ILD), vasculitis (e.g., ANCA-associated vasculitis), severe refractory Sjögren's, severe refractory neuroimmune disease (e.g. myasthenia gravis (MG), neuromyelitis optica (NMO), MOG associated disease (MOGAD), multiple sclerosis (MS)), severe refractory rheumatoid arthritis, antibody mediated neuroimmune diseases (e.g., AChR+ and MuSK+myasthenia gravis (MG), AQP4+ neuromyelitis optica (NMO), MOGAD (anti-MOG associated disease), NMDAR+ encephalitis, or antibody-associated neurological paraneoplastic diseases), Addison's disease, Goodpasture's syndrome, thyrotoxicosis, chronic active hepatitis, relapsing polychondritis, pemphigus vulgaris, or amyotrophic lateral sclerosis in a subject.

In one aspect, the disclosure provides a method of treating a subject having an autoimmune disease or disorder, e.g., lupus (e.g., systemic lupus erythematosus (SLE), e.g., severe refractory systemic lupus erythematosus (srSLE), or lupus nephritis), systemic sclerosis (e.g., rapidly progressing systemic sclerosis (SSc) with significant lung involvement (e.g. as for autoHSCT)), idiopathic inflammatory myopathies (e.g., polymyositis, dermatomyositis, anti-synthetase syndrome, immune-mediated necrotizing myopathy, inclusion body myositis, overlap myositis, cancer associated myositis, e.g. anti-synthetase syndrome with ILD), vasculitis (e.g., ANCA-associated vasculitis), severe refractory Sjögren's, severe refractory neuroimmune disease (e.g. myasthenia gravis (MG), neuromyelitis optica (NMO), MOG associated disease (MOGAD), multiple sclerosis (MS)), severe refractory rheumatoid arthritis, antibody mediated neuroimmune diseases (e.g., AChR+ and MuSK+myasthenia gravis (MG), AQP4+ neuromyelitis optica (NMO), MOGAD (anti-MOG associated disease), NMDAR+encephalitis, or antibody-associated neurological paraneoplastic diseases), Addison's disease, Goodpasture's syndrome, thyrotoxicosis, chronic active hepatitis, relapsing polychondritis, pemphigus vulgaris, or amyotrophic lateral sclerosis, the method comprising administering to the subject a population of cells (for example, T cells) that express, or comprise a nucleic acid configured to express, a CD19 chimeric antigen receptor (CAR), wherein the population of cells was made by a method comprising:
(i) contacting (for example, binding) a population of cells (for example, T cells, for example, T cells isolated from a frozen or fresh leukapheresis product) with an agent that stimulates a CD3/TCR complex and/or an agent that stimulates a costimulatory molecule on the surface of the cells;
(ii) contacting the population of cells (for example, T cells) with a nucleic acid molecule (for example, a DNA or RNA molecule) encoding the CAR, thereby providing a population of cells (for example, T cells) comprising the nucleic acid molecule, wherein the CAR comprises a CD19 antigen binding domain (“CD19 CAR”); and
(iii) harvesting the population of cells (for example, T cells) for storage (for example, reformulating the population of cells in cryopreservation media) or administration, wherein:
(a) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and
   step (iii) is performed no later than 30 (for example, 26) hours after the beginning of step (i), for example, no later than 22, 23, 24, 25, 26, 27, 28, 29, or 30 hours after the beginning of step (i), for example, no later than 24 hours after the beginning of step (i),
(b) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and
   step (iii) is performed no later than 30 hours after the beginning of step (ii), for example, no later than 22, 23, 24, 25, 26, 27, 28, 29, or 30 hours after the beginning of step (ii), or
(c) the population of cells from step (iii) are not expanded, or expanded by no more than 5, 10, 15, 20, 25, 30, 35, or 40%, for example, no more than 10%, for example, as assessed by the number of living cells, compared to the population of cells at the beginning of step (i),
optionally wherein the nucleic acid molecule in step (ii) is on a viral vector, optionally wherein the nucleic acid molecule in step (ii) is an RNA molecule on a viral vector, optionally wherein step (ii) comprises transducing the population of cells (for example, T cells) with a viral vector comprising a nucleic acid molecule encoding the CAR.

In some embodiments, the agent that stimulates a CD3/TCR complex is an agent that stimulates CD3 (for example, an anti-CD3 antibody) and wherein the agent that stimulates a costimulatory molecule is an agent that stimulates CD28, ICOS, CD27, HVEM, LIGHT, CD40, 4-1BB, OX40, DR3, GITR, CD30, TIM1, CD2, CD226, or any combination thereof, optionally wherein the agent that stimulates a CD3/TCR complex or the agent that stimulates a costimulatory molecule is chosen from an antibody (for example, a single-domain antibody (for example, a heavy chain variable domain antibody), a peptibody, a Fab fragment, or a scFv), a small molecule, or a ligand (for example, a naturally-existing, recombinant, or chimeric ligand), optionally wherein the agent that stimulates a CD3/TCR complex or the agent that stimulates a costimulatory molecule does not comprise a bead, optionally wherein the agent that stimulates a CD3/TCR complex comprises an anti-CD3 antibody and the agent that stimulates a costimulatory molecule comprises an anti-CD28 antibody, optionally wherein the agent that stimulates a CD3/TCR complex comprises an anti-CD3 antibody covalently attached to a colloidal polymeric nanomatrix and the agent that stimulates a costimulatory molecule comprises an anti-CD28 antibody covalently attached to a colloidal polymeric nanomatrix, optionally wherein the agent that stimulates a CD3/TCR complex and the agent that stimulates a costimulatory molecule comprise T Cell TransAct™.

In some embodiments, step (i) increases the percentage of CAR-expressing cells in the population of cells from step (iii), for example, the population of cells from step (iii) shows a higher percentage of CAR-expressing cells (for example, at least 10, 20, 30, 40, 50, or 60% higher), compared with cells made by an otherwise similar method without step (i).

In some embodiments:
(a) the percentage of naïve cells, for example, naïve T cells, for example, CD45RA+ CD45RO− CCR7+ T cells, in the population of cells from step (iii) is the same as or differs by no more than 5 or 10% from the percentage of naïve cells, for example, naïve T cells, for example, CD45RA+ CD45RO− CCR7+ cells, in the population of cells at the beginning of step (i);
(b) the percentage of naïve cells, for example, naïve T cells, for example, CD45RA+ CD45RO− CCR7+ T cells, in the population of cells from step (iii) is increased by, for example, at least 1.2, 1.4, 1.6, 1.8, 2.0, 2.2, 2.4, 2.6, 2.8, or 3-fold, as compared to the percentage of naïve cells, for example, naïve T cells, for example, CD45RA+CD45RO− CCR7+ cells, in the population of cells at the beginning of step (i);
(c) the percentage of CAR-expressing naïve T cells, for example, CAR-expressing CD45RA+ CD45RO− CCR7+ T cells in the population of cells increases during the duration of step (ii), for example, increases by, for example, at least 30, 35, 40, 45, 50, 55, or 60%, between 18-24 hours after the beginning of step (ii); or
(d) the percentage of naïve cells, for example, naïve T cells, for example, CD45RA+CD45RO− CCR7+ T cells, in the population of cells from step (iii) does not decrease, or decreases by no more than 5 or 10%, as compared to the percentage of naïve cells, for example, naïve T cells, for example, CD45RA+CD45RO− CCR7+ cells, in the population of cells at the beginning of step (i).

In some embodiments:
(a) the population of cells from step (iii) shows a higher percentage of naïve cells, for example, naïve T cells, for example, CD45RA+CD45RO− CCR7+ T cells (for example, at least 10, 20, 30, or 40% higher), compared with cells made by an otherwise similar method in which step (iii) is performed more than 26 hours after the beginning of step (i), for example, more than 5, 6, 7, 8, 9, 10, 11, or 12 days after the beginning of step (i);
(b) the percentage of naïve cells, for example, naïve T cells, for example, CD45RA+ CD45RO− CCR7+ T cells, in the population of cells from step (iii) is higher (for example, at least 1.2, 1.4, 1.6, 1.8, 2.0, 2.2, 2.4, 2.6, 2.8, or 3-fold higher) than the percentage of naïve cells, for example, naïve T cells, for example, CD45RA+CD45RO− CCR7+ T cells, in cells made by an otherwise similar method in which step (iii) is performed more than 26 hours after the beginning of step (i), for example, more than 5, 6, 7, 8, 9, 10, 11, or 12 days after the beginning of step (i);
(c) the percentage of CAR-expressing naïve T cells, for example, CAR-expressing CD45RA+CD45RO− CCR7+ T cells, in the population of cells from step (iii) is higher (for example, at least 4, 6, 8, 10, or 12-fold higher) than the percentage of CAR-expressing naïve T cells, for example, CAR-expressing CD45RA+CD45RO− CCR7+ T cells, in cells made by an otherwise similar method in which step (iii) is performed more than 26 hours after the beginning of step (i), for example, more than 5, 6, 7, 8, 9, 10, 11, or 12 days after the beginning of step (i);
(d) the population of cells from step (iii) shows a higher percentage of naïve cells, for example, naïve T cells, for example, CD45RA+CD45RO− CCR7+ T cells (for example, at least 10, 20, 30, or 40% higher), compared with cells made by an otherwise similar method which further comprises, after step (ii) and prior to step (iii), expanding the population of cells (for example, T cells) in vitro for more than 3 days, for example, for 5, 6, 7, 8 or 9 days;
(e) the percentage of naïve cells, for example, naïve T cells, for example, CD45RA+ CD45RO− CCR7+ T cells, in the population of cells from step (iii) is higher (for example, at least 1.2, 1.4, 1.6, 1.8, 2.0, 2.2, 2.4, 2.6, 2.8, or 3-fold higher) than the percentage of naïve cells, for example, naïve T cells, for example, CD45RA+CD45RO− CCR7+ T cells, in cells made by an otherwise similar method which further comprises, after step (ii) and prior to step (iii), expanding the population of cells (for example, T cells) in vitro for more than 3 days, for example, for 5, 6, 7, 8 or 9 days; or
(f) the percentage of CAR-expressing naïve T cells, for example, CAR-expressing CD45RA+CD45RO− CCR7+ T cells, in the population of cells from step (iii) is higher (for example, at least 4, 6, 8, 10, or 12-fold higher) than the percentage of CAR-expressing naïve T cells, for example, CAR-expressing CD45RA+CD45RO− CCR7+ T cells, in cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments:
(a) the percentage of central memory cells, for example, central memory T cells, for example, CD95+ central memory T cells, in the population of cells from step (iii) is the same as or differs by no more than 5 or 10% from the percentage of central memory cells, for example, central memory T cells, for example, CD95+ central memory T cells, in the population of cells at the beginning of step (i);
(b) the percentage of central memory cells, for example, central memory T cells, for example, CCR7+CD45RO+ T cells, in the population of cells from step (iii) is reduced by at least 20, 25, 30, 35, 40, 45, or 50%, as compared to the percentage of central memory cells, for example, central memory T cells, for example, CCR7+CD45RO+ T cells, in the population of cells at the beginning of step (i);
(c) the percentage of CAR-expressing central memory T cells, for example, CAR-expressing CCR7+CD45RO+ cells, decreases during the duration of step (ii), for example, decreases by, for example, at least 8, 10, 12, 14, 16, 18, or 20%, between 18-24 hours after the beginning of step (ii); or
(d) the percentage of central memory cells, for example, central memory T cells, for example, CCR7+CD45RO+ T cells, in the population of cells from step (iii) does not increase, or increases by no more than 5 or 10%, as compared to the percentage of central memory cells, for example, central memory T cells, for example, CCR7+CD45RO+ T cells, in the population of cells at the beginning of step (i).

In some embodiments:
(a) the population of cells from step (iii) shows a lower percentage of central memory cells, for example, central memory T cells, for example, CD95+ central memory T cells (for example, at least 10, 20, 30, or 40% lower), compared with cells made by an otherwise similar method in which step (iii) is performed more than 26 hours after the beginning of step (i), for example, more than 5, 6, 7, 8, 9, 10, 11, or 12 days after the beginning of step (i);
(b) the percentage of central memory cells, for example, central memory T cells, for example, CCR7+CD45RO+ T cells in the population of cells from step (iii) is lower (for example, at least 20, 30, 40, or 50% lower) than the percentage of central memory cells, for example, central memory T cells, for example, CCR7+CD45RO+ T cells, in cells made by an otherwise similar method in which step (iii) is performed more than 26 hours after the beginning of step (i), for example, more than 5, 6, 7, 8, 9, 10, 11, or 12 days after the beginning of step (i);
(c) the percentage of CAR-expressing central memory T cells, for example, CAR-expressing CCR7+CD45RO+ T cells in the population of cells from step (iii) is lower (for example, at least 10, 20, 30, or 40% lower) than the percentage of CAR-expressing central memory T cells, for example, CAR-expressing CCR7+CD45RO+ T cells, in cells made by an otherwise similar method in which step (iii) is performed more than 26 hours after the beginning of step (i), for example, more than 5, 6, 7, 8, 9, 10, 11, or 12 days after the beginning of step (i);
(d) the population of cells from step (iii) shows a lower percentage of central memory cells, for example, central memory T cells, for example, CD95+ central memory T cells (for example, at least 10, 20, 30, or 40% lower), compared with cells made by an otherwise similar method which further comprises, after step (ii) and prior to step (iii), expanding the population of cells (for example, T cells) in vitro for more than 3 days, for example, for 5, 6, 7, 8 or 9 days;
(e) the percentage of central memory cells, for example, central memory T cells, for example, CCR7+CD45RO+ T cells in the population of cells from step (iii) is lower (for example, at least 20, 30, 40, or 50% lower) than the percentage of central memory cells, for example, central memory T cells, for example, CCR7+CD45RO+ T cells, in cells made by an otherwise similar method which further comprises, after step (ii) and prior to step (iii), expanding the population of cells (for example, T cells) in vitro for more than 3 days, for example, for 5, 6, 7, 8 or 9 days; or
(f) the percentage of CAR-expressing central memory T cells, for example, CAR-expressing CCR7+CD45RO+ T cells in the population of cells from step (iii) is lower (for example, at least 10, 20, 30, or 40% lower) than the percentage of CAR-expressing central memory T cells, for example, CAR-expressing CCR7+CD45RO+ T cells, in cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments:
(a) the percentage of stem memory T cells, for example, CD45RA+CD95+IL-2 receptor β+CCR7+CD62L+ T cells, in the population of cells from step (iii) is increased, as compared to the percentage of stem memory T cells, for example, CD45RA+CD95+IL-2 receptor β+CCR7+CD62L+ T cells, in the population of cells at the beginning of step (i);
(b) the percentage of CAR-expressing stem memory T cells, for example, CAR-expressing CD45RA+CD95+IL-2 receptor β+CCR7+CD62L+ T cells, in the population of cells from step (iii) is increased, as compared to the percentage of CAR-expressing stem memory T cells, for example, CAR-expressing CD45RA+CD95+IL-2 receptor β+CCR7+CD62L+ T cells, in the population of cells at the beginning of step (i);
(c) the percentage of stem memory T cells, for example, CD45RA+CD95+IL-2 receptor β+CCR7+CD62L+ T cells, in the population of cells from step (iii) is higher than the percentage of stem memory T cells, for example, CD45RA+CD95+IL-2 receptor β+CCR7+CD62L+ T cells, in cells made by an otherwise similar method in which step (iii) is performed more than 26 hours after the beginning of step (i), for example, more than 5, 6, 7, 8, 9, 10, 11, or 12 days after the beginning of step (i); or
(d) the percentage of CAR-expressing stem memory T cells, for example, CAR-expressing CD45RA+CD95+IL-2 receptor β+CCR7+CD62L+ T cells, in the population of cells from step (iii) is higher than the percentage of CAR-expressing stem memory T cells, for example, CAR-expressing CD45RA+CD95+IL-2 receptor β+CCR7+CD62L+ T cells, in cells made by an otherwise similar method in which step (iii) is performed more than 26 hours after the beginning of step (i), for example, more than 5, 6, 7, 8, 9, 10, 11, or 12 days after the beginning of step (i);
(e) the percentage of stem memory T cells, for example, CD45RA+CD95+IL-2 receptor β+CCR7+CD62L+ T cells, in the population of cells from step (iii) is higher than the percentage of stem memory T cells, for example, CD45RA+CD95+IL-2 receptor β+CCR7+CD62L+ T cells, in cells made by an otherwise similar method which further comprises, after step (ii) and prior to step (iii), expanding the population of cells (for example, T cells) in vitro for more than 3 days, for example, for 5, 6, 7, 8 or 9 days; or
(f) the percentage of CAR-expressing stem memory T cells, for example, CAR-expressing CD45RA+CD95+IL-2 receptor β+CCR7+CD62L+ T cells, in the population of cells from step (iii) is higher than the percentage of CAR-expressing stem memory T cells, for example, CAR-expressing CD45RA+CD95+IL-2 receptor β+CCR7+CD62L+ T cells, in cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments:
(a) the median GeneSetScore (Up TEM vs. Down TSCM) of the population of cells from step (iii) is about the same as or differs by no more than (for example, increased by no more than) about 25, 50, 75, 100, or 125% from the median GeneSetScore (Up TEM vs. Down TSCM) of the population of cells at the beginning of step (i);
(b) the median GeneSetScore (Up TEM vs. Down TSCM) of the population of cells from step (iii) is lower (for example, at least about 100, 150, 200, 250, or 300% lower) than the median GeneSetScore (Up TEM vs. Down TSCM) of:
   cells made by an otherwise similar method in which step (iii) is performed more than 26 hours after the beginning of step (i), for example, more than 5, 6, 7, 8, 9, 10, 11, or 12 days after the beginning of step (i), or
   cells made by an otherwise similar method which further comprises, after step (ii) and prior to step (iii), expanding the population of cells (for example, T cells) in vitro for more than 3 days, for example, for 5, 6, 7, 8 or 9 days;
(c) the median GeneSetScore (Up Treg vs. Down Teff) of the population of cells from step (iii) is about the same as or differs by no more than (for example, increased by no more than) about 25, 50, 100, 150, or 200% from the median GeneSetScore (Up Treg vs. Down Teff) of the population of cells at the beginning of step (i);
(d) the median GeneSetScore (Up Treg vs. Down Teff) of the population of cells from step (iii) is lower (for example, at least about 50, 100, 125, 150, or 175% lower) than the median GeneSetScore (Up Treg vs. Down Teff) of:
   cells made by an otherwise similar method in which step (iii) is performed more than 26 hours after the beginning of step (i), for example, more than 5, 6, 7, 8, 9, 10, 11, or 12 days after the beginning of step (i), or
   cells made by an otherwise similar method which further comprises, after step (ii) and prior to step (iii), expanding the population of cells (for example, T cells) in vitro for more than 3 days, for example, for 5, 6, 7, 8 or 9 days;
(e) the median GeneSetScore (Down stemness) of the population of cells from step (iii) is about the same as or differs by no more than (for example, increased by no more than) about 25, 50, 100, 150, 200, or 250% from the median GeneSetScore (Down stemness) of the population of cells at the beginning of step (i);
(f) the median GeneSetScore (Down stemness) of the population of cells from step (iii) is lower (for example, at least about 50, 100, or 125% lower) than the median GeneSetScore (Down stemness) of:
   cells made by an otherwise similar method in which step (iii) is performed more than 26 hours after the beginning of step (i), for example, more than 5, 6, 7, 8, 9, 10, 11, or 12 days after the beginning of step (i), or
   cells made by an otherwise similar method which further comprises, after step (ii) and prior to step (iii), expanding the population of cells (for example, T cells) in vitro for more than 3 days, for example, for 5, 6, 7, 8 or 9 days;
(g) the median GeneSetScore (Up hypoxia) of the population of cells from step (iii) is about the same as or differs by no more than (for example, increased by no more than) about 125, 150, 175, or 200% from the median GeneSetScore (Up hypoxia) of the population of cells at the beginning of step (i);
(h) the median GeneSetScore (Up hypoxia) of the population of cells from step (iii) is lower (for example, at least about 40, 50, 60, 70, or 80% lower) than the median GeneSetScore (Up hypoxia) of:
   cells made by an otherwise similar method in which step (iii) is performed more than 26 hours after the beginning of step (i), for example, more than 5, 6, 7, 8, 9, 10, 11, or 12 days after the beginning of step (i), or
   cells made by an otherwise similar method which further comprises, after step (ii) and prior to step (iii), expanding the population of cells (for example, T cells) in vitro for more than 3 days, for example, for 5, 6, 7, 8 or 9 days;
(j) the median GeneSetScore (Up autophagy) of the population of cells from step (iii) is about the same as or differs by no more than (for example, increased by no more than) about 180, 190, 200, or 210% from the median GeneSetScore (Up autophagy) of the population of cells at the beginning of step (i); or
(k) the median GeneSetScore (Up autophagy) of the population of cells from step (iii) is lower (for example, at least 20, 30, or 40% lower) than the median GeneSetScore (Up autophagy) of:
   cells made by an otherwise similar method in which step (iii) is performed more than 26 hours after the beginning of step (i), for example, more than 5, 6, 7, 8, 9, 10, 11, or 12 days after the beginning of step (i), or
   cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the population of cells from step (iii), after being incubated with a cell expressing an antigen recognized by the CAR, secretes IL-2 at a higher level (for example, at least 2, 4, 6, 8, 10, 12, or 14-fold higher) than cells made by an otherwise similar method in which step (iii) is performed more than 26 hours after the beginning of step (i), for example, more than 5, 6, 7, 8, 9, 10, 11, or 12 days after the beginning of step (i), or cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the population of cells from step (iii), after being administered to the subject in vivo, persists longer or expands at a higher level, compared with cells made by an otherwise similar method in which step (iii) is performed more than 26 hours after the beginning of step (i), for example, more than 5, 6, 7, 8, 9, 10, 11, or 12 days after the beginning of step (i), or compared with cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the population of cells from step (iii), after being administered to the subject in vivo, shows a stronger activity (for example, a stronger activity at a low dose, for example, a dose no more than 0.15×10⁶, 0.2×10⁶, 0.25×10⁶, or 0.3×10⁶ viable CAR-expressing cells) than cells made by an otherwise similar method in which step (iii) is performed more than 26 hours after the beginning of step (i), for example, more than 5, 6, 7, 8, 9, 10, 11, or 12 days after the beginning of step (i), or cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the population of cells from step (iii) are not expanded, or expanded by no more than 5, 10, 15, 20, 25, 30, 35, or 40%, for example, no more than 10%, for example, as assessed by the number of living cells, compared to the population of cells at the beginning of step (i), optionally wherein the number of living cells in the population of cells from step (iii) decreases from the number of living cells in the population of cells at the beginning of step (i).

In some embodiments, the population of cells from step (iii) are not expanded, or expanded by less than 2 hours, for example, less than 1 or 1.5 hours, compared to the population of cells at the beginning of step (i).

In some embodiments, steps (i) and/or (ii) are performed in cell media (for example, serum-free media) comprising IL-2, IL-15 (for example, hetIL-15 (IL15/sIL-15Ra)), IL-7, IL-21, IL-6 (for example, IL-6/sIL-6Ra), a LSD1 inhibitor, a MALT1 inhibitor, or a combination thereof.

In some embodiments, steps (i) and/or (ii) are performed in serum-free cell media comprising a serum replacement. In some embodiments, the serum replacement is CTS™ Immune Cell Serum Replacement (ICSR).
In some embodiments, the method further comprises, prior to step (i):
(iv) (optionally) receiving a fresh leukapheresis product (or an alternative source of hematopoietic tissue such as a fresh whole blood product, a fresh bone marrow product, or a fresh organ biopsy or removal (for example, a fresh product from thymectomy)) from an entity, for example, a laboratory, hospital, or healthcare provider, and
(v) isolating the population of cells (for example, T cells, for example, CD8+ and/or CD4+ T cells) contacted in step (i) from a fresh leukapheresis product (or an alternative source of hematopoietic tissue such as a fresh whole blood product, a fresh bone marrow product, or a fresh organ biopsy or removal (for example, a fresh product from thymectomy)), optionally wherein:
step (iii) is performed no later than 35 hours after the beginning of step (v), for example, no later than 27, 28, 29, 30, 31, 32, 33, 34, or 35 hours after the beginning of step (v), for example, no later than 30 hours after the beginning of step (v), or
the population of cells from step (iii) are not expanded, or expanded by no more than 5, 10, 15, 20, 25, 30, 35, or 40%, for example, no more than 10%, for example, as assessed by the number of living cells, compared to the population of cells at the end of step (v).

In some embodiments, the method further comprises prior to step (i): receiving cryopreserved T cells isolated from a leukapheresis product (or an alternative source of hematopoietic tissue such as cryopreserved T cells isolated from whole blood, bone marrow, or organ biopsy or removal (for example, thymectomy)) from an entity, for example, a laboratory, hospital, or healthcare provider.

In some embodiments, the method further comprises prior to step (i):
(iv) (optionally) receiving a cryopreserved leukapheresis product (or an alternative source of hematopoietic tissue such as a cryopreserved whole blood product, a cryopreserved bone marrow product, or a cryopreserved organ biopsy or removal (for example, a cryopreserved product from thymectomy)) from an entity, for example, a laboratory, hospital, or healthcare provider, and
(v) isolating the population of cells (for example, T cells, for example, CD8+ and/or CD4+ T cells) contacted in step (i) from a cryopreserved leukapheresis product (or an alternative source of hematopoietic tissue such as a cryopreserved whole blood product, a cryopreserved bone marrow product, or a cryopreserved organ biopsy or removal (for example, a cryopreserved product from thymectomy)), optionally wherein:
step (iii) is performed no later than 35 hours after the beginning of step (v), for example, no later than 27, 28, 29, 30, 31, 32, 33, 34, or 35 hours after the beginning of step (v), for example, no later than 30 hours after the beginning of step (v), or
the population of cells from step (iii) are not expanded, or expanded by no more than 5, 10, 15, 20, 25, 30, 35, or 40%, for example, no more than 10%, for example, as assessed by the number of living cells, compared to the population of cells at the end of step (v).

In some embodiments, the method further comprises step (vi):
culturing a portion of the population of cells from step (iii) for at least 2, 2.5, 3, 3.5, 4, 4.5, 5, 5.5, 6, 6.5, or 7 days, for example, at least 2 days and no more than 7 days, and measuring CAR expression level in the portion (for example, measuring the percentage of viable, CAR-expressing cells in the portion), optionally wherein:
step (iii) comprises harvesting and freezing the population of cells (for example, T cells) and step (vi) comprises thawing a portion of the population of cells from step (iii), culturing the portion for at least 2, 2.5, 3, 3.5, 4, 4.5, 5, 5.5, 6, 6.5, or 7 days, for example, at least 2 days and no more than 7 days, and measuring CAR expression level in the portion (for example, measuring the percentage of viable, CAR-expressing cells in the portion).

In some embodiments, the population of cells at the beginning of step (i) or step (1) has been enriched for IL6R-expressing cells (for example, cells that are positive for IL6Ra and/or IL6RB). In some embodiments, the population of cells at the beginning of step (i) or step (1) comprises no less than 50, 60, or 70% of IL6R-expressing cells (for example, cells that are positive for IL6Ra and/or IL6Rβ).

In some embodiments, steps (i) and (ii) or steps (1) and (2) are performed in cell media comprising IL-15 (for example, hetIL-15 (IL15/sIL-15Ra)). In some embodiments, IL-15 increases the ability of the population of cells to expand, for example, 10, 15, 20, or 25 days later. In some embodiments, IL-15 increases the percentage of IL6Rβ-expressing cells in the population of cells.

In one aspect, the disclosure provides a method of treating a subject having an autoimmune disease or disorder, e.g., lupus (e.g., systemic lupus erythematosus (SLE), e.g., severe refractory systemic lupus erythematosus (srSLE), or lupus nephritis), systemic sclerosis (e.g., rapidly progressing systemic sclerosis (SSc) with significant lung involvement (e.g. as for autoHSCT)), idiopathic inflammatory myopathies (e.g., polymyositis, dermatomyositis, anti-synthetase syndrome, immune-mediated necrotizing myopathy, inclusion body myositis, overlap myositis, cancer associated myositis, e.g. anti-synthetase syndrome with ILD), vasculitis (e.g., ANCA-associated vasculitis), severe refractory Sjögren's, severe refractory neuroimmune disease (e.g. myasthenia gravis (MG), neuromyelitis optica (NMO), MOG associated disease (MOGAD), multiple sclerosis (MS)), severe refractory rheumatoid arthritis, antibody mediated neuroimmune diseases (e.g., AChR+ and MuSK+myasthenia gravis (MG), AQP4+ neuromyelitis optica (NMO), MOGAD (anti-MOG associated disease), NMDAR+encephalitis, or antibody-associated neurological paraneoplastic diseases), Addison's disease, Goodpasture's syndrome, thyrotoxicosis, chronic active hepatitis, relapsing polychondritis, pemphigus vulgaris, or amyotrophic lateral sclerosis, the method comprising administering to the subject a population of cells engineered to express a CD19 CAR (“a population of CAR-expressing cells”), said population comprising:
(a) about the same percentage of naïve cells, for example, naïve T cells, for example, CD45RO− CCR7+ T cells, as compared to the percentage of naïve cells, for example, naïve T cells, for example, CD45RO− CCR7+ cells, in the same population of cells prior to being engineered to express the CAR;
(b) a change within about 5% to about 10% of naïve cells, for example, naïve T cells, for example, CD45RO− CCR7+ T cells, for example, as compared to the percentage of naïve cells, for example, naïve T cells, for example, CD45RO− CCR7+ cells, in the same population of cells prior to being engineered to express the CAR;
(c) an increased percentage of naïve cells, for example, naïve T cells, for example, CD45RO− CCR7+ T cells, for example, increased by at least 1.2, 1.4, 1.6, 1.8, 2.0, 2.2, 2.4, 2.6, 2.8, or 3-fold, as compared to the percentage of naïve cells, for example, naïve T cells, for example, CD45RO− CCR7+ cells, in the same population of cells prior to being engineered to express the CAR;
(d) about the same percentage of central memory cells, for example, central memory T cells, for example, CCR7+CD45RO+ T cells, as compared to the percentage of central memory cells, for example, central memory T cells, for example, CCR7+CD45RO+ T cells, in the same population of cells prior to being engineered to express the CAR;
(e) a change within about 5% to about 10% of central memory cells, for example, central memory T cells, for example, CCR7+CD45RO+ T cells, as compared to the percentage of central memory cells, for example, central memory T cells, for example, CCR7+CD45RO+ T cells, in the same population of cells prior to being engineered to express the CAR;
(f) a decreased percentage of central memory cells, for example, central memory T cells, for example, CCR7+CD45RO+ T cells, for example, decreased by at least 20, 25, 30, 35, 40, 45, or 50%, as compared to the percentage of central memory cells, for example, central memory T cells, for example, CCR7+CD45RO+ T cells, in the same population of cells prior to being engineered to express the CAR;
(g) about the same percentage of stem memory T cells, for example, CD45RA+CD95+IL-2 receptor β+CCR7+CD62L+ T cells, as compared to the percentage of stem memory T cells, for example, CD45RA+CD95+IL-2 receptor β+CCR7+CD62L+ T cells, in the same population of cells prior to being engineered to express the CAR;
(h) a change within about 5% to about 10% of stem memory T cells, for example, CD45RA+CD95+IL-2 receptor β+CCR7+CD62L+ T cells, as compared to the percentage of stem memory T cells, for example, CD45RA+CD95+IL-2 receptor β+CCR7+CD62L+ T cells, in the same population of cells prior to being engineered to express the CAR; or
(i) an increased percentage of stem memory T cells, for example, CD45RA+CD95+IL-2 receptor β+CCR7+CD62L+ T cells, as compared to the percentage of stem memory T cells, for example, CD45RA+CD95+IL-2 receptor β+CCR7+CD62L+ T cells, in the same population of cells prior to being engineered to express the CAR.

In one aspect, the disclosure provides a method of treating a subject having an autoimmune disease or disorder, e.g., lupus (e.g., systemic lupus erythematosus (SLE), e.g., severe refractory systemic lupus erythematosus (srSLE), or lupus nephritis), systemic sclerosis (e.g., rapidly progressing systemic sclerosis (SSc) with significant lung involvement (e.g. as for autoHSCT)), idiopathic inflammatory myopathies (e.g., polymyositis, dermatomyositis, anti-synthetase syndrome, immune-mediated necrotizing myopathy, inclusion body myositis, overlap myositis, cancer associated myositis, e.g. anti-synthetase syndrome with ILD), vasculitis (e.g., ANCA-associated vasculitis), severe refractory Sjögren's, severe refractory neuroimmune disease (e.g. myasthenia gravis (MG), neuromyelitis optica (NMO), MOG associated disease (MOGAD), multiple sclerosis (MS)), severe refractory rheumatoid arthritis, antibody mediated neuroimmune diseases (e.g., AChR+ and MuSK+myasthenia gravis (MG), AQP4+ neuromyelitis optica (NMO), MOGAD (anti-MOG associated disease), NMDAR+encephalitis, or antibody-associated neurological paraneoplastic diseases), Addison's disease, Goodpasture's syndrome, thyrotoxicosis, chronic active hepatitis, relapsing polychondritis, pemphigus vulgaris, or amyotrophic lateral sclerosis, the method comprising administering to the subject a population of cells engineered to express a CD19 CAR (“a population of CAR-expressing cells”), wherein:
(a) the median GeneSetScore (Up TEM vs. Down TSCM) of the population of cells is about the same as or differs by no more than (for example, increased by no more than) about 25, 50, 75, 100, or 125% from the median GeneSetScore (Up TEM vs. Down TSCM) of the same population of cells prior to being engineered to express the CAR;
(b) the median GeneSetScore (Up Treg vs. Down Teff) of the population of cells is about the same as or differs by no more than (for example, increased by no more than) about 25, 50, 100, 150, or 200% from the median GeneSetScore (Up Treg vs. Down Teff) of the population of cells prior to being engineered to express the CAR;
(c) the median GeneSetScore (Down stemness) of the population of cells is about the same as or differs by no more than (for example, increased by no more than) about 25, 50, 100, 150, 200, or 250% from the median GeneSetScore (Down stemness) of the population of cells prior to being engineered to express the CAR;
(d) the median GeneSetScore (Up hypoxia) of the population of cells is about the same as or differs by no more than (for example, increased by no more than) about 125, 150, 175, or 200% from the median GeneSetScore (Up hypoxia) of the population of cells prior to being engineered to express the CAR; or
(e) the median GeneSetScore (Up autophagy) of the population of cells is about the same as or differs by no more than (for example, increased by no more than) about 180, 190, 200, or 210% from the median GeneSetScore (Up autophagy) of the population of cells prior to being engineered to express the CAR.

In one aspect, the disclosure provides a method of treating a subject having an autoimmune disease or disorder, e.g., lupus (e.g., systemic lupus erythematosus (SLE), e.g., severe refractory systemic lupus erythematosus (srSLE), or lupus nephritis), systemic sclerosis (e.g., rapidly progressing systemic sclerosis (SSc) with significant lung involvement (e.g. as for autoHSCT)), idiopathic inflammatory myopathies (e.g., polymyositis, dermatomyositis, anti-synthetase syndrome, immune-mediated necrotizing myopathy, inclusion body myositis, overlap myositis, cancer associated myositis, e.g. anti-synthetase syndrome with ILD), vasculitis (e.g., ANCA-associated vasculitis), severe refractory Sjögren's, severe refractory neuroimmune disease (e.g. myasthenia gravis (MG), neuromyelitis optica (NMO), MOG associated disease (MOGAD), multiple sclerosis (MS)), severe refractory rheumatoid arthritis, antibody mediated neuroimmune diseases (e.g., AChR+ and MuSK+myasthenia gravis (MG), AQP4+ neuromyelitis optica (NMO), MOGAD (anti-MOG associated disease), NMDAR+encephalitis, or antibody-associated neurological paraneoplastic diseases), Addison's disease, Goodpasture's syndrome, thyrotoxicosis, chronic active hepatitis, relapsing polychondritis, pemphigus vulgaris, or amyotrophic lateral sclerosis, the method comprising administering to the subject rapcabtagene autoleucel.

In one aspect, the disclosure provides a method of treating a subject having a severe refractory autiommune disease, the method comprising administering to the subject rapcabtagene autoleucel.

In some embodiments, the severe refractory autiommune disease is selected from systemic lupus erythematosus, lupus nephritis, idiopathic inflammatory myopathy, systemic sclerosis and ANCA-associated vasculitis.

In some embodiments, the lupus is systemic lupus erythematosus. In some embodiments, the SLE is a severe refractory SLE (srSLE).

In some embodiments, the CD19 CAR comprises a CD19 binding domain, a transmembrane domain, and an intracellular signaling domain.

In some embodiments:
(a) the transmembrane domain comprises a transmembrane domain of a protein chosen from the alpha, beta, or zeta chain of T-cell receptor, CD28, CD3 epsilon, CD45, CD4, CD5, CD8, CD9, CD16, CD22, CD33, CD37, CD64, CD80, CD86, CD134, CD137 and CD154,
(b) the transmembrane domain comprises a transmembrane domain of CD8,
(c) the transmembrane domain comprises the amino acid sequence of SEQ ID NO: 6, or an amino acid sequence having at least about 85%, 90%, 95%, or 99% sequence identity thereof, or
(d) the nucleic acid molecule comprises a nucleic acid sequence encoding the transmembrane domain, wherein the nucleic acid sequence comprises the nucleic acid sequence of SEQ ID NO: 17, or a nucleic acid sequence having at least about 85%, 90%, 95%, or 99% sequence identity thereof.

In one aspect, the disclosure provides method of treating a subject having severe refractory systemic lupus erythematosus (srSLE), the method comprising administering to the subject a population of cells comprising a CD19 chimeric antigen receptor (CD19 CAR), or comprising a nucleic acid encoding the CD19 CAR,
wherein the CAR comprises an CD19 binding domain, a transmembrane domain, and an intracellular signaling domain, and wherein the transmembrane domain comprises a transmembrane domain of a CD8 protein;
in an amount sufficient to treat the srSLE,
thereby treating the srSLE.

In some embodiments:
(a) the transmembrane domain comprises the amino acid sequence of SEQ ID NO: 6, or an amino acid sequence having at least about 85%, 90%, 95%, or 99% sequence identity thereof, or
(a) the nucleic acid molecule comprises a nucleic acid sequence encoding the transmembrane domain, wherein the nucleic acid sequence comprises the nucleic acid sequence of SEQ ID NO: 17, or a nucleic acid sequence having at least about 85%, 90%, 95%, or 99% sequence identity thereof.

In some embodiments, the population of CAR-expressing cells (for example, CD19 CAR-expressing cells) is administered at a dose of about 0.5×10⁶ to 50×10⁶ viable CAR-expressing cells, for example, about 5×10⁶ viable CAR-expressing cells, optionally wherein the population of CAR-expressing cells (for example, CD19 CAR-expressing cells) is administered at a dose of 5×10⁶ viable CAR-expressing cells.

In some embodiments, the population of CAR-expressing cells (for example, CD19 CAR-expressing cells) is administered at a dose of about 2.5×10⁶ to 2.5×10⁸ viable CAR-expressing cells, for example, about 1.25×10⁷ viable CAR-expressing cells, optionally wherein the population of CAR-expressing cells (for example, CD19 CAR-expressing cells) is administered at a dose of 1.25×10⁷ viable CAR-expressing cells.

In some embodiments, the population of CAR-expressing cells (for example, CD19 CAR-expressing cells) is administered at a dose of about 1.25×10⁷ to 1.25×10⁹ viable CAR-expressing cells, for example, about 1.25×10⁸ viable CAR-expressing cells, optionally wherein the population of CAR-expressing cells (for example, CD19 CAR-expressing cells) is administered at a dose of 1.25×10⁸ viable CAR-expressing cells.

In some embodiments, the population of CAR-expressing cells (for example, CD19 CAR-expressing cells) is administered at a dose of about 2.5×10⁶ to 2.5×10⁸ viable CAR-expressing cells, for example, about 1×10⁷ or 5×10⁷ viable CAR-expressing cells.

In one aspect, the disclosure provides a method of treating a subject having severe refractory systemic lupus erythematosus (srSLE), the method comprising administering to the subject rapcabtagene autoleucel
in an amount sufficient to treat the srSLE,
thereby treating the srSLE.

In some embodiments, rapcabtagene autoleucel is administered at a dose of about 0.5×10⁶ to 50×10⁶ viable CAR-positive cells, for example, about 5×10⁶ viable CAR-positive cells, optionally wherein rapcabtagene autoleucel is administered at a dose of 5×10⁶ viable CAR-positive cells.

In some embodiments, rapcabtagene autoleucel is administered at a dose of about 2.5×10⁶ to 2.5×10⁸ viable CAR-positive cells, for example, about 1.25×10⁷ viable CAR-positive cells, optionally wherein rapcabtagene autoleucel is administered at a dose of 1.25×10⁷ viable CAR-positive cells.

In some embodiments, rapcabtagene autoleucel is administered at a dose of about 1.25×107 to 1.25×10⁹ viable CAR-positive cells, for example, about 1.25×10⁸ viable CAR-positive cells, optionally wherein rapcabtagene autoleucel is administered at a dose of 1.25×10⁸ viable CAR-positive cells.

In some embodiments, rapcabtagene autoleucel is administered at a dose of about 2.5×10⁶ to 2.5×10⁸ viable CAR-positive cells, for example, about 1×10⁷ or 5×10⁷ viable CAR-positive cells.

In one aspect, the disclosure provides a method of treating a subject having lupus (e.g., systemic lupus erythematosus (SLE), e.g., severe refractory systemic lupus erythematosus (srSLE) or lupus nephritis), the method comprising administering to the subject a population of cells that express, or comprise a nucleic acid configured to express, a CD19 chimeric antigen receptor (CD19 CAR), wherein the cells are administered at a dose of 0.5−50×10⁶ viable CAR+ T cells (e.g., 5−12.5×10⁶ viable CAR+ T cells).

In one aspect, the disclosure provides a method of treating a subject having lupus (e.g., systemic lupus erythematosus (SLE), e.g., severe refractory systemic lupus erythematosus (srSLE) or lupus nephritis), the method comprising administering to the subject rapcabtagene autoleucel, wherein rapcabtagene autoleucel is administered at a dose of 0.5−50×10⁶ viable CAR+ T cells (e.g., 5−12.5×10⁶ viable CAR+ T cells).

In some embodiments, the lupus is systemic lupus erythematosus. In some embodiments, the SLE is a severe refractory SLE (srSLE), wherein optionally the subject has renal involvement.

In some embodiments, the CAR comprises a CD19 binding domain, a transmembrane domain, and an intracellular signaling domain.

In some embodiments:
(a) the transmembrane domain comprises a transmembrane domain of a protein chosen from the alpha, beta or zeta chain of T-cell receptor, CD28, CD3 epsilon, CD45, CD4, CD5, CD8, CD9, CD16, CD22, CD33, CD37, CD64, CD80, CD86, CD134, CD137 and CD154,
(b) the transmembrane domain comprises a transmembrane domain of CD8,
(c) the transmembrane domain comprises the amino acid sequence of SEQ ID NO: 6, or an amino acid sequence having at least about 85%, 90%, 95%, or 99% sequence identity thereof, or
(d) the nucleic acid molecule comprises a nucleic acid sequence encoding the transmembrane domain, wherein the nucleic acid sequence comprises the nucleic acid sequence of SEQ ID NO: 17, or a nucleic acid sequence having at least about 85%, 90%, 95%, or 99% sequence identity thereof.

In some embodiments, the CD19 binding domain comprises a heavy chain complementarity determining region 1 (HC CDR1), an HC CDR2, an HC CDR3, a light chain complementarity determining region 1 (LC CDR 1), an LC CDR2, and an LC CDR3, wherein:
(a) the HC CDR1 comprises the amino acid sequence of SEQ ID NO: 295;
(b) the HC CDR2 comprising the amino acid sequence of SEQ ID NO: 296;
(c) the HC CDR3 comprising the amino acid sequence of SEQ ID NO: 297;
(d) the LC CDR1 comprising the amino acid sequence of SEQ ID NO: 298;
(e) the LC CDR2 comprising the amino acid sequence of SEQ ID NO: 299; and
(f) the LC CDR3 comprising the amino acid sequence of SEQ ID NO: 300.

In some embodiments, the CD19 binding domain comprises a VH and a VL, wherein the VH and VL are connected by a linker, optionally wherein the linker comprises the amino acid sequence of SEQ ID NO: 63 or 104.

In some embodiments, the CD19 binding domain is connected to the transmembrane domain by a hinge region, optionally wherein:
(a) the hinge region comprises the amino acid sequence of SEQ ID NO: 2, 3, or 4, or an amino acid sequence having at least about 85%, 90%, 95%, or 99% sequence identity thereof, or
(b) the nucleic acid molecule comprises a nucleic acid sequence encoding the hinge region, wherein the nucleic acid sequence comprises the nucleic acid sequence of SEQ ID NO: 13, 14, or 15, or a nucleic acid sequence having at least about 85%, 90%, 95%, or 99% sequence identity thereof.

In some embodiments, the intracellular signaling domain comprises a primary signaling domain, optionally wherein the primary signaling domain comprises a functional signaling domain derived from CD3 zeta, TCR zeta, FcR gamma, FcR beta, CD3 gamma, CD3 delta, CD3 epsilon, CD5, CD22, CD79a, CD79b, CD278 (ICOS), FcERI, DAP10, DAP12, or CD66d, optionally wherein:
(a) the primary signaling domain comprises a functional signaling domain derived from CD3 zeta,
(b) the primary signaling domain comprises the amino acid sequence of SEQ ID NO: 9 or 10, or an amino acid sequence having at least about 85%, 90%, 95%, or 99% sequence identity thereof, or
(c) the nucleic acid molecule comprises a nucleic acid sequence encoding the primary signaling domain, wherein the nucleic acid sequence comprises the nucleic acid sequence of SEQ ID NO: 20 or 21, or a nucleic acid sequence having at least about 85%, 90%, 95%, or 99% sequence identity thereof.

In some embodiments, the intracellular signaling domain comprises a costimulatory signaling domain, optionally wherein the costimulatory signaling domain comprises a functional signaling domain derived from a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, 4-1BB (CD137), B7-H3, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD28-OX40, CD28-4-1BB, or a ligand that specifically binds with CD83, optionally wherein:
(a) the costimulatory signaling domain comprises a functional signaling domain derived from 4-1BB,
(b) the costimulatory signaling domain comprises the amino acid sequence of SEQ ID NO: 7, or an amino acid sequence having at least about 85%, 90%, 95%, or 99% sequence identity thereof, or
(c) the nucleic acid molecule comprises a nucleic acid sequence encoding the costimulatory signaling domain, wherein the nucleic acid sequence comprises the nucleic acid sequence of SEQ ID NO: 18, or a nucleic acid sequence having at least about 85%, 90%, 95%, or 99% sequence identity thereof.

In some embodiments, the intracellular signaling domain comprises a functional signaling domain derived from 4-1BB and a functional signaling domain derived from CD3 zeta, optionally wherein the intracellular signaling domain comprises the amino acid sequence of SEQ ID NO: 7 (or an amino acid sequence having at least about 85%, 90%, 95%, or 99% sequence identity thereof) and the amino acid sequence of SEQ ID NO: 9 or 10 (or an amino acid sequence having at least about 85%, 90%, 95%, or 99% sequence identity thereof), optionally wherein the intracellular signaling domain comprises the amino acid sequence of SEQ ID NO: 7 and the amino acid sequence of SEQ ID NO: 9 or 10.

In some embodiments, the CAR further comprises a leader sequence comprising the amino acid sequence of SEQ ID NO: 1.

In some embodiments, the CD19 CAR comprises the amino acid sequence of SEQ ID NO: 301, or a sequence having at least 80%, 85%, 90%, 95%, or 99% identity thereto.

In some embodiments, the nucleic acid molecule encoding the CD19 CAR comprises the nucleotide sequence of SEQ ID NO: 302, or a sequence having at least 80%, 85%, 90%, 95%, or 99% identity thereto.

In some embodiments, the subject has been previously treated with, or is concurrently treated with, one or more of an antimalarial (e.g., hydroxychloroquine or quinacrine), a glucocorticoid (e.g., prednisone), a calcineurin inhibitor, an immunomodulatory agent (e.g., methotrexate, azathioprine, mycophenolate moefetil, cyclophosphamide, or tacrolimus), a biological agent (e.g., belimumab, rituximab, a disease-modifying antirheumatic drug (DMARD) (e.g., leflunomide).

In some embodiments, the subject has been identified as not responding to treatment comprising two or more immunosuppressive therapies (e.g., mycophenolate or cyclophosphamide) in combination with a glucocorticoid) and one biological agent.

In some embodiments, the subject has not previously received a therapy comprising a CD19 CAR (e.g., rapcabtagene autoleucel), an adoptive T cell therapy, or a gene therapy product.

In some embodiments, prior to administration of the CD19 CAR (e.g., rapcabtagene autoleucel), the subject receives lymphodepleting therapy.

In some embodiments, the subject receives a lympodepleting therapy about two weeks prior to administration of the CD19 CAR (e.g., rapcabtagene autoleucel).

In some embodiments, the lympodepleting therapy comprises fludarabine (e.g., 25 mg/m² IV daily for three doses) and cyclophosphamide (e.g., 250 mg/m² IV daily for three doses).

In some embodiments, the method further comprises administering a second therapeutic agent to the subject.

In some embodiments, the second therapeutic agent is administered prior to, concurrently with, or after the administration of the population of CAR-expressing cells or rapcabtagene autoleucel.

In some embodiments, the subject is monitored for a sign of Cytokine Release Syndrome, for example, for at least 2, 2.5, 3, 3.5, or 4 days, for example, for about 3 days.

In some embodiments, leukapheresis occurs (i) prior to administration of corticosteroids and/or (ii) when absolute T cell count is ≥300/mm³.

In one aspect, the disclosure provides a method of making a population of cells (for example, T cells) that express a chimeric antigen receptor (CAR), the method comprising:
(i) contacting (for example, binding) a population of cells (for example, T cells, for example, T cells isolated from a frozen or fresh leukapheresis product) with an agent that stimulates a CD3/TCR complex and/or an agent that stimulates a costimulatory molecule on the surface of the cells, wherein the population of cells is from a subject having an autoimmune disease or disorder, e.g., lupus (e.g., systemic lupus erythematosus (SLE), e.g., severe refractory systemic lupus erythematosus (srSLE), or lupus nephritis), systemic sclerosis (e.g., rapidly progressing systemic sclerosis (SSc) with significant lung involvement (e.g. as for autoHSCT)), idiopathic inflammatory myopathies (e.g., polymyositis, dermatomyositis, anti-synthetase syndrome, immune-mediated necrotizing myopathy, inclusion body myositis, overlap myositis, cancer associated myositis, e.g. anti-synthetase syndrome with ILD), vasculitis (e.g., ANCA-associated vasculitis), severe refractory Sjögren's, severe refractory neuroimmune disease (e.g. myasthenia gravis (MG), neuromyelitis optica (NMO), MOG associated disease (MOGAD), multiple sclerosis (MS)), severe refractory rheumatoid arthritis, antibody mediated neuroimmune diseases (e.g., AChR+ and MuSK+myasthenia gravis (MG), AQP4+ neuromyelitis optica (NMO), MOGAD (anti-MOG associated disease), NMDAR+encephalitis, or antibody-associated neurological paraneoplastic diseases), Addison's disease, Goodpasture's syndrome, thyrotoxicosis, chronic active hepatitis, relapsing polychondritis, pemphigus vulgaris, or amyotrophic lateral sclerosis;
(ii) contacting the population of cells (for example, T cells) with a nucleic acid molecule (for example, a DNA or RNA molecule) encoding the CAR, thereby providing a population of cells (for example, T cells) comprising the nucleic acid molecule, wherein optionally the CAR comprises a CD19 antigen binding domain; and
(iii) harvesting the population of cells (for example, T cells) for storage (for example, reformulating the population of cells in cryopreservation media) or administration, wherein:
(a) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and
   step (iii) is performed no later than 30 (for example, 26) hours after the beginning of step (i), for example, no later than 22, 23, 24, 25, 26, 27, 28, 29, or 30 hours after the beginning of step (i), for example, no later than 24 hours after the beginning of step (i),
(b) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and
   step (iii) is performed no later than 30 hours after the beginning of step (ii), for example, no later than 22, 23, 24, 25, 26, 27, 28, 29, or 30 hours after the beginning of step (ii), or
(c) the population of cells from step (iii) are not expanded, or expanded by no more than 5, 10, 15, 20, 25, 30, 35, or 40%, for example, no more than 10%, for example, as assessed by the number of living cells, compared to the population of cells at the beginning of step (i),
optionally wherein the nucleic acid molecule in step (ii) is on a viral vector, optionally wherein the nucleic acid molecule in step (ii) is an RNA molecule on a viral vector, optionally wherein step (ii) comprises transducing the population of cells (for example, T cells) with a viral vector comprising a nucleic acid molecule encoding the CAR.

In some embodiments, the agent that stimulates a CD3/TCR complex is an agent that stimulates CD3 (for example, an anti-CD3 antibody) and wherein the agent that stimulates a costimulatory molecule is an agent that stimulates CD28, ICOS, CD27, HVEM, LIGHT, CD40, 4-1BB, OX40, DR3, GITR, CD30, TIM1, CD2, CD226, or any combination thereof, optionally wherein the agent that stimulates a CD3/TCR complex or the agent that stimulates a costimulatory molecule is chosen from an antibody (for example, a single-domain antibody (for example, a heavy chain variable domain antibody), a peptibody, a Fab fragment, or a scFv), a small molecule, or a ligand (for example, a naturally-existing, recombinant, or chimeric ligand), optionally wherein the agent that stimulates a CD3/TCR complex or the agent that stimulates a costimulatory molecule does not comprise a bead, optionally wherein the agent that stimulates a CD3/TCR complex comprises an anti-CD3 antibody and the agent that stimulates a costimulatory molecule comprises an anti-CD28 antibody, optionally wherein the agent that stimulates a CD3/TCR complex comprises an anti-CD3 antibody covalently attached to a colloidal polymeric nanomatrix and the agent that stimulates a costimulatory molecule comprises an anti-CD28 antibody covalently attached to a colloidal polymeric nanomatrix, optionally wherein the agent that stimulates a CD3/TCR complex and the agent that stimulates a costimulatory molecule comprise T Cell TransAct™.

In some embodiments, step (i) increases the percentage of CAR-expressing cells in the population of cells from step (iii), for example, the population of cells from step (iii) shows a higher percentage of CAR-expressing cells (for example, at least 10, 20, 30, 40, 50, or 60% higher), compared with cells made by an otherwise similar method without step (i).

In some embodiments:
(a) the percentage of naïve cells, for example, naïve T cells, for example, CD45RA+ CD45RO− CCR7+ T cells, in the population of cells from step (iii) is the same as or differs by no more than 5 or 10% from the percentage of naïve cells, for example, naïve T cells, for example, CD45RA+CD45RO− CCR7+ cells, in the population of cells at the beginning of step (i);
(b) the percentage of naïve cells, for example, naïve T cells, for example, CD45RA+ CD45RO− CCR7+ T cells, in the population of cells from step (iii) is increased by, for example, at least 1.2, 1.4, 1.6, 1.8, 2.0, 2.2, 2.4, 2.6, 2.8, or 3-fold, as compared to the percentage of naïve cells, for example, naïve T cells, for example, CD45RA+CD45RO− CCR7+ cells, in the population of cells at the beginning of step (i);
(c) the percentage of CAR-expressing naïve T cells, for example, CAR-expressing CD45RA+CD45RO− CCR7+ T cells in the population of cells increases during the duration of step (ii), for example, increases by, for example, at least 30, 35, 40, 45, 50, 55, or 60%, between 18-24 hours after the beginning of step (ii); or
(d) the percentage of naïve cells, for example, naïve T cells, for example, CD45RA+ CD45RO− CCR7+ T cells, in the population of cells from step (iii) does not decrease, or decreases by no more than 5 or 10%, as compared to the percentage of naïve cells, for example, naïve T cells, for example, CD45RA+CD45RO− CCR7+ cells, in the population of cells at the beginning of step (i).

In some embodiments:
(a) the population of cells from step (iii) shows a higher percentage of naïve cells, for example, naïve T cells, for example, CD45RA+CD45RO− CCR7+ T cells (for example, at least 10, 20, 30, or 40% higher), compared with cells made by an otherwise similar method in which step (iii) is performed more than 26 hours after the beginning of step (i), for example, more than 5, 6, 7, 8, 9, 10, 11, or 12 days after the beginning of step (i);
(b) the percentage of naïve cells, for example, naïve T cells, for example, CD45RA+CD45RO− CCR7+ T cells, in the population of cells from step (iii) is higher (for example, at least 1.2, 1.4, 1.6, 1.8, 2.0, 2.2, 2.4, 2.6, 2.8, or 3-fold higher) than the percentage of naïve cells, for example, naïve T cells, for example, CD45RA+CD45RO− CCR7+ T cells, in cells made by an otherwise similar method in which step (iii) is performed more than 26 hours after the beginning of step (i), for example, more than 5, 6, 7, 8, 9, 10, 11, or 12 days after the beginning of step (i);
(c) the percentage of CAR-expressing naïve T cells, for example, CAR-expressing CD45RA+CD45RO− CCR7+ T cells, in the population of cells from step (iii) is higher (for example, at least 4, 6, 8, 10, or 12-fold higher) than the percentage of CAR-expressing naïve T cells, for example, CAR-expressing CD45RA+CD45RO− CCR7+ T cells, in cells made by an otherwise similar method in which step (iii) is performed more than 26 hours after the beginning of step (i), for example, more than 5, 6, 7, 8, 9, 10, 11, or 12 days after the beginning of step (i);
(d) the population of cells from step (iii) shows a higher percentage of naïve cells, for example, naïve T cells, for example, CD45RA+CD45RO− CCR7+ T cells (for example, at least 10, 20, 30, or 40% higher), compared with cells made by an otherwise similar method which further comprises, after step (ii) and prior to step (iii), expanding the population of cells (for example, T cells) in vitro for more than 3 days, for example, for 5, 6, 7, 8 or 9 days;
(e) the percentage of naïve cells, for example, naïve T cells, for example, CD45RA+ CD45RO− CCR7+ T cells, in the population of cells from step (iii) is higher (for example, at least 1.2, 1.4, 1.6, 1.8, 2.0, 2.2, 2.4, 2.6, 2.8, or 3-fold higher) than the percentage of naïve cells, for example, naïve T cells, for example, CD45RA+CD45RO− CCR7+ T cells, in cells made by an otherwise similar method which further comprises, after step (ii) and prior to step (iii), expanding the population of cells (for example, T cells) in vitro for more than 3 days, for example, for 5, 6, 7, 8 or 9 days; or
(f) the percentage of CAR-expressing naïve T cells, for example, CAR-expressing CD45RA+CD45RO− CCR7+ T cells, in the population of cells from step (iii) is higher (for example, at least 4, 6, 8, 10, or 12-fold higher) than the percentage of CAR-expressing naïve T cells, for example, CAR-expressing CD45RA+CD45RO− CCR7+ T cells, in cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments:
(a) the percentage of central memory cells, for example, central memory T cells, for example, CD95+ central memory T cells, in the population of cells from step (iii) is the same as or differs by no more than 5 or 10% from the percentage of central memory cells, for example, central memory T cells, for example, CD95+ central memory T cells, in the population of cells at the beginning of step (i);
(b) the percentage of central memory cells, for example, central memory T cells, for example, CCR7+CD45RO+ T cells, in the population of cells from step (iii) is reduced by at least 20, 25, 30, 35, 40, 45, or 50%, as compared to the percentage of central memory cells, for example, central memory T cells, for example, CCR7+CD45RO+ T cells, in the population of cells at the beginning of step (i);
(c) the percentage of CAR-expressing central memory T cells, for example, CAR-expressing CCR7+CD45RO+ cells, decreases during the duration of step (ii), for example, decreases by, for example, at least 8, 10, 12, 14, 16, 18, or 20%, between 18-24 hours after the beginning of step (ii); or
(d) the percentage of central memory cells, for example, central memory T cells, for example, CCR7+CD45RO+ T cells, in the population of cells from step (iii) does not increase, or increases by no more than 5 or 10%, as compared to the percentage of central memory cells, for example, central memory T cells, for example, CCR7+CD45RO+ T cells, in the population of cells at the beginning of step (i).

In some embodiments:
(a) the population of cells from step (iii) shows a lower percentage of central memory cells, for example, central memory T cells, for example, CD95+ central memory T cells (for example, at least 10, 20, 30, or 40% lower), compared with cells made by an otherwise similar method in which step (iii) is performed more than 26 hours after the beginning of step (i), for example, more than 5, 6, 7, 8, 9, 10, 11, or 12 days after the beginning of step (i);
(b) the percentage of central memory cells, for example, central memory T cells, for example, CCR7+CD45RO+ T cells in the population of cells from step (iii) is lower (for example, at least 20, 30, 40, or 50% lower) than the percentage of central memory cells, for example, central memory T cells, for example, CCR7+CD45RO+ T cells, in cells made by an otherwise similar method in which step (iii) is performed more than 26 hours after the beginning of step (i), for example, more than 5, 6, 7, 8, 9, 10, 11, or 12 days after the beginning of step (i);
(c) the percentage of CAR-expressing central memory T cells, for example, CAR-expressing CCR7+CD45RO+ T cells in the population of cells from step (iii) is lower (for example, at least 10, 20, 30, or 40% lower) than the percentage of CAR-expressing central memory T cells, for example, CAR-expressing CCR7+CD45RO+ T cells, in cells made by an otherwise similar method in which step (iii) is performed more than 26 hours after the beginning of step (i), for example, more than 5, 6, 7, 8, 9, 10, 11, or 12 days after the beginning of step (i);
(d) the population of cells from step (iii) shows a lower percentage of central memory cells, for example, central memory T cells, for example, CD95+ central memory T cells (for example, at least 10, 20, 30, or 40% lower), compared with cells made by an otherwise similar method which further comprises, after step (ii) and prior to step (iii), expanding the population of cells (for example, T cells) in vitro for more than 3 days, for example, for 5, 6, 7, 8 or 9 days;
(e) the percentage of central memory cells, for example, central memory T cells, for example, CCR7+CD45RO+ T cells in the population of cells from step (iii) is lower (for example, at least 20, 30, 40, or 50% lower) than the percentage of central memory cells, for example, central memory T cells, for example, CCR7+CD45RO+ T cells, in cells made by an otherwise similar method which further comprises, after step (ii) and prior to step (iii), expanding the population of cells (for example, T cells) in vitro for more than 3 days, for example, for 5, 6, 7, 8 or 9 days; or
(f) the percentage of CAR-expressing central memory T cells, for example, CAR-expressing CCR7+CD45RO+ T cells in the population of cells from step (iii) is lower (for example, at least 10, 20, 30, or 40% lower) than the percentage of CAR-expressing central memory T cells, for example, CAR-expressing CCR7+CD45RO+ T cells, in cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments:
(a) the percentage of stem memory T cells, for example, CD45RA+CD95+IL-2 receptor β+CCR7+CD62L+ T cells, in the population of cells from step (iii) is increased, as compared to the percentage of stem memory T cells, for example, CD45RA+CD95+IL-2 receptor β+CCR7+CD62L+ T cells, in the population of cells at the beginning of step (i);
(b) the percentage of CAR-expressing stem memory T cells, for example, CAR-expressing CD45RA+CD95+IL-2 receptor β+CCR7+CD62L+ T cells, in the population of cells from step (iii) is increased, as compared to the percentage of CAR-expressing stem memory T cells, for example, CAR-expressing CD45RA+CD95+IL-2 receptor β+CCR7+CD62L+ T cells, in the population of cells at the beginning of step (i);
(c) the percentage of stem memory T cells, for example, CD45RA+CD95+IL-2 receptor β+CCR7+CD62L+ T cells, in the population of cells from step (iii) is higher than the percentage of stem memory T cells, for example, CD45RA+CD95+IL-2 receptor β+CCR7+CD62L+ T cells, in cells made by an otherwise similar method in which step (iii) is performed more than 26 hours after the beginning of step (i), for example, more than 5, 6, 7, 8, 9, 10, 11, or 12 days after the beginning of step (i); or
(d) the percentage of CAR-expressing stem memory T cells, for example, CAR-expressing CD45RA+CD95+IL-2 receptor β+CCR7+CD62L+ T cells, in the population of cells from step (iii) is higher than the percentage of CAR-expressing stem memory T cells, for example, CAR-expressing CD45RA+CD95+IL-2 receptor β+CCR7+CD62L+ T cells, in cells made by an otherwise similar method in which step (iii) is performed more than 26 hours after the beginning of step (i), for example, more than 5, 6, 7, 8, 9, 10, 11, or 12 days after the beginning of step (i);
(e) the percentage of stem memory T cells, for example, CD45RA+CD95+IL-2 receptor β+CCR7+CD62L+ T cells, in the population of cells from step (iii) is higher than the percentage of stem memory T cells, for example, CD45RA+CD95+IL-2 receptor β+CCR7+CD62L+ T cells, in cells made by an otherwise similar method which further comprises, after step (ii) and prior to step (iii), expanding the population of cells (for example, T cells) in vitro for more than 3 days, for example, for 5, 6, 7, 8 or 9 days; or
(f) the percentage of CAR-expressing stem memory T cells, for example, CAR-expressing CD45RA+CD95+IL-2 receptor β+CCR7+CD62L+ T cells, in the population of cells from step (iii) is higher than the percentage of CAR-expressing stem memory T cells, for example, CAR-expressing CD45RA+CD95+IL-2 receptor β+CCR7+CD62L+ T cells, in cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments:
(a) the median GeneSetScore (Up TEM vs. Down TSCM) of the population of cells from step (iii) is about the same as or differs by no more than (for example, increased by no more than) about 25, 50, 75, 100, or 125% from the median GeneSetScore (Up TEM vs. Down TSCM) of the population of cells at the beginning of step (i);
(b) the median GeneSetScore (Up TEM vs. Down TSCM) of the population of cells from step (iii) is lower (for example, at least about 100, 150, 200, 250, or 300% lower) than the median GeneSetScore (Up TEM vs. Down TSCM) of:
   cells made by an otherwise similar method in which step (iii) is performed more than 26 hours after the beginning of step (i), for example, more than 5, 6, 7, 8, 9, 10, 11, or 12 days after the beginning of step (i), or
   cells made by an otherwise similar method which further comprises, after step (ii) and prior to step (iii), expanding the population of cells (for example, T cells) in vitro for more than 3 days, for example, for 5, 6, 7, 8 or 9 days;
(c) the median GeneSetScore (Up Treg vs. Down Teff) of the population of cells from step (iii) is about the same as or differs by no more than (for example, increased by no more than) about 25, 50, 100, 150, or 200% from the median GeneSetScore (Up Treg vs. Down Teff) of the population of cells at the beginning of step (i);
(d) the median GeneSetScore (Up Treg vs. Down Teff) of the population of cells from step (iii) is lower (for example, at least about 50, 100, 125, 150, or 175% lower) than the median GeneSetScore (Up Treg vs. Down Teff) of:
   cells made by an otherwise similar method in which step (iii) is performed more than 26 hours after the beginning of step (i), for example, more than 5, 6, 7, 8, 9, 10, 11, or 12 days after the beginning of step (i), or
   cells made by an otherwise similar method which further comprises, after step (ii) and prior to step (iii), expanding the population of cells (for example, T cells) in vitro for more than 3 days, for example, for 5, 6, 7, 8 or 9 days;
(e) the median GeneSetScore (Down stemness) of the population of cells from step (iii) is about the same as or differs by no more than (for example, increased by no more than) about 25, 50, 100, 150, 200, or 250% from the median GeneSetScore (Down stemness) of the population of cells at the beginning of step (i);
(f) the median GeneSetScore (Down stemness) of the population of cells from step (iii) is lower (for example, at least about 50, 100, or 125% lower) than the median GeneSetScore (Down stemness) of:
   cells made by an otherwise similar method in which step (iii) is performed more than 26 hours after the beginning of step (i), for example, more than 5, 6, 7, 8, 9, 10, 11, or 12 days after the beginning of step (i), or
   cells made by an otherwise similar method which further comprises, after step (ii) and prior to step (iii), expanding the population of cells (for example, T cells) in vitro for more than 3 days, for example, for 5, 6, 7, 8 or 9 days;
(g) the median GeneSetScore (Up hypoxia) of the population of cells from step (iii) is about the same as or differs by no more than (for example, increased by no more than) about 125, 150, 175, or 200% from the median GeneSetScore (Up hypoxia) of the population of cells at the beginning of step (i);
(h) the median GeneSetScore (Up hypoxia) of the population of cells from step (iii) is lower (for example, at least about 40, 50, 60, 70, or 80% lower) than the median GeneSetScore (Up hypoxia) of:
   cells made by an otherwise similar method in which step (iii) is performed more than 26 hours after the beginning of step (i), for example, more than 5, 6, 7, 8, 9, 10, 11, or 12 days after the beginning of step (i), or
   cells made by an otherwise similar method which further comprises, after step (ii) and prior to step (iii), expanding the population of cells (for example, T cells) in vitro for more than 3 days, for example, for 5, 6, 7, 8 or 9 days;
(j) the median GeneSetScore (Up autophagy) of the population of cells from step (iii) is about the same as or differs by no more than (for example, increased by no more than) about 180, 190, 200, or 210% from the median GeneSetScore (Up autophagy) of the population of cells at the beginning of step (i); or
(k) the median GeneSetScore (Up autophagy) of the population of cells from step (iii) is lower (for example, at least 20, 30, or 40% lower) than the median GeneSetScore (Up autophagy) of:
   cells made by an otherwise similar method in which step (iii) is performed more than 26 hours after the beginning of step (i), for example, more than 5, 6, 7, 8, 9, 10, 11, or 12 days after the beginning of step (i), or
   cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the population of cells from step (iii), after being incubated with a cell expressing an antigen recognized by the CAR, secretes IL-2 at a higher level (for example, at least 2, 4, 6, 8, 10, 12, or 14-fold higher) than cells made by an otherwise similar method in which step (iii) is performed more than 26 hours after the beginning of step (i), for example, more than 5, 6, 7, 8, 9, 10, 11, or 12 days after the beginning of step (i), or cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the population of cells from step (iii), after being administered to the subject in vivo, persists longer or expands at a higher level, compared with cells made by an otherwise similar method in which step (iii) is performed more than 26 hours after the beginning of step (i), for example, more than 5, 6, 7, 8, 9, 10, 11, or 12 days after the beginning of step (i), or compared with cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the population of cells from step (iii), after being administered to the subject in vivo, shows a stronger activity (for example, a stronger activity at a low dose, for example, a dose no more than 0.15×10⁶, 0.2×10⁶, 0.25×10⁶, or 0.3×10⁶ viable CAR-expressing cells) than cells made by an otherwise similar method in which step (iii) is performed more than 26 hours after the beginning of step (i), for example, more than 5, 6, 7, 8, 9, 10, 11, or 12 days after the beginning of step (i), or cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the population of cells from step (iii) are not expanded, or expanded by no more than 5, 10, 15, 20, 25, 30, 35, or 40%, for example, no more than 10%, for example, as assessed by the number of living cells, compared to the population of cells at the beginning of step (i), optionally wherein the number of living cells in the population of cells from step (iii) decreases from the number of living cells in the population of cells at the beginning of step (i).

In some embodiments, the population of cells from step (iii) are not expanded, or expanded by less than 2 hours, for example, less than 1 or 1.5 hours, compared to the population of cells at the beginning of step (i).

In some embodiments, steps (i) and/or (ii) are performed in cell media (for example, serum-free media) comprising IL-2, IL-15 (for example, hetIL-15 (IL15/sIL-15Ra)), IL-7, IL-21, IL-6 (for example, IL-6/sIL-6Ra), a LSD1 inhibitor, a MALT1 inhibitor, or a combination thereof.

In some embodiments, steps (i) and/or (ii) are performed in serum-free cell media comprising a serum replacement. In some embodiments, the serum replacement is CTS™ Immune Cell Serum Replacement (ICSR).

In some embodiments, the method further comprises prior to step (i):
(iv) (optionally) receiving a fresh leukapheresis product (or an alternative source of hematopoietic tissue such as a fresh whole blood product, a fresh bone marrow product, or a fresh organ biopsy or removal (for example, a fresh product from thymectomy)) from an entity, for example, a laboratory, hospital, or healthcare provider, and
(v) isolating the population of cells (for example, T cells, for example, CD8+ and/or CD4+ T cells) contacted in step (i) from a fresh leukapheresis product (or an alternative source of hematopoietic tissue such as a fresh whole blood product, a fresh bone marrow product, or a fresh organ biopsy or removal (for example, a fresh product from thymectomy)), optionally wherein:
step (iii) is performed no later than 35 hours after the beginning of step (v), for example, no later than 27, 28, 29, 30, 31, 32, 33, 34, or 35 hours after the beginning of step (v), for example, no later than 30 hours after the beginning of step (v), or
the population of cells from step (iii) are not expanded, or expanded by no more than 5, 10, 15, 20, 25, 30, 35, or 40%, for example, no more than 10%, for example, as assessed by the number of living cells, compared to the population of cells at the end of step (v).

In some embodiments, the method further comprises prior to step (i): receiving cryopreserved T cells isolated from a leukapheresis product (or an alternative source of hematopoietic tissue such as cryopreserved T cells isolated from whole blood, bone marrow, or organ biopsy or removal (for example, thymectomy)) from an entity, for example, a laboratory, hospital, or healthcare provider.

In some embodiments, the method further comprises prior to step (i):
(iv) (optionally) receiving a cryopreserved leukapheresis product (or an alternative source of hematopoietic tissue such as a cryopreserved whole blood product, a cryopreserved bone marrow product, or a cryopreserved organ biopsy or removal (for example, a cryopreserved product from thymectomy)) from an entity, for example, a laboratory, hospital, or healthcare provider, and
(v) isolating the population of cells (for example, T cells, for example, CD8+ and/or CD4+ T cells) contacted in step (i) from a cryopreserved leukapheresis product (or an alternative source of hematopoietic tissue such as a cryopreserved whole blood product, a cryopreserved bone marrow product, or a cryopreserved organ biopsy or removal (for example, a cryopreserved product from thymectomy)), optionally wherein:
step (iii) is performed no later than 35 hours after the beginning of step (v), for example, no later than 27, 28, 29, 30, 31, 32, 33, 34, or 35 hours after the beginning of step (v), for example, no later than 30 hours after the beginning of step (v), or the population of cells from step (iii) are not expanded, or expanded by no more than 5, 10, 15, 20, 25, 30, 35, or 40%, for example, no more than 10%, for example, as assessed by the number of living cells, compared to the population of cells at the end of step (v).

In some embodiments, the method further comprises step (vi):
culturing a portion of the population of cells from step (iii) for at least 2, 2.5, 3, 3.5, 4, 4.5, 5, 5.5, 6, 6.5, or 7 days, for example, at least 2 days and no more than 7 days, and measuring CAR expression level in the portion (for example, measuring the percentage of viable, CAR-expressing cells in the portion), optionally wherein:
step (iii) comprises harvesting and freezing the population of cells (for example, T cells) and step (vi) comprises thawing a portion of the population of cells from step (iii), culturing the portion for at least 2, 2.5, 3, 3.5, 4, 4.5, 5, 5.5, 6, 6.5, or 7 days, for example, at least 2 days and no more than 7 days, and measuring CAR expression level in the portion (for example, measuring the percentage of viable, CAR-expressing cells in the portion).

In some embodiments, the population of cells at the beginning of step (i) or step (1) has been enriched for IL6R-expressing cells (for example, cells that are positive for IL6Rα and/or IL6Rβ). In some embodiments, the population of cells at the beginning of step (i) or step (1) comprises no less than 50, 60, or 70% of IL6R-expressing cells (for example, cells that are positive for IL6Rα and/or IL6Rβ).

In some embodiments, steps (i) and (ii) or steps (1) and (2) are performed in cell media comprising IL-15 (for example, hetIL-15 (IL15/sIL-15Ra)). In some embodiments, IL-15 increases the ability of the population of cells to expand, for example, 10, 15, 20, or 25 days later. In some embodiments, IL-15 increases the percentage of IL6Rβ-expressing cells in the population of cells.

In some embodiments, the lupus is systemic lupus erythematosus. In some embodiments, the SLE is a severe refractory SLE (srSLE).

In some embodiments, the CAR comprises an antigen binding domain, a transmembrane domain, and an intracellular signaling domain.

In some embodiments, the antigen binding domain binds to a B cell antigen associated with lupus (e.g., CD19).

In some embodiments, the antigen binding domain comprises a CDR, VH, VL, scFv or CAR sequence disclosed herein.

In some embodiments, the antigen binding domain comprises a CD19 binding domain comprising a heavy chain complementarity determining region 1 (HC CDR1), an HC CDR2, an HC CDR3, a light chain complementarity determining region 1 (LC CDR 1), an LC CDR2, and an LC CDR3, wherein:
(a) the HC CDR1 comprises the amino acid sequence of SEQ ID NO: 295;
(b) the HC CDR2 comprising the amino acid sequence of SEQ ID NO: 296;
(c) the HC CDR3 comprising the amino acid sequence of SEQ ID NO: 297;
(d) the LC CDR1 comprising the amino acid sequence of SEQ ID NO: 298;
(e) the LC CDR2 comprising the amino acid sequence of SEQ ID NO: 299; and
(f) the LC CDR3 comprising the amino acid sequence of SEQ ID NO: 300.

In some embodiments, the antigen binding domain comprises a VH and a VL, wherein the VH and VL are connected by a linker, optionally wherein the linker comprises the amino acid sequence of SEQ ID NO: 63 or 104.

In some embodiments:
(a) the transmembrane domain comprises a transmembrane domain of a protein chosen from the alpha, beta, or zeta chain of T-cell receptor, CD28, CD3 epsilon, CD45, CD4, CD5, CD8, CD9, CD16, CD22, CD33, CD37, CD64, CD80, CD86, CD134, CD137 and CD154,
(b) the transmembrane domain comprises a transmembrane domain of CD8,
(c) the transmembrane domain comprises the amino acid sequence of SEQ ID NO: 6, or an amino acid sequence having at least about 85%, 90%, 95%, or 99% sequence identity thereof, or
(d) the nucleic acid molecule comprises a nucleic acid sequence encoding the transmembrane domain, wherein the nucleic acid sequence comprises the nucleic acid sequence of SEQ ID NO: 17, or a nucleic acid sequence having at least about 85%, 90%, 95%, or 99% sequence identity thereof.

In some embodiments, the antigen binding domain is connected to the transmembrane domain by a hinge region, optionally wherein:
(a) the hinge region comprises the amino acid sequence of SEQ ID NO: 2, 3, or 4, or an amino acid sequence having at least about 85%, 90%, 95%, or 99% sequence identity thereof, or
(b) the nucleic acid molecule comprises a nucleic acid sequence encoding the hinge region, wherein the nucleic acid sequence comprises the nucleic acid sequence of SEQ ID NO: 13, 14, or 15, or a nucleic acid sequence having at least about 85%, 90%, 95%, or 99% sequence identity thereof.

In some embodiments, the intracellular signaling domain comprises a primary signaling domain, optionally wherein the primary signaling domain comprises a functional signaling domain derived from CD3 zeta, TCR zeta, FcR gamma, FcR beta, CD3 gamma, CD3 delta, CD3 epsilon, CD5, CD22, CD79a, CD79b, CD278 (ICOS), FcERI, DAP10, DAP12, or CD66d, optionally wherein:
(a) the primary signaling domain comprises a functional signaling domain derived from CD3 zeta,
(b) the primary signaling domain comprises the amino acid sequence of SEQ ID NO: 9 or 10, or an amino acid sequence having at least about 85%, 90%, 95%, or 99% sequence identity thereof, or
(c) the nucleic acid molecule comprises a nucleic acid sequence encoding the primary signaling domain, wherein the nucleic acid sequence comprises the nucleic acid sequence of SEQ ID NO: 20 or 21, or a nucleic acid sequence having at least about 85%, 90%, 95%, or 99% sequence identity thereof.

In some embodiments, the intracellular signaling domain comprises a costimulatory signaling domain, optionally wherein the costimulatory signaling domain comprises a functional signaling domain derived from a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, 4-1BB (CD137), B7-H3, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD28-OX40, CD28-4-1BB, or a ligand that specifically binds with CD83, optionally wherein:
(a) the costimulatory signaling domain comprises a functional signaling domain derived from 4-1BB,
(b) the costimulatory signaling domain comprises the amino acid sequence of SEQ ID NO: 7, or an amino acid sequence having at least about 85%, 90%, 95%, or 99% sequence identity thereof, or
(c) the nucleic acid molecule comprises a nucleic acid sequence encoding the costimulatory signaling domain, wherein the nucleic acid sequence comprises the nucleic acid sequence of SEQ ID NO: 18, or a nucleic acid sequence having at least about 85%, 90%, 95%, or 99% sequence identity thereof.

In some embodiments, the intracellular signaling domain comprises a functional signaling domain derived from 4-1BB and a functional signaling domain derived from CD3 zeta, optionally wherein the intracellular signaling domain comprises the amino acid sequence of SEQ ID NO: 7 (or an amino acid sequence having at least about 85%, 90%, 95%, or 99% sequence identity thereof) and the amino acid sequence of SEQ ID NO: 9 or 10 (or an amino acid sequence having at least about 85%, 90%, 95%, or 99% sequence identity thereof), optionally wherein the intracellular signaling domain comprises the amino acid sequence of SEQ ID NO: 7 and the amino acid sequence of SEQ ID NO: 9 or 10.

In some embodiments, the CAR further comprises a leader sequence comprising the amino acid sequence of SEQ ID NO: 1.

In some embodiments, the CAR comprises a CD19 CAR comprising the amino acid sequence of SEQ ID NO: 301, or a sequence having at least 80%, 85%, 90%, 95%, or 99% identity thereto.

In some embodiments, the nucleic acid molecule encoding the CD19 CAR comprises the nucleotide sequence of SEQ ID NO: 302, or a sequence having at least 80%, 85%, 90%, 95%, or 99% identity thereto.

In some embodiments, the subject has been previously treated with one or more of an antimalarial (e.g., hydroxychloroquine or quinacrine), a glucocorticoid (e.g., prednisone), a calcineurin inhibitor, an immunomodulatory agent (e.g., methotrexate, azathioprine, mycophenolate moefetil, cyclophosphamide, or tacrolimus), a biological agent (e.g., belimumab, rituximab, a disease-modifying antirheumatic drug (DMARD) (e.g., leflunomide).

In some embodiments, the subject has been identified as not responding to treatment comprising two or more immunosuppressive therapies (e.g., mycophenolate or cyclophosphamide) in combination with a glucocorticoid) and one biological agent.

In some embodiments, the subject has not previously received a therapy comprising a CD19 CAR, an adoptive T cell therapy, or a gene therapy product.

In some embodiments, leukapheresis occurs (i) prior to administration of corticosteroids and/or (ii) when absolute T cell count is ≥300/mm³.

In one aspect, the disclosure provides a population of CAR-expressing cells (for example, autologous or allogeneic CAR-expressing T cells or NK cells) made by the method described herein.

In some embodiments, the population comprises autoreactive B cells (e.g., autoreactive B cells that do not express a CAR).

In one aspect, the disclosure provides a pharmaceutical composition comprising the population of CAR-expressing cells described herein and a pharmaceutically acceptable carrier.

In one aspect, the disclosure provides a population of CAR-expressing cells or a pharmaceutical composition comprising the same for use in a method of modulating an immune response in a subject having lupus (e.g., systemic lupus erythematosus (SLE), e.g., severe refractory systemic lupus erythematosus (srSLE) or lupus nephritis), said method comprising administering to the subject an effective amount of the population of CAR-expressing cells or an effective amount of the pharmaceutical composition.

In one aspect, the disclosure provides a method of treating a subject having an autoimmune disease, e.g., lupus (e.g., systemic lupus erythematosus (SLE), e.g., severe refractory systemic lupus erythematosus (srSLE) or lupus nephritis), the method comprising administering to the subject:
a population of cells that express, or comprise a nucleic acid configured to express, a CD19 chimeric antigen receptor (CD19 CAR), and
a second therapy chosen from an antimalarial agent or a stable immunosuppressive,
wherein the second therapy and CD19 CAR cells are present in the subject at the same time, e.g., wherein the second therapy is administered at a time when the CD19 CAR cells are present in the subject.

In one aspect, the disclosure provides a method of treating a subject having an autoimmune disease, e.g., lupus (e.g., systemic lupus erythematosus (SLE), e.g., severe refractory systemic lupus erythematosus (srSLE) or lupus nephritis), the method comprising administering to the subject:
rapcabtagene autoleucel, and
a second therapy chosen from an antimalarial agent or a stable immunosuppressive,
wherein the second therapy and rapcabtagene autoleucel are present in the subject at the same time, e.g., wherein the second therapy is administered at a time when rapcabtagene autoleucel is present in the subject.

In some aspects, the disclosure provides rapcabtagene autoleucel, which was made from autologous cells from a subject having an autoimmune disease or disorder, e.g., lupus (e.g., systemic lupus erythematosus (SLE), e.g., severe refractory systemic lupus erythematosus (srSLE), or lupus nephritis), systemic sclerosis (e.g., rapidly progressing systemic sclerosis (SSc) with significant lung involvement (e.g. as for autoHSCT)), idiopathic inflammatory myopathies (e.g., polymyositis, dermatomyositis, anti-synthetase syndrome, immune-mediated necrotizing myopathy, inclusion body myositis, overlap myositis, cancer associated myositis, e.g. anti-synthetase syndrome with ILD), vasculitis (e.g., ANCA-associated vasculitis), severe refractory Sjögren's, severe refractory neuroimmune disease (e.g. myasthenia gravis (MG), neuromyelitis optica (NMO), MOG associated disease (MOGAD), multiple sclerosis (MS)), severe refractory rheumatoid arthritis, antibody mediated neuroimmune diseases (e.g., AChR+ and MuSK+myasthenia gravis (MG), AQP4+ neuromyelitis optica (NMO), MOGAD (anti-MOG associated disease), NMDAR+ encephalitis, or antibody-associated neurological paraneoplastic diseases), Addison's disease, Goodpasture's syndrome, thyrotoxicosis, chronic active hepatitis, relapsing polychondritis, pemphigus vulgaris, or amyotrophic lateral sclerosis.

In one aspect, the disclosure provides a pharmaceutical composition comprising rapcabtagene autoleucel and a pharmaceutically acceptable carrier.

In one aspect, the disclosure provides rapcabtagene autoleucel or a pharmaceutical composition comprising the same for use in a method of modulating an immune response in a subject having an autoimmune disease or disorder, e.g., lupus (e.g., systemic lupus erythematosus (SLE), e.g., severe refractory systemic lupus erythematosus (srSLE), or lupus nephritis), systemic sclerosis (e.g., rapidly progressing systemic sclerosis (SSc) with significant lung involvement (e.g. as for autoHSCT)), idiopathic inflammatory myopathies (e.g., polymyositis, dermatomyositis, anti-synthetase syndrome, immune-mediated necrotizing myopathy, inclusion body myositis, overlap myositis, cancer associated myositis, e.g. anti-synthetase syndrome with ILD), vasculitis (e.g., ANCA-associated vasculitis), severe refractory Sjögren's, severe refractory neuroimmune disease (e.g. myasthenia gravis (MG), neuromyelitis optica (NMO), MOG associated disease (MOGAD), multiple sclerosis (MS)), severe refractory rheumatoid arthritis, antibody mediated neuroimmune diseases (e.g., AChR+ and MuSK+myasthenia gravis (MG), AQP4+ neuromyelitis optica (NMO), MOGAD (anti-MOG associated disease), NMDAR+ encephalitis, or antibody-associated neurological paraneoplastic diseases), Addison's disease, Goodpasture's syndrome, thyrotoxicosis, chronic active hepatitis, relapsing polychondritis, pemphigus vulgaris, or amyotrophic lateral sclerosis, said method comprising administering to the subject an effective amount of the population of rapcaptagene autoleucel or an effective amount of the pharmaceutical composition

In one aspect, the disclosure provides rapcabtagene autoleucel or a pharmaceutical composition comprising the same for use in a method of modulating an immune response in a subject having lupus (e.g., systemic lupus erythematosus (SLE), e.g., severe refractory systemic lupus erythematosus (srSLE) or lupus nephritis), said method comprising administering to the subject an effective amount of rapcabtagene autoleucel or an effective amount of the pharmaceutical composition.

Rapcabtagene autoleucel for use in treating a subject having an autoimmune disease or disorder, e.g., lupus (e.g., systemic lupus erythematosus (SLE), e.g., severe refractory systemic lupus erythematosus (srSLE), or lupus nephritis), systemic sclerosis (e.g., rapidly progressing systemic sclerosis (SSc) with significant lung involvement (e.g. as for autoHSCT)), idiopathic inflammatory myopathies (e.g., polymyositis, dermatomyositis, anti-synthetase syndrome, immune-mediated necrotizing myopathy, inclusion body myositis, overlap myositis, cancer associated myositis, e.g. anti-synthetase syndrome with ILD), vasculitis (e.g., ANCA-associated vasculitis), severe refractory Sjögren's, severe refractory neuroimmune disease (e.g. myasthenia gravis (MG), neuromyelitis optica (NMO), MOG associated disease (MOGAD), multiple sclerosis (MS)), severe refractory rheumatoid arthritis, antibody mediated neuroimmune diseases (e.g., AChR+ and MuSK+myasthenia gravis (MG), AQP4+neuromyelitis optica (NMO), MOGAD (anti-MOG associated disease), NMDAR+ encephalitis, or antibody-associated neurological paraneoplastic diseases), Addison's disease, Goodpasture's syndrome, thyrotoxicosis, chronic active hepatitis, relapsing polychondritis, pemphigus vulgaris, or amyotrophic lateral sclerosis.

Rapcabtagene autoleucel for use in treating a subject having severe refractory systemic lupus erythematosus (srSLE), wherein rapcabtagene autoleucel is formulated for administration in an amount sufficient to treat the srSLE

Rapcabtagene autoleucel for use in treating a subject having lupus (e.g., systemic lupus erythematosus (SLE), e.g., severe refractory systemic lupus erythematosus (srSLE) or lupus nephritis), rapcabtagene autoleucel is formulated for administration at a dose of 0.5−50×10⁶ viable CAR+ T cells (e.g., 5−12.5×10⁶ viable CAR+ T cells).

Rapcabtagene autoleucel and a second therapy for use in treating a subject having an autoimmune disease, e.g., lupus (e.g., systemic lupus erythematosus (SLE), e.g., severe refractory systemic lupus erythematosus (srSLE) or lupus nephritis),
wherein the second therapy is chosen from an antimalarial agent or a stable immunosuppressive, and
wherein the second therapy and rapcabtagene autoleucel are present in the subject at the same time, e.g., wherein the second therapy is administered at a time when rapcabtagene autoleucel is present in the subject.

Although methods and materials similar or equivalent to those described herein can be used in the practice or testing of the present invention, suitable methods and materials are described below. All publications, patent applications, patents, and other references (for example, sequence database reference numbers) mentioned herein are incorporated by reference in their entirety. For example, all GenBank, Unigene, and Entrez sequences referred to herein, for example, in any Table herein, are incorporated by reference. When one gene or protein references a plurality of sequence accession numbers, all of the sequence variants are encompassed.

In addition, the materials, methods, and examples are illustrative only and not intended to be limiting. Headings, sub-headings or numbered or lettered elements, for example, (a), (b), (i) etc., are presented merely for ease of reading. The use of headings or numbered or lettered elements in this document does not require the steps or elements be performed in alphabetical order or that the steps or elements are necessarily discrete from one another. Other features, objects, and advantages of the invention will be apparent from the description and drawings, and from the claims.

### BRIEF DESCRIPTION OF THE FIGURES

FIG. 1 is a schematic showing the clinical trial design for a phase 1/2 study, open-label, multi-center, to assess safety, efficacy and cellular kinetics of ARM-CD19 CAR T cells in participants with severe, refractory autoimmune disorders.

### DETAILED DESCRIPTION

### Definitions

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

The compositions and methods of the present invention encompass polypeptides and nucleic acids having the sequences specified, or sequences substantially identical or similar thereto, for example, sequences at least 85%, 90%, or 95% identical or higher to the sequence specified. In the context of an amino acid sequence, the term “substantially identical” is used herein to refer to a first amino acid sequence that contains a sufficient or minimum number of amino acid residues that are i) identical to, or ii) conservative substitutions of aligned amino acid residues in a second amino acid sequence such that the first and second amino acid sequences can have a common structural domain and/or common functional activity, for example, amino acid sequences that contain a common structural domain having at least about 85%, 90%, 91%, 92%, 93%, 94%, 95%, 96%, 97%, 98% or 99% identity to a reference sequence, for example, a sequence provided herein.

In the context of a nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity, for example, nucleotide sequences having at least about 75%, 80%, 85%, 90%, 91%, 92%, 93%, 94%, 95%, 96%, 97%, 98% or 99% identity to a reference sequence, for example, a sequence provided herein.

The term “variant” refers to a polypeptide that has a substantially identical amino acid sequence to a reference amino acid sequence, or is encoded by a substantially identical nucleotide sequence. In some embodiments, the variant is a functional variant.

The term “functional variant” refers to a polypeptide that has a substantially identical amino acid sequence to a reference amino acid sequence, or is encoded by a substantially identical nucleotide sequence, and is capable of having one or more activities of the reference amino acid sequence.

The term cytokine (for example, IL-2, IL-7, IL-15, IL-21, or IL-6) includes full length, a fragment or a variant, for example, a functional variant, of a naturally-occurring cytokine (including fragments and functional variants thereof having at least 10%, 30%, 50%, or 80% of the activity, e.g., the immunomodulatory activity, of the naturally-occurring cytokine). In some embodiments, the cytokine has an amino acid sequence that is substantially identical (e.g., at least about 85%, 90%, 91%, 92%, 93%, 94%, 95%, 96%, 97%, 98% or 99% identity) to a naturally-occurring cytokine, or is encoded by a nucleotide sequence that is substantially identical (e.g., at least about 75%, 80%, 85%, 90%, 91%, 92%, 93%, 94%, 95%, 96%, 97%, 98% or 99% identity) to a naturally-occurring nucleotide sequence encoding a cytokine. In some embodiments, as understood in context, the cytokine further comprises a receptor domain, e.g., a cytokine receptor domain (e.g., an IL-15/IL-15R).

The term “Chimeric Antigen Receptor” or alternatively a “CAR” refers to a recombinant polypeptide construct comprising at least an extracellular antigen-binding domain, a transmembrane domain and a cytoplasmic signaling domain (also referred to herein as “an intracellular signaling domain”) comprising a functional signaling domain derived from a stimulatory molecule as defined below. In some embodiments, the domains in the CAR polypeptide construct are in the same polypeptide chain, for example, comprise a chimeric fusion protein. In some embodiments, the domains in the CAR polypeptide construct are not contiguous with each other, for example, are in different polypeptide chains, for example, as provided in an RCAR as described herein.

In some embodiments, the cytoplasmic signaling domain comprises a primary signaling domain (for example, a primary signaling domain of CD3-zeta). In some embodiments, the cytoplasmic signaling domain further comprises one or more functional signaling domains derived from at least one costimulatory molecule as defined below. In some embodiments, the costimulatory molecule is chosen from 41BB (i.e., CD137), CD27, ICOS, and/or CD28. In some embodiments, the CAR comprises a chimeric fusion protein comprising an extracellular antigen recognition domain, a transmembrane domain and an intracellular signaling domain comprising a functional signaling domain derived from a stimulatory molecule. In some embodiments, the CAR comprises a chimeric fusion protein comprising an extracellular antigen recognition domain, a transmembrane domain and an intracellular signaling domain comprising a functional signaling domain derived from a costimulatory molecule and a functional signaling domain derived from a stimulatory molecule. In some embodiments, the CAR comprises a chimeric fusion protein comprising an extracellular antigen recognition domain, a transmembrane domain and an intracellular signaling domain comprising two functional signaling domains derived from one or more costimulatory molecule(s) and a functional signaling domain derived from a stimulatory molecule. In some embodiments, the CAR comprises a chimeric fusion protein comprising an extracellular antigen recognition domain, a transmembrane domain and an intracellular signaling domain comprising at least two functional signaling domains derived from one or more costimulatory molecule(s) and a functional signaling domain derived from a stimulatory molecule. In some embodiments the CAR comprises an optional leader sequence at the amino-terminus (N-terminus) of the CAR fusion protein. In some embodiments, the CAR further comprises a leader sequence at the N-terminus of the extracellular antigen recognition domain, wherein the leader sequence is optionally cleaved from the antigen recognition domain (for example, an scFv) during cellular processing and localization of the CAR to the cellular membrane.

A CAR that comprises an antigen-binding domain (for example, an scFv, a single domain antibody, or TCR (for example, a TCR alpha binding domain or TCR beta binding domain)) that targets a specific antigen X, wherein X can be an antigen as described herein, is also referred to as XCAR. For example, a CAR that comprises an antigen-binding domain that targets CD19 is referred to as CD19 CAR. The CAR can be expressed in any cell, for example, an immune effector cell as described herein (for example, a T cell or an NK cell).

The term “signaling domain” refers to the functional portion of a protein which acts by transmitting information within the cell to regulate cellular activity via defined signaling pathways by generating second messengers or functioning as effectors by responding to such messengers.

The term “antibody,” as used herein, refers to a protein, or polypeptide sequence derived from an immunoglobulin molecule, which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

The term “antibody fragment” refers to at least one portion of an intact antibody, or recombinant variants thereof, and refers to the antigen-binding domain, for example, an antigenic determining variable region of an intact antibody, that is sufficient to confer recognition and specific binding of the antibody fragment to a target, such as an antigen. Examples of antibody fragments include, but are not limited to, Fab, Fab′, F(ab′)2, and Fv fragments, scFv antibody fragments, linear antibodies, single domain antibodies such as sdAb (either VL or VH), camelid VHH domains, and multi-specific molecules formed from antibody fragments such as a bivalent fragment comprising two or more, for example, two, Fab fragments linked by a disulfide bridge at the hinge region, or two or more, for example, two isolated CDR or other epitope binding fragments of an antibody linked. An antibody fragment can also be incorporated into single domain antibodies, maxibodies, minibodies, nanobodies, intrabodies, diabodies, triabodies, tetrabodies, v-NAR and bis-scFv (see, for example, Hollinger and Hudson, Nature Biotechnology 23:1126-1136, 2005). Antibody fragments can also be grafted into scaffolds based on polypeptides such as a fibronectin type III (Fn3) (see U.S. Pat. No. 6,703,199, which describes fibronectin polypeptide minibodies).

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein a scFv may have the VL and VH variable regions in either order, for example, with respect to the N-terminal and C-terminal ends of the polypeptide, the scFv may comprise VL-linker-VH or may comprise VH-linker-VL. In some embodiments, the scFv may comprise the structure of NH₂-V_{L}-linker-V_{H}-COOH or NH₂-V_{H}-linker-V_{L}-COOH.

The terms “complementarity determining region” or “CDR,” as used herein, refer to the sequences of amino acids within antibody variable regions which confer antigen specificity and binding affinity. For example, in general, there are three CDRs in each heavy chain variable region (for example, HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme), or a combination thereof. In a combined Kabat and Chothia numbering scheme, in some embodiments, the CDRs correspond to the amino acid residues that are part of a Kabat CDR, a Chothia CDR, or both.

The portion of the CAR composition of the invention comprising an antibody or antibody fragment thereof may exist in a variety of forms, for example, where the antigen-binding domain is expressed as part of a polypeptide chain including, for example, a single domain antibody fragment (sdAb), a single chain antibody (scFv), or for example, a human or humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426). In some embodiments, the antigen-binding domain of a CAR composition of the invention comprises an antibody fragment. In some embodiments, the CAR comprises an antibody fragment that comprises n scFv.

As used herein, the term “binding domain” or “antibody molecule” (also referred to herein as “anti-target binding domain”) refers to a protein, for example, an immunoglobulin chain or fragment thereof, comprising at least one immunoglobulin variable domain sequence. The term “binding domain” or “antibody molecule” encompasses antibodies and antibody fragments. In some embodiments, an antibody molecule is a multispecific antibody molecule, for example, it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In some embodiments, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope.

The terms “bispecific antibody” and “bispecific antibodies” refer to molecules that combine the antigen-binding sites of two antibodies within a single molecule. Thus, a bispecific antibody is able to bind two different antigens simultaneously or sequentially. Methods for making bispecific antibodies are well known in the art. Various formats for combining two antibodies are also known in the art. Forms of bispecific antibodies of the invention include, but are not limited to, a diabody, a single-chain diabody, Fab dimerization (Fab-Fab), Fab-scFv, and a tandem antibody, as known to those of skill in the art.

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (κ) and lambda (λ) light chains refer to the two major antibody light chain isotypes.

The term “recombinant antibody” refers to an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that provokes an immune response. This immune response may involve either antibody production, or the activation of specific immunologically competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell, or a fluid with other biological components.

The term “autologous” refers to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some embodiments, allogeneic material from individuals of the same species may be sufficiently unlike genetically to interact antigenically.

The term “xenogeneic” refers to a graft derived from an animal of a different species.

The term “apheresis” as used herein refers to the art-recognized extracorporeal process by which the blood of a donor or patient is removed from the donor or patient and passed through an apparatus that separates out selected particular constituent(s) and returns the remainder to the circulation of the donor or patient, for example, by re-transfusion. Thus, in the context of “an apheresis sample” refers to a sample obtained using apheresis.

As used herein, “lupus” refers to all types and manifestations of lupus. Manifestations of lupus include, without limitation, systemic lupus erythematosus (including severe refractory SLE (srSLE); lupus nephritis; cutaneous manifestations (e.g., manifestations seen in cutaneous lupus erythematosus, e.g., a skin lesion or rash); CNS lupus; cardiovascular, pulmonary, hepatic, haematological, gastrointestinal and musculoskeletal manifestations; neonatal lupus erythematosus; childhood systemic lupus erythematosus; drug-induced lupus erythematosus; anti-phospholipid syndrome; and complement deficiency syndromes resulting in lupus manifestations.

“Derived from” as that term is used herein, indicates a relationship between a first and a second molecule. It generally refers to structural similarity between the first molecule and a second molecule and does not connotate or include a process or source limitation on a first molecule that is derived from a second molecule. For example, in the case of an intracellular signaling domain that is derived from a CD3zeta molecule, the intracellular signaling domain retains sufficient CD3zeta structure such that is has the required function, namely, the ability to generate a signal under the appropriate conditions. It does not connotate or include a limitation to a particular process of producing the intracellular signaling domain, for example, it does not mean that, to provide the intracellular signaling domain, one must start with a CD3zeta sequence and delete unwanted sequence, or impose mutations, to arrive at the intracellular signaling domain.

The term “conservative sequence modifications” refers to amino acid modifications that do not significantly affect or alter the binding characteristics of the antibody or antibody fragment containing the amino acid sequence. Such conservative modifications include amino acid substitutions, additions, and deletions. Modifications can be introduced into an antibody or antibody fragment of the invention by standard techniques known in the art, such as site-directed mutagenesis and PCR-mediated mutagenesis. Conservative substitutions are ones in which the amino acid residue is replaced with an amino acid residue having a similar side chain. Families of amino acid residues having similar side chains have been defined in the art. These families include amino acids with basic side chains (for example, lysine, arginine, histidine), acidic side chains (for example, aspartic acid, glutamic acid), uncharged polar side chains (for example, glycine, asparagine, glutamine, serine, threonine, tyrosine, cysteine, tryptophan), nonpolar side chains (for example, alanine, valine, leucine, isoleucine, proline, phenylalanine, methionine), beta-branched side chains (for example, threonine, valine, isoleucine) and aromatic side chains (for example, tyrosine, phenylalanine, tryptophan, histidine). Thus, one or more amino acid residues within a CAR of the invention can be replaced with other amino acid residues from the same side chain family and the altered CAR can be tested using the functional assays described herein.

The term “stimulation” in the context of stimulation by a stimulatory and/or costimulatory molecule refers to a response, for example, a primary or secondary response, induced by binding of a stimulatory molecule (for example, a TCR/CD3 complex) and/or a costimulatory molecule (for example, CD28 or 4-1BB) with its cognate ligand thereby mediating a signal transduction event, such as, but not limited to, signal transduction via the TCR/CD3 complex. Stimulation can mediate altered expression of certain molecules and/or reorganization of cytoskeletal structures, and the like.

The term “stimulatory molecule,” refers to a molecule expressed by a T cell that provides the primary cytoplasmic signaling sequence(s) that regulate primary activation of the TCR complex in a stimulatory way for at least some aspect of the T cell signaling pathway. In some embodiments, the ITAM-containing domain within the CAR recapitulates the signaling of the primary TCR independently of endogenous TCR complexes. In some embodiments, the primary signal is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM. Examples of an ITAM containing primary cytoplasmic signaling sequence that is of particular use in the invention includes, but is not limited to, those derived from TCR zeta, FcR gamma, FcR beta, CD3 gamma, CD3 delta, CD3 epsilon, CD5, CD22, CD79a, CD79b, CD278 (also known as “ICOS”), FcεRI and CD66d, DAP10 and DAP12. In a specific CAR of the invention, the intracellular signaling domain in any one or more CARS of the invention comprises an intracellular signaling sequence, for example, a primary signaling sequence of CD3-zeta. The term “antigen presenting cell” or “APC” refers to an immune system cell such as an accessory cell (for example, a B-cell, a dendritic cell, and the like) that displays a foreign antigen complexed with major histocompatibility complexes (MHC's) on its surface. T-cells may recognize these complexes using their T-cell receptors (TCRs). APCs process antigens and present them to T-cells.

An “intracellular signaling domain,” as the term is used herein, refers to an intracellular portion of a molecule. In embodiments, the intracellular signal domain transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

The intracellular signaling domain generates a signal that promotes an immune effector function of the CAR containing cell, for example, a CART cell. Examples of immune effector function, for example, in a CART cell, include cytolytic activity and helper activity, including the secretion of cytokines.

In some embodiments, the intracellular signaling domain can comprise a primary intracellular signaling domain. Exemplary primary intracellular signaling domains include those derived from the molecules responsible for primary stimulation, or antigen dependent simulation. In some embodiments, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, CD278 (also known as “ICOS”), FcεRI, CD66d, DAP10 and DAP12.

The term “zeta” or alternatively “zeta chain”, “CD3-zeta” or “TCR-zeta” refers to CD247. Swiss-Prot accession number P20963 provides exemplary human CD3 zeta amino acid sequences. A “zeta stimulatory domain” or alternatively a “CD3-zeta stimulatory domain” or a “TCR-zeta stimulatory domain” refers to a stimulatory domain of CD3-zeta or a variant thereof (for example, a molecule having mutations, for example, point mutations, fragments, insertions, or deletions). In some embodiments, the cytoplasmic domain of zeta comprises residues 52 through 164 of GenBank Acc. No. BAG36664.1 or a variant thereof (for example, a molecule having mutations, for example, point mutations, fragments, insertions, or deletions). In some embodiments, the “zeta stimulatory domain” or a “CD3-zeta stimulatory domain” is the sequence provided as SEQ ID NO: 9 or 10, or a variant thereof (for example, a molecule having mutations, for example, point mutations, fragments, insertions, or deletions).

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to an MHC class I molecule, TNF receptor proteins, Immunoglobulin-like proteins, cytokine receptors, integrins, signaling lymphocytic activation molecules (SLAM proteins), activating NK cell receptors, BTLA, Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD28-OX40, CD28-4-1BB, and a ligand that specifically binds with CD83.

A costimulatory intracellular signaling domain refers to the intracellular portion of a costimulatory molecule.

The intracellular signaling domain can comprise the entire intracellular portion, or the entire native intracellular signaling domain, of the molecule from which it is derived, or a functional fragment thereof.

The term “4-1BB” refers to CD137 or Tumor necrosis factor receptor superfamily member 9. Swiss-Prot accession number P20963 provides exemplary human 4-1BB amino acid sequences. A “4-1BB costimulatory domain” refers to a costimulatory domain of 4-1BB, or a variant thereof (for example, a molecule having mutations, for example, point mutations, fragments, insertions, or deletions). In some embodiments, the “4-1BB costimulatory domain” is the sequence provided as SEQ ID NO: 7 or a variant thereof (for example, a molecule having mutations, for example, point mutations, fragments, insertions, or deletions).

“Immune effector cell,” as that term is used herein, refers to a cell that is involved in an immune response, for example, in the promotion of an immune effector response. Examples of immune effector cells include T cells, for example, alpha/beta T cells and gamma/delta T cells, B cells, natural killer (NK) cells, natural killer T (NKT) cells, mast cells, and myeloid-derived phagocytes.

“Immune effector function or immune effector response,” as that term is used herein, refers to function or response, for example, of an immune effector cell, that enhances or promotes an immune attack of a target cell. For example, an immune effector function or response refers a property of a T or NK cell that promotes killing or the inhibition of growth or proliferation, of a target cell. In the case of a T cell, primary stimulation and costimulation are examples of immune effector function or response.

The term “effector function” refers to a specialized function of a cell. Effector function of a T cell, for example, may be cytolytic activity or helper activity including the secretion of cytokines.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (for example, rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “effective amount” or “therapeutically effective amount” are used interchangeably herein, and refer to an amount of a compound, formulation, material, or composition, as described herein effective to achieve a particular biological result.

The term “endogenous” refers to any material from or produced inside an organism, cell, tissue, or system.

The term “exogenous” refers to any material introduced from or produced outside an organism, cell, tissue, or system.

The term “expression” refers to the transcription and/or translation of a particular nucleotide sequence. In some embodiments, expression comprises translation of an mRNA introduced into a cell.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (for example, naked or contained in liposomes) and viruses (for example, lentiviruses, retroviruses, adenoviruses, and adeno-associated viruses) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17 (8): 1453-1464 (2009). Other examples of lentivirus vectors that may be used in the clinic, include but are not limited to, for example, the LENTIVECTOR® gene delivery technology from Oxford BioMedica, the LENTIMAX™ vector system from Lentigen and the like. Nonclinical types of lentiviral vectors are also available and would be known to one skilled in the art.

The term “homologous” or “identity” refers to the subunit sequence identity between two polymeric molecules, for example, between two nucleic acid molecules, such as, two DNA molecules or two RNA molecules, or between two polypeptide molecules. When a subunit position in both of the two molecules is occupied by the same monomeric subunit; for example, if a position in each of two DNA molecules is occupied by adenine, then they are homologous or identical at that position. The homology between two sequences is a direct function of the number of matching or homologous positions; for example, if half (for example, five positions in a polymer ten subunits in length) of the positions in two sequences are homologous, the two sequences are 50% homologous; if 90% of the positions (for example, 9 of 10), are matched or homologous, the two sequences are 90% homologous.

“Humanized” forms of non-human (for example, murine) antibodies are chimeric immunoglobulins, immunoglobulin chains or fragments thereof (such as Fv, Fab, Fab′, F(ab′) 2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321:522-525, 1986; Reichmann et al., Nature, 332:323-329, 1988; Presta, Curr. Op. Struct. Biol., 2:593-596, 1992.

“Fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, for example, where necessary to join two protein coding regions, are in the same reading frame.

The term “parenteral” administration of an immunogenic composition includes, for example, subcutaneous (s.c.), intravenous (i.v.), intramuscular (i.m.), or intrasternal injection, intratumoral, or infusion techniques.

The term “nucleic acid,” “nucleic acid molecule,” “polynucleotide,” or “polynucleotide molecule” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. In some embodiments, a “nucleic acid,” “nucleic acid molecule,” “polynucleotide,” or “polynucleotide molecule” comprise a nucleotide/nucleoside derivative or analog. Unless otherwise indicated, a particular nucleic acid sequence also implicitly encompasses conservatively modified variants thereof (for example, degenerate codon substitutions, for example, conservative substitutions), alleles, orthologs, SNPs, and complementary sequences as well as the sequence explicitly indicated. Specifically, degenerate codon substitutions, for example, conservative substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605−2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

As used herein, “B cell antigen” refers to an antigen associated with a B cell. Non-limiting examples of molecules associated with a B cell include proteins expressed on the surface of B cells, e.g. CD19, BCMA, CD22, CD20, CD10, CD34, CD123, FLT-3, ROR1, CD79b, CD179b, or CD79a.

As used herein, the term “CD19” refers to the Cluster of Differentiation 19 protein. The human and murine amino acid and nucleic acid sequences can be found in a public database, such as GenBank, UniProt and Swiss-Prot. For example, the amino acid sequence of human CD19 can be found as UniProt/Swiss-Prot Accession No. P15391 and the nucleic acid sequence encoding of the human CD19 can be found at Accession No. NM_001178098. It is also an early marker of B cell progenitors. See, e.g., Nicholson et al. Mol. Immun. 34 (16-17): 1157-1165 (1997). In one aspect the antigen-binding portion of the CART recognizes and binds an antigen within the extracellular domain of the CD19 protein. In one aspect, the CD19 protein is expressed on an autoreactive B-cell. As used herein, “CD19” includes proteins comprising mutations, e.g., point mutations, fragments, insertions, deletions and splice variants of full length wild-type CD19.

The term “flexible polypeptide linker” or “linker” as used in the context of an scFv refers to a peptide linker that consists of amino acids such as glycine and/or serine residues used alone or in combination, to link variable heavy and variable light chain regions together. In some embodiments, the flexible polypeptide linker is a Gly/Ser linker and comprises the amino acid sequence (Gly-Gly-Gly-Ser) n, where n is a positive integer equal to or greater than 1 (SEQ ID NO: 41). For example, n=1, n=2, n=3. n=4, n=5 and n=6, n=7, n=8, n=9 and n=10 In some embodiments, the flexible polypeptide linkers include, but are not limited to, (Gly4 Ser) 4 (SEQ ID NO: 27) or (Gly4 Ser) 3 (SEQ ID NO: 28). In some embodiments, the linkers include multiple repeats of (Gly2Ser), (GlySer) or (Gly3Ser) (SEQ ID NO: 29). Also included within the scope of the invention are linkers described in WO2012/138475, incorporated herein by reference.

As used herein, a 5′ cap (also termed an RNA cap, an RNA 7-methylguanosine cap or an RNA m⁷G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA that has been synthesized in vitro. In some embodiments the RNA is mRNA. Generally, the in vitro transcribed RNA is generated from an in vitro transcription vector. The in vitro transcription vector comprises a template that is used to generate the in vitro transcribed RNA.

As used herein, a “poly (A)” is a series of adenosines attached by polyadenylation to the mRNA. In some embodiments of a construct for transient expression, the poly(A) is between 50 and 5000 (SEQ ID NO: 30). In some embodiments the poly(A) is greater than 64. In some embodiments the poly(A) is greater than 100. In some embodiments the poly(A) is greater than 300. In some embodiments the poly(A) is greater than 400. poly (A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly (A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

As used herein, the terms “treat”, “treatment” and “treating” refer to the reduction or amelioration of the progression, severity and/or duration of an autoimmune disorder, or the amelioration of one or more symptoms (preferably, one or more discernible symptoms) of an autoimmune disorder resulting from the administration of one or more therapies (for example, one or more therapeutic agents such as a CAR of the invention). In specific embodiments, the terms “treat,” “treatment,” and “treating” refer to the amelioration of at least one measurable physical parameter of an autoimmune disorder, such as the level of autoantibodies, not necessarily discernible by the patient. In other embodiments the terms “treat”, “treatment” and “treating”-refer to the inhibition of the progression of an autoimmune disorder, either physically by, for example, stabilization of a discernible symptom, physiologically by, for example, stabilization of a physical parameter, or both. The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (for example, mammals, for example, human).

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In some embodiments, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

The term “transfected” or “transformed” or “transduced” refers to a process by which exogenous nucleic acid is transferred or introduced into the host cell. A “transfected” or “transformed” or “transduced” cell is one which has been transfected, transformed, or transduced with exogenous nucleic acid. The cell includes the primary subject cell and its progeny.

The term “specifically binds,” refers to an antibody, or a ligand, which recognizes and binds with a cognate binding partner (for example, a stimulatory and/or costimulatory molecule present on a T cell) protein present in a sample, but which antibody or ligand does not substantially recognize or bind other molecules in the sample.

“Membrane anchor” or “membrane tethering domain”, as that term is used herein, refers to a polypeptide or moiety, for example, a myristoyl group, sufficient to anchor an extracellular or intracellular domain to the plasma membrane.

“Refractory” as used herein refers to an autoimmune disease or disorder, for example, SLE, which does not respond to a treatment. In embodiments, a refractory autoimmune disease or disorder can be resistant to a treatment before or at the beginning of the treatment. In other embodiments, the refractory autoimmune disease or disorder can become resistant during a treatment. A refractory autoimmune disease or disorder is also called a resistant autoimmune disease or disorder.

As used herein, “severe refractory autoimmune disease” refers to a manifestation of an autoimmune disease that has failed to respond (e.g., remains charactericterized by high disease activity) following at least one standard immunosuppressive therapy or at least one biological agent. One example of a severe refractory autoimmune disease is severe refractory systemic lupus erythematosus.

As used herein, “severe refractory systemic lupus erythematosus” or “srSLE” refers to a manifestation of SLE that has failed to respond (e.g., remains characterized by high disease activity) following at least one standard immunosuppressive therapy (e.g., mycophenolate, cyclophosphamide), glucocorticoids, or at least one biological agent. In some embodiments, the srSLE comprises a manifestation of SLE that has failed to respond to two or more standard immunosuppressive therapies in combination with glucocorticoids. In some embodiments, the srSLE comprises a manifestation of SLE that has failed to respond to at least one biological agent.

“Relapsed” or “relapse” as used herein refers to the return or reappearance of a disease (for example, an autoimmune disease or disorder) or the signs and symptoms of a disease such as an autoimmune disease or disorder after a period of improvement or responsiveness, for example, after prior treatment of a therapy, for example, standard of care therapy. The initial period of responsiveness may involve the level of autoantibodies cells falling below a certain threshold. The reappearance may involve the level of autoantibodies rising above a certain threshold.

Ranges: throughout this disclosure, various embodiments of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98%, or 99% identity, and includes subranges such as 96-99%, 96-98%, 96-97%, 97-99%, 97-98%, and 98-99% identity. This applies regardless of the breadth of the range.

Administered “in combination”, as used herein, means that two (or more) different treatments are delivered to the subject during the course of the subject's affliction with the disorder, for example, the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, for example, an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The term “depletion” or “depleting”, as used interchangeably herein, refers to the decrease or reduction of the level or amount of a cell, a protein, or macromolecule in a sample after a process, for example, a selection step, for example, a negative selection, is performed. The depletion can be a complete or partial depletion of the cell, protein, or macromolecule. In some embodiments, the depletion is at least a 1%, 2%, 5%, 10%, 15%, 20%, 25%, 30%, 35%, 40%, 45%, 50%, 55%, 60%, 65%, 70%, 75%, 80%, 85%, 90%, 95%, or 99% decrease or reduction of the level or amount of a cell, a protein, or macromolecule, as compared to the level or amount of the cell, protein or macromolecule in the sample before the process was performed.

As used herein, a “naïve T cell” refers to a T cell that is antigen-inexperienced. In some embodiments, an antigen-inexperienced T cell has encountered its cognate antigen in the thymus but not in the periphery. In some embodiments, naïve T cells are precursors of memory cells. In some embodiments, naïve T cells express both CD45RA and CCR7, but do not express CD45RO. In some embodiments, naïve T cells may be characterized by expression of CD62L, CD27, CCR7, CD45RA, CD28, and CD127, and the absence of CD95 or CD45RO isoform. In some embodiments, naïve T cells express CD62L, IL-7 receptor-α, IL-6 receptor, and CD132, but do not express CD25, CD44, CD69, or CD45RO. In some embodiments, naïve T cells express CD45RA, CCR7, and CD62L and do not express CD95 or IL-2 receptor β. In some embodiments, surface expression levels of markers are assessed using flow cytometry.

The term “central memory T cells” refers to a subset of T cells that in humans are CD45RO positive and express CCR7. In some embodiments, central memory T cells express CD95. In some embodiments, central memory T cells express IL-2R, IL-7R, and/or IL-15R. In some embodiments, central memory T cells express CD45RO, CD95, IL-2 receptor β, CCR7, and CD62L. In some embodiments, surface expression levels of markers are assessed using flow cytometry.

The term “stem memory T cells,” “stem cell memory T cells,” “stem cell-like memory T cells,” “memory stem T cells,” “T memory stem cells,” “T stem cell memory cells,” or “TSCM cells” refers to a subset of memory T cells with stem cell-like ability, for example, the ability to self-renew and/or the multipotent capacity to reconstitute memory and/or effector T cell subsets. In some embodiments, stem memory T cells express CD45RA, CD95, IL-2 receptor β, CCR7, and CD62L. In some embodiments, surface expression levels of markers are assessed using flow cytometry. In some embodiments, exemplary stem memory T cells are disclosed in Gattinoni et al., Nat Med. 2017 Jan. 6; 23 (1): 18-27, herein incorporated by reference in its entirety.

For clarity purposes, unless otherwise noted, classifying a cell or a population of cells as “not expressing,” or having an “absence of” or being “negative for” a particular marker may not necessarily mean an absolute absence of the marker. The skilled artisan can readily compare the cell against a positive and/or a negative control, and/or set a predetermined threshold, and classify the cell or population of cells as not expressing or being negative for the marker when the cell has an expression level below the predetermined threshold or a population of cells has an overall expression level below the predetermined threshold using conventional detection methods, e.g., using flow cytometry, for example, as described in the Examples herein. As used herein, the term “GeneSetScore (Up TEM vs. Down TSCM)” of a cell refers to a score that reflects the degree at which the cell shows an effector memory T cell (TEM) phenotype vs. a stem cell memory T cell (TSCM) phenotype. A higher GeneSetScore (Up TEM vs. Down TSCM) indicates an increasing TEM phenotype, whereas a lower GeneSetScore (Up TEM vs. Down TSCM) indicates an increasing TSCM phenotype. In some embodiments, the GeneSetScore (Up TEM vs. Down TSCM) is determined by measuring the expression of one or more genes that are up-regulated in TEM cells and/or down-regulated in TSCM cells, for example, one or more genes selected from the group consisting of MXRA7, CLIC1, NAT13, TBC1D2B, GLCCI1, DUSP10, APOBEC3D, CACNB3, ANXA2P2, TPRG1, EOMES, MATK, ARHGAP10, ADAM8, MAN1A1, SLFN12L, SH2D2A, EIF2C4, CD58, MYO1F, RAB27B, ERN1, NPC1, NBEAL2, APOBEC3G, SYTL2, SLC4A4, PIK3AP1, PTGDR, MAF, PLEKHA5, ADRB2, PLXND1, GNAO1, THBS1, PPP2R2B, CYTH3, KLRF1, FLJ16686, AUTS2, PTPRM, GNLY, and GFPT2. In some embodiments, the GeneSetScore (Up TEM vs. Down TSCM) is determined for each cell using RNA-seq, for example, single-cell RNA-seq (scRNA-seq), for example, as exemplified of WO/2020/047452 in Example 10 with respect to FIG. 39A, hereby incorporated by reference in its entirety. In some embodiments, the GeneSetScore (Up TEM vs. Down TSCM) is calculated by taking the mean log normalized gene expression value of all of the genes in the gene set.

As used herein, the term “GeneSetScore (Up Treg vs. Down Teff)” of a cell refers to a score that reflects the degree at which the cell shows a regulatory T cell (Treg) phenotype vs. an effector T cell (Teff) phenotype. A higher GeneSetScore (Up Treg vs. Down Teff) indicates an increasing Treg phenotype, whereas a lower GeneSetScore (Up Treg vs. Down Teff) indicates an increasing Teff phenotype. In some embodiments, the GeneSetScore (Up Treg vs. Down Teff) is determined by measuring the expression of one or more genes that are up-regulated in Treg cells and/or down-regulated in Teff cells, for example, one or more genes selected from the group consisting of C12orf75, SELPLG, SWAP70, RGS1, PRR11, SPATS2L, SPATS2L, TSHR, C14orf145, CASP8, SYT11, ACTN4, ANXA5, GLRX, HLA-DMB, PMCH, RAB11FIP1, IL32, FAM160B1, SHMT2, FRMD4B, CCR3, TNFRSF13B, NTNG2, CLDND1, BARD1, FCERIG, TYMS, ATP1B1, GJB6, FGL2, TK1, SLC2A8, CDKN2A, SKAP2, GPR55, CDCA7, S100A4, GDPD5, PMAIP1, ACOT9, CEP55, SGMS1, ADPRH, AKAP2, HDAC9, IKZF4, CARD17, VAV3, OBFC2A, ITGB1, CIITA, SETD7, HLA-DMA, CCR10, KIAA0101, SLC14A1, PTTG3P, DUSP10, FAM164A, PYHIN1, MYO1F, SLC1A4, MYBL2, PTTG1, RRM2, TP53INP1, CCR5, ST8SIA6, TOX, BFSP2, ITPRIPL1, NCAPH, HLA-DPB2, SYT4, NINJ2, FAM46C, CCR4, GBP5, C15orf53, LMCD1, MKI67, NUSAP1, PDE4A, E2F2, CD58, ARHGEF12, LOC100188949, FAS, HLA-DPB1, SELP, WEE1, HLA-DPA1, FCRL1, ICA1, CNTNAP1, OAS1, METTL7A, CCR6, HLA-DRB4, ANXA2P3, STAM, HLA-DQB2, LGALS1, ANXA2, PI16, DUSP4, LAYN, ANXA2P2, PTPLA, ANXA2P1, ZNF365, LAIR2, LOC541471, RASGRP4, BCAS1, UTS2, MIAT, PRDM1, SEMA3G, FAM129A, HPGD, NCF4, LGALS3, CEACAM4, JAKMIP1, TIGIT, HLA-DRA, IKZF2, HLA-DRB1, FANK1, RTKN2, TRIB1, FCRL3, and FOXP3. In some embodiments, the GeneSetScore (Up Treg vs. Down Teff) is determined using RNA-seq, for example, single-cell RNA-seq (scRNA-seq), for example, as exemplified of WO/2020/047452 in Example 10 with respect to FIG. 39B, hereby incorporated by reference in its entirety. In some embodiments, the GeneSetScore (Up Treg vs. Down Teff) is calculated by taking the mean log normalized gene expression value of all of the genes in the gene set.

As used herein, the term “GeneSetScore (Down stemness)” of a cell refers to a score that reflects the degree at which the cell shows a stemness phenotype. A lower GeneSetScore (Down stemness) indicates an increasing stemness phenotype. In some embodiments, the GeneSetScore (Down stemness) is determined by measuring the expression of one or more genes that are upregulated in a differentiating stem cell vs downregulated in a hematopoietic stem cell, for example, one or more genes selected from the group consisting of ACE, BATF, CDK6, CHD2, ERCC2, HOXB4, MEOX1, SFRP1, SP7, SRF, TAL1, and XRCC5. In some embodiments, the GeneSetScore (Down stemness) is determined using RNA-seq, for example, single-cell RNA-seq (scRNA-seq), for example, as exemplified of WO/2020/047452 in Example 10 with respect to FIG. 39C, hereby incorporated by reference in its entirety. In some embodiments, the GeneSetScore (Down stemness) is calculated by taking the mean log normalized gene expression value of all of the genes in the gene set.

As used herein, the term “GeneSetScore (Up hypoxia)” of a cell refers to a score that reflects the degree at which the cell shows a hypoxia phenotype. A higher GeneSetScore (Up hypoxia) indicates an increasing hypoxia phenotype. In some embodiments, the GeneSetScore (Up hypoxia) is determined by measuring the expression of one or more genes that are up-regulated in cells undergoing hypoxia, for example, one or more genes selected from the group consisting of ABCB1, ACAT1, ADM, ADORA2B, AK2, AK3, ALDH1A1, ALDH1A3, ALDOA, ALDOC, ANGPT2, ANGPTL4, ANXA1, ANXA2, ANXA5, ARHGAP5, ARSE, ART1, BACE2, BATF3, BCL2L1, BCL2L2, BHLHE40, BHLHE41, BIK, BIRC2, BNIP3, BNIP3L, BPI, BTG1, C11orf2, C7orf68, CA12, CA9, CALD1, CCNG2, CCT6A, CD99, CDK1, CDKNIA, CDKNIB, CITED2, CLK1, CNOT7, COL4A5, COL5A1, COL5A2, COL5A3, CP, CTSD, CXCR4, D4S234E, DDIT3, DDIT4, 1-Dec, DKC1, DR1, EDN1, EDN2, EFNA1, EGF, EGR1, EIF4A3, ELF3, ELL2, ENG, ENO1, ENO3, ENPEP, EPO, ERRFI1, ETS1, F3, FABP5, FGF3, FKBP4, FLT1, FN1, FOS, FTL, GAPDH, GBE1, GLRX, GPI, GPRC5A, HAP1, HBP1, HDAC1, HDAC9, HERC3, HERPUD1, HGF, HIFIA, HK1, HK2, HLA-DQB1, HMOX1, HMOX2, HSPA5, HSPD1, HSPH1, HYOU1, ICAM1, ID2, IFI27, IGF2, IGFBP1, IGFBP2, IGFBP3, IGFBP5, IL6, IL8, INSIG1, IRF6, ITGA5, JUN, KDR, KRT14, KRT18, KRT19, LDHA, LDHB, LEP, LGALS1, LONP1, LOX, LRP1, MAP4, MET, MIF, MMP13, MMP2, MMP7, MPI, MTIL, MTL3P, MUC1, MXII, NDRG1, NFIL3, NFKB1, NFKB2, NOS1, NOS2, NOS2P1, NOS2P2, NOS3, NR3C1, NR4A1, NT5E, ODC1, P4HA1, P4HA2, PAICS, PDGFB, PDK3, PFKFB1, PFKFB3, PFKFB4, PFKL, PGAM1, PGF, PGK1, PGK2, PGM1, PIM1, PIM2, PKM2, PLAU, PLAUR, PLIN2, PLOD2, PNN, PNP, POLM, PPARA, PPAT, PROK1, PSMA3, PSMD9, PTGS1, PTGS2, QSOX1, RBPJ, RELA, RIOK3, RNASEL, RPL36A, RRP9, SAT1, SERPINB2, SERPINE1, SGSM2, SIAH2, SIN3A, SIRPA, SLC16A1, SLC16A2, SLC20A1, SLC2A1, SLC2A3, SLC3A2, SLC6A10P, SLC6A16, SLC6A6, SLC6A8, SORLI, SPP1, SRSF6, SSSCA1, STC2, STRA13, SYT7, TBPL1, TCEAL1, TEK, TF, TFF3, TFRC, TGFA, TGFB1, TGFB3, TGFB1, TGM2, TH, THBS1, THBS2, TIMM17A, TNFAIP3, TP53, TPBG, TPD52, TPI1, TXN, TXNIP, UMPS, VEGFA, VEGFB, VEGFC, VIM, VPS11, and XRCC6. In some embodiments, the GeneSetScore (Up hypoxia) is determined using RNA-seq, for example, single-cell RNA-seq (scRNA-seq), for example, as exemplified of WO/2020/047452 in Example 10 with respect to FIG. 39D, hereby incorporated by reference in its entirety. In some embodiments, the GeneSetScore (Up hypoxia) is calculated by taking the mean log normalized gene expression value of all of the genes in the gene set.

As used herein, the term “GeneSetScore (Up autophagy)” of a cell refers to a score that reflects the degree at which the cell shows an autophagy phenotype. A higher GeneSetScore (Up autophagy) indicates an increasing autophagy phenotype. In some embodiments, the GeneSetScore (Up autophagy) is determined by measuring the expression of one or more genes that are up-regulated in cells undergoing autophagy, for example, one or more genes selected from the group consisting of ABL1, ACBD5, ACIN1, ACTRT1, ADAMTS7, AKR1E2, ALKBH5, ALPK1, AMBRA1, ANXA5, ANXA7, ARSB, ASB2, ATG10, ATG12, ATG13, ATG14, ATG16L1, ATG16L2, ATG2A, ATG2B, ATG3, ATG4A, ATG4B, ATG4C, ATG4D, ATG5, ATG7, ATG9A, ATG9B, ATP13A2, ATP1B1, ATPAF1-AS1, ATPIF1, BECN1, BECN1P1, BLOC1S1, BMP2KL, BNIP1, BNIP3, BOC, C11orf2, C11orf41, C12orf44, C12orf5, C14orf133, Clorf210, C5, C6orf10⁶, C7orf59, C7orf68, C8orf59, C9orf72, CA7, CALCB, CALCOCO2, CAPS, CCDC36, CD163L1, CD93, CDC37, CDKN2A, CHAFIB, CHMP2A, CHMP2B, CHMP3, CHMP4A, CHMP4B, CHMP4C, CHMP6, CHST3, CISD2, CLDN7, CLEC16A, CLN3, CLVS1, COX8A, CPA3, CRNKL1, CSPG5, CTSA, CTSB, CTSD, CXCR7, DAP, DKKL1, DNAAF2, DPF3, DRAM1, DRAM2, DYNLL1, DYNLL2, DZANK1, EI24, EIF2S1, EPG5, EPM2A, FABP1, FAM125A, FAM131B, FAM134B, FAM13B, FAM176A, FAM176B, FAM48A, FANCC, FANCF, FANCL, FBX07, FCGR3B, FGF14, FGF7, FGFBP1, FIS1, FNBPIL, FOXO1, FUNDC1, FUNDC2, FXR2, GABARAP, GABARAPL1, GABARAPL2, GABARAPL3, GABRA5, GDF5, GMIP, HAP1, HAPLN1, HBXIP, HCAR1, HDAC6, HGS, HIST1H3A, HIST1H3B, HIST1H3C, HIST1H3D, HIST1H3E, HIST1H3F, HIST1H3G, HIST1H3H, HIST1H3I, HIST1H3J, HK2, HMGB1, HPR, HSF2BP, HSP90AA1, HSPA8, IFI16, IPPK, IRGM, IST1, ITGB4, ITPKC, KCNK3, KCNQ1, KIAA0226, KIAA1324, KRCC1, KRT15, KRT73, LAMP1, LAMP2, LAMTOR1, LAMTOR2, LAMTOR3, LARPIB, LENG9, LGALS8, LIX1, LIXIL, LMCD1, LRRK2, LRSAM1, LSM4, MAPIA, MAPILC3A, MAPILC3B, MAP1LC3B2, MAPILC3C, MAPIS, MAP2K1, MAP3K12, MARK2, MBD5, MDH1, MEX3C, MFN1, MFN2, MLST8, MRPS10, MRPS2, MSTN, MTERFD1, MTMR14, MTMR3, MTOR, MTSS1, MYH11, MYLK, MYOM1, NBR1, NDUFB9, NEFM, NHLRC1, NME2, NPC1, NR2C2, NRBF2, NTHL1, NUP93, OBSCN, OPTN, P2RX5, PACS2, PARK2, PARK7, PDK1, PDK4, PEX13, PEX3, PFKP, PGK2, PHF23, PHYHIP, PI4K2A, PIK3C3, PIK3CA, PIK3CB, PIK3R4, PINK1, PLEKHM1, PLOD2, PNPO, PPARGCIA, PPY, PRKAA1, PRKAA2, PRKAB1, PRKAB2, PRKAG1, PRKAG2, PRKAG3, PRKD2, PRKG1, PSEN1, PTPN22, RAB12, RABIA, RAB1B, RAB23, RAB24, RAB33B, RAB39, RAB7A, RB1CC1, RBM18, REEP2, REP15, RFWD3, RGS19, RHEB, RIMS3, RNF185, RNF41, RPS27A, RPTOR, RRAGA, RRAGB, RRAGC, RRAGD, S100A8, S100A9, SCNIA, SERPINB10, SESN2, SFRP4, SH3GLB1, SIRT2, SLC1A3, SLC1A4, SLC22A3, SLC25A19, SLC35B3, SLC35C1, SLC37A4, SLC6A1, SLCO1A2, SMURF1, SNAP29, SNAPIN, SNF8, SNRPB, SNRPB2, SNRPD1, SNRPF, SNTG1, SNX14, SPATA18, SQSTM1, SRPX, STAM, STAM2, STAT2, STBD1, STK11, STK32A, STOM, STX12, STX17, SUPT3H, TBC1D17, TBC1D25, TBC1D5, TCIRG1, TEAD4, TECPR1, TECPR2, TFEB, TM9SF1, TMBIM6, TMEM203, TMEM208, TMEM39A, TMEM39B, TMEM59, TMEM74, TMEM93, TNIK, TOLLIP, TOMM20, TOMM22, TOMM40, TOMM5, TOMM6, TOMM7, TOMM70A, TP53INP1, TP53INP2, TRAPPC8, TREM1, TRIM17, TRIM5, TSG101, TXLNA, UBA52, UBB, UBC, UBQLN1, UBQLN2, UBQLN4, ULK1, ULK2, ULK3, USP10, USP13, USP30, UVRAG, VAMP7, VAMP8, VDAC1, VMP1, VPS11, VPS16, VPS18, VPS25, VPS28, VPS33A, VPS33B, VPS36, VPS37A, VPS37B, VPS37C, VPS37D, VPS39, VPS41, VPS4A, VPS4B, VTA1, VTIIA, VTI1B, WDFY3, WDR45, WDR45L, WIPI1, WIPI2, XBP1, YIPF1, ZCCHC17, ZFYVE1, ZKSCAN3, ZNF189, ZNF593, and ZNF681. In some embodiments, the GeneSetScore (Up autophagy) is determined using RNA-seq, for example, single-cell RNA-seq (scRNA-seq), for example, as exemplified of WO/2020/047452 in Example 10 with respect to FIG. 39E, hereby incorporated by reference in its entirety. In some embodiments, the GeneSetScore (Up autophagy) is calculated by taking the mean log normalized gene expression value of all of the genes in the gene set.

As used herein, the term “GeneSetScore (Up resting vs. Down activated)” of a cell refers to a score that reflects the degree at which the cell shows a resting T cell phenotype vs. an activated T cell phenotype. A higher GeneSetScore (Up resting vs. Down activated) indicates an increasing resting T cell phenotype, whereas a lower GeneSetScore (Up resting vs. Down activated) indicates an increasing activated T cell phenotype. In some embodiments, the GeneSetScore (Up resting vs. Down activated) is determined by measuring the expression of one or more genes that are up-regulated in resting T cells and/or down-regulated in activated T cells, for example, one or more genes selected from the group consisting of ABCA7, ABCF3, ACAP2, AMT, ANKH, ATF7IP2, ATG14, ATP1A1, ATXN7, ATXN7L3B, BCL7A, BEX4, BSDC1, BTG1, BTG2, BTN3A1, C11orf21, C19orf22, C21orf2, CAMK2G, CARS2, CCNL2, CD248, CD5, CD55, CEP164, CHKB, CLK1, CLK4, CTSL1, DBP, DCUNID2, DENNDIC, DGKD, DLG1, DUSP1, EAPP, ECE1, ECHDC2, ERBB2IP, FAM117A, FAM134B, FAM134C, FAM169A, FAM190B, FAU, FLJ10038, FOXJ2, FOXJ3, FOXL1, FOXO1, FXYD5, FYB, HLA-E, HSPAIL, HYAL2, ICAM2, IFIT5, IFITM1, IKBKB, IQSEC1, IRS4, KIAA0664L3, KIAA0748, KLF3, KLF9, KRT18, LEF1, LINC00342, LIPA, LIPT1, LLGL2, LMBRIL, LPAR2, LTBP3, LYPD3, LZTFL1, MANBA, MAP2K6, MAP3K1, MARCH8, MAU2, MGEA5, MMP8, MPO, MSL1, MSL3, MYH3, MYLIP, NAGPA, NDST2, NISCH, NKTR, NLRP1, NOSIP, NPIP, NUMA1, PAIP2B, PAPD7, PBXIP1, PCIF1, PI4KA, PLCL2, PLEKHA1, PLEKHF2, PNISR, PPFIBP2, PRKCA, PRKCZ, PRKD3, PRMT2, PTP4A3, PXN, RASA2, RASA3, RASGRP2, RBM38, REPIN1, RNF38, RNF44, ROR1, RPL30, RPL32, RPLP1, RPS20, RPS24, RPS27, RPS6, RPS9, RXRA, RYK, SCAND2, SEMA4C, SETD1B, SETD6, SETX, SF3B1, SH2B1, SLC2A4RG, SLC35E2B, SLC46A3, SMAGP, SMARCE1, SMPD1, SNPH, SP140L, SPATA6, SPG7, SREK1IP1, SRSF5, STAT5B, SVIL, SYF2, SYNJ2BP, TAFIC, TBC1D4, TCF20, TECTA, TES, TMEM127, TMEM159, TMEM30B, TMEM66, TMEM8B, TP53TG1, TPCN1, TRIM22, TRIM44, TSC1, TSC22D1, TSC22D3, TSPYL2, TTC9, TTN, UBE2G2, USP33, USP34, VAMP1, VILL, VIPR1, VPS13C, ZBED5, ZBTB25, ZBTB40, ZC3H3, ZFP161, ZFP36L1, ZFP36L2, ZHX2, ZMYM5, ZNF136, ZNF148, ZNF318, ZNF350, ZNF512B, ZNF609, ZNF652, ZNF83, ZNF862, and ZNF91. In some embodiments, the GeneSetScore (Up resting vs. Down activated) is determined using RNA-seq, for example, single-cell RNA-seq (scRNA-seq), for example, as exemplified of WO/2020/047452 in Example 10 with respect to FIG. 38D, hereby incorporated by reference in its entirety. In some embodiments, the GeneSetScore (Up resting vs. Down activated) is calculated by taking the mean log normalized gene expression value of all of the genes in the gene set.

As used herein, the term “GeneSetScore (Progressively up in memory differentiation)” of a cell refers to a score that reflects the stage of the cell in memory differentiation. A higher GeneSetScore (Progressively up in memory differentiation) indicates an increasing late memory T cell phenotype, whereas a lower GeneSetScore (Progressively up in memory differentiation) indicates an increasing early memory T cell phenotype. In some embodiments, the GeneSetScore (Up autophagy) is determined by measuring the expression of one or more genes that are up-regulated during memory differentiation, for example, one or more genes selected from the group consisting of MTCH2, RAB6C, KIAA0195, SETD2, C2orf24, NRD1, GNA13, COPA, SELT, TNIP1, CBFA2T2, LRP10, PRKCI, BRE, ANKSIA, PNPLA6, ARL6IP1, WDFY1, MAPK1, GPR153, SHKBP1, MAPILC3B2, PIP4K2A, HCN3, GTPBP1, TLN1, C4orf34, KIF3B, TCIRG1, PPP3CA, ATG4D, TYMP, TRAF6, C17orf76, WIPF1, FAM108A1, MYL6, NRM, SPCS2, GGT3P, GALK1, CLIP4, ARL4C, YWHAQ, LPCAT4, ATG2A, IDS, TBC1D5, DMPK, ST6GALNAC6, REEP5, ABHD6, KIAA0247, EMB, TSEN54, SPIRE2, PIWIL4, ZSCAN22, ICAM1, CHD9, LPIN2, SETD8, ZC3H12A, ULBP3, IL15RA, HLA-DQB2, LCP1, CHP, RUNX3, TMEM43, REEP4, MEF2D, ABL1, TMEM39A, PCBP4, PLCD1, CHST12, RASGRP1, Clorf58, C11orf63, C6orf129, FHOD1, DKFZp434F142, PIK3CG, ITPR3, BTG3, C4orf50, CNNM3, IFI16, AK1, CDK2AP1, REL, BCL2L1, MVD, TTC39C, PLEKHA2, FKBP11, EML4, FANCA, CDCA4, FUCA2, MFSD10, TBCD, CAPN2, IQGAP1, CHST11, PIK3R1, MYO5A, KIR2DL3, DLG3, MXD4, RALGDS, S1PR5, WSB2, CCR3, TIPARP, SP140, CD151, SOX13, KRTAP5−2, NF1, PEA15, PARP8, RNF166, UEVLD, LIMK1, CACNB1, TMX4, SLC6A6, LBA1, SV2A, LLGL2, IRF1, PPP2R5C, CD99, RAPGEF1, PPP4R1, OSBPL7, FOXP4, SLA2, TBC1D2B, ST7, JAZF1, GGA2, PI4K2A, CD68, LPGAT1, STX11, ZAK, FAM160B1, RORA, C8orf80, APOBEC3F, TGFB1, DNAJC1, GPR114, LRP8, CD69, CMIP, NAT13, TGFB1, FLJ00049, ANTXR2, NR4A3, IL12RB1, NTNG2, RDX, MLLT4, GPRIN3, ADCY9, CD300A, SCD5, ABI3, PTPN22, LGALS1, SYTL3, BMPRIA, TBK1, PMAIP1, RASGEFIA, GCNT1, GABARAPL1, STOM, CALHM2, ABCA2, PPP1R16B, SYNE2, PAM, C12orf75, CLCF1, MXRA7, APOBEC3C, CLSTN3, ACOT9, HIP1, LAG3, TNFAIP3, DCBLD1, KLF6, CACNB3, RNF19A, RAB27A, FADS3, DLG5, APOBEC3D, TNFRSFIB, ACTN4, TBKBP1, ATXN1, ARAP2, ARHGEF12, FAM53B, MAN1A1, FAM38A, PLXNC1, GRLF1, SRGN, HLA-DRB5, B4GALT5, WIPI1, PTPRJ, SLFN11, DUSP2, ANXA5, AHNAK, NEO1, CLIC1, EIF2C4, MAP3K5, IL2RB, PLEKHG1, MY06, GTDC1, EDARADD, GALM, TARP, ADAM8, MSC, HNRPLL, SYT11, ATP2B4, NHSL2, MATK, ARHGAP18, SLFN12L, SPATS2L, RAB27B, PIK3R3, TP53INP1, MBOAT1, GYG1, KATNAL1, FAM46C, ZC3HAVIL, ANXA2P2, CTNNA1, NPC1, C3AR1, CRIM1, SH2D2A, ERN1, YPEL1, TBX21, SLC1A4, FASLG, PHACTR2, GALNT3, ADRB2, PIK3AP1, TLR3, PLEKHA5, DUSP10, GNAO1, PTGDR, FRMD4B, ANXA2, EOMES, CADM1, MAF, TPRG1, NBEAL2, PPP2R2B, PELO, SLC4A4, KLRF1, FOSL2, RGS2, TGFBR3, PRF1, MYO1F, GAB3, C17orf66, MICAL2, CYTH3, TOX, HLA-DRA, SYNE1, WEE1, PYHIN1, F2R, PLD1, THBS1, CD58, FAS, NETO2, CXCR6, ST6GALNAC2, DUSP4, AUTS2, Clorf21, KLRG1, TNIP3, GZMA, PRR5L, PRDM1, ST8SIA6, PLXND1, PTPRM, GFPT2, MYBL1, SLAMF7, FLJ16686, GNLY, ZEB2, CST7, IL18RAP, CCL5, KLRD1, and KLRB1. In some embodiments, the GeneSetScore (Progressively up in memory differentiation) is determined using RNA-seq, for example, as exemplified of WO/2020/047452 in Example 10 with respect to FIG. 40B, hereby incorporated by reference in its entirety. In some embodiments, the GeneSetScore (Progressively up in memory differentiation) is calculated by taking the mean log normalized gene expression value of all of the genes in the gene set.

As used herein, the term “GeneSetScore (Up TEM vs. Down TN)” of a cell refers to a score that reflects the degree at which the cell shows an effector memory T cell (TEM) phenotype vs. a naïve T cell (TN) phenotype. A higher GeneSetScore (Up TEM vs. Down TN) indicates an increasing TEM phenotype, whereas a lower GeneSetScore (Up TEM vs. Down TN) indicates an increasing TN phenotype. In some embodiments, the GeneSetScore (Up TEM vs. Down TN) is determined by measuring the expression of one or more genes that are up-regulated in TEM cells and/or down-regulated in TN cells, for example, one or more genes selected from the group consisting of MY05A, MXD4, STK3, S1PR5, GLCCI1, CCR3, SOX13, KRTAP5-2, PEA15, PARP8, RNF166, UEVLD, LIMK1, SLC6A6, SV2A, KPNA2, OSBPL7, ST7, GGA2, PI4K2A, CD68, ZAK, RORA, TGFB1, DNAJC1, JOSD1, ZFYVE28, LRP8, OSBPL3, CMIP, NAT13, TGFB1, ANTXR2, NR4A3, RDX, ADCY9, CHN1, CD300A, SCD5, PTPN22, LGALS1, RASGEFIA, GCNT1, GLUL, ABCA2, CLDND1, PAM, CLCF1, MXRA7, CLSTN3, ACOT9, METRNL, BMPRIA, LRIG1, APOBEC3G, CACNB3, RNF19A, RAB27A, FADS3, ACTN4, TBKBP1, FAM53B, MAN1A1, FAM38A, GRLF1, B4GALT5, WIPI1, DUSP2, ANXA5, AHNAK, CLIC1, MAP3K5, ST8SIA1, TARP, ADAM8, MATK, SLFN12L, PIK3R3, FAM46C, ANXA2P2, CTNNA1, NPC1, SH2D2A, ERN1, YPEL1, TBX21, STOM, PHACTR2, GBP5, ADRB2, PIK3AP1, DUSP10, PTGDR, EOMES, MAF, TPRG1, NBEAL2, NCAPH, SLC4A4, FOSL2, RGS2, TGFBR3, MYOIF, C17orf66, CYTH3, WEE1, PYHIN1, F2R, THBS1, CD58, AUTS2, FAM129A, TNIP3, GZMA, PRR5L, PRDM1, PLXND1, PTPRM, GFPT2, MYBL1, SLAMF7, ZEB2, CST7, CCL5, GZMK, and KLRB1. In some embodiments, the GeneSetScore (Up TEM vs. Down TN) is determined using RNA-seq, for example, single-cell RNA-seq (scRNA-seq), for example, as exemplified of WO/2020/047452 in Example 10 with respect to FIG. 40C, hereby incorporated by reference in its entirety. In some embodiments, the GeneSetScore (Up TEM vs. Down TN) is calculated by taking the mean log normalized gene expression value of all of the genes in the gene set.

In the context of GeneSetScore values (e.g., median GeneSetScore values), when a positive GeneSetScore is reduced by 100%, the value becomes 0. When a negative GeneSetScore is increased by 100%, the value becomes 0. For example, as disclosed in WO/2020/047452, the median GeneSetScore of the Dayl sample is-0.084; the median GeneSetScore of the Day9 sample is 0.035; and the median GeneSetScore of the input sample is-0.1. In WO/2020/047452 in FIG. 39A of, increasing the median GeneSetScore of the input sample by 100% leads to a GeneSetScore value of 0; and increasing the median GeneSetScore of the input sample by 200% leads to a GeneSetScore value of 0.1. In WO/2020/047452 in FIG. 39A, decreasing the median GeneSetScore of the Day9 sample by 100% leads to a GeneSetScore value of 0; and decreasing the median GeneSetScore of the Day9 sample by 200% leads to a GeneSetScore value of −0.035.

As used herein, the term “bead” refers to a discrete particle with a solid surface, ranging in size from approximately 0.1 μm to several millimeters in diameter. Beads may be spherical (for example, microspheres) or have an irregular shape. Beads may comprise a variety of materials including, but not limited to, paramagnetic materials, ceramic, plastic, glass, polystyrene, methylstyrene, acrylic polymers, titanium, latex, Sepharose™, cellulose, nylon and the like. In some embodiments, the beads are relatively uniform, about 4.5 μm in diameter, spherical, superparamagnetic polystyrene beads, for example, coated, for example, covalently coupled, with a mixture of antibodies against CD3 (for example, CD3 epsilon) and CD28. In some embodiments, the beads are Dynabeads®. In some embodiments, both anti-CD3 and anti-CD28 antibodies are coupled to the same bead, mimicking stimulation of T cells by antigen presenting cells. The property of Dynabeads® and the use of Dynabeads® for cell isolation and expansion are well known in the art, for example, see, Neurauter et al., Cell isolation and expansion using Dynabeads, Adv Biochem Eng Biotechnol. 2007; 10⁶:41-73, herein incorporated by reference in its entirety.

The term “multispecific binding molecule” refers to a molecule that specifically binds to at least two antigens and comprise two or more antigen-binding domains. The antigen-binding domains can each independently be an antibody fragment (e.g., scFv, Fab, nanobody), a ligand, or a non-antibody derived binder (e.g., fibronectin, Fynomer, DARPin).

The term “monovalent” as used herein in the context of a multispecific binding molecule, antibody (e.g., bispecific antibody), or antibody fragment refers to a multispecific binding molecule, antibody (e.g., bispecific antibody), or antibody fragment in which there is a single antigen binding domain for each antigen to which the multispecific binding molecule, antibody (e.g., bispecific antibody), or antibody fragment binds.

The term “bivalent” as used herein in the context of a multispecific binding molecule, antibody (e.g., bispecific antibody), or antibody fragment refers to a multispecific binding molecule, antibody (e.g., bispecific antibody), or antibody fragment in which there are two antigen binding domains for each antigen to which the multispecific binding molecule, antibody (e.g., bispecific antibody), or antibody fragment binds.

The term “Fc silent” refers to an Fc domain that has been modified to have minimal interaction with effector cells. Silenced effector functions may be obtained by mutation in the Fc region of the antibodies and have been described in the art, such as, but not limited to, LALA and N297A (Strohl, W., 2009, Curr. Opin. Biotechnol. vol. 20 (6): 685−691); and D265A (Baudino et al., 2008, J. Immunol. 181:6664-69) see also Heusser et al., WO2012065950. Examples of Fc silencing mutations include the LALA mutant comprising L234A and L235A mutation in the IgG1 Fc amino acid sequence, DAPA (D265A, P329A) (see, e.g., U.S. Pat. No. 6,737,056), N297A, DANAPA (D265A, N297A, and P329A), and/or LALADANAPS (L234A, L235A, D265A, N297A and P331S).

The term “CD3/TCR complex” refers to a complex on the T-cell surface comprising a TCR including a TCR alpha and TCR beta chain; CD3 including one CD3 gamma chain, one CD3 delta chain, and two CD3 epsilon chains; and a zeta domain. UniProt accession numbers P01848 (TCR alpha, constant domain), P01850 (TCR beta, constant domain 1), A0A5B9 (TCR beta, constant domain 2), P09693 (CD3 gamma), P04234 (CD3 delta), P07766 (CD3 epsilon) provide exemplary human sequences for these chains, with the exception of the zeta chain, responsible for intracellular signaling, which is discussed in further detail below. Further relevant accession numbers include A0A075B662 (murine TCR alpha, constant domain), A0A0A6YWV4 and/or A0A075B5J3 (murine TCR beta, constant domain 1), A0A075B5J4 (murine TCR beta, constant domain 2), P11942 (murine CD3 gamma), P04235 (murine CD3 delta), P22646 (murine CD3 epsilon).

The term “CD28” refers to a T-cell specific glycoprotein CD28, also referred to as Tp44, as well as all alternate names thereof, which functions as a costimulatory molecule. UniProt accession number P10747 provides exemplary human CD28 amino acid sequences (see also HGNC: 1653, Entrez Gene: 940, Ensembl: ENSG00000178562, and OMIM: 186760). Further relevant CD28 sequences include UniProt accession number P21041 (murine CD28).

The term “CD2” refers to T-cell surface antigen T11/Leu-5/CD2, lymphocyte function antigen 2, T11, or erythrocyte/rosette/LFA-3 receptor, as well as alternate names thereof, which functions as a growth factor receptor. UniProt accession number P06729 provides exemplary human CD2 amino acid sequences (see also HGNC: 1639, Entrez Gene: 914, Ensembl: ENSG00000116824, and OMIM: 186990). Further relevant CD2 sequences include UniProt accession number P08920 (murine CD2).

As used herein, the term “nanomatrix” refers to a nanostructure comprising a matrix of mobile polymer chains. The nanomatrix is 1 to 500 nm, for example, 10 to 200 nm, in size. In some embodiments, the matrix of mobile polymer chains is attached to one or more agonists which provide activation signals to T cells, for example, agonist anti-CD3 and/or anti-CD28 antibodies. In some embodiments, the nanomatrix comprises a colloidal polymeric nanomatrix attached, for example, covalently attached, to an agonist of one or more stimulatory molecules and/or an agonist of one or more costimulatory molecules. In some embodiments, the agonist of one or more stimulatory molecules is a CD3 agonist (for example, an anti-CD3 agonistic antibody). In some embodiments, the agonist of one or more costimulatory molecules is a CD28 agonist (for example, an anti-CD28 agonistic antibody). In some embodiments, the nanomatrix is characterized by the absence of a solid surface, for example, as the attachment point for the agonists, such as anti-CD3 and/or anti-CD28 antibodies. In some embodiments, the nanomatrix is the nanomatrix disclosed in WO2014/048920A1 or as given in the MACS® GMP T Cell TransAct™ kit from Miltenyi Biotcc GmbH, herein incorporated by reference in their entirety. MACS® GMP T Cell TransAct™ consists of a colloidal polymeric nanomatrix covalently attached to humanized recombinant agonist antibodies against human CD3 and CD28.

Various embodiments of the compositions and methods herein are described in further detail below. Additional definitions are set out throughout the specification.

### DESCRIPTION

Provided herein are methods of manufacturing immune effector cells (for example, T cells or NK cells) engineered to express a CAR, for example, a CAR described herein, compositions comprising such cells, and methods of using such cells for treating a disease, such as an autoimmune disease or disorder, e.g., lupus (e.g., systemic lupus erythematosus (SLE), e.g., severe refractory systemic lupus erythematosus (srSLE), or lupus nephritis), systemic sclerosis (e.g., rapidly progressing systemic sclerosis (SSc) with significant lung involvement (e.g. as for autoHSCT)), idiopathic inflammatory myopathies (e.g., polymyositis, dermatomyositis, anti-synthetase syndrome, immune-mediated necrotizing myopathy, inclusion body myositis, overlap myositis, cancer associated myositis, e.g. anti-synthetase syndrome with ILD), vasculitis (e.g., ANCA-associated vasculitis), severe refractory Sjögren's, severe refractory neuroimmune disease (e.g. myasthenia gravis (MG), neuromyelitis optica (NMO), MOG associated disease (MOGAD), multiple sclerosis (MS)), severe refractory rheumatoid arthritis, antibody mediated neuroimmune diseases (e.g., AChR+ and MuSK+myasthenia gravis (MG), AQP4+ neuromyelitis optica (NMO), MOGAD (anti-MOG associated disease), NMDAR+encephalitis, or antibody-associated neurological paraneoplastic diseases), Addison's disease, Goodpasture's syndrome, thyrotoxicosis, chronic active hepatitis, relapsing polychondritis, pemphigus vulgaris, or amyotrophic lateral sclerosis, in a subject. In some embodiments, the methods disclosed herein may manufacture immune effector cells engineered to express a CAR in less than 24 hours. Without wishing to be bound by theory, the methods provided herein preserve the undifferentiated phenotype of T cells, such as naïve T cells, during the manufacturing process. These CAR-expressing cells with an undifferentiated phenotype may persist longer and/or expand better in vivo after infusion. In some embodiments, CART cells produced by the manufacturing methods provided herein comprise a higher percentage of stem cell memory T cells, compared to CART cells produced by the traditional manufacturing process, e.g., as measured using scRNA-seq. In some embodiments, CART cells produced by the manufacturing methods provided herein comprise a higher percentage of effector T cells, compared to CART cells produced by the traditional manufacturing process, e.g., as measured using scRNA-seq. In some embodiments, CART cells produced by the manufacturing methods provided herein better preserve the stemness of T cells, compared to CART cells produced by the traditional manufacturing process. In some embodiments, CART cells produced by the manufacturing methods provided herein show a lower level of hypoxia, compared to CART cells produced by the traditional manufacturing process, e.g., as measured using scRNA-seq (. In some embodiments, CART cells produced by the manufacturing methods provided herein show a lower level of autophagy, compared to CART cells produced by the traditional manufacturing process.

In some embodiments, the methods disclosed herein do not involve using a bead, such as Dynabeads® (for example, CD3/CD28 Dynabeads®), and do not involve a de-beading step. In some embodiments, the CART cells manufactured by the methods disclosed herein may be administered to a subject with minimal ex vivo expansion, for example, less than 1 day, less than 12 hours, less than 8 hours, less than 6 hours, less than 4 hours, less than 3 hours, less than 2 hours, less than 1 hour, or no ex vivo expansion. Accordingly, the methods described herein provide a fast manufacturing process of making improved CAR-expressing cell products for use in treating a disease in a subject. Furthermore, the present invention provides CAR compositions and their use in medicaments or methods for treating, among other diseases, autoimmune disease or disorder, e.g., lupus (e.g., systemic lupus erythematosus (SLE), e.g., severe refractory systemic lupus erythematosus (srSLE), or lupus nephritis), systemic sclerosis (e.g., rapidly progressing systemic sclerosis (SSc) with significant lung involvement (e.g. as for autoHSCT)), idiopathic inflammatory myopathies (e.g., polymyositis, dermatomyositis, anti-synthetase syndrome, immune-mediated necrotizing myopathy, inclusion body myositis, overlap myositis, cancer associated myositis, e.g. anti-synthetase syndrome with ILD), vasculitis (e.g., ANCA-associated vasculitis), severe refractory Sjögren's, severe refractory neuroimmune disease (e.g. myasthenia gravis (MG), neuromyelitis optica (NMO), MOG associated disease (MOGAD), multiple sclerosis (MS)), severe refractory rheumatoid arthritis, antibody mediated neuroimmune diseases (e.g., AChR+ and MuSK+myasthenia gravis (MG), AQP4+ neuromyelitis optica (NMO), MOGAD (anti-MOG associated disease), NMDAR+encephalitis, or antibody-associated neurological paraneoplastic diseases), Addison's disease, Goodpasture's syndrome, thyrotoxicosis, chronic active hepatitis, relapsing polychondritis, pemphigus vulgaris, or amyotrophic lateral sclerosis.

### Activation Process

In some embodiments, the present disclosure provides methods of making a population of cells (for example, T cells) that express a chimeric antigen receptor (CAR) comprising: (i) contacting a population of cells (for example, T cells, for example, T cells isolated from a frozen or fresh leukapheresis product from a subject having an autoimmune disease or disorder, e.g., lupus (e.g., systemic lupus erythematosus (SLE), e.g., severe refractory systemic lupus erythematosus (srSLE), or lupus nephritis), systemic sclerosis (e.g., rapidly progressing systemic sclerosis (SSc) with significant lung involvement (e.g. as for autoHSCT)), idiopathic inflammatory myopathies (e.g., polymyositis, dermatomyositis, anti-synthetase syndrome, immune-mediated necrotizing myopathy, inclusion body myositis, overlap myositis, cancer associated myositis, e.g. anti-synthetase syndrome with ILD), vasculitis (e.g., ANCA-associated vasculitis), severe refractory Sjögren's, severe refractory neuroimmune disease (e.g. myasthenia gravis (MG), neuromyelitis optica (NMO), MOG associated disease (MOGAD), multiple sclerosis (MS)), severe refractory rheumatoid arthritis, antibody mediated neuroimmune diseases (e.g., AChR+ and MuSK+ myasthenia gravis (MG), AQP4+ neuromyelitis optica (NMO), MOGAD (anti-MOG associated disease), NMDAR+ encephalitis, or antibody-associated neurological paraneoplastic diseases), Addison's disease, Goodpasture's syndrome, thyrotoxicosis, chronic active hepatitis, relapsing polychondritis, pemphigus vulgaris, or amyotrophic lateral sclerosis) with (A) an agent that stimulates a CD3/TCR complex and/or (B) an agent that stimulates a costimulatory molecule and/or growth factor receptor on the surface of the cells; (ii) contacting the population of cells (for example, T cells) with a nucleic acid molecule (for example, a DNA or RNA molecule) encoding the CAR, thereby providing a population of cells (for example, T cells) comprising the nucleic acid molecule, and (iii) harvesting the population of cells (for example, T cells) for storage (for example, reformulating the population of cells in cryopreservation media) or administration, wherein: (a) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and step (iii) is performed no later than 26 hours after the beginning of step (i), for example, no later than 22, 23, or 24 hours after the beginning of step (i), for example, no later than 24 hours after the beginning of step (i); (b) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and step (iii) is performed no later than 30, 36, or 48 hours after the beginning of step (ii), for example, no later than 22, 23, 24, 25, 26, 27, 28, 29, 30, 31, 32, 33, 34, 35, 36, 37, 38, 39, 40, 41, 42, 43, 44, 45, 46, 47, or 48 hours after the beginning of step (ii); or (c) the population of cells from step (iii) are not expanded, or expanded by no more than 5, 10, 15, 20, 25, 30, 35, or 40%, for example, no more than 10%, for example, as assessed by the number of living cells, compared to the population of cells at the beginning of step (i). In some embodiments, the nucleic acid molecule in step (ii) is a DNA molecule. In some embodiments, the nucleic acid molecule in step (ii) is an RNA molecule. In some embodiments, the nucleic acid molecule in step (ii) is on a viral vector, for example, a viral vector chosen from a lentivirus vector, an adenoviral vector, or a retrovirus vector. In some embodiments, the nucleic acid molecule in step (ii) is on a non-viral vector. In some embodiments, the nucleic acid molecule in step (ii) is on a plasmid. In some embodiments, the nucleic acid molecule in step (ii) is not on any vector. In some embodiments, step (ii) comprises transducing the population of cells (for example, T cells) a viral vector comprising a nucleic acid molecule encoding the CAR.

In some embodiments, the population of cells (for example, T cells) is collected from an apheresis sample (for example, a leukapheresis sample) from a subject having an autoimmune disease or disorder, e.g., lupus (e.g., systemic lupus erythematosus (SLE), e.g., severe refractory systemic lupus erythematosus (srSLE), or lupus nephritis), systemic sclerosis (e.g., rapidly progressing systemic sclerosis (SSc) with significant lung involvement (e.g. as for autoHSCT)), idiopathic inflammatory myopathies (e.g., polymyositis, dermatomyositis, anti-synthetase syndrome, immune-mediated necrotizing myopathy, inclusion body myositis, overlap myositis, cancer associated myositis, e.g. anti-synthetase syndrome with ILD), vasculitis (e.g., ANCA-associated vasculitis), severe refractory Sjögren's, severe refractory neuroimmune disease (e.g. myasthenia gravis (MG), neuromyelitis optica (NMO), MOG associated disease (MOGAD), multiple sclerosis (MS)), severe refractory rheumatoid arthritis, antibody mediated neuroimmune diseases (e.g., AChR+ and MuSK+ myasthenia gravis (MG), AQP4+ neuromyelitis optica (NMO), MOGAD (anti-MOG associated disease), NMDAR+ encephalitis, or antibody-associated neurological paraneoplastic diseases), Addison's disease, Goodpasture's syndrome, thyrotoxicosis, chronic active hepatitis, relapsing polychondritis, pemphigus vulgaris, or amyotrophic lateral sclerosis.

In some embodiments, the apheresis sample (for example, a leukapheresis sample) is collected from the subject and shipped as a frozen sample (for example, a cryopreserved sample) to a cell manufacturing facility. Then the frozen apheresis sample is thawed, and T cells (for example, CD4+ T cells and/or CD8+ T cells) are selected from the apheresis sample, for example, using a cell sorting machine (for example, a CliniMACS® Prodigy® device). The selected T cells (for example, CD4+ T cells and/or CD8+ T cells) are then seeded for CART manufacturing using the activation process described herein. In some embodiments, the selected T cells (for example, CD4+ T cells and/or CD8+ T cells) undergo one or more rounds of freeze-thaw before being seeded for CART manufacturing.

In some embodiments, the apheresis sample (for example, a leukapheresis sample) is collected from the subject and shipped as a fresh product (for example, a product that is not frozen) to a cell manufacturing facility. T cells (for example, CD4+ T cells and/or CD8+ T cells) are selected from the apheresis sample, for example, using a cell sorting machine (for example, a CliniMACS® Prodigy® device). The selected T cells (for example, CD4+ T cells and/or CD8+ T cells) are then seeded for CART manufacturing using the activation process described herein. In some embodiments, the selected T cells (for example, CD4+ T cells and/or CD8+ T cells) undergo one or more rounds of freeze-thaw before being seeded for CART manufacturing.

In some embodiments, the apheresis sample (for example, a leukapheresis sample) is collected from the subject. T cells (for example, CD4+ T cells and/or CD8+ T cells) are selected from the apheresis sample, for example, using a cell sorting machine (for example, a CliniMACS® Prodigy® device). The selected T cells (for example, CD4+ T cells and/or CD8+ T cells) are then shipped as a frozen sample (for example, a cryopreserved sample) to a cell manufacturing facility. The selected T cells (for example, CD4+ T cells and/or CD8+ T cells) are later thawed and seeded for CART manufacturing using the activation process described herein.

In some embodiments, cells (for example, T cells) are contacted with anti-CD3 and anti-CD28 antibodies for, for example, 12 hours, followed by transduction with a vector (for example, a lentiviral vector) encoding a CAR. 24 hours after culture initiation, the cells are washed and formulated for storage or administration.

Without wishing to be bound by theory, brief CD3 and CD28 stimulation may promote efficient transduction of self-renewing T cells. Compared to traditional CART manufacturing approaches, the activation process provided herein does not involve prolonged ex vivo expansion. Similar to the cytokine process, the activation process provided herein also preserves undifferentiated T cells during CART manufacturing.

In some embodiments, the population of cells is contacted with a multispecific binding molecule, e.g., as described herein.

In some embodiments, the population of cells is contacted with (A) an agent that stimulates a CD3/TCR complex and/or (B) an agent that stimulates a costimulatory molecule and/or growth factor receptor on the surface of the cells.

In some embodiments, the agent that stimulates a CD3/TCR complex is an agent that stimulates CD3. In some embodiments, the agent that stimulates a costimulatory molecule and/or growth factor receptor is an agent that stimulates CD28, ICOS, CD27, HVEM, LIGHT, CD40, 4-1BB, OX40, DR3, GITR, CD30, TIM1, CD2, CD226, or any combination thereof. In some embodiments, the agent that stimulates a costimulatory molecule and/or growth factor receptor is an agent that stimulates CD28. In some embodiments, the agent that stimulates a CD3/TCR complex is chosen from an antibody (for example, a single-domain antibody (for example, a heavy chain variable domain antibody), a peptibody, a Fab fragment, or a scFv), a small molecule, or a ligand (for example, a naturally existing, recombinant, or chimeric ligand). In some embodiments, the agent that stimulates a costimulatory molecule and/or growth factor receptor is chosen from an antibody (for example, a single-domain antibody (for example, a heavy chain variable domain antibody), a peptibody, a Fab fragment, or a scFv), a small molecule, or a ligand (for example, a naturally existing, recombinant, or chimeric ligand). In some embodiments, the agent that stimulates a CD3/TCR complex does not comprise a bead. In some embodiments, the agent that stimulates a costimulatory molecule and/or growth factor receptor does not comprise a bead. In some embodiments, the agent that stimulates a CD3/TCR complex comprises an anti-CD3 antibody. In some embodiments, the agent that stimulates a costimulatory molecule and/or growth factor receptor comprises an anti-CD28 antibody. In some embodiments, the agent that stimulates a CD3/TCR complex comprises an anti-CD3 antibody covalently attached to a colloidal polymeric nanomatrix. In some embodiments, the agent that stimulates CD3 comprises one or more of a CD3 or TCR antigen binding domain, such as but not limited to an anti-CD3 or anti-TCR antibody or an antibody fragment comprising one or more CDRs, heavy chain, and/or light chain thereof-such as but not limited to an anti-CD3 or anti-TCR antibody provided in Table 27 of WO/2021/173985, hereby incorporated by reference in its entirety. In some embodiments, the agent that stimulates a costimulatory molecule and/or growth factor receptor comprises an anti-CD28 antibody covalently attached to a colloidal polymeric nanomatrix. In some embodiments, the agent that stimulates a costimulatory molecule and/or growth factor receptor is an agent that stimulates CD28, ICOS, CD27, CD25, 4-1BB, IL6RA, IL6Rβ, or CD2. In some embodiments, the agent that stimulates a costimulatory molecule and/or growth factor receptor comprises one or more of a CD28, ICOS, CD27, CD25, 4-1BB, IL6Rβ, and/or CD2 antigen binding domain, such as but not limited to an anti-CD28, anti-ICOS, anti-CD27, anti-CD25, anti-4-1BB, anti-IL6RA, anti-IL6Rβ, or anti-CD2 antibody or an antibody fragment comprising one or more CDRs, heavy chain, and/or light chain thereof-such as but not limited to an anti-CD28, anti-ICOS, anti-CD27, anti-CD25, anti-4-1BB, anti-IL6RA, anti-IL6Rβ, or anti-CD2 antibody provided in Table 27 of WO/2021/173985, hereby incorporated by reference in its entirety. In some embodiments, the agent that stimulates a CD3/TCR complex and the agent that stimulates a costimulatory molecule and/or growth factor receptor comprise T Cell TransAct™. In some embodiments, the agent that stimulates a CD3/TCR complex and the agent that stimulates a costimulatory molecule and/or growth factor receptor are comprised in a multispecific binding molecule. In some embodiments, the multispecific binding molecule comprises a CD3 antigen binding domain and a CD28 or CD2 antigen-binding domain. In some embodiments, the multispecific binding molecules comprise one or more heavy and/or light chains-such as but not limited to the heavy and/or light chains provided in Table 28 of WO/2021/173985, hereby incorporated by reference in its entirety. In some embodiments, the multispecific binding molecule comprises a bispecific antibody. In some embodiments, the bispecific antibody is configured in any one of the schema provided in FIG. 50A of WO/2021/173985, hereby incorporated by reference in its entirety. In some embodiments, the bispecific antibody is monovalent or bivalent. In some embodiments, the bispecific antibody comprises an Fc region. In some embodiments, the Fc region of the bispecific antibody is silenced. In some embodiments, the multispecific binding molecule comprises a plurality of bispecific antibodies. In some embodiments, one or more of the plurality of bispecific antibodies is monovalent. In some embodiments, one or more of the plurality of bispecific antibodies comprises an Fc region. In some embodiments, the Fc region of the one or more of the plurality of bispecific antibodies is silenced. In some embodiments, one or more of the plurality of bispecific antibodies are conjugated together into a multimer. In some embodiments, the multimer is configured in any one of the schema provided in FIG. 50B of WO/2021/173985, hereby incorporated by reference in its entirety.

In some embodiments, the matrix comprises or consists of a polymeric, for example, biodegradable or biocompatible inert material, for example, which is non-toxic to cells. In some embodiments, the matrix is composed of hydrophilic polymer chains, which obtain maximal mobility in aqueous solution due to hydration of the chains. In some embodiments, the mobile matrix may be of collagen, purified proteins, purified peptides, polysaccharides, glycosaminoglycans, or extracellular matrix compositions. A polysaccharide may include for example, cellulose ethers, starch, gum arabic, agarose, dextran, chitosan, hyaluronic acid, pectins, xanthan, guar gum, or alginate. Other polymers may include polyesters, polyethers, polyacrylates, polyacrylamides, polyamines, polyethylene imines, polyquaternium polymers, polyphosphazenes, polyvinylalcohols, polyvinylacetates, polyvinylpyrrolidones, block copolymers, or polyurethanes. In some embodiments, the mobile matrix is a polymer of dextran.

In some embodiments, the population of cells is contacted with a nucleic acid molecule encoding a CAR. In some embodiments, the population of cells is transduced with a DNA molecule encoding a CAR.

In some embodiments, contacting the population of cells with the nucleic acid molecule encoding the CAR occurs simultaneously with contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or 0.5 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 20 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 19 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 18 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 17 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 16 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 15 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 14 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 14 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 13 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 12 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 11 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 10 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 9 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 8 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 7 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 6 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 5 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 4 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 3 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 2 hours after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 1 hour after the beginning of contacting the population of cells with the agent that stimulates a CD3/TCR complex and/or the agent that stimulates a costimulatory molecule and/or growth factor receptor on the surface of the cells described above. In some embodiments, contacting the population of cells with the nucleic acid molecule encoding the CAR occurs no later than 30 minutes after the beginning of contacting the population of cells with the agent that stimulates a CD3/TCR complex and/or the agent that stimulates a costimulatory molecule and/or growth factor receptor on the surface of the cells described above.

In some embodiments, the population of cells is harvested for storage or administration.

In some embodiments, the population of cells is harvested for storage or administration no later than 72, 60, 48, 36, 32, 31, 30, 29, 28, 27, 26, 25, 24, 23, 22, 21, 20, 19, or 18 hours after the beginning of contacting the population of cells with the agent that stimulates a CD3/TCR complex and/or the agent that stimulates a costimulatory molecule and/or growth factor receptor on the surface of the cells described above. In some embodiments, the population of cells is harvested for storage or administration no later than 26 hours after the beginning of contacting the population of cells with the agent that stimulates a CD3/TCR complex and/or the agent that stimulates a costimulatory molecule and/or growth factor receptor on the surface of the cells described above. In some embodiments, the population of cells is harvested for storage or administration no later than 25 hours after the beginning of contacting the population of cells with the agent that stimulates a CD3/TCR complex and/or the agent that stimulates a costimulatory molecule and/or growth factor receptor on the surface of the cells described above. In some embodiments, the population of cells is harvested for storage or administration no later than 24 hours after the beginning of contacting the population of cells with the agent that stimulates a CD3/TCR complex and/or the agent that stimulates a costimulatory molecule and/or growth factor receptor on the surface of the cells described above. In some embodiments, the population of cells is harvested for storage or administration no later than 23 hours after the beginning of contacting the population of cells with the agent that stimulates a CD3/TCR complex and/or the agent that stimulates a costimulatory molecule and/or growth factor receptor on the surface of the cells described above. In some embodiments, the population of cells is harvested for storage or administration no later than 22 hours after the beginning of contacting the population of cells with the agent that stimulates a CD3/TCR complex and/or the agent that stimulates a costimulatory molecule and/or growth factor receptor on the surface of the cells described above.

In some embodiments, the population of cells is not expanded ex vivo.

In some embodiments, the population of cells is expanded by no more than 5, 6, 7, 8, 9, 10, 11, 12, 13, 14, 15, 16, 17, 18, 19, 20, 25, 30, 35, 40, 45, 50, 55, or 60%, for example, as assessed by the number of living cells, compared to the population of cells before it is contacted with the agent that stimulates a CD3/TCR complex and/or the agent that stimulates a costimulatory molecule and/or growth factor receptor on the surface of the cells described above. In some embodiments, the population of cells is expanded by no more than 5%, for example, as assessed by the number of living cells, compared to the population of cells before it is contacted with the agent that stimulates a CD3/TCR complex and/or the agent that stimulates a costimulatory molecule and/or growth factor receptor on the surface of the cells described above. In some embodiments, the population of cells is expanded by no more than 10%, for example, as assessed by the number of living cells, compared to the population of cells before it is contacted with the agent that stimulates a CD3/TCR complex and/or the agent that stimulates a costimulatory molecule and/or growth factor receptor on the surface of the cells described above. In some embodiments, the population of cells is expanded by no more than 15%, for example, as assessed by the number of living cells, compared to the population of cells before it is contacted with the agent that stimulates a CD3/TCR complex and/or the agent that stimulates a costimulatory molecule and/or growth factor receptor on the surface of the cells described above. In some embodiments, the population of cells is expanded by no more than 20%, for example, as assessed by the number of living cells, compared to the population of cells before it is contacted with the agent that stimulates a CD3/TCR complex and/or the agent that stimulates a costimulatory molecule and/or growth factor receptor on the surface of the cells described above. In some embodiments, the population of cells is expanded by no more than 25%, for example, as assessed by the number of living cells, compared to the population of cells before it is contacted with the agent that stimulates a CD3/TCR complex and/or the agent that stimulates a costimulatory molecule and/or growth factor receptor on the surface of the cells described above. In some embodiments, the population of cells is expanded by no more than 30%, for example, as assessed by the number of living cells, compared to the population of cells before it is contacted with the agent that stimulates a CD3/TCR complex and/or the agent that stimulates a costimulatory molecule and/or growth factor receptor on the surface of the cells described above. In some embodiments, the population of cells is expanded by no more than 35%, for example, as assessed by the number of living cells, compared to the population of cells before it is contacted with the agent that stimulates a CD3/TCR complex and/or the agent that stimulates a costimulatory molecule and/or growth factor receptor on the surface of the cells described above. In some embodiments, the population of cells is expanded by no more than 40%, for example, as assessed by the number of living cells, compared to the population of cells before it is contacted with the agent that stimulates a CD3/TCR complex and/or the agent that stimulates a costimulatory molecule and/or growth factor receptor on the surface of the cells described above.

In some embodiments, the population of cells is expanded by no more than 1, 1.5, 2, 2.5, 3, 3.5, 4, 4.5, 5, 6, 7, 8, 9, 10, 11, 12, 16, 20, 24, 36, or 48 hours, for example, as assessed by the number of living cells, compared to the population of cells before it is contacted with the one or more cytokines described above.

In some embodiments, the activation process is conducted in serum free cell media. In some embodiments, the activation process is conducted in cell media comprising one or more cytokines chosen from: IL-2, IL-15 (for example, hetIL-15 (IL15/sIL-15Ra)), or IL-6 (for example, IL-6/sIL-6Ra). In some embodiments, hetIL-15 comprises the amino acid sequence of NWVNVISDLKKIEDLIQSMHIDATLYTESDVHPSCKVTAMKCFLLELQVISLESGDASIH DTVENLIILANNSLSSNGNVTESGCKECEELEEKNIKEFLQSFVHIVQMFINTSITCPPPM SVEHADIWVKSYSLYSRERYICNSGFKRKAGTSSLTECVLNKATNVAHWTTPSLKCIR DPALVHQRPAPPSTVTTAGVTPQPESLSPSGKEPAASSPSSNNTAATTAAIVPGSQLMPS KSPSTGTTEISSHESSHGTPSQTTAKNWELTASASHQPPGVYPQG (SEQ ID NO: 309). In some embodiments, hetIL-15 comprises an amino acid sequence having at least about 70, 75, 80, 85, 90, 95, or 99% identity to SEQ ID NO: 309. In some embodiments, the activation process is conducted in cell media comprising a LSD1 inhibitor. In some embodiments, the activation process is conducted in cell media comprising a MALT1 inhibitor. In some embodiments, the serum free cell media comprises a serum replacement. In some embodiments, the serum replacement is CTS™ Immune Cell Serum Replacement (ICSR). In some embodiments, the level of ICSR can be, for example, up to 5%, for example, about 1%, 2%, 3%, 4%, or 5%. Without wishing to be bound by theory, using cell media, for example, Rapid Media shown in Table 21 or Table 25, comprising ICSR, for example, 2% ICSR, may improve cell viability during a manufacture process described herein.

In some embodiments, the present disclosure provides methods of making a population of cells (for example, T cells) that express a chimeric antigen receptor (CAR) comprising: (a) providing an apheresis sample (for example, a fresh or cryopreserved leukapheresis sample) collected from a subject, e.g., lupus (e.g., systemic lupus erythematosus (SLE), e.g., severe refractory systemic lupus erythematosus (srSLE), or lupus nephritis), systemic sclerosis (e.g., rapidly progressing systemic sclerosis (SSc) with significant lung involvement (e.g. as for autoHSCT)), idiopathic inflammatory myopathies (e.g., polymyositis, dermatomyositis, anti-synthetase syndrome, immune-mediated necrotizing myopathy, inclusion body myositis, overlap myositis, cancer associated myositis, e.g. anti-synthetase syndrome with ILD), vasculitis (e.g., ANCA-associated vasculitis), severe refractory Sjögren's, severe refractory neuroimmune disease (e.g. myasthenia gravis (MG), neuromyelitis optica (NMO), MOG associated disease (MOGAD), multiple sclerosis (MS)), severe refractory rheumatoid arthritis, antibody mediated neuroimmune diseases (e.g., AChR+ and MuSK+myasthenia gravis (MG), AQP4+ neuromyelitis optica (NMO), MOGAD (anti-MOG associated disease), NMDAR+encephalitis, or antibody-associated neurological paraneoplastic diseases), Addison's disease, Goodpasture's syndrome, thyrotoxicosis, chronic active hepatitis, relapsing polychondritis, pemphigus vulgaris, or amyotrophic lateral sclerosis; (b) selecting T cells from the apheresis sample (for example, using negative selection, positive selection, or selection without beads); (c) seeding isolated T cells at, for example, 1×10⁶ to 1×10⁷ cells/mL; (d) contacting T cells with an agent that stimulates T cells, for example, an agent that stimulates a CD3/TCR complex and/or an agent that stimulates a costimulatory molecule and/or growth factor receptor on the surface of the cells (for example, contacting T cells with anti-CD3 and/or anti-CD28 antibody, for example, contacting T cells with TransAct); (e) contacting T cells with a nucleic acid molecule (for example, a DNA or RNA molecule) encoding the CAR (for example, contacting T cells with a virus comprising a nucleic acid molecule encoding the CAR) for, for example, 6-48 hours, for example, 20-28 hours; and (f) washing and harvesting T cells for storage (for example, reformulating T cells in cryopreservation media) or administration. In some embodiments, step (f) is performed no later than 30, 36, or 48 hours after the beginning of step (d) or (e), for example, no later than 22, 23, 24, 25, 26, 27, 28, 29, 30, 31, 32, 33, 34, 35, 36, 37, 38, 39, 40, 41, 42, 43, 44, 45, 46, 47, or 48 hours after the beginning of step (d) or (e).

In some embodiments of the aforementioned methods, the methods are performed in a closed system. In some embodiments, T cell separation, activation, transduction, incubation, and washing are all performed in a closed system. In some embodiments of the aforementioned methods, the methods are performed in separate devices. In some embodiments, T cell separation, activation and transduction, incubation, and washing are performed in separate devices.

In some embodiments of the aforementioned methods, the methods further comprise adding an adjuvant or a transduction enhancement reagent in the cell culture medium to enhance transduction efficiency. In some embodiments, the adjuvant or transduction enhancement reagent comprises a cationic polymer. In some embodiments, the adjuvant or transduction enhancement reagent is chosen from: LentiBOOST™ (Sirion Biotech), vectofusin-1, F108 (Poloxamer 338 or Pluronic® F-38), protamine sulfate, hexadimethrine bromide (Polybrene), PEA, Pluronic F68, Pluronic F127, Synperonic or LentiTrans™. In some embodiments, the transduction enhancement reagent is LentiBOOST™ (Sirion Biotech). In some embodiments, the transduction enhancement reagent is F108 (Poloxamer 338 or Pluronic® F-38)

In some embodiments of the aforementioned methods, the transducing the population of cells (for example, T cells) with a viral vector comprises subjecting the population of cells and viral vector to a centrifugal force under conditions such that transduction efficiency is enhanced. In an embodiment, the cells are transduced by spinoculation.

In some embodiments of the aforementioned methods, cells (e.g., T cells) are activated and transduced in a cell culture flask comprising a gas-permeable membrane at the base that supports large media volumes without substantially compromising gas exchange. In some embodiments, cell growth is achieved by providing access, e.g., substantially uninterrupted access, to nutrients through convection.

### Multispecific Binding Molecule

A method of making CAR-expressing cells may make use of an agent that stimulates a CD3/TCR complex and the agent that stimulates a costimulatory molecule and/or growth factor receptor. In some embodiments, the agent that stimulates a CD3/TCR complex and the agent that stimulates a costimulatory molecule and/or growth factor receptor are comprised in a multispecific binding molecule. In some embodiments, a multispecific binding molecule of the present disclosure is a multispecific binding molecule described in any of WO 2021/173985 (incorporated by reference in its entirety), WO 2022/040586 (incorporated by reference in its entirety), and PCT/IB2022/057799 (incorporated by reference in its entirety).

In some embodiments, the multispecific binding molecule comprises a first binding domain and a second binding domain. For instance, the first binding domain may be an anti-CD3 binding domain and the second binding domain may be a costimulatory molecule binding domain, or the first binding domain may be a costimulatory molecule binding domain and the second binding domain may be an anti-CD3 binding domain. In some embodiments, the costimulatory molecule binding domain binds to CD2, CD28, CD25, CD27, IL6Rb, ICOS, or 41BB. Non-limiting examples of such binding domains, as noted above, are provided, for example in Table 27 of WO 2021/173985, the contents of which are hereby incorporated by reference in their entirety. In some embodiments, the multispecific binding molecule is configured in any one of the schema provided in FIGS. 50A-50B, FIGS. 51A-51B, and FIGS. 61A-61B, and FIGS. 63A-63B of WO 2021/173985 (incorporated by reference in its entirety).

In some embodiments, the multispecific binding molecule comprises a CD3 antigen binding domain and a CD28 or CD2 antigen binding domain. In some embodiments, the CD3 antigen binding domain is an anti-CD3 antibody, optionally the anti-CD3 (1), anti-CD3 (2), anti-CD3 (3), or anti-CD3 (4) provided in Table 27 of WO 2021/173985 (the contents of which are hereby incorporated by reference in their entirety), or an antibody fragment comprising one or more CDRs, VH, and/or VL thereof. In some embodiments, the CD28 antigen binding domain is an anti-CD28 antibody, optionally the anti-CD28 (1) or anti-CD28 (2) provided in Table 27 of WO 2021/173985 (the contents of which are hereby incorporated by reference in their entirety), or an antibody fragment comprising one or more CDRs, VH, heavy chain, VL, and/or light chain thereof. In some embodiments, the CD2 antigen binding domain is an anti-CD2 antibody, optionally the anti-CD2 (1), provided in Table 27 of WO 2021/173985 (incorporated by reference in their entirety), or an antibody fragment comprising one or more CDRs, VH, heavy chain, VL, and/or light chain thereof. In some embodiments, a multispecific binding molecule described herein comprises a CDR, VH, VL, HC, and/or LC disclosed in Table 27 of WO 2021/173985 (incorporated by reference in its entirety), or sequence having at least 70%, 75%, 80%, 85%, 90%, 95%, 96%, 97%, 98%, or 99% identity thereto.

In some embodiments, the multispecific binding molecule comprises one or more heavy and/or light chains. Non-limiting exemplary heavy and light chain sequences that may be comprised in a multispecific binding molecule described herein are provided in Table 28 of WO 2021/173985 (incorporated by reference in its entirety) or Table 20 of WO 2022/040586 (incorporated by reference in its entirety). In some embodiments, the multispecific binding molecule comprises one or more heavy and/or light chain sequences disclosed in Table 20 of WO 2022/040586 (incorporated by reference in its entirety), or a sequence having at least 70%, 75%, 80%, 85%, 90%, 95%, or 99% identity thereto

In some embodiments, a multispecific binding molecule described herein comprises an Fc region, e.g., wherein the Fc region is Fc silent, e.g., an Fc region described for example in WO 2021/173985 (incorporated by reference in its entirety) or WO 2022/040586 (incorporated by reference in its entirety). In some embodiments, the Fc region comprises a mutation at one or more of (e.g., all of) D265, N297, and P329, numbered according to the Eu numbering system. In some embodiments, the Fc region comprises a mutation at one, two, three or all of positions L234 (e.g. L234A), L235 (e.g. L235A), S267 (e.g. S267K), and P239 (e.g. P329A), numbered according to the Eu numbering system. In some embodiments, the Fc region comprises a mutation at L234 (e.g. L234A), L235 (e.g. L235A), S267 (e.g. S267K), and P239 (e.g. P329A) (LALASKPA), numbered according to the EU numbering system. In some embodiments, the Fc region comprises one or more mutations as described for example in WO 2021/173985 (incorporated by reference in its entirety) or WO 2022/040586 (incorporated by reference in its entirety).

In some embodiments, the multispecific binding molecule comprises (A) an anti-CD3 binding domain, and (B) a costimulatory molecule binding domain (e.g., an anti-CD2 binding domain or an anti-CD28 binding domain). In some embodiments, the anti-CD3 binding domain, e.g., an anti-CD3 scFv, is situated N-terminal of the costimulatory molecule binding domain, e.g., an anti-CD2 Fab or an anti-CD28 Fab. In some embodiments, the anti-CD3 binding domain, e.g., an anti-CD3 scFv, is situated C-terminal of the costimulatory molecule binding domain, e.g., an anti-CD2 Fab or an anti-CD28 Fab.

In some embodiments, an Fc region is situated between the anti-CD3 binding domain and the costimulatory molecule binding domain. In some embodiments, the anti-CD3 binding domain is situated C-terminal of the costimulatory molecule binding domain, wherein an Fc region is situated between the anti-CD3 binding domain and the costimulatory molecule binding domain.

In some embodiments, the multispecific binding molecule comprises a CH2, and the anti-CD3 binding domain is situated N-terminal of the CH2. In some embodiments, the anti-CD3 binding domain is linked to the CH2 by a peptide linker, e.g., a glycine-serine linker, e.g., a (G4S) 4 (SEQ ID NO: 27) linker.

In some embodiments, the multispecific binding molecule further comprises a CL. In some embodiments, the CL is C-terminal of the VL of the costimulatory molecule binding domain. In some embodiments, the CL domain is linked to the CH1, e.g., via a disulfide bridge.

In some embodiments, the multispecific binding molecule comprises: (i) a first polypeptide comprising from N-terminal to C-terminal: VH of the costimulatory molecule binding domain, CH1, CH2, CH3, VH of the anti-CD3 binding domain, and VL of the anti-CD3 binding domain; and (ii) a second polypeptide comprising from N-terminal to C-terminal: VL of the costimulatory molecule binding domain and CL. In some embodiments, the anti-CD3 binding domain comprises an scFv. In some embodiments, the costimulatory molecule binding domain is part of a Fab fragment, e.g., a Fab fragment that is part of a polypeptide sequence that comprises an Fc domain. In some embodiments, the anti-CD3 binding domain is linked to the CH3 by a peptide linker, e.g., a glycine-serine linker, e.g., a (G4S) 4 (SEQ ID NO: 27) linker.

In some embodiments the multispecific binding molecule comprises: (i) a first polypeptide comprising from N-terminal to C-terminal: VH of the anti-CD3 binding domain, VL of the anti-CD3 binding domain, VH of the costimulatory molecule binding domain, CH1, CH2, and CH3; and (ii) a second polypeptide comprising from N-terminal to C-terminal: VL of the costimulatory molecule binding domain and CL. In some embodiments, the anti-CD3 binding domain is linked to the costimulatory molecule binding domain by a peptide linker, e.g., a glycine-serine linker, e.g., a (G4S) 4 (SEQ ID NO: 27) linker.

In some embodiments, the multispecific binding molecule comprises: (i) a first polypeptide comprising from N-terminal to C-terminal: VH of the costimulatory molecule binding domain, CH1, VH of the anti-CD3 binding domain, VL of the anti-CD3 binding domain, CH2, and CH3; and (ii) a second polypeptide comprising from N-terminal to C-terminal: VL of the costimulatory molecule binding domain and CL. In some embodiments, anti-CD3 binding domain is linked to the CH1 by a peptide linker, e.g., a glycine-serine linker, e.g., a (G4S) 2 (SEQ ID NO: 5) linker.

In some embodiments, the multispecific binding molecule comprises a heavy chain comprising the amino acid sequence of SEQ ID NO: 726 of WO 2022/040586 (incorporated by reference in its entirety), or an amino acid sequence at least 70%, 75%, 80%, 85%, 90%, 95%, 96%, 97%, 98%, or 99% identical thereto, and/or a light chain comprising the amino acid sequence of SEQ ID NO: 728 of WO 2022/040586 (incorporated by reference in its entirety), or an amino acid sequence at least 70%, 75%, 80%, 85%, 90%, 95%, 96%, 97%, 98%, or 99% identical thereto.

It is understood that in many of the embodiments herein, a multispecific binding molecule comprises two or more polypeptide chains that are covalently linked to each other, e.g., via a disulfide bridge. However, in some embodiments, the two or more polypeptide chains of the multispecific binding molecule may be noncovalently bound to each other.

It is also understood that a Fab fragment may be present as part of a larger protein, for instance, a Fab fragment may be fused with CH2 and CH3 and thus be part of full-length antibody.

The multispecific binding molecule comprising an agent that stimulates a CD3/TCR complex and an agent that stimulates a costimulatory molecule and/or growth factor receptor disclosed herein is contemplated for use in the manufacturing embodiments disclosed herein, e.g., traditional manufacture or activated rapid manufacture.

### Population of CAR-Expressing Cells Manufactured by the Processes Disclosed Herein

In some embodiments, the disclosure features an immune effector cell (for example, T cell or NK cell), for example, made by any of the manufacturing methods described herein, engineered to express a CAR (e.g., a B cell antigen, e.g., CD19), wherein the engineered immune effector cell exhibits an immunosuppressive property. In some embodiments, the CAR comprises an antigen-binding domain, a transmembrane domain, and an intracellular signaling domain. An exemplary antigen is a B cell antigen described herein. In some embodiments, the cell (for example, T cell or NK cell) is transformed with the CAR and the CAR is expressed on the cell surface. In some embodiments, the cell (for example, T cell or NK cell) is transduced with a viral vector encoding the CAR. In some embodiments, the viral vector is a retroviral vector. In some embodiments, the viral vector is a lentiviral vector. In some such embodiments, the cell may stably express the CAR. In some embodiments, the cell (for example, T cell or NK cell) is transfected with a nucleic acid, for example, mRNA, cDNA, or DNA, encoding a CAR. In some such embodiments, the cell may transiently express the CAR.

In some embodiments, provided herein is a population of cells (for example, immune effector cells, for example, T cells or NK cells) made by any of the manufacturing processes described herein (for example, the activation process described herein), engineered to express a CAR.

In some embodiments, the percentage of naïve cells, for example, naïve T cells, for example, CD45RA+ CD45RO− CCR7+ T cells, in the population of cells at the end of the manufacturing process (for example, at the end of the activation process described herein) (1) is the same as, (2) differs, for example, by no more than 4, 5, 6, 7, 8, 9, 10, 11, 12, 13, 14, or 15%, from, or (3) is increased, for example, by at least 5, 6, 7, 8, 9, 10, 11, 12, 13, 14, 15, 16, 17, 18, 19, 20, 21, 22, 23, 24, or 25%, as compared to, the percentage of naïve cells, for example, naïve T cells, for example, CD45RA+ CD45RO− CCR7+ cells, in the population of cells at the beginning of the manufacturing process (for example, at the beginning of the activation process described herein). In some embodiments, the population of cells at the end of the manufacturing process (for example, at the end of the activation process described herein) shows a higher percentage of naïve cells, for example, naïve T cells, for example, CD45RA+ CD45RO− CCR7+ T cells (for example, at least 5, 6, 7, 8, 9, 10, 11, 12, 13, 14, 15, 16, 17, 18, 19, 20, 25, 30, 35, 40, 45, or 50% higher), compared with cells made by an otherwise similar method which lasts, for example, more than 26 hours (for example, which lasts more than 5, 6, 7, 8, 9, 10, 11, or 12 days) or which involves expanding the population of cells in vitro for, for example, more than 3 days (for example, expanding the population of cells in vitro for 3, 4, 5, 6, 7, 8, 9, 10, 11, 12, 13, 14, or 15 days).

In some embodiments, the percentage of naïve cells, for example, naïve T cells, for example, CD45RA+CD45RO− CCR7+ T cells, in the population of cells at the end of the manufacturing process (for example, at the end of the activation process described herein) is not less than 20, 25, 30, 35, 40, 45, 50, 55, or 60%.

In some embodiments, the percentage of central memory cells, for example, central memory T cells, for example, CD95+ central memory T cells, in the population of cells at the end of the manufacturing process (for example, at the end of the activation process described herein) (1) is the same as, (2) differs, for example, by no more than 4, 5, 6, 7, 8, 9, 10, 11, 12, 13, 14, or 15% from, or (3) is decreased, for example, by at least 5, 6, 7, 8, 9, 10, 11, 12, 13, 14, 15, 16, 17, 18, 19, 20, 21, 22, 23, 24, or 25%, as compared to, the percentage of central memory cells, for example, central memory T cells, for example, CD95+ central memory T cells, in the population of cells at the beginning of the manufacturing process (for example, at the beginning of the activation process described herein). In some embodiments, the population of cells at the end of the manufacturing process (for example, at the end of the activation process described herein) shows a lower percentage of central memory cells, for example, central memory T cells, for example, CD95+ central memory T cells (for example, at least 5, 6, 7, 8, 9, 10, 11, 12, 13, 14, 15, 16, 17, 18, 19, 20, 25, 30, 35, 40, 45, or 50% lower), compared with cells made by an otherwise similar method which lasts, for example, more than 26 hours (for example, which lasts more than 5, 6, 7, 8, 9, 10, 11, or 12 days) or which involves expanding the population of cells in vitro for, for example, more than 3 days (for example, expanding the population of cells in vitro for 3, 4, 5, 6, 7, 8, 9, 10, 11, 12, 13, 14, or 15 days).

In some embodiments, the percentage of central memory cells, for example, central memory T cells, for example, CD95+ central memory T cells, in the population of cells at the end of the manufacturing process (for example, at the end of the activation process described herein) is no more than 40, 45, 50, 55, 60, 65, 70, 75, or 80%.

In some embodiments, the population of cells at the end of the manufacturing process (for example, at the end of the activation process described herein) after being administered in vivo, persists longer or expands at a higher level (for example, at least 20, 25, 30, 35, 40, 45, 50, 55, 60, 65, 70, 75, 80, 85, or 90% higher), compared with cells made by an otherwise similar method which lasts, for example, more than 26 hours (for example, which lasts more than 5, 6, 7, 8, 9, 10, 11, or 12 days) or which involves expanding the population of cells in vitro for, for example, more than 3 days (for example, expanding the population of cells in vitro for 3, 4, 5, 6, 7, 8, 9, 10, 11, 12, 13, 14, or 15 days).

In some embodiments, the population of cells has been enriched for IL6R-expressing cells (for example, cells that are positive for IL6Rα and/or IL6Rβ) prior to the beginning of the manufacturing process (for example, prior to the beginning of the activation process described herein). In some embodiments, the population of cells comprises, for example, no less than 30, 35, 40, 45, 50, 55, 60, 65, 70, 75, or 80% of IL6R-expressing cells (for example, cells that are positive for IL6Rα and/or IL6Rβ) at the beginning of the manufacturing process (for example, at the beginning of the activation process described herein).

### Pharmaceutical Composition

Furthermore, the present disclosure provides CAR-expressing cell compositions and their use in medicaments or methods for treating, among other diseases, autoimmune diseases (e.g., lupus (e.g., systemic lupus erythematosus (SLE), e.g., severe refractory systemic lupus erythematosus (srSLE), or lupus nephritis), systemic sclerosis (e.g., rapidly progressing systemic sclerosis (SSc) with significant lung involvement (e.g. as for autoHSCT)), idiopathic inflammatory myopathies (e.g., polymyositis, dermatomyositis, anti-synthetase syndrome, immune-mediated necrotizing myopathy, inclusion body myositis, overlap myositis, cancer associated myositis, e.g. anti-synthetase syndrome with ILD), vasculitis (e.g., ANCA-associated vasculitis), severe refractory Sjögren's, severe refractory neuroimmune disease (e.g. myasthenia gravis (MG), neuromyelitis optica (NMO), MOG associated disease (MOGAD), multiple sclerosis (MS)), severe refractory rheumatoid arthritis, antibody mediated neuroimmune diseases (e.g., AChR+ and MuSK+myasthenia gravis (MG), AQP4+ neuromyelitis optica (NMO), MOGAD (anti-MOG associated disease), NMDAR+ encephalitis, or antibody-associated neurological paraneoplastic diseases), Addison's disease, Goodpasture's syndrome, thyrotoxicosis, chronic active hepatitis, relapsing polychondritis, pemphigus vulgaris, or amyotrophic lateral sclerosis involving cells or tissues which express an antigen as described herein. In some embodiments, provided herein are pharmaceutical compositions comprising a CAR-expressing cell, for example, a plurality of CAR-expressing cells, made by a manufacturing process described herein (for example, the activation process described herein), in combination with one or more pharmaceutically or physiologically acceptable carriers, diluents or excipients.

### Chimeric Antigen Receptor (CAR)

The present invention provides immune effector cells (for example, T cells or NK cells) that are engineered to contain one or more CARs that direct the immune effector cells to cells associated with autoimmune disorders. This is achieved through an antigen-binding domain on the CAR that is specific for a B cell-associated antigen. There are two classes of B cell antigens that can be targeted by the CARs described herein: (1) B cell antigens that are expressed on the surface of B cells; and (2) B cell antigens that themselves are intracellular, however, fragments (peptides) of such antigens are presented on the surface of the B cells by MHC (major histocompatibility complex).

Accordingly, an immune effector cell, for example, obtained by a method described herein, can be engineered to contain a CAR that targets one or more of the following B cell antigens: CD19.

Sequences of non-limiting examples of various components that can be part of a CAR molecule described herein are listed in Table 1, where “aa” stands for amino acids, and “na” stands for nucleic acids that encode the corresponding peptide.

TABLE 1
Sequences of various components of CAR

| SEQ ID NO | Description | Sequence |
|---|---|---|
| SEQ ID NO: | EF-1α promoter | CGTGAGGCTCCGGTGCCCGTCAGTGGGCAGAGCGCACAT |
| 11 | (na) | CGCCCACAGTCCCCGAGAAGTTGGGGGGAGGGGTCGGC |
| | | AATTGAACCGGTGCCTAGAGAAGGTGGCGCGGGGTAAA |
| | | CTGGGAAAGTGATGTCGTGTACTGGCTCCGCCTTTTTCCC |
| | | GAGGGTGGGGGAGAACCGTATATAAGTGCAGTAGTCGC |
| | | CGTGAACGTTCTTTTTCGCAACGGGTTTGCCGCCAGAAC |
| | | ACAGGTAAGTGCCGTGTGTGGTTCCCGCGGGCCTGGCCT |
| | | CTTTACGGGTTATGGCCCTTGCGTGCCTTGAATTACTTCC |
| | | ACCTGGCTGCAGTACGTGATTCTTGATCCCGAGCTTCGG |
| | | GTTGGAAGTGGGTGGGAGAGTTCGAGGCCTTGCGCTTAA |
| | | GGAGCCCCTTCGCCTCGTGCTTGAGTTGAGGCCTGGCCT |
| | | GGGCGCTGGGGCCGCCGCGTGCGAATCTGGTGGCACCTT |
| | | CGCGCCTGTCTCGCTGCTTTCGATAAGTCTCTAGCCATTT |
| | | AAAATTTTTGATGACCTGCTGCGACGCTTTTTTTCTGGCA |
| | | AGATAGTCTTGTAAATGCGGGCCAAGATCTGCACACTGG |
| | | TATTTCGGTTTTTGGGGCCGCGGGCGGCGACGGGGCCCG |
| | | TGCGTCCCAGCGCACATGTTCGGCGAGGCGGGGCCTGCG |
| | | AGCGCGGCCACCGAGAATCGGACGGGGGTAGTCTCAAG |
| | | CTGGCCGGCCTGCTCTGGTGCCTGGCCTCGCGCCGCCGT |
| | | GTATCGCCCCGCCCTGGGCGGCAAGGCTGGCCCGGTCGG |
| | | CACCAGTTGCGTGAGCGGAAAGATGGCCGCTTCCCGGCC |
| | | CTGCTGCAGGGAGCTCAAAATGGAGGACGCGGCGCTCG |
| | | GGAGAGCGGGCGGGTGAGTCACCCACACAAAGGAAAAG |
| | | GGCCTTTCCGTCCTCAGCCGTCGCTTCATGTGACTCCACG |
| | | GAGTACCGGGCGCCGTCCAGGCACCTCGATTAGTTCTCG |
| | | AGCTTTTGGAGTACGTCGTCTTTAGGTTGGGGGGAGGGG |
| | | TTTTATGCGATGGAGTTTCCCCACACTGAGTGGGTGGAG |
| | | ACTGAAGTTAGGCCAGCTTGGCACTTGATGTAATTCTCC |
| | | TTGGAATTTGCCCTTTTTGAGTTTGGATCTTGGTTCATTC |
| | | TCAAGCCTCAGACAGTGGTTCAAAGTTTTTTTCTTCCATT |
| | | TCAGGTGTCGTGA |
| SEQ ID NO: | Leader (aa) | MALPVTALLLPLALLLHAARP |
| 1 | | |
| SEQ ID NO: | Leader (na) | ATGGCCCTGCCTGTGACAGCCCTGCTGCTGCCTCTGGCTC |
| 12 | | TGCTGCTGCATGCCGCTAGACCC |
| SEQ ID NO: | Leader (na) | ATGGCCCTCCCTGTCACCGCCCTGCTGCTTCCGCTGGCTC |
| 199 | | TTCTGCTCCACGCCGCTCGGCCC |
| SEQ ID NO: | CD 8 hinge (aa) | TTTPAPRPPTPAPTIASQPLSLRPEACRPAAGGAVHTRGLDF |
| 2 | | ACD |
| SEQ ID NO: | CD8 hinge (na) | ACCACGACGCCAGCGCCGCGACCACCAACACCGGCGCC |
| 13 | | CACCATCGCGTCGCAGCCCCTGTCCCTGCGCCCAGAGGC |
| | | GTGCCGGCCAGCGGCGGGGGGCGCAGTGCACACGAGGG |
| | | GGCTGGACTTCGCCTGTGAT |
| SEQ ID NO: | Ig4 hinge (aa) | ESKYGPPCPPCPAPEFLGGPSVFLFPPKPKDTLMISRTPEVTC |
| 3 | | VVVDVSQEDPEVQFNWYVDGVEVHNAKTKPREEQFNSTY |
| | | RVVSVLTVLHQDWLNGKEYKCKVSNKGLPSSIEKTISKAK |
| | | GQPREPQVYTLPPSQEEMTKNQVSLTCLVKGFYPSDIAVEW |
| | | ESNGQPENNYKTTPPVLDSDGSFFLYSRLTVDKSRWQEGN |
| | | VFSCSVMHEALHNHYTQKSLSLSLGKM |
| SEQ ID NO: | Ig4 hinge (na) | GAGAGCAAGTACGGCCCTCCCTGCCCCCCTTGCCCTGCC |
| 14 | | CCCGAGTTCCTGGGCGGACCCAGCGTGTTCCTGTTCCCC |
| | | CCCAAGCCCAAGGACACCCTGATGATCAGCCGGACCCCC |
| | | GAGGTGACCTGTGTGGTGGTGGACGTGTCCCAGGAGGAC |
| | | CCCGAGGTCCAGTTCAACTGGTACGTGGACGGCGTGGAG |
| | | GTGCACAACGCCAAGACCAAGCCCCGGGAGGAGCAGTT |
| | | CAATAGCACCTACCGGGTGGTGTCCGTGCTGACCGTGCT |
| | | GCACCAGGACTGGCTGAACGGCAAGGAATACAAGTGTA |
| | | AGGTGTCCAACAAGGGCCTGCCCAGCAGCATCGAGAAA |
| | | ACCATCAGCAAGGCCAAGGGCCAGCCTCGGGAGCCCCA |
| | | GGTGTACACCCTGCCCCCTAGCCAAGAGGAGATGACCAA |
| | | GAACCAGGTGTCCCTGACCTGCCTGGTGAAGGGCTTCTA |
| | | CCCCAGCGACATCGCCGTGGAGTGGGAGAGCAACGGCC |
| | | AGCCCGAGAACAACTACAAGACCACCCCCCCTGTGCTGG |
| | | ACAGCGACGGCAGCTTCTTCCTGTACAGCCGGCTGACCG |
| | | TGGACAAGAGCCGGTGGCAGGAGGGCAACGTCTTTAGC |
| | | TGCTCCGTGATGCACGAGGCCCTGCACAACCACTACACC |
| | | CAGAAGAGCCTGAGCCTGTCCCTGGGCAAGATG |
| SEQ ID NO: | IgD hinge (aa) | RWPESPKAQASSVPTAQPQAEGSLAKATTAPATTRNTGRG |
| 4 | | GEEKKKEKEKEEQEERETKTPECPSHTQPLGVYLLTPAVQD |
| | | LWLRDKATFTCFVVGSDLKDAHLTWEVAGKVPTGGVEEG |
| | | LLERHSNGSQSQHSRLTLPRSLWNAGTSVTCTLNHPSLPPQ |
| | | RLMALREPAAQAPVKLSLNLLASSDPPEAASWLLCEVSGFS |
| | | PPNILLMWLEDQREVNTSGFAPARPPPQPGSTTFWAWSVLR |
| | | VPAPPSPQPATYTCVVSHEDSRTLLNASRSLEVSYVTDH |
| SEQ ID NO: | IgD hinge (na) | AGGTGGCCCGAAAGTCCCAAGGCCCAGGCATCTAGTGTT |
| 15 | | CCTACTGCACAGCCCCAGGCAGAAGGCAGCCTAGCCAA |
| | | AGCTACTACTGCACCTGCCACTACGCGCAATACTGGCCG |
| | | TGGCGGGGAGGAGAAGAAAAAGGAGAAAGAGAAAGAA |
| | | GAACAGGAAGAGAGGGAGACCAAGACCCCTGAATGTCC |
| | | ATCCCATACCCAGCCGCTGGGCGTCTATCTCTTGACTCCC |
| | | GCAGTACAGGACTTGTGGCTTAGAGATAAGGCCACCTTT |
| | | ACATGTTTCGTCGTGGGCTCTGACCTGAAGGATGCCCAT |
| | | TTGACTTGGGAGGTTGCCGGAAAGGTACCCACAGGGGG |
| | | GGTTGAGGAAGGGTTGCTGGAGCGCCATTCCAATGGCTC |
| | | TCAGAGCCAGCACTCAAGACTCACCCTTCCGAGATCCCT |
| | | GTGGAACGCCGGGACCTCTGTCACATGTACTCTAAATCA |
| | | TCCTAGCCTGCCCCCACAGCGTCTGATGGCCCTTAGAGA |
| | | GCCAGCCGCCCAGGCACCAGTTAAGCTTAGCCTGAATCT |
| | | GCTCGCCAGTAGTGATCCCCCAGAGGCCGCCAGCTGGCT |
| | | CTTATGCGAAGTGTCCGGCTTTAGCCCGCCCAACATCTT |
| | | GCTCATGTGGCTGGAGGACCAGCGAGAAGTGAACACCA |
| | | GCGGCTTCGCTCCAGCCCGGCCCCCACCCCAGCCGGGTT |
| | | CTACCACATTCTGGGCCTGGAGTGTCTTAAGGGTCCCAG |
| | | CACCACCTAGCCCCCAGCCAGCCACATACACCTGTGTTG |
| | | TGTCCCATGAAGATAGCAGGACCCTGCTAAATGCTTCTA |
| | | GGAGTCTGGAGGTTTCCTACGTGACTGACCATT |
| SEQ ID NO: | CD8 | IYIWAPLAGTCGVLLLSLVITLYC |
| 6 | Transmembrane | |
| | (aa) | |
| SEQ ID NO: | CD8 | ATCTACATCTGGGCGCCCTTGGCCGGGACTTGTGGGGTC |
| 17 | Transmembrane | CTTCTCCTGTCACTGGTTATCACCCTTTACTGC |
| | (na) | |
| SEQ ID NO: | 4-1BB intracellular | KRGRKKLLYIFKQPFMRPVQTTQEEDGCSCRFPEEEEGGCE |
| 7 | domain (aa) | L |
| SEQ ID NO: | 4-1BB intracellular | AAACGGGGCAGAAAGAAACTCCTGTATATATTCAAACA |
| 18 | domain (na) | ACCATTTATGAGACCAGTACAAACTACTCAAGAGGAAG |
| | | ATGGCTGTAGCTGCCGATTTCCAGAAGAAGAAGAAGGA |
| | | GGATGTGAACTG |
| SEQ ID NO: | CD27 (aa) | QRRKYRSNKGESPVEPAEPCRYSCPREEEGSTIPIQEDYRKP |
| 8 | | EPACSP |
| SEQ ID NO: | CD27 (na) | AGGAGTAAGAGGAGCAGGCTCCTGCACAGTGACTACAT |
| 19 | | GAACATGACTCCCCGCCGCCCCGGGCCCACCCGCAAGCA |
| | | TTACCAGCCCTATGCCCCACCACGCGACTTCGCAGCCTA |
| | | TCGCTCC |
| SEQ ID NO: | CD3-zeta (aa) | RVKFSRSADAPAYKQGQNQLYNELNLGRREEYDVLDKRR |
| 9 | (Q/K mutant) | GRDPEMGGKPRRKNPQEGLYNELQKDKMAEAYSEIGMKG |
| | | ERRRGKGHDGLYQGLSTATKDTYDALHMQALPPR |
| SEQ ID NO: | CD3-zeta (na) | AGAGTGAAGTTCAGCAGGAGCGCAGACGCCCCCGCGTA |
| 20 | (Q/K mutant) | CAAGCAGGGCCAGAACCAGCTCTATAACGAGCTCAATCT |
| | | AGGACGAAGAGAGGAGTACGATGTTTTGGACAAGAGAC |
| | | GTGGCCGGGACCCTGAGATGGGGGGAAAGCCGAGAAGG |
| | | AAGAACCCTCAGGAAGGCCTGTACAATGAACTGCAGAA |
| | | AGATAAGATGGCGGAGGCCTACAGTGAGATTGGGATGA |
| | | AAGGCGAGCGCCGGAGGGGCAAGGGGCACGATGGCCTT |
| | | TACCAGGGTCTCAGTACAGCCACCAAGGACACCTACGAC |
| | | GCCCTTCACATGCAGGCCCTGCCCCCTCGC |
| SEQ ID NO: | CD3-zeta (aa) | RVKFSRSADAPAYQQGQNQLYNELNLGRREEYDVLDKRR |
| 10 | (NCBI Reference | GRDPEMGGKPRRKNPQEGLYNELQKDKMAEAYSEIGMKG |
| | Sequence | ERRRGKGHDGLYQGLSTATKDTYDALHMQALPPR |
| | NM_000734.3) | |
| SEQ ID NO: | CD3-zeta (na) | AGAGTGAAGTTCAGCAGGAGCGCAGACGCCCCCGCGTA |
| 21 | (NCBI Reference | CCAGCAGGGCCAGAACCAGCTCTATAACGAGCTCAATCT |
| | Sequence | AGGACGAAGAGAGGAGTACGATGTTTTGGACAAGAGAC |
| | NM_000734.3) | GTGGCCGGGACCCTGAGATGGGGGGAAAGCCGAGAAGG |
| | | AAGAACCCTCAGGAAGGCCTGTACAATGAACTGCAGAA |
| | | AGATAAGATGGCGGAGGCCTACAGTGAGATTGGGATGA |
| | | AAGGCGAGCGCCGGAGGGGCAAGGGGCACGATGGCCTT |
| | | TACCAGGGTCTCAGTACAGCCACCAAGGACACCTACGAC |
| | | GCCCTTCACATGCAGGCCCTGCCCCCTCGC |
| SEQ ID NO: | CD28 Intracellular | RSKRSRLLHSDYMNMTPRRPGPTRKHYQPYAPPRDFAAYR |
| 36 | domain (amino acid | S |
| | sequence) | |
| SEQ ID NO: | CD28 Intracellular | AGGAGTAAGAGGAGCAGGCTCCTGCACAGTGACTACAT |
| 37 | domain (nucleotide | GAACATGACTCCCCGCCGCCCCGGGCCCACCCGCAAGCA |
| | sequence) | TTACCAGCCCTATGCCCCACCACGCGACTTCGCAGCCTA |
| | | TCGCTCC |
| SEQ ID NO: | ICOS Intracellular | T K K K Y S S S V H D P N G E Y M F M R A V N T A K K S R |
| 38 | domain (amino acid | L T D V T L |
| | sequence) | |
| SEQ ID NO: | ICOS Intracellular | ACAAAAAAGAAGTATTCATCCAGTGTGCACGACCCTAAC |
| 39 | domain (nucleotide | GGTGAATACATGTTCATGAGAGCAGTGAACACAGCCAA |
| | sequence) | AAAATCCAGACTCACAGATGTGACCCTA |
| SEQ ID NO: | GS hinge/linker | GGGGSGGGGS |
| 5 | (aa) | |
| SEQ ID NO: | GS hinge/linker | GGTGGCGGAGGTTCTGGAGGTGGAGGTTCC |
| 16 | (na) | |
| SEQ ID NO: | GS hinge/linker | GGTGGCGGAGGTTCTGGAGGTGGGGGTTCC |
| 40 | (na) | |
| SEQ ID NO: | linker | GGGGS |
| 25 | | |
| SEQ ID NO: | linker | (Gly-Gly-Gly-Gly-Ser)n, where n = 1-6, for example, |
| 26 | | GGGGSGGGGS GGGGSGGGGS GGGGSGGGGS |
| SEQ ID NO: | linker | GGGGSGGGGSGGGGSGGGGS |
| 27 | | |
| SEQ ID NO: | linker | GGGGSGGGGSGGGGS |
| 28 | | |
| SEQ ID NO: | linker | GGGS |
| 29 | | |
| SEQ ID NO: | linker | (Gly-Gly-Gly-Ser)n where n is a positive integer equal to or |
| 41 | | greater than 1 |
| SEQ ID NO: | linker | (Gly-Gly-Gly-Ser)n, where n = 1-10, for example, |
| 42 | | GGGSGGGSGG GSGGGSGGGS GGGSGGGSGG |
| | | GSGGGSGGGS |
| SEQ ID NO: | linker | GSTSGSGKPGSGEGSTKG |
| 43 | | |
| SEQ ID NO: | poly(A) | (A)₅₀₀₀ |
| 30 | | This sequence may encompass 50-5000 adenines. |
| SEQ ID NO: | polyT | (T)₁₀₀ |
| 31 | | |
| SEQ ID NO: | polyT | (T)₅₀₀₀ |
| 32 | | This sequence may encompass 50-5000 thymines. |
| SEQ ID NO: | poly(A) | (A)₅₀₀₀ |
| 33 | | This sequence may encompass 100-5000 adenines. |
| SEQ ID NO: | poly(A) | (A)₄₀₀ |
| 34 | | This sequence may encompass 100-400 adenines. |
| SEQ ID NO: | poly(A) | (A)₂₀₀₀ |
| 35 | | This sequence may encompass 50-2000 adenines. |

### Bispecific CARs

In some embodiments a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In some embodiments the first and second epitopes are on the same antigen, for example, the same protein (or subunit of a multimeric protein). In some embodiments the first and second epitopes overlap. In some embodiments the first and second epitopes do not overlap. In some embodiments the first and second epitopes are on different antigens, for example, different proteins (or different subunits of a multimeric protein). In some embodiments a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In some embodiments a bispecific antibody molecule comprises a half antibody having binding specificity for a first epitope and a half antibody having binding specificity for a second epitope. In some embodiments a bispecific antibody molecule comprises a half antibody, or fragment thereof, having binding specificity for a first epitope and a half antibody, or fragment thereof, having binding specificity for a second epitope. In some embodiments a bispecific antibody molecule comprises a scFv, or fragment thereof, have binding specificity for a first epitope and a scFv, or fragment thereof, have binding specificity for a second epitope.

In certain embodiments, the antibody molecule is a multi-specific (for example, a bispecific or a trispecific) antibody molecule. Protocols for generating bispecific or heterodimeric antibody molecules, and various configurations for bispecific antibody molecules, are described in, for example, paragraphs 455-458 of WO2015/142675, filed Mar. 13, 2015, which is incorporated by reference in its entirety.

In some embodiments, the bispecific antibody molecule is characterized by a first immunoglobulin variable domain sequence, for example, a scFv, which has binding specificity for CD19, for example, comprises a scFv as described herein, or comprises the light chain CDRs and/or heavy chain CDRs from a scFv described herein, and a second immunoglobulin variable domain sequence that has binding specificity for a second epitope on a different antigen.

### Chimeric TCR

In some embodiments, the antibodies and antibody fragments of the present invention (for example, CD19 antibodies and fragments) can be grafted to one or more constant domain of a T cell receptor (“TCR”) chain, for example, a TCR alpha or TCR beta chain, to create a chimeric TCR. Without being bound by theory, it is believed that chimeric TCRs will signal through the TCR complex upon antigen-binding. For example, an scFv as disclosed herein, can be grafted to the constant domain, for example, at least a portion of the extracellular constant domain, the transmembrane domain and the cytoplasmic domain, of a TCR chain, for example, the TCR alpha chain and/or the TCR beta chain. As another example, an antibody fragment, for example a VL domain as described herein, can be grafted to the constant domain of a TCR alpha chain, and an antibody fragment, for example a VH domain as described herein, can be grafted to the constant domain of a TCR beta chain (or alternatively, a VL domain may be grafted to the constant domain of the TCR beta chain and a VH domain may be grafted to a TCR alpha chain). As another example, the CDRs of an antibody or antibody fragment may be grafted into a TCR alpha and/or beta chain to create a chimeric TCR. For example, the LCDRs disclosed herein may be grafted into the variable domain of a TCR alpha chain and the HCDRs disclosed herein may be grafted to the variable domain of a TCR beta chain, or vice versa. Such chimeric TCRs may be produced, for example, by methods known in the art (For example, Willemsen R A et al, Gene Therapy 2000; 7:1369-1377; Zhang T et al, Cancer Gene Ther 2004; 11:487-496; Aggen et al, Gene Ther. 2012 April;19 (4): 365−74).

### Non-Antibody Scaffolds

In embodiments, the antigen-binding domain comprises a non-antibody scaffold, for example, a fibronectin, ankyrin, domain antibody, lipocalin, small modular immuno-pharmaceutical, maxybody, Protein A, or affilin. The non-antibody scaffold has the ability to bind to target antigen on a cell. In embodiments, the antigen-binding domain is a polypeptide or fragment thereof of a naturally occurring protein expressed on a cell. In some embodiments, the antigen-binding domain comprises a non-antibody scaffold. A wide variety of non-antibody scaffolds can be employed so long as the resulting polypeptide includes at least one binding region which specifically binds to the target antigen on a target cell.

Non-antibody scaffolds include: fibronectin (Novartis, MA), ankyrin (Molecular Partners AG, Zurich, Switzerland), domain antibodies (Domantis, Ltd., Cambridge, MA, and Ablynx nv, Zwijnaarde, Belgium), lipocalin (Pieris Proteolab AG, Freising, Germany), small modular immuno-pharmaceuticals (Trubion Pharmaceuticals Inc., Seattle, WA), maxybodies (Avidia, Inc., Mountain View, CA), Protein A (Affibody AG, Sweden), and affilin (gamma-crystallin or ubiquitin) (Scil Proteins GmbH, Halle, Germany).

In some embodiments the antigen-binding domain comprises the extracellular domain, or a counter-ligand binding fragment thereof, of molecule that binds a counterligand on the surface of a target cell.

The immune effector cells can comprise a recombinant DNA construct comprising sequences encoding a CAR, wherein the CAR comprises an antigen-binding domain (for example, antibody or antibody fragment, TCR or TCR fragment) that binds specifically to a B cell antigen, for example, a B cell antigen described herein, and an intracellular signaling domain. The intracellular signaling domain can comprise a costimulatory signaling domain and/or a primary signaling domain, for example, a zeta chain. As described elsewhere, the methods described herein can include transducing a cell, for example, from the population of T regulatory-depleted cells, with a nucleic acid encoding a CAR, for example, a CAR described herein.

In some embodiments, a CAR comprises a scFv domain, wherein the scFv may be preceded by an optional leader sequence such as provided in SEQ ID NO: 1, and followed by an optional hinge sequence such as provided in SEQ ID NO:2 or SEQ ID NO:36 or SEQ ID NO: 38, a transmembrane region such as provided in SEQ ID NO:6, an intracellular signaling domain that includes SEQ ID NO:7 or SEQ ID NO:16 and a CD3 zeta sequence that includes SEQ ID NO:9 or SEQ ID NO:10, for example, wherein the domains are contiguous with and in the same reading frame to form a single fusion protein.

In some embodiments, an exemplary CAR constructs comprise an optional leader sequence (for example, a leader sequence described herein), an extracellular antigen-binding domain (for example, an antigen-binding domain described herein), a hinge (for example, a hinge region described herein), a transmembrane domain (for example, a transmembrane domain described herein), and an intracellular stimulatory domain (for example, an intracellular stimulatory domain described herein). In some embodiments, an exemplary CAR construct comprises an optional leader sequence (for example, a leader sequence described herein), an extracellular antigen-binding domain (for example, an antigen-binding domain described herein), a hinge (for example, a hinge region described herein), a transmembrane domain (for example, a transmembrane domain described herein), an intracellular costimulatory signaling domain (for example, a costimulatory signaling domain described herein) and/or an intracellular primary signaling domain (for example, a primary signaling domain described herein).

An exemplary leader sequence is provided as SEQ ID NO: 1. An exemplary hinge/spacer sequence is provided as SEQ ID NO: 2 or SEQ ID NO:36 or SEQ ID NO:38. An exemplary transmembrane domain sequence is provided as SEQ ID NO:6. An exemplary sequence of the intracellular signaling domain of the 4-1BB protein is provided as SEQ ID NO: 7. An exemplary sequence of the intracellular signaling domain of CD27 is provided as SEQ ID NO: 16. An exemplary CD3zeta domain sequence is provided as SEQ ID NO: 9 or SEQ ID NO: 10.

In some embodiments, the immune effector cell comprises a recombinant nucleic acid construct comprising a nucleic acid molecule encoding a CAR, wherein the nucleic acid molecule comprises a nucleic acid sequence encoding an antigen-binding domain, wherein the sequence is contiguous with and in the same reading frame as the nucleic acid sequence encoding an intracellular signaling domain. An exemplary intracellular signaling domain that can be used in the CAR includes, but is not limited to, one or more intracellular signaling domains of, for example, CD3-zeta, CD28, CD27, 4-1BB, and the like. In some instances, the CAR can comprise any combination of CD3-zeta, CD28, 4-1BB, and the like.

The nucleic acid sequences coding for the desired molecules can be obtained using recombinant methods known in the art, such as, for example by screening libraries from cells expressing the nucleic acid molecule, by deriving the nucleic acid molecule from a vector known to include the same, or by isolating directly from cells and tissues containing the same, using standard techniques. Alternatively, the nucleic acid of interest can be produced synthetically, rather than cloned.

Nucleic acids encoding a CAR can be introduced into the immune effector cells using, for example, a retroviral or lentiviral vector construct.

Nucleic acids encoding a CAR can also be introduced into the immune effector cell using, for example, an RNA construct that can be directly transfected into a cell. A method for generating mRNA for use in transfection involves in vitro transcription (IVT) of a template with specially designed primers, followed by poly (A) addition, to produce a construct containing 3′ and 5′ untranslated sequence (“UTR”) (for example, a 3′ and/or 5′ UTR described herein), a 5′ cap (for example, a 5′ cap described herein) and/or Internal Ribosome Entry Site (IRES) (for example, an IRES described herein), the nucleic acid to be expressed, and a poly(A) tail, typically 50-2000 bases in length (for example, herein, for example, SEQ ID NO: 35). RNA so produced can efficiently transfect different kinds of cells. In some embodiments, the template includes sequences for the CAR. In some embodiments, an RNA CAR vector is transduced into a cell, for example, a T cell by electroporation.

### Antigen-Binding Domain

In some embodiments, a plurality of the immune effector cells, for example, the population of T regulatory-depleted cells, include a nucleic acid encoding a CAR that comprises a target-specific binding element otherwise referred to as an antigen-binding domain. The choice of binding element depends upon the type and number of ligands that define the surface of a target cell. For example, the antigen-binding domain may be chosen to recognize a ligand that acts as a cell surface marker on target cells associated with a particular disease state. Thus, examples of cell surface markers that may act as ligands for the antigen-binding domain in a CAR described herein include those associated autoimmune disease.

In some embodiments, the portion of the CAR comprising the antigen-binding domain comprises an antigen-binding domain that targets a B cell antigen, for example, a B cell antigen described herein.

The antigen-binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen-binding domain, such as a recombinant fibronectin domain, a T cell receptor (TCR), or a fragment there of, for example, single chain TCR, and the like. In some instances, it is beneficial for the antigen-binding domain to be derived from the same species in which the CAR will ultimately be used in. For example, for use in humans, it may be beneficial for the antigen-binding domain of the CAR to comprise human or humanized residues for the antigen-binding domain of an antibody or antibody fragment.

### CD19 CAR

In some embodiments, the CAR-expressing cell described herein is a CD19 CAR-expressing cell (for example, a cell expressing a CAR that binds to human CD19).

In some embodiments, the antigen-binding domain of the CD19 CAR has the same or a similar binding specificity as the FMC63 scFv fragment described in Nicholson et al. Mol. Immun. 34 (16-17): 1157-1165 (1997). In some embodiments, the antigen-binding domain of the CD19 CAR includes the scFv fragment described in Nicholson et al. Mol. Immun. 34 (16-17): 1157-1165 (1997).

In some embodiments, the CD19 CAR includes an antigen-binding domain (for example, a humanized antigen-binding domain) according to Table 3 of WO2014/153270, incorporated herein by reference. WO2014/153270 also describes methods of assaying the binding and efficacy of various CAR constructs.

In some embodiments, the parental murine scFv sequence is the CAR19 construct provided in PCT publication WO2012/079000 (incorporated herein by reference). In some embodiments, the anti-CD19 binding domain is a scFv described in WO2012/079000.

In some embodiments, the CAR molecule comprises the fusion polypeptide sequence provided as SEQ ID NO: 12 in PCT publication WO2012/079000, which provides an scFv fragment of murine origin that specifically binds to human CD19.

In some embodiments, the CD19 CAR comprises an amino acid sequence provided as SEQ ID NO: 12 in PCT publication WO2012/079000.

In some embodiments, the amino acid sequence is:

(SEQ ID NO: 292)
Diqmtqttsslsaslgdrvtiscrasqdiskylnwyqqkpdgtvklliyh
tsrlhsgvpsrfsgsgsgtdysltisnleqediatyfcqqgntlpytfgg
gtkleitggggsggggsggggsevklqesgpglvapsqslsvtctvsgvs
lpdygvswirqpprkglewlgviwgsettyynsalksrltiikdnsksqv
flkmnslqtddtaiyycakhyyyggsyamdywgqgtsvtvsstttpaprp
ptpaptiasqplslrpeacrpaaggavhtrgldfacdiyiwaplagtcgv
lllslvitlyckrgrkkllyifkqpfmrpvqttqeedgcscrfpeeeegg
celrvkfsrsadapaykqgqnqlynelnlgrreeydvldkrrgrdpemgg
kprrknpqeglynelqkdkmaeayseigmkgerrrgkghdglyqglstat
kdtydalhmqalppr,
or a sequence substantially homologous thereto.

In some embodiments, the CD19 CAR has the USAN designation TISAGENLECLEUCEL-T. In embodiments, CTL019 is made by a gene modification of T cells is mediated by stable insertion via transduction with a self-inactivating, replication deficient Lentiviral (LV) vector containing the CTL019 transgene under the control of the EF-1 alpha promoter. CTL019 can be a mixture of transgene positive and negative T cells that are delivered to the subject on the basis of percent transgene positive T cells.

In some embodiments, the population of CAR T cells that specifically bind to CD19 comprises rapcabtagene autoleucel. The rapcabtagene autoleucel is made using autologous T cells obtained from peripheral blood mononuclear cells (e.g., from a subject having an autoimmune disease or disorder) by leukapheresis and subsequently transduced with a self-inactivating, non-replicating lentiviral vector encoding a T cell chimeric antigen receptor targeting CD19. The expressed transgene comprises a CD8a leader sequence, a murine anti-CD19 single chain variable fragment (scFv) derived from the mouse hybridoma FMC63, a CD8a hinge and transmembrane region, and a 4-1BB (CD137) and CD3ζ (TCRζ) signaling domain, and is under control of the elongation factor 1 alpha (EF1α) promoter. The construct is flanked by 5′ and 3′ long terminal repeats (LTRs) and also contains a ψ packaging signal, a Rev response element (RRE), a central polypurine tract (cPPT) sequence, and an optimized Woodchuck hepatitis virus posttranscriptional regulatory element (WPRE). The leukapheresis material is enriched for CD4/CD8 T cells by positive immunoselection, activated by CD3 and CD28 agonists and transduced with the vector. Without further cell propagation, the T cells are washed, formulated for infusion, and cryopreserved. Rapcabtagene autoleucel is composed of ≥80% T cells and ≤1% B cells, with a mixture of transgene positive (>3.4%) and negative T cells. The CD4+ and CD8+naïve T cell subsets (CD45RA+CCR7+) present in the leukapheresis material are largely retained.

In some embodiments, CAR-expressing cells described herein or CAR-positive cells (e.g., CD19 CAR-expressing cells or CD19 CAR-positive cells) are rapcabtagene autoleucel. In some embodiments, the population of ARM-CD19 CAR T cells is rapcaptagene autoleucel.

In some embodiments, rapcabtagene autoleucel is made from autologous T cells obtained from a subject having an autoimmune disease or disorder, e.g., lupus (e.g., systemic lupus erythematosus (SLE), e.g., severe refractory systemic lupus erythematosus (srSLE), or lupus nephritis), systemic sclerosis (e.g., rapidly progressing systemic sclerosis (SSc) with significant lung involvement (e.g. as for autoHISCT)), idiopathic inflammatory myopathies (e.g., polymyositis, dermatomyositis, anti-synthetase syndrome, immune-mediated necrotizing myopathy, inclusion body myositis, overlap myositis, cancer associated myositis, e.g. anti-synthetase syndrome with ILD), vasculitis (e.g., ANCA-associated vasculitis), severe refractory Sjögren's, severe refractory neuroimmune disease (e.g. myasthenia gravis (MG), neuromyelitis optica (NMO), MOG associated disease (MOGAD), multiple sclerosis (MS)), severe refractory rheumatoid arthritis, antibody mediated neuroimmune diseases (e.g., AChR+ and MuSK+myasthenia gravis (MG), AQP4+ neuromyelitis optica (NMO), MOGAD (anti-MOG associated disease), NMDAR+ encephalitis, or antibody-associated neurological paraneoplastic diseases), Addison's disease, Goodpasture's syndrome, thyrotoxicosis, chronic active hepatitis, relapsing polychondritis, pemphigus vulgaris, or amyotrophic lateral sclerosis.

In other embodiments, the CD19 CAR comprises an antigen-binding domain (for example, a humanized antigen-binding domain) according to Table 3 of WO2014/153270, incorporated herein by reference.

Humanization of murine CD19 antibody is desired for the clinical setting, where the mouse-specific residues may induce a human-anti-mouse antigen (HAMA) response in patients who receive CART19 treatment, i.e., treatment with T cells transduced with the CAR19 construct. The production, characterization, and efficacy of humanized CD19 CAR sequences is described in International Application WO2014/153270 which is herein incorporated by reference in its entirety, including Examples 1-5 (p. 115-159).

In some embodiments, the CAR molecule is a humanized CD19 CAR comprising the amino acid sequence of:

(SEQ ID NO: 293)
EIVMTQSPATLSLSPGERATLSCRASQDISKYLNWYQQKPGQAPRLLIYH
TSRLHSGIPARFSGSGSGTDYTLTISSLQPEDFAVYFCQQGNTLPYTFGQ
GTKLEIKGGGGSGGGGSGGGGSQVQLQESGPGLVKPSETLSLTCTVSGVS
LPDYGVSWIRQPPGKGLEWIGVIWGSETTYYQSSLKSRVTISKDNSKNQV
SLKLSSVTAADTAVYYCAKHYYYGGSYAMDYWGQGTLVTVSS

In some embodiments, the CAR molecule is a humanized CD19 CAR comprising the amino acid sequence of:

(SEQ ID NO: 294)
EIVMTQSPATLSLSPGERATLSCRASQDISKYLNWYQQKPGQAPRLLIYH
TSRLHSGIPARFSGSGSGTDYTLTISSLQPEDFAVYFCQQGNTLPYTFGQ
GTKLEIKGGGGSGGGGSGGGGSQVQLQESGPGLVKPSETLSLTCTVSGVS
LPDYGVSWIRQPPGKGLEWIGVIWGSETTYYQSSLKSRVTISKDNSKNQV
SLKLSSVTAADTAVYYCAKHYYYGGSYAMDYWGQGTLVTVSSTTTPAPRP
PTPAPTIASQPLSLRPEACRPAAGGAVHTRGLDFACDIYIWAPLAGTCGV
LLLSLVITLYCKRGRKKLLYIFKQPFMRPVQTTQEEDGCSCRFPEEEEGG
CELRVKFSRSADAPAYKQGQNQLYNELNLGRREEYDVLDKRRGRDPEMGG
KPRRKNPQEGLYNELQKDKMAEAYSEIGMKGERRRGKGHDGLYQGLSTAT
KDTYDALHMQALPPR

Any known CD19 CAR, for example, the CD19 antigen-binding domain of any known CD19 CAR, in the art can be used in accordance with the present disclosure. For example, LG-740; CD19 CAR described in the U.S. Pat. Nos. 8,399,645; 7,446,190; Xu et al., Leuk Lymphoma. 2013 54 (2): 255-260 (2012); Cruz et al., Blood 122 (17): 2965-2973 (2013); Brentjens et al., Blood, 118 (18): 4817-4828 (2011); Kochenderfer et al., Blood 116 (20): 4099-102 (2010); Kochenderfer et al., Blood 122 (25): 4129-39 (2013); and 16th Annu Meet Am Soc Gen Cell Ther (ASGCT) (May 15-18, Salt Lake City) 2013, Abst 10.

Exemplary CD19 CARs include CD19 CARs described herein or an anti-CD19 CAR described in Xu et al. Blood 123.24 (2014): 3750-9; Kochenderfer et al. Blood 122.25 (2013): 4129-39, Cruz et al. Blood 122.17 (2013): 2965-73, NCT00586391, NCT01087294, NCT02456350, NCT00840853, NCT02659943, NCT02650999, NCT02640209, NCT01747486, NCT02546739, NCT02656147, NCT02772198, NCT00709033, NCT02081937, NCT00924326, NCT02735083, NCT02794246, NCT02746952, NCT01593696, NCT02134262, NCT01853631, NCT02443831, NCT02277522, NCT02348216, NCT02614066, NCT02030834, NCT02624258, NCT02625480, NCT02030847, NCT02644655, NCT02349698, NCT02813837, NCT02050347, NCT01683279, NCT02529813, NCT02537977, NCT02799550, NCT02672501, NCT02819583, NCT02028455, NCT01840566, NCT01318317, NCT01864889, NCT02706405, NCT01475058, NCT01430390, NCT02146924, NCT02051257, NCT02431988, NCT01815749, NCT02153580, NCT01865617, NCT02208362, NCT02685670, NCT02535364, NCT02631044, NCT02728882, NCT02735291, NCT01860937, NCT02822326, NCT02737085, NCT02465983, NCT02132624, NCT02782351, NCT01493453, NCT02652910, NCT02247609, NCT01029366, NCT01626495, NCT02721407, NCT01044069, NCT00422383, NCT01680991, NCT02794961, or NCT02456207, each of which is incorporated herein by reference in its entirety.

In some embodiments, CD19 CARs comprise a sequence, for example, a CDR, VH, VL, scFv, or full-CAR sequence, disclosed in Table 2, or a sequence having at least 80%, 85%, 90%, 95%, or 99% identity thereto.

TABLE 2
Amino acid sequences of exemplary anti-CD19 molecules

| | | |
|---|---|---|
| SEQ ID | | |
| NO | Region | Sequence |

CTL019

| | | |
|---|---|---|
| 295 | HCDR1 | DYGVS |
| | (Kabat) | |
| 296 | HCDR2 | VIWGSETTYYNSALKS |
| | (Kabat) | |
| 297 | HCDR3 | HYYYGGSYAMDY |
| | (Kabat) | |
| 298 | LCDR1 | RASQDISKYLN |
| | (Kabat) | |
| 299 | LCDR2 | HTSRLHS |
| | (Kabat) | |
| 300 | LCDR3 | QQGNTLPYT |
| | (Kabat) | |
| 301 | CTL019 | MALPVTALLLPLALLLHAARPDIQMTQTTSSLSASLGDRVTISCRA |
| | Full amino | SQDISKYLNWYQQKPDGTVKLLIYHTSRLHSGVPSRFSGSGSGTD |
| | acid | YSLTISNLEQEDIATYFCQQGNTLPYTFGGGTKLEITGGGGSGGGG |
| | sequence | SGGGGSEVKLQESGPGLVAPSQSLSVTCTVSGVSLPDYGVSWIRQ |
| | | PPRKGLEWLGVIWGSETTYYNSALKSRLTIIKDNSKSQVFLKMNS |
| | | LQTDDTAIYYCAKHYYYGGSYAMDYWGQGTSVTVSSTTTPAPR |
| | | PPTPAPTIASQPLSLRPEACRPAAGGAVHTRGLDFACDIYIWAPLA |
| | | GTCGVLLLSLVITLYCKRGRKKLLYIFKQPFMRPVQTTQEEDGCS |
| | | CRFPEEEEGGCELRVKFSRSADAPAYKQGQNQLYNELNLGRREE |
| | | YDVLDKRRGRDPEMGGKPRRKNPQEGLYNELQKDKMAEAYSEI |
| | | GMKGERRRGKGHDGLYQGLSTATKDTYDALHMQALPPR |
| 302 | CTL019 | ATGGCCTTACCAGTGACCGCCTTGCTCCTGCCGCTGGCCTTGCT |
| | Full | GCTCCACGCCGCCAGGCCGGACATCCAGATGACACAGACTAC |
| | nucleotide | ATCCTCCCTGTCTGCCTCTCTGGGAGACAGAGTCACCATCAGT |
| | sequence | TGCAGGGCAAGTCAGGACATTAGTAAATATTTAAATTGGTATC |
| | | AGCAGAAACCAGATGGAACTGTTAAACTCCTGATCTACCATAC |
| | | ATCAAGATTACACTCAGGAGTCCCATCAAGGTTCAGTGGCAGT |
| | | GGGTCTGGAACAGATTATTCTCTCACCATTAGCAACCTGGAGC |
| | | AAGAAGATATTGCCACTTACTTTTGCCAACAGGGTAATACGCT |
| | | TCCGTACACGTTCGGAGGGGGGACCAAGCTGGAGATCACAGG |
| | | TGGCGGTGGCTCGGGCGGTGGTGGGTCGGGTGGCGGCGGATC |
| | | TGAGGTGAAACTGCAGGAGTCAGGACCTGGCCTGGTGGCGCC |
| | | CTCACAGAGCCTGTCCGTCACATGCACTGTCTCAGGGGTCTCA |
| | | TTACCCGACTATGGTGTAAGCTGGATTCGCCAGCCTCCACGAA |
| | | AGGGTCTGGAGTGGCTGGGAGTAATATGGGGTAGTGAAACCA |
| | | CATACTATAATTCAGCTCTCAAATCCAGACTGACCATCATCAA |
| | | GGACAACTCCAAGAGCCAAGTTTTCTTAAAAATGAACAGTCTG |
| | | CAAACTGATGACACAGCCATTTACTACTGTGCCAAACATTATT |
| | | ACTACGGTGGTAGCTATGCTATGGACTACTGGGGCCAAGGAAC |
| | | CTCAGTCACCGTCTCCTCAACCACGACGCCAGCGCCGCGACCA |
| | | CCAACACCGGCGCCCACCATCGCGTCGCAGCCCCTGTCCCTGC |
| | | GCCCAGAGGCGTGCCGGCCAGCGGCGGGGGGCGCAGTGCACA |
| | | CGAGGGGGCTGGACTTCGCCTGTGATATCTACATCTGGGCGCC |
| | | CTTGGCCGGGACTTGTGGGGTCCTTCTCCTGTCACTGGTTATCA |
| | | CCCTTTACTGCAAACGGGGCAGAAAGAAACTCCTGTATATATT |
| | | CAAACAACCATTTATGAGACCAGTACAAACTACTCAAGAGGA |
| | | AGATGGCTGTAGCTGCCGATTTCCAGAAGAAGAAGAAGGAGG |
| | | ATGTGAACTGAGAGTGAAGTTCAGCAGGAGCGCAGACGCCCC |
| | | CGCGTACAAGCAGGGCCAGAACCAGCTCTATAACGAGCTCAA |
| | | TCTAGGACGAAGAGAGGAGTACGATGTTTTGGACAAGAGACG |
| | | TGGCCGGGACCCTGAGATGGGGGGAAAGCCGAGAAGGAAGA |
| | | ACCCTCAGGAAGGCCTGTACAATGAACTGCAGAAAGATAAGA |
| | | TGGCGGAGGCCTACAGTGAGATTGGGATGAAAGGCGAGCGCC |
| | | GGAGGGGCAAGGGGCACGATGGCCTTTACCAGGGTCTCAGTA |
| | | CAGCCACCAAGGACACCTACGACGCCCTTCACATGCAGGCCCT |
| | | GCCCCCTCGC |
| 303 | CTL019 | DIQMTQTTSSLSASLGDRVTISCRASQDISKYLNWYQQKPDGTVK |
| | scFv | LLIYHTSRLHSGVPSRFSGSGSGTDYSLTISNLEQEDIATYFCQQGN |
| | domain | TLPYTFGGGTKLEITGGGGSGGGGSGGGGSEVKLQESGPGLVAPS |
| | | QSLSVTCTVSGVSLPDYGVSWIRQPPRKGLEWLGVIWGSETTYY |
| | | NSALKSRLTIIKDNSKSQVFLKMNSLQTDDTAIYYCAKHYYYGGS |
| | | YAMDYWGQGTSVTVSS |

Humanized CAR2

| | | |
|---|---|---|
| 295 | HCDR1 | DYGVS |
| | (Kabat) | |
| 304 | HCDR2 | VIWGSETTYYQSSLKS |
| | (Kabat) | |
| 297 | HCDR3 | HYYYGGSYAMDY |
| | (Kabat) | |
| 298 | LCDR1 | RASQDISKYLN |
| | (Kabat) | |
| 299 | LCDR2 | HTSRLHS |
| | (Kabat) | |
| 300 | LCDR3 | QQGNTLPYT |
| | (Kabat) | |
| 293 | CAR2 scFv | EIVMTQSPATLSLSPGERATLSCRASQDISKYLNWYQQKPGQAPR |
| | domain - aa | LLIYHTSRLHSGIPARFSGSGSGTDYTLTISSLQPEDFAVYFCQQGN |
| | (Linker is | TLPYTFGQGTKLEIKGGGGSGGGGSGGGGSQVQLQESGPGLVKP |
| | underlined) | SETLSLTCTVSGVSLPDYGVSWIRQPPGKGLEWIGVIWGSETTYY |
| | | QSSLKSRVTISKDNSKNQVSLKLSSVTAADTAVYYCAKHYYYGG |
| | | SYAMDYWGQGTLVTVSS |
| 305 | CAR2 scFv | atggccctccctgtcaccgccctgctgcttccgctggctcttctgctccacgccgctcggcccgaaattgt |
| | domain - nt | gatgacccagtcacccgccactcttagcctttcacccggtgagcgcgcaaccctgtcttgcagagcctcc |
| | | caagacatctcaaaataccttaattggtatcaacagaagcccggacaggctcctcgccttctgatctacca |
| | | caccagccggctccattctggaatccctgccaggttcagcggtagcggatctgggaccgactacaccct |
| | | cactatcagctcactgcagccagaggacttcgctgtctatttctgtcagcaagggaacaccctgccctac |
| | | acctttggacagggcaccaagctcgagattaaaggtggaggtggcagcggaggaggtgggtccggc |
| | | ggtggaggaagccaggtccaactccaagaaagcggaccgggtcttgtgaagccatcagaaactctttc |
| | | actgacttgtactgtgagcggagtgtctctccccgattacggggtgtcttggatcagacagccaccgggg |
| | | aagggtctggaatggattggagtgatttggggctctgagactacttactaccaatcatccctcaagtcacg |
| | | cgtcaccatctcaaaggacaactctaagaatcaggtgtcactgaaactgtcatctgtgaccgcagccgac |
| | | accgccgtgtactattgcgctaagcattactattatggcgggagctacgcaatggattactggggacagg |
| | | gtactctggtcaccgtgtccagccaccaccatcatcaccatcaccat |
| 306 | CAR 2 - | MALPVTALLLPLALLLHAARPEIVMTQSPATLSLSPGERATLSCRA |
| | Full - aa | SQDISKYLNWYQQKPGQAPRLLIYHTSRLHSGIPARFSGSGSGTD |
| | | YTLTISSLQPEDFAVYFCQQGNTLPYTFGQGTKLEIKGGGGSGGG |
| | | GSGGGGSQVQLQESGPGLVKPSETLSLTCTVSGVSLPDYGVSWIR |
| | | QPPGKGLEWIGVIWGSETTYYQSSLKSRVTISKDNSKNQVSLKLS |
| | | SVTAADTAVYYCAKHYYYGGSYAMDYWGQGTLVTVSSTTTPAP |
| | | RPPTPAPTIASQPLSLRPEACRPAAGGAVHTRGLDFACDIYIWAPL |
| | | AGTCGVLLLSLVITLYCKRGRKKLLYIFKQPFMRPVQTTQEEDGC |
| | | SCRFPEEEEGGCELRVKFSRSADAPAYKQGQNQLYNELNLGRRE |
| | | EYDVLDKRRGRDPEMGGKPRRKNPQEGLYNELQKDKMAEAYSE |
| | | IGMKGERRRGKGHDGLYQGLSTATKDTYDALHMQALPPR |
| 307 | CAR 2 - | atggccctccctgtcaccgccctgctgcttccgctggctcttctgctccacgccgctcggcccgaaattgt |
| | Full - nt | gatgacccagtcacccgccactcttagcctttcacccggtgagcgcgcaaccctgtcttgcagagcctcc |
| | | caagacatctcaaaataccttaattggtatcaacagaagcccggacaggctcctcgccttctgatctacca |
| | | caccagccggctccattctggaatccctgccaggttcagcggtagcggatctgggaccgactacaccct |
| | | cactatcagctcactgcagccagaggacttcgctgtctatttctgtcagcaagggaacaccctgccctac |
| | | acctttggacagggcaccaagctcgagattaaaggtggaggtggcagcggaggaggtgggtccggc |
| | | ggtggaggaagccaggtccaactccaagaaagcggaccgggtcttgtgaagccatcagaaactctttc |
| | | actgacttgtactgtgagcggagtgtctctccccgattacggggtgtcttggatcagacagccaccgggg |
| | | aagggtctggaatggattggagtgatttggggctctgagactacttactaccaatcatccctcaagtcacg |
| | | cgtcaccatctcaaaggacaactctaagaatcaggtgtcactgaaactgtcatctgtgaccgcagccgac |
| | | accgccgtgtactattgcgctaagcattactattatggcgggagctacgcaatggattactggggacagg |
| | | gtactctggtcaccgtgtccagcaccactaccccagcaccgaggccacccaccccggctcctaccatc |
| | | gcctcccagcctctgtccctgcgtccggaggcatgtagacccgcagctggtggggccgtgcatacccg |
| | | gggtcttgacttcgcctgcgatatctacatttgggcccctctggctggtacttgcggggtcctgctgctttc |
| | | actcgtgatcactctttactgtaagcgcggtcggaagaagctgctgtacatctttaagcaacccttcatgag |
| | | gcctgtgcagactactcaagaggaggacggctgttcatgccggttcccagaggaggaggaaggcggc |
| | | tgcgaactgcgcgtgaaattcagccgcagcgcagatgctccagcctacaagcaggggcagaaccagc |
| | | tctacaacgaactcaatcttggtcggagagaggagtacgacgtgctggacaagcggagaggacggga |
| | | cccagaaatgggcgggaagccgcgcagaaagaatccccaagagggcctgtacaacgagctccaaaa |
| | | ggataagatggcagaagcctatagcgagattggtatgaaaggggaacgcagaagaggcaaaggcca |
| | | cgacggactgtaccagggactcagcaccgccaccaaggacacctatgacgctcttcacatgcaggccc |
| | | tgccgcctcgg |
| 349 | CAR 2A- | MALPVTALLLPLALLLHAARPEIVMTQSPATLSLSPGERATLSCRASQDIS |
| | Full amino | KYLNWYQQKPGQAPRLLIYHTSRLHSGIPARFSGSGSGTDYTLTISSLQPE |
| | acid | DFAVYFCQQGNTLPYTFGQGTKLEIKGGGGSGGGGSGGGGSQVQLQES |
| | sequence; | GPGLVKPSETLSLTCTVSGVSLPDYGVSWIRQPPGKGLEWIGVIWGSETT |
| | signal | YYQSSLKSRVTISKDNSKNQVSLKLSSVTAADTAVYYCAKHYYYGGSY |
| | peptide | AMDYWGQGTLVTVSSTTTPAPRPPTPAPTIASQPLSLRPEACRPAAGGAV |
| | underlined | HTRGLDFACDIYIWAPLAGTCGVLLLSLVITLYCKRGRKKLLYIFKQPFM |
| | | RPVQTTQEEDGCSCRFPEEEEGGCELRVKFSRSADAPAYQQGQNQLYNE |
| | | LNLGRREEYDVLDKRRGRDPEMGGKPRRKNPQEGLYNELQKDKMAEA |
| | | YSEIGMKGERRRGKGHDGLYQGLSTATKDTYDALHMQALPPR |
| 225 | CAR 2A - | EIVMTQSPATLSLSPGERATLSCRASQDISKYLNWYQQKPGQAPRLLIYH |
| | amino acid | TSRLHSGIPARFSGSGSGTDYTLTISSLQPEDFAVYFCQQGNTLPYTFGQG |
| | sequence; | TKLEIKGGGGSGGGGSGGGGSQVQLQESGPGLVKPSETLSLTCTVSGVS |
| | no signal | LPDYGVSWIRQPPGKGLEWIGVIWGSETTYYQSSLKSRVTISKDNSKNQ |
| | peptide | VSLKLSSVTAADTAVYYCAKHYYYGGSYAMDYWGQGTLVTVSSTTTP |
| | | APRPPTPAPTIASQPLSLRPEACRPAAGGAVHTRGLDFACDIYIWAPLAG |
| | | TCGVLLLSLVITLYCKRGRKKLLYIFKQPFMRPVQTTQEEDGCSCRFPEE |
| | | EEGGCELRVKFSRSADAPAYQQGQNQLYNELNLGRREEYDVLDKRRGR |
| | | DPEMGGKPRRKNPQEGLYNELQKDKMAEAYSEIGMKGERRRGKGHDG |
| | | LYQGLSTATKDTYDALHMQALPPR |
| 354 | CAR 2A | atggccctccctgtcaccgccctgctgcttccgctggctcttctgctccacgccgctcggcccgaaattgtgatgacc |
| | full nucleic | cagtcacccgccactcttagcctttcacccggtgagcgcgcaaccctgtcttgcagagcctcccaagacatctcaaa |
| | acid | ataccttaattggtatcaacagaagcccggacaggctcctcgccttctgatctaccacaccagccggctccattctgg |
| | sequence; | aatccctgccaggttcagcggtagcggatctgggaccgactacaccctcactatcagctcactgcagccagagga |
| | signal | cttcgctgtctatttctgtcagcaagggaacaccctgccctacacctttggacagggcaccaagctcgagattaaag |
| | peptide and | gtggaggtggcagcggaggaggtgggtccggcggtggaggaagccaggtccaactccaagaaagcggaccg |
| | stop codon | ggtcttgtgaagccatcagaaactctttcactgacttgtactgtgagcggagtgtctctccccgattacggggtgtctt |
| | underlined | ggatcagacagccaccggggaagggtctggaatggattggagtgatttggggctctgagactacttactaccaatc |
| | | atccctcaagtcacgcgtcaccatctcaaaggacaactctaagaatcaggtgtcactgaaactgtcatctgtgaccg |
| | | cagccgacaccgccgtgtactattgcgctaagcattactattatggcgggagctacgcaatggattactggggaca |
| | | gggtactctggtcaccgtgtccagcaccactaccccagcaccgaggccacccaccceggctcctaccatcgcctc |
| | | ccagcctctgtccctgcgtccggaggcatgtagacccgcagctggtggggccgtgcatacccggggtcttgacttc |
| | | gcctgcgatatctacatttgggcccctctggctggtacttgggggtcctgctgctttcactcgtgatcactctttactgt |
| | | aagcgcggtcggaagaagctgctgtacatctttaagcaacccttcatgaggcctgtgcagactactcaagaggagg |
| | | acggctgttcatgccggttcccagaggaggaggaaggcggctgcgaactgcgcgtgaaattcagccgcagcgca |
| | | gatgctccagcctaccagcaggggcagaaccagctctacaacgaactcaatcttggtcggagagaggagtacga |
| | | cgtgctggacaagcggagaggacgggacccagaaatgggcgggaagccgcgcagaaagaatccccaagagg |
| | | gcctgtacaacgagctccaaaaggataagatggcagaagcctatagcgagattggtatgaaaggggaacgcaga |
| | | agaggcaaaggccacgacggactgtaccagggactcagcaccgccaccaaggacacctatgacgctcttcacat |
| | | gcaggccctgccgcctcggtaa |
| 355 | CAR 2A | atggccctccctgtcaccgccctgctgcttccgctggctcttctgctccacgccgctcggcccgaaattgtgatgacc |
| | nucleic | cagtcacccgccactcttagcctttcacccggtgagcgcgcaaccctgtcttgcagagcctcccaagacatctcaaa |
| | acid | ataccttaattggtatcaacagaagcccggacaggctcctcgccttctgatctaccacaccagccggctccattctgg |
| | sequence; | aatccctgccaggttcagcggtagcggatctgggaccgactacaccctcactatcagctcactgcagccagagga |
| | signal | cttcgctgtctatttctgtcagcaagggaacaccctgccctacacctttggacagggcaccaagctcgagattaaag |
| | peptide | gtggaggtggcagcggaggaggtgggtccggcggtggaggaagccaggtccaactccaagaaagcggaccg |
| | underlined; | ggtcttgtgaagccatcagaaactctttcactgacttgtactgtgagcggagtgtctctccccgattacggggtgtctt |
| | no stop | ggatcagacagccaccggggaagggtctggaatggattggagtgatttggggctctgagactacttactaccaatc |
| | codon | atccctcaagtcacgcgtcaccatctcaaaggacaactctaagaatcaggtgtcactgaaactgtcatctgtgaccg |
| | | cagccgacaccgccgtgtactattgcgctaagcattactattatggcgggagctacgcaatggattactggggaca |
| | | gggtactctggtcaccgtgtccagcaccactaccccagcaccgaggccacccaccccggctcctaccatcgcctc |
| | | ccagcctctgtccctgcgtccggaggcatgtagacccgcagctggtggggccgtgcatacccggggtcttgacttc |
| | | gcctgcgatatctacatttgggcccctctggctggtacttgcggggtcctgctgctttcactcgtgatcactctttactgt |
| | | aagcgcggtcggaagaagctgctgtacatctttaagcaacccttcatgaggcctgtgcagactactcaagaggagg |
| | | acggctgttcatgccggttcccagaggaggaggaaggcggctgcgaactgcgcgtgaaattcagccgcagcgca |
| | | gatgctccagcctaccagcaggggcagaaccagctctacaacgaactcaatcttggtcggagagaggagtacga |
| | | cgtgctggacaagcggagaggacgggacccagaaatgggcgggaagccgcgcagaaagaatccccaagagg |
| | | gcctgtacaacgagctccaaaaggataagatggcagaagcctatagcgagattggtatgaaaggggaacgcaga |
| | | agaggcaaaggccacgacggactgtaccagggactcagcaccgccaccaaggacacctatgacgctcttcacat |
| | | gcaggccctgccgcctcgg |
| 356 | CAR 2A | gaaattgtgatgacccagtcacccgccactcttagcctttcacccggtgagcgcgcaaccctgtcttgcagagcctc |
| | nucleic | ccaagacatctcaaaataccttaattggtatcaacagaagcccggacaggctcctcgccttctgatctaccacacca |
| | acid | gccggctccattctggaatccctgccaggttcagcggtagoggatctgggaccgactacaccctcactatcagctc |
| | sequence; | actgcagccagaggacttcgctgtctatttctgtcagcaagggaacaccctgccctacacctttggacagggcacca |
| | no signal | agctcgagattaaaggtggaggtggcagcggaggaggtgggtccggcggtggaggaagccaggtccaactcca |
| | peptide; | agaaagcggaccgggtcttgtgaagccatcagaaactctttcactgacttgtactgtgagcggagtgtctctccccg |
| | stop codon | attacggggtgtcttggatcagacagccaccggggaagggtctggaatggattggagtgatttggggctctgagac |
| | underlined | tacttactaccaatcatccctcaagtcacgcgtcaccatctcaaaggacaactctaagaatcaggtgtcactgaaact |
| | | gtcatctgtgaccgcagccgacaccgccgtgtactattgcgctaagcattactattatggcgggagctacgcaatgg |
| | | attactggggacagggtactctggtcaccgtgtccagcaccactaccccagcaccgaggccacccaccccggctc |
| | | ctaccatcgcctcccagcctctgtccctgcgtccggaggcatgtagacccgcagctggtggggccgtgcataccc |
| | | ggggtcttgacttcgcctgcgatatctacatttgggcccctctggctggtacttgcggggtcctgctgctttcactcgtg |
| | | atcactctttactgtaagcgcggtcggaagaagctgctgtacatctttaagcaacccttcatgaggcctgtgcagact |
| | | actcaagaggaggacggctgttcatgccggttcccagaggaggaggaaggcggctgcgaactgcgcgtgaaatt |
| | | cagccgcagcgcagatgctccagcctaccagcaggggcagaaccagctctacaacgaactcaatcttggtcgga |
| | | gagaggagtacgacgtgctggacaagcggagaggacgggacccagaaatgggcgggaagccgcgcagaaa |
| | | gaatccccaagagggcctgtacaacgagctccaaaaggataagatggcagaagcctatagcgagattggtatgaa |
| | | aggggaacgcagaagaggcaaaggccacgacggactgtaccagggactcagcaccgccaccaaggacaccta |
| | | tgacgctcttcacatgcaggccctgccgcctcggtaa |
| SEQ ID | CAR 2A | gaaattgtgatgacccagtcacccgccactcttagcctttcacccggtgagcgcgcaaccctgtcttgca |
| NO: 417 | nucleic | gagcctcccaagacatctcaaaataccttaattggtatcaacagaagcccggacaggctcctcgccttct |
| | acid | gatctaccacaccagccggctccattctggaatccctgccaggttcagcggtagcggatctgggaccga |
| | sequence; | ctacaccctcactatcagctcactgcagccagaggacttcgctgtctatttctgtcagcaagggaacaccc |
| | no signal | tgccctacacctttggacagggcaccaagctcgagattaaaggtggaggtggcagcggaggaggtgg |
| | peptide; no | gtccggcggtggaggaagccaggtccaactccaagaaagcggaccgggtcttgtgaagccatcagaa |
| | stop codon | actctttcactgacttgtactgtgagcggagtgtctctccccgattacggggtgtcttggatcagacagcca |
| | | ccggggaagggtctggaatggattggagtgatttggggctctgagactacttactaccaatcatccctca |
| | | agtcacgcgtcaccatctcaaaggacaactctaagaatcaggtgtcactgaaactgtcatctgtgaccgc |
| | | agccgacaccgccgtgtactattgcgctaagcattactattatggcgggagctacgcaatggattactgg |
| | | ggacagggtactctggtcaccgtgtccagcaccactaccccagcaccgaggccacccaccccggctc |
| | | ctaccatcgcctcccagcctctgtccctgcgtccggaggcatgtagacccgcagctggtggggccgtg |
| | | catacccggggtcttgacttcgcctgcgatatctacatttgggcccctctggctggtacttgcggggtcct |
| | | gctgctttcactcgtgatcactctttactgtaagcgcggtcggaagaagctgctgtacatctttaagcaacc |
| | | cttcatgaggcctgtgcagactactcaagaggaggacggctgttcatgccggttcccagaggaggagg |
| | | aaggcggctgcgaactgcgcgtgaaattcagccgcagcgcagatgctccagcctaccagcaggggc |
| | | agaaccagctctacaacgaactcaatcttggtcggagagaggagtacgacgtgctggacaagcggag |
| | | aggacgggacccagaaatgggcgggaagccgcgcagaaagaatccccaagagggcctgtacaacg |
| | | agctccaaaaggataagatggcagaagcctatagcgagattggtatgaaaggggaacgcagaagagg |
| | | caaaggccacgacggactgtaccagggactcagcaccgccaccaaggacacctatgacgctcttcac |
| | | atgcaggccctgccgcctcgg |
| SEQ ID | Anti-CD19 | QVQLQESGPGLVKPSETLSLTCTVSGVSLPDYGVSWIRQPPGKGLEWIGV |
| NO: 250 | VH | IWGSETTYYQSSLKSRVTISKDNSKNQVSLKLSSVTAADTAVYYCAKHY |
| | | YYGGSYAMDYWGQGTLVTVSS |
| SEQ ID | Anti-CD19 | EIVMTQSPATLSLSPGERATLSCRASQDISKYLNWYQQKPGQAPRLLIYH |
| NO: 251 | VL | TSRLHSGIPARFSGSGSGTDYTLTISSLQPEDFAVYFCQQGNTLPYTFGQG |
| | | TKLEIK |
| SEQ ID | VH | QVQLQESGPGLVKPSETLSLTCTVSGVSLPDYGVSWIRQPPGKCLEWIGV |
| NO: 331 | | IWGSETTYYQSSLKSRVTISKDNSKNQVSLKLSSVTAADTAVYYCAKHY |
| | | YYGGSYAMDYWGQGTLVTVSS |
| SEQ ID | VL | EIVMTQSPATLSLSPGERATLSCRASQDISKYLNWYQQKPGQAPRLLIYH |
| NO: 332 | | TSRLHSGIPARFSGSGSGTDYTLTISSLQPEDFAVYFCQQGNTLPYTFGCG |
| | | TKLEIK |

### Other Exemplary CAR Properties

In some embodiments, the B cell antigen-binding domain is a fragment, for example, a single chain variable fragment (scFv). In some embodiments, the B cell antigen binding domain is a Fv, a Fab, a (Fab′)2, or a bi-functional (for example bi-specific) hybrid antibody (for example, Lanzavecchia et al., Eur. J. Immunol. 17, 105 (1987)). In some embodiments, the antibodies and fragments thereof of the invention binds a B cell antigen as described herein protein with wild-type or enhanced affinity.

In some instances, scFvs can be prepared according to a method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (for example, a Ser-Gly linker) with an optimized length and/or amino acid composition. The linker length can greatly affect how the variable regions of a scFv fold and interact. In fact, if a short polypeptide linker is employed (for example, between 5-10 amino acids) intrachain folding is prevented. Interchain folding is also required to bring the two variable regions together to form a functional epitope binding site. For examples of linker orientation and size see, for example, Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, which are incorporated herein by reference.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In some embodiments, the linker sequence comprises sets of glycine and serine repeats such as (Gly₄Ser) n, where n is a positive integer equal to or greater than 1 (SEQ ID NO: 25). In some embodiments, the linker can be (Gly₄Ser)₄ (SEQ ID NO: 27) or (Gly₄Ser)₃ (SEQ ID NO: 28). Variation in the linker length may retain or enhance activity, giving rise to superior efficacy in activity studies.

In some embodiments, the antigen-binding domain is a T cell receptor (“TCR”), or a fragment thereof, for example, a single chain TCR (scTCR). Methods to make such TCRs are known in the art. See, for example, Willemsen R A et al, Gene Therapy 7:1369-1377 (2000); Zhang T et al, Cancer Gene Ther 11:487-496 (2004); Aggen et al, Gene Ther. 19 (4): 365-74 (2012) (references are incorporated herein by its entirety). For example, scTCR can be engineered that contains the Va and VB genes from a T cell clone linked by a linker (for example, a flexible peptide).

### Transmembrane Domain

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, for example, one or more amino acid associated with the extracellular region of the protein from which the transmembrane was derived (for example, 1, 2, 3, 4, 5, 6, 7, 8, 9, 10 up to 15 amino acids of the extracellular region) and/or one or more additional amino acids associated with the intracellular region of the protein from which the transmembrane protein is derived (for example, 1, 2, 3, 4, 5, 6, 7, 8, 9, 10 up to 15 amino acids of the intracellular region). In some embodiments, the transmembrane domain is one that is associated with one of the other domains of the CAR is used. In some instances, the transmembrane domain can be selected or modified by amino acid substitution to avoid binding of such domains to the transmembrane domains of the same or different surface membrane proteins, for example, to minimize interactions with other members of the receptor complex. In some embodiments, the transmembrane domain is capable of homodimerization with another CAR on the CAR-expressing cell, for example, CART cell, surface. In some embodiments the amino acid sequence of the transmembrane domain may be modified or substituted so as to minimize interactions with the binding domains of the native binding partner present in the same CAR-expressing cell, for example, CART.

The transmembrane domain may be derived either from a natural or from a recombinant source. Where the source is natural, the domain may be derived from any membrane-bound or transmembrane protein. In some embodiments the transmembrane domain is capable of signaling to the intracellular domain(s) whenever the CAR has bound to a target. A transmembrane domain of particular use in this invention may include at least the transmembrane region(s) of, for example, the alpha, beta or zeta chain of T-cell receptor, CD28, CD3 epsilon, CD45, CD4, CD5, CD8 (for example, CD8 alpha, CD8 beta), CD9, CD16, CD22, CD33, CD37, CD64, CD80, CD86, CD134, CD137, CD154. In some embodiments, a transmembrane domain may include at least the transmembrane region(s) of a costimulatory molecule, for example, MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

In some instances, the transmembrane domain can be attached to the extracellular region of the CAR, for example, the antigen-binding domain of the CAR, via a hinge, for example, a hinge from a human protein. For example, in some embodiments, the hinge can be a human Ig (immunoglobulin) hinge, for example, an IgG4 hinge, or a CD8a hinge. In some embodiments, the hinge or spacer comprises (for example, consists of) the amino acid sequence of SEQ ID NO: 2. In some embodiments, the transmembrane domain comprises (for example, consists of) a transmembrane domain of SEQ ID NO: 6.

In some embodiments, the hinge or spacer comprises an IgG4 hinge. For example, in some embodiments, the hinge or spacer comprises a hinge of SEQ ID NO: 3. In some embodiments, the hinge or spacer comprises a hinge encoded by the nucleotide sequence of SEQ ID NO: 14.

In some embodiments, the hinge or spacer comprises an IgD hinge. For example, in some embodiments, the hinge or spacer comprises a hinge of the amino acid sequence of SEQ ID NO: 4. In some embodiments, the hinge or spacer comprises a hinge encoded by the nucleotide sequence of SEQ ID NO:15.

In some embodiments, the transmembrane domain may be recombinant, in which case it will comprise predominantly hydrophobic residues such as leucine and valine. In some embodiments, a triplet of phenylalanine, tryptophan and valine can be found at each end of a recombinant transmembrane domain.

Optionally, a short oligo- or polypeptide linker, between 2 and 10 amino acids in length may form the linkage between the transmembrane domain and the cytoplasmic region of the CAR. A glycine-serine doublet provides a particularly suitable linker. For example, in some embodiments, the linker comprises the amino acid sequence of SEQ ID NO: 5. In some embodiments, the linker is encoded by a nucleotide sequence of SEQ ID NO: 16.

In some embodiments, the hinge or spacer comprises a KIR2DS2 hinge.

### Cytoplasmic Domain

The cytoplasmic domain or region of a CAR of the present invention includes an intracellular signaling domain. An intracellular signaling domain is generally responsible for activation of at least one of the normal effector functions of the immune cell in which the CAR has been introduced.

Examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, for example, a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAM containing primary intracellular signaling domains that are of particular use in the invention include those of TCR zeta, FcR gamma, FcR beta, CD3 gamma, CD3 delta, CD3 epsilon, CD5, CD22, CD79a, CD79b, CD278 (also known as “ICOS”), FcεRI, DAP10, DAP12, and CD66d. In some embodiments, a CAR of the invention comprises an intracellular signaling domain, for example, a primary signaling domain of CD3-zeta.

In some embodiments, a primary signaling domain comprises a modified ITAM domain, for example, a mutated ITAM domain which has altered (for example, increased or decreased) activity as compared to the native ITAM domain. In some embodiments, a primary signaling domain comprises a modified ITAM-containing primary intracellular signaling domain, for example, an optimized and/or truncated ITAM-containing primary intracellular signaling domain. In some embodiments, a primary signaling domain comprises one, two, three, four or more ITAM motifs.

Further examples of molecules containing a primary intracellular signaling domain that are of particular use in the invention include those of DAP10, DAP12, and CD32.

The intracellular signaling domain of the CAR can comprise the primary signaling domain, for example, CD3-zeta signaling domain, by itself or it can be combined with any other desired intracellular signaling domain(s) useful in the context of a CAR of the invention. For example, the intracellular signaling domain of the CAR can comprise a primary signaling domain, for example, CD3 zeta chain portion, and a costimulatory signaling domain. The costimulatory signaling domain refers to a portion of the CAR comprising the intracellular domain of a costimulatory molecule. A costimulatory molecule is a cell surface molecule other than an antigen receptor or its ligands that is required for an efficient response of lymphocytes to an antigen. Examples of such molecules include MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83, and the like. For example, CD27 costimulation has been demonstrated to enhance expansion, effector function, and survival of human CART cells in vitro and augments human T cell persistence and antitumor activity in vivo (Song et al. Blood. 2012; 119 (3): 696-706). The intracellular signaling sequences within the cytoplasmic portion of the CAR of the invention may be linked to each other in a random or specified order. Optionally, a short oligo- or polypeptide linker, for example, between 2 and 10 amino acids (for example, 2, 3, 4, 5, 6, 7, 8, 9, or 10 amino acids) in length may form the linkage between intracellular signaling sequence. In some embodiments, a glycine-serine doublet can be used as a suitable linker. In some embodiments, a single amino acid, for example, an alanine, a glycine, can be used as a suitable linker.

In some embodiments, the intracellular signaling domain is designed to comprise two or more, for example, 2, 3, 4, 5, or more, costimulatory signaling domains. In some embodiments, the two or more, for example, 2, 3, 4, 5, or more, costimulatory signaling domains, are separated by a linker molecule, for example, a linker molecule described herein. In some embodiments, the intracellular signaling domain comprises two costimulatory signaling domains. In some embodiments, the linker molecule is a glycine residue. In some embodiments, the linker is an alanine residue.

In some embodiments, the intracellular signaling domain is designed to comprise the signaling domain of CD3-zeta and the signaling domain of CD28. In some embodiments, the intracellular signaling domain is designed to comprise the signaling domain of CD3-zeta and the signaling domain of 4-1BB. In some embodiments, the signaling domain of 4-1BB is a signaling domain of SEQ ID NO: 7. In some embodiments, the signaling domain of CD3-zeta is a signaling domain of SEQ ID NO: 9 (mutant CD3zeta) or SEQ ID NO: 10 (wild type human CD3zeta).

In some embodiments, the intracellular signaling domain is designed to comprise the signaling domain of CD3-zeta and the signaling domain of CD27. In some embodiments, the signaling domain of CD27 comprises the amino acid sequence of SEQ ID NO: 8. In some embodiments, the signaling domain of CD27 is encoded by the nucleic acid sequence of SEQ ID NO: 19.

In some embodiments, the intracellular is designed to comprise the signaling domain of CD3-zeta and the signaling domain of CD28. In some embodiments, the signaling domain of CD28 comprises the amino acid sequence of SEQ ID NO: 36. In some embodiments, the signaling domain of CD28 is encoded by the nucleic acid sequence of SEQ ID NO: 37. In some embodiments, the intracellular is designed to comprise the signaling domain of CD3-zeta and the signaling domain of ICOS. In some embodiments, the signaling domain of ICOS comprises the amino acid sequence of SEQ ID NO: 38. In some embodiments, the signaling domain of ICOS is encoded by the nucleic acid sequence of SEQ ID NO: 39.

Co-Expression of CAR with Other Molecules or Agents

### Co-Expression of a Second CAR

In some embodiments, the CAR-expressing cell described herein can further comprise a second CAR, for example, a second CAR that includes a different antigen-binding domain, for example, to the same target (for example, CD19) or a different target (for example, a target other than CD19, for example, a target described herein).

In some embodiments, the CAR-expressing cell comprises a first CAR that targets a first antigen and includes an intracellular signaling domain having a costimulatory signaling domain but not a primary signaling domain, and a second CAR that targets a second, different, antigen and includes an intracellular signaling domain having a primary signaling domain but not a costimulatory signaling domain. Placement of a costimulatory signaling domain, for example, 4-1BB, CD28, CD27, OX-40 or ICOS, onto the first CAR, and the primary signaling domain, for example, CD3 zeta, on the second CAR can limit the CAR activity to cells where both targets are expressed. In some embodiments, the CAR expressing cell comprises a first CAR that includes an antigen-binding domain, a transmembrane domain and a costimulatory domain and a second CAR that targets another antigen and includes an antigen-binding domain, a transmembrane domain and a primary signaling domain. In some embodiments, the CAR expressing cell comprises a first CAR that includes an antigen-binding domain, a transmembrane domain and a primary signaling domain and a second CAR that targets another antigen and includes an antigen-binding domain to the antigen, a transmembrane domain and a costimulatory signaling domain.

In some embodiments, when the CAR-expressing cell comprises two or more different CARs, the antigen-binding domains of the different CARs can be such that the antigen-binding domains do not interact with one another. For example, a cell expressing a first and second CAR can have an antigen-binding domain of the first CAR, for example, as a fragment, for example, an scFv, that does not form an association with the antigen-binding domain of the second CAR, for example, the antigen-binding domain of the second CAR is a VHH.

In some embodiments, the antigen-binding domain comprises a single domain antigen-binding (SDAB) molecules include molecules whose complementary determining regions are part of a single domain polypeptide. Examples include, but are not limited to, heavy chain variable domains, binding molecules naturally devoid of light chains, single domains derived from conventional 4-chain antibodies, engineered domains and single domain scaffolds other than those derived from antibodies. SDAB molecules may be any of the art, or any future single domain molecules. SDAB molecules may be derived from any species including, but not limited to mouse, human, camel, llama, lamprey, fish, shark, goat, rabbit, and bovine. This term also includes naturally occurring single domain antibody molecules from species other than Camelidae and sharks.

In some embodiments, an SDAB molecule can be derived from a variable region of the immunoglobulin found in fish, such as, for example, that which is derived from the immunoglobulin isotype known as Novel Antigen Receptor (NAR) found in the serum of shark. Methods of producing single domain molecules derived from a variable region of NAR (“IgNARs”) are described in WO 03/014161 and Streltsov (2005) Protein Sci. 14:2901-2909.

In some embodiments, an SDAB molecule is a naturally occurring single domain antigen-binding molecule known as heavy chain devoid of light chains. Such single domain molecules are disclosed in WO 9404678 and Hamers-Casterman, C. et al. (1993) Nature 363:446-448, for example. For clarity reasons, this variable domain derived from a heavy chain molecule naturally devoid of light chain is known herein as a VHH or nanobody to distinguish it from the conventional VH of four chain immunoglobulins. Such a VHH molecule can be derived from Camelidae species, for example in camel, llama, dromedary, alpaca and guanaco. Other species besides Camelidae may produce heavy chain molecules naturally devoid of light chain; such VHHs are within the scope of the invention.

The SDAB molecules can be recombinant, CDR-grafted, humanized, camelized, de-immunized and/or in vitro generated (for example, selected by phage display).

It has also been discovered, that cells having a plurality of chimeric membrane embedded receptors comprising an antigen-binding domain that interactions between the antigen-binding domain of the receptors can be undesirable, for example, because it inhibits the ability of one or more of the antigen-binding domains to bind its cognate antigen. Accordingly, disclosed herein are cells having a first and a second non naturally occurring chimeric membrane embedded receptor comprising antigen-binding domains that minimize such interactions. Also disclosed herein are nucleic acids encoding a first and a second non naturally occurring chimeric membrane embedded receptor comprising an antigen-binding domains that minimize such interactions, as well as methods of making and using such cells and nucleic acids. In some embodiments the antigen-binding domain of one of the first and the second non-naturally occurring chimeric membrane embedded receptor, comprises an scFv, and the other comprises a single VH domain, for example, a camelid, shark, or lamprey single VH domain, or a single VH domain derived from a human or mouse sequence.

In some embodiments, a composition herein comprises a first and second CAR, wherein the antigen-binding domain of one of the first and the second CAR does not comprise a variable light domain and a variable heavy domain. In some embodiments, the antigen-binding domain of one of the first and the second CAR is an scFv, and the other is not an scFv. In some embodiments, the antigen-binding domain of one of the first and the second CAR comprises a single VH domain, for example, a camelid, shark, or lamprey single VH domain, or a single VH domain derived from a human or mouse sequence. In some embodiments, the antigen-binding domain of one of the first and the second CAR comprises a nanobody. In some embodiments, the antigen-binding domain of one of the first and the second CAR comprises a camelid VHH domain.

In some embodiments, the antigen-binding domain of one of the first and the second CAR comprises an scFv, and the other comprises a single VH domain, for example, a camelid, shark, or lamprey single VH domain, or a single VH domain derived from a human or mouse sequence. In some embodiments, the antigen-binding domain of one of the first and the second CAR comprises an scFv, and the other comprises a nanobody. In some embodiments, the antigen-binding domain of one of the first and the second CAR comprises an scFv, and the other comprises a camelid VHH domain.

In some embodiments, when present on the surface of a cell, binding of the antigen-binding domain of the first CAR to its cognate antigen is not substantially reduced by the presence of the second CAR. In some embodiments, binding of the antigen-binding domain of the first CAR to its cognate antigen in the presence of the second CAR is at least 85%, 90%, 95%, 96%, 97%, 98% or 99%, for example, 85%, 90%, 95%, 96%, 97%, 98% or 99% of binding of the antigen-binding domain of the first CAR to its cognate antigen in the absence of the second CAR.

In some embodiments, when present on the surface of a cell, the antigen-binding domains of the first and the second CAR, associate with one another less than if both were scFv antigen-binding domains. In some embodiments, the antigen-binding domains of the first and the second CAR, associate with one another at least 85%, 90%, 95%, 96%, 97%, 98% or 99% less than, for example, 85%, 90%, 95%, 96%, 97%, 98% or 99% less than if both were scFv antigen-binding domains.

Co-Expression of an Agent that Enhances CAR Activity

In some embodiments, the CAR-expressing cell described herein can further express another agent, for example, an agent that enhances the activity or fitness of a CAR-expressing cell.

For example, in some embodiments, the agent can be an agent which inhibits a molecule that modulates or regulates, for example, inhibits, T cell function. In some embodiments, the molecule that modulates or regulates T cell function is an inhibitory molecule. Inhibitory molecules, for example, PD1, can, in some embodiments, decrease the ability of a CAR-expressing cell to mount an immune effector response. Examples of inhibitory molecules include PD1, PD-L1, CTLA4, TIM3, LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, or TGF beta.

In embodiments, an agent, for example, an inhibitory nucleic acid, for example, a dsRNA, for example, an siRNA or shRNA; or for example, an inhibitory protein or system, for example, a clustered regularly interspaced short palindromic repeats (CRISPR), a transcription-activator like effector nuclease (TALEN), or a zinc finger endonuclease (ZFN), for example, as described herein, can be used to inhibit expression of a molecule that modulates or regulates, for example, inhibits, T-cell function in the CAR-expressing cell. In some embodiments the agent is an shRNA, for example, an shRNA described herein. In some embodiments, the agent that modulates or regulates, for example, inhibits, T-cell function is inhibited within a CAR-expressing cell. For example, a dsRNA molecule that inhibits expression of a molecule that modulates or regulates, for example, inhibits, T-cell function is linked to the nucleic acid that encodes a component, for example, all of the components, of the CAR.

In some embodiments, the agent which inhibits an inhibitory molecule comprises a first polypeptide, for example, an inhibitory molecule, associated with a second polypeptide that provides a positive signal to the cell, for example, an intracellular signaling domain described herein. In some embodiments, the agent comprises a first polypeptide, for example, of an inhibitory molecule such as PD1, PD-L1, CTLA4, TIM3, LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, or TGF beta, or a fragment of any of these (for example, at least a portion of an extracellular domain of any of these), and a second polypeptide which is an intracellular signaling domain described herein (for example, comprising a costimulatory domain (for example, 41BB, CD27 or CD28, for example, as described herein) and/or a primary signaling domain (for example, a CD3 zeta signaling domain described herein). In some embodiments, the agent comprises a first polypeptide of PD1 or a fragment thereof (for example, at least a portion of an extracellular domain of PD1), and a second polypeptide of an intracellular signaling domain described herein (for example, a CD28 signaling domain described herein and/or a CD3 zeta signaling domain described herein). PD1 is an inhibitory member of the CD28 family of receptors that also includes CD28, CTLA-4, ICOS, and BTLA. PD-1 is expressed on activated B cells, T cells and myeloid cells (Agata et al. 1996 Int. Immunol 8:765−75). Two ligands for PD1, PD-L1 and PD-L2 have been shown to downregulate T cell activation upon binding to PD1 (Freeman et a. 2000 J Exp Med 192:1027-34; Latchman et al. 2001 Nat Immunol 2:261-8; Carter et al. 2002 Eur J Immunol 32:634-43).

In some embodiments, the agent comprises the extracellular domain (ECD) of an inhibitory molecule, for example, Programmed Death 1 (PD1), can be fused to a transmembrane domain and intracellular signaling domains such as 41BB and CD3 zeta (also referred to herein as a PD1 CAR). In some embodiments, the PD1 CAR, when used in combinations with an XCAR described herein, improves the persistence of the T cell. In some embodiments, the CAR is a PD1 CAR comprising the extracellular domain of PD1 indicated as underlined in SEQ ID NO: 24. In some embodiments, the PD1 CAR comprises the amino acid sequence of SEQ ID NO: 24.

In some embodiments, the PD1 CAR comprises the amino acid sequence of SEQ ID NO: 22.

In some embodiments, the agent comprises a nucleic acid sequence encoding the PD1 CAR, for example, the PD1 CAR described herein. In some embodiments, the nucleic acid sequence for the PD1 CAR is provided as SEQ ID NO: 23, with the PD1 ECD underlined.

In another example, in some embodiments, the agent which enhances the activity of a CAR-expressing cell can be a costimulatory molecule or costimulatory molecule ligand. Examples of costimulatory molecules include MHC class I molecule, BTLA and a Toll ligand receptor, as well as OX40, CD27, CD28, CDS, ICAM-1, LFA-1 (CD11a/CD18), ICOS (CD278), and 4-1BB (CD137). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83., for example, as described herein. Examples of costimulatory molecule ligands include CD80, CD86, CD40L, ICOSL, CD70, OX40L, 4-1BBL, GITRL, and LIGHT. In embodiments, the costimulatory molecule ligand is a ligand for a costimulatory molecule different from the costimulatory molecule domain of the CAR. In embodiments, the costimulatory molecule ligand is a ligand for a costimulatory molecule that is the same as the costimulatory molecule domain of the CAR. In some embodiments, the costimulatory molecule ligand is 4-1BBL. In some embodiments, the costimulatory ligand is CD80 or CD86. In some embodiments, the costimulatory molecule ligand is CD70. In embodiments, a CAR-expressing immune effector cell described herein can be further engineered to express one or more additional costimulatory molecules or costimulatory molecule ligands.

### Nucleic Acid Constructs Encoding a CAR

The present invention also provides an immune effector cell, for example, made by a method described herein, that includes a nucleic acid molecule encoding one or more CAR constructs described herein. In some embodiments, the nucleic acid molecule is provided as a messenger RNA transcript. In some embodiments, the nucleic acid molecule is provided as a DNA construct.

The nucleic acid molecules described herein can be a DNA molecule, an RNA molecule, or a combination thereof. In some embodiments, the nucleic acid molecule is an mRNA encoding a CAR polypeptide as described herein. In other embodiments, the nucleic acid molecule is a vector that includes any of the aforesaid nucleic acid molecules.

In some embodiments, the antigen-binding domain of a CAR of the invention (for example, a scFv) is encoded by a nucleic acid molecule whose sequence has been codon optimized for expression in a mammalian cell. In some embodiments, entire CAR construct of the invention is encoded by a nucleic acid molecule whose entire sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, for example, methods disclosed in at least U.S. Pat. Nos. 5,786,464 and 6,114,148.

Accordingly, in some embodiments, an immune effector cell, for example, made by a method described herein, includes a nucleic acid molecule encoding a chimeric antigen receptor (CAR), wherein the CAR comprises an antigen-binding domain that binds to a B cell antigen described herein, a transmembrane domain (for example, a transmembrane domain described herein), and an intracellular signaling domain (for example, an intracellular signaling domain described herein) comprising a stimulatory domain, for example, a costimulatory signaling domain (for example, a costimulatory signaling domain described herein) and/or a primary signaling domain (for example, a primary signaling domain described herein, for example, a zeta chain described herein).

The present invention also provides vectors in which a nucleic acid molecule encoding a CAR, for example, a nucleic acid molecule described herein, is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A retroviral vector may also be, for example, a gammaretroviral vector. A gammaretroviral vector may include, for example, a promoter, a packaging signal (v), a primer binding site (PBS), one or more (for example, two) long terminal repeats (LTR), and a transgene of interest, for example, a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, for example, in Tobias Maetzig et al., “Gammaretroviral Vectors: Biology, Technology and Application” Viruses. 2011 June; 3 (6): 677-713.

In some embodiments, the vector comprising the nucleic acid encoding the desired CAR is an adenoviral vector (A5/35). In some embodiments, the expression of nucleic acids encoding CARs can be accomplished using of transposons such as sleeping beauty, crisper, CAS9, and zinc finger nucleases. See below June et al. 2009 Nature Reviews Immunology 9.10:704-716, is incorporated herein by reference.

In brief summary, the expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (for example, WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used.

Additional promoter elements, for example, enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription. Exemplary promoters include the CMV IE gene, EF-1a, ubiquitin C, or phosphoglycerokinase (PGK) promoters.

An example of a promoter that is capable of expressing a CAR encoding nucleic acid molecule in a mammalian T cell is the EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving CAR expression from nucleic acid molecules cloned into a lentiviral vector. See, for example, Milone et al., Mol. Ther. 17 (8): 1453-1464 (2009). In some embodiments, the EF1a promoter comprises the sequence provided in the Examples.

Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1α promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Another example of a promoter is the phosphoglycerate kinase (PGK) promoter. In embodiments, a truncated PGK promoter (for example, a PGK promoter with one or more, for example, 1, 2, 5, 10, 100, 200, 300, or 400, nucleotide deletions when compared to the wild-type PGK promoter sequence) may be desired.

The nucleotide sequences of exemplary PGK promoters are provided below.

(SEQ ID NO: 190)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACGAGGGACCGCGACAGGCAGACGCTCCCATG
ATCACTCTGCACGCCGAAGGCAAATAGTGCAGGCCGTGCGGCGCTTGGCG
TTCCTTGGAAGGGCTGAATCCCCGCCTCGTCCTTCGCAGCGGCCCCCCGG
GTGTTCCCATCGCCGCTTCTAGGCCCACTGCGACGCTTGCCTGCACTTCT
TACACGCTCTGGGTCCCAGCCGCGGCGACGCAAAGGGCCTTGGTGCGGGT
CTCGTCGGCGCAGGGACGCGTTTGGGTCCCGACGGAACCTTTTCCGCGTT
GGGGTTGGGGCACCATAAGCT

### Exemplary Truncated PGK Promoters:

PGK100:
(SEQ ID NO: 198)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTG
PGK200:
(SEQ ID NO: 191)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACG
PGK300:
(SEQ ID NO: 192)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACGAGGGACCGCGACAGGCAGACGCTCCCATG
ATCACTCTGCACGCCGAAGGCAAATAGTGCAGGCCGTGCGGCGCTTGGCG
TTCCTTGGAAGGGCTGAATCCCCG
PGK400:
(SEQ ID NO: 193)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACGAGGGACCGCGACAGGCAGACGCTCCCATG
ATCACTCTGCACGCCGAAGGCAAATAGTGCAGGCCGTGCGGCGCTTGGCG
TTCCTTGGAAGGGCTGAATCCCCGCCTCGTCCTTCGCAGCGGCCCCCCGG
GTGTTCCCATCGCCGCTTCTAGGCCCACTGCGACGCTTGCCTGCACTTCT
TACACGCTCTGGGTCCCAGCCG

A vector may also include, for example, a signal sequence to facilitate secretion, a polyadenylation signal and transcription terminator (for example, from Bovine Growth Hormone (BGH) gene), an element allowing episomal replication and replication in prokaryotes (for example SV40 origin and ColEl or others known in the art) and/or elements to allow selection (for example, ampicillin resistance gene and/or zeocin marker).

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In some embodiments, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, for example, enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (for example, Ui-Tei et al., 2000 FEBS Letters 479:79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In embodiments, the vector may comprise two or more nucleic acid sequences encoding a CAR, for example, a CAR described herein, for example, a CD19 CAR, and a second CAR, for example, a CAR that specifically binds to an antigen other than CD19. In such embodiments, the two or more nucleic acid sequences encoding the CAR are encoded by a single nucleic molecule in the same frame and as a single polypeptide chain. In some embodiments, the two or more CARs, can, for example, be separated by one or more peptide cleavage sites. (for example, an auto-cleavage site or a substrate for an intracellular protease). Examples of peptide cleavage sites include T2A, P2A, E2A, or F2A sites.

Methods of introducing and expressing genes into a cell are known in the art. In the context of an expression vector, the vector can be readily introduced into a host cell, for example, mammalian, bacterial, yeast, or insect cell by any method, for example, one known in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY). A suitable method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, for example, human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (for example, an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In some embodiments, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL.). Stock solutions of lipids in chloroform or chloroform/methanol can be stored at about-20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5:505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant nucleic acid sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, for example, by immunological means (ELISAs and Western blots) or by assays described herein to identify agents falling within the scope of the invention.

### Natural Killer Cell Receptor (NKR) CARs

In some embodiments, the CAR molecule described herein comprises one or more components of a natural killer cell receptor (NKR), thereby forming an NKR-CAR. The NKR component can be a transmembrane domain, a hinge domain, or a cytoplasmic domain from any of the following natural killer cell receptors: killer cell immunoglobulin-like receptor (KIR), for example, KIR2DL1, KIR2DL2/L3, KIR2DL4, KIR2DL5A, KIR2DL5B, KIR2DS1, KIR2DS2, KIR2DS3, KIR2DS4, DIR2DS5, KIR3DL1/S1, KIR3DL2, KIR3DL3, KIR2DP1, and KIR3DP1; natural cytotoxicity receptor (NCR), for example, NKp30, NKp44, NKp46; signaling lymphocyte activation molecule (SLAM) family of immune cell receptors, for example, CD48, CD229, 2B4, CD84, NTB-A, CRACC, BLAME, and CD2F-10; Fc receptor (FcR), for example, CD16, and CD64; and Ly49 receptors, for example, LY49A, LY49C. The NKR-CAR molecules described herein may interact with an adaptor molecule or intracellular signaling domain, for example, DAP12. Exemplary configurations and sequences of CAR molecules comprising NKR components are described in International Publication No. WO2014/145252, the contents of which are hereby incorporated by reference.

### Split CAR

In some embodiments, the CAR-expressing cell uses a split CAR. The split CAR approach is described in more detail in publications WO2014/055442 and WO2014/055657. Briefly, a split CAR system comprises a cell expressing a first CAR having a first antigen-binding domain and a costimulatory domain (for example, 41BB), and the cell also expresses a second CAR having a second antigen-binding domain and an intracellular signaling domain (for example, CD3 zeta). When the cell encounters the first antigen, the costimulatory domain is activated, and the cell proliferates. When the cell encounters the second antigen, the intracellular signaling domain is activated and cell-killing activity begins. Thus, the CAR-expressing cell is only fully activated in the presence of both antigens.

### Strategies for Regulating Chimeric Antigen Receptors

In some embodiments, a regulatable CAR (RCAR) where the CAR activity can be controlled is desirable to optimize the safety and efficacy of a CAR therapy. There are many ways CAR activities can be regulated. For example, inducible apoptosis using, for example, a caspase fused to a dimerization domain (see, for example, Di Stasa et al., N Engl. J. Med. 2011 Nov. 3; 365 (18): 1673-1683), can be used as a safety switch in the CAR therapy of the instant invention. In some embodiments, the cells (for example, T cells or NK cells) expressing a CAR of the present invention further comprise an inducible apoptosis switch, wherein a human caspase (for example, caspase 9) or a modified version is fused to a modification of the human FKB protein that allows conditional dimerization. In the presence of a small molecule, such as a rapalog (for example, AP 1903, AP20187), the inducible caspase (for example, caspase 9) is activated and leads to the rapid apoptosis and death of the cells (for example, T cells or NK cells) expressing a CAR of the present invention. Examples of a caspase-based inducible apoptosis switch (or one or more aspects of such a switch) have been described in, for example, US2004040047; US20110286980; US20140255360; WO1997031899; WO2014151960; WO2014164348; WO2014197638; WO2014197638; all of which are incorporated by reference herein.

In another example, CAR-expressing cells can also express an inducible Caspase-9 (iCaspase-9) molecule that, upon administration of a dimerizer drug (for example, rimiducid (also called AP1903 (Bellicum Pharmaceuticals) or AP20187 (Ariad)) leads to activation of the Caspase-9 and apoptosis of the cells. The iCaspase-9 molecule contains a chemical inducer of dimerization (CID) binding domain that mediates dimerization in the presence of a CID. This results in inducible and selective depletion of CAR-expressing cells. In some cases, the iCaspase-9 molecule is encoded by a nucleic acid molecule separate from the CAR-encoding vector(s). In some cases, the iCaspase-9 molecule is encoded by the same nucleic acid molecule as the CAR-encoding vector. The iCaspase-9 can provide a safety switch to avoid any toxicity of CAR-expressing cells. See, for example, Song et al. Cancer Gene Ther. 2008; 15 (10): 667-75; Clinical Trial Id. No. NCT02107963; and Di Stasi et al. N. Engl. J. Med. 2011; 365:1673-83.

Alternative strategies for regulating the CAR therapy of the instant invention include utilizing small molecules or antibodies that deactivate or turn off CAR activity, for example, by deleting CAR-expressing cells, for example, by inducing antibody dependent cell-mediated cytotoxicity (ADCC). For example, CAR-expressing cells described herein may also express an antigen that is recognized by molecules capable of inducing cell death, for example, ADCC or complement-induced cell death. For example, CAR expressing cells described herein may also express a receptor capable of being targeted by an antibody or antibody fragment. Examples of such receptors include EpCAM, VEGFR, integrins (for example, integrins avβ3, a4, a13/4B3, a47, a5B1, avβ3, av), members of the TNF receptor superfamily (for example, TRAIL-R1, TRAIL-R2), PDGF Receptor, interferon receptor, folate receptor, GPNMB, ICAM-1, HLA-DR, CEA, CA-125, MUC1, TAG-72, IL-6 receptor, 5T4, GD2, GD3, CD2, CD3, CD4, CD5, CD11, CD1 1 a/LFA-1, CD15, CD18/ITGB2, CD19, CD20, CD22, CD23/1gE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5, CD319/SLAMF7, and EGFR, and truncated versions thereof (for example, versions preserving one or more extracellular epitopes but lacking one or more regions within the cytoplasmic domain).

For example, a CAR-expressing cell described herein may also express a truncated epidermal growth factor receptor (EGFR) which lacks signaling capacity but retains the epitope that is recognized by molecules capable of inducing ADCC, for example, cetuximab (ERBITUX®), such that administration of cetuximab induces ADCC and subsequent depletion of the CAR-expressing cells (see, for example, WO2011/056894, and Jonnalagadda et al., Gene Ther. 2013; 20 (8) 853-860). Another strategy includes expressing a highly compact marker/suicide gene that combines target epitopes from both CD32 and CD20 antigens in the CAR-expressing cells described herein, which binds rituximab, resulting in selective depletion of the CAR-expressing cells, for example, by ADCC (see, for example, Philip et al., Blood. 2014; 124 (8) 1277-1287). Other methods for depleting CAR-expressing cells described herein include administration of CAMPATH, a monoclonal anti-CD52 antibody that selectively binds and targets mature lymphocytes, for example, CAR-expressing cells, for destruction, for example, by inducing ADCC. In other embodiments, the CAR-expressing cell can be selectively targeted using a CAR ligand, for example, an anti-idiotypic antibody. In some embodiments, the anti-idiotypic antibody can cause effector cell activity, for example, ADCC or ADC activities, thereby reducing the number of CAR-expressing cells. In other embodiments, the CAR ligand, for example, the anti-idiotypic antibody, can be coupled to an agent that induces cell killing, for example, a toxin, thereby reducing the number of CAR-expressing cells. Alternatively, the CAR molecules themselves can be configured such that the activity can be regulated, for example, turned on and off, as described below.

In other embodiments, a CAR-expressing cell described herein may also express a target protein recognized by the T cell depleting agent. In some embodiments, the target protein is CD20 and the T cell depleting agent is an anti-CD20 antibody, for example, rituximab. In some embodiments, the T cell depleting agent is administered once it is desirable to reduce or eliminate the CAR-expressing cell, for example, to mitigate the CAR induced toxicity. In other embodiments, the T cell depleting agent is an anti-CD52 antibody, for example, alemtuzumab, as described in the Examples herein.

In other embodiments, an RCAR comprises a set of polypeptides, typically two in the simplest embodiments, in which the components of a standard CAR described herein, for example, an antigen-binding domain and an intracellular signaling domain, are partitioned on separate polypeptides or members. In some embodiments, the set of polypeptides include a dimerization switch that, upon the presence of a dimerization molecule, can couple the polypeptides to one another, for example, can couple an antigen-binding domain to an intracellular signaling domain. In some embodiments, a CAR of the present invention utilizes a dimerization switch as those described in, for example, WO2014127261, which is incorporated by reference herein. Additional description and exemplary configurations of such regulatable CARs are provided herein and in, for example, paragraphs 527-551 of International Publication No. WO 2015/090229 filed Mar. 13, 2015, which is incorporated by reference in its entirety. In some embodiments, an RCAR involves a switch domain, for example, a FKBP switch domain, as set out SEQ ID NO: 275, or comprise a fragment of FKBP having the ability to bind with FRB, for example, as set out in SEQ ID NO: 276. In some embodiments, the RCAR involves a switch domain comprising a FRB sequence, for example, as set out in SEQ ID NO: 277, or a mutant FRB sequence, for example, as set out in any of SEQ ID NOs. 278-283.

(SEQ ID NO: 275)
DVPDYASLGGPSSPKKKRKVSRGVQVETISPGDGRTFPKRGQTCVVHYTG
MLEDGKKFDSSRDRNKPFKFMLGKQEVIRGWEEGVAQMSVGQRAKLTISP
DYAYGATGHPGIIPPHATLVFDVELLKLETSY
(SEQ ID NO: 276)
VQVETISPGDGRTFPKRGQTCVVHYTGMLEDGKKFDSSRDRNKPFKFMLG
KQEVIRGWEEGVAQMSVGQRAKLTISPDYAYGATGHPGIIPPHATLVFDV
ELLKLETS
(SEQ ID NO: 277)
ILWHEMWHEGLEEASRLYFGERNVKGMFEVLEPLHAMMERGPQTLKETSF
NQAYGRDLMEAQEWCRKYMKSGNVKDLTQAWDLYYHVFRRISK

TABLE 18
Exemplary mutant FRB having increased affinity
for a dimerization molecule.

| | | SEQ |
|---|---|---|
| FRB | | ID |
| mutant | Amino Acid Sequence | NO: |
| E2032I | ILWHEMWHEGLIEASRLYFGERNVKGMFEVLEPLHAMM | 278 |
| mutant | ERGPQTLKETSFNQAYGRDLMEAQEWCRKYMKSGNVKD | |
| | LTQAWDLYYHVFRRISKTS | |
| E2032L | ILWHEMWHEGLLEASRLYFGERNVKGMFEVLEPLHAMM | 279 |
| mutant | ERGPQTLKETSFNQAYGRDLMEAQEWCRKYMKSGNVKD | |
| | LTQAWDLYYHVFRRISKTS | |
| T2098L | ILWHEMWHEGLEEASRLYFGERNVKGMFEVLEPLHAMM | 280 |
| mutant | ERGPQTLKETSFNQAYGRDLMEAQEWCRKYMKSGNVKD | |
| | LLQAWDLYYHVFRRISKTS | |
| E2032, | ILWHEMWHEGLXEASRLYFGERNVKGMFEVLEPLHAMM | 281 |
| T2098 | ERGPQTLKETSFNQAYGRDLMEAQEWCRKYMKSGNVKD | |
| mutant | LXQAWDLYYHVFRRISKTS | |
| E2032I, | ILWHEMWHEGLIEASRLYFGERNVKGMFEVLEPLHAMM | 282 |
| T2098L | ERGPQTLKETSFNQAYGRDLMEAQEWCRKYMKSGNVKD | |
| mutant | LLQAWDLYYHVFRRISKTS | |
| E2032L, | ILWHEMWHEGLLEASRLYFGERNVKGMFEVLEPLHAMM | 283 |
| T2098L | ERGPQTLKETSFNQAYGRDLMEAQEWCRKYMKSGNVKD | |
| mutant | LLQAWDLYYHVFRRISKTS | |

### RNA Transfection

Disclosed herein are methods for producing an in vitro transcribed RNA CAR. RNA CAR and methods of using the same are described, for example, in paragraphs 553-570 of in International Application WO2015/142675, filed Mar. 13, 2015, which is herein incorporated by reference in its entirety.

An immune effector cell can include a CAR encoded by a messenger RNA (mRNA). In some embodiments, the mRNA encoding a CAR described herein is introduced into an immune effector cell, for example, made by a method described herein, for production of a CAR-expressing cell.

In some embodiments, the in vitro transcribed RNA CAR can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired temple for in vitro transcription is a CAR described herein. For example, the template for the RNA CAR comprises an extracellular region comprising a single chain variable domain of an antibody to a B cell associated antigen described herein; a hinge region (for example, a hinge region described herein), a transmembrane domain (for example, a transmembrane domain described herein such as a transmembrane domain of CD8a); and a cytoplasmic region that includes an intracellular signaling domain, for example, an intracellular signaling domain described herein, for example, comprising the signaling domain of CD3-zeta and the signaling domain of 4-1BB.

In some embodiments, the DNA to be used for PCR contains an open reading frame. The DNA can be from a naturally occurring DNA sequence from the genome of an organism. In some embodiments, the nucleic acid can include some or all of the 5′ and/or 3′ untranslated regions (UTRs). The nucleic acid can include exons and introns. In some embodiments, the DNA to be used for PCR is a human nucleic acid sequence. In some embodiments, the DNA to be used for PCR is a human nucleic acid sequence including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

PCR is used to generate a template for in vitro transcription of mRNA which is used for transfection. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a nucleic acid that is normally transcribed in cells (the open reading frame), including 5′ and 3′ UTRs. The primers can also be designed to amplify a portion of a nucleic acid that encodes a particular domain of interest. In some embodiments, the primers are designed to amplify the coding region of a human cDNA, including all or portions of the 5′ and 3′ UTRs. Primers useful for PCR can be generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Any DNA polymerase useful for PCR can be used in the methods disclosed herein. The reagents and polymerase are commercially available from a number of sources.

Chemical structures with the ability to promote stability and/or translation efficiency may also be used. The RNA in embodiments has 5′ and 3′ UTRs. In some embodiments, the 5′ UTR is between one and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the nucleic acid of interest. Alternatively, UTR sequences that are not endogenous to the nucleic acid of interest can be added by incorporating the UTR sequences into the forward and reverse primers or by any other modifications of the template. The use of UTR sequences that are not endogenous to the nucleic acid of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In some embodiments, the 5′ UTR can contain the Kozak sequence of the endogenous nucleic acid. Alternatively, when a 5′ UTR that is not endogenous to the nucleic acid of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be 5′UTR of an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In some embodiments, the promoter is a T7 polymerase promoter, as described elsewhere herein. Other useful promoters include, but are not limited to, T3 and SP6 RNA polymerase promoters. Consensus nucleotide sequences for T7, T3, and SP6 promoters are known in the art.

In some embodiments, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485−65 (2003).

The conventional method of integration of poly (A)/T stretches into a DNA template is molecular cloning. However, poly (A)/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with poly (A)/T 3′ stretch without cloning highly desirable.

The poly (A)/T segment of the transcriptional DNA template can be produced during PCR by using a reverse primer containing a polyT tail, such as 100T tail (SEQ ID NO: 31) (size can be 50-5000 T (SEQ ID NO: 32)), or after PCR by any other method, including, but not limited to, DNA ligation or in vitro recombination. Poly (A) tails also provide stability to RNAs and reduce their degradation. Generally, the length of a poly(A) tail positively correlates with the stability of the transcribed RNA. In some embodiments, the poly(A) tail is between 100 and 5000 adenosines (for example, SEQ ID NO: 33).

Poly (A) tails of RNAs can be further extended following in vitro transcription with the use of a poly (A) polymerase, such as E. coli poly (A) polymerase (E-PAP). In some embodiments, increasing the length of a poly(A) tail from 100 nucleotides to between 300 and 400 nucleotides (SEQ ID NO: 34) results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly (A) tail using poly (A) polymerase. ATP analogs can further increase the stability of the RNA.

5′ caps on also provide stability to RNA molecules. In some embodiments, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12 (8): 861-70 (2001).

### Non-Viral Delivery Methods

In some embodiments, non-viral methods can be used to deliver a nucleic acid encoding a CAR described herein into a cell or tissue or a subject.

In some embodiments, the non-viral method includes the use of a transposon (also called a transposable element). In some embodiments, a transposon is a piece of DNA that can insert itself at a location in a genome, for example, a piece of DNA that is capable of self-replicating and inserting its copy into a genome, or a piece of DNA that can be spliced out of a longer nucleic acid and inserted into another place in a genome. For example, a transposon comprises a DNA sequence made up of inverted repeats flanking genes for transposition.

Exemplary methods of nucleic acid delivery using a transposon include a Sleeping Beauty transposon system (SBTS) and a piggyBac™ (PB) transposon system. See, for example, Aronovich et al. Hum. Mol. Genet. 20.R1(2011): R14-20; Singh et al. Cancer Res. 15(2008): 2961-2971; Huang et al. Mol. Ther. 16(2008): 580-589; Grabundzija et al. Mol. Ther. 18(2010): 1200-1209; Kebriaei et al. Blood. 122.21(2013): 166; Williams. Molecular Therapy 16.9(2008): 1515−16; Bell et al. Nat. Protoc. 2.12(2007): 3153-65; and Ding et al. Cell. 122.3(2005): 473-83, all of which are incorporated herein by reference.

The SBTS includes two components: 1) a transposon containing a transgene and 2) a source of transposase enzyme. The transposase can transpose the transposon from a carrier plasmid (or other donor DNA) to a target DNA, such as a host cell chromosome/genome. For example, the transposase binds to the carrier plasmid/donor DNA, cuts the transposon (including transgene(s)) out of the plasmid, and inserts it into the genome of the host cell. See, for example, Aronovich et al. supra.

Exemplary transposons include a pT2-based transposon. See, for example, Grabundzija et al. Nucleic Acids Res. 41.3(2013): 1829-47; and Singh et al. Cancer Res. 68.8(2008): 2961-2971, all of which are incorporated herein by reference. Exemplary transposases include a Tc1/mariner-type transposase, for example, the SB10 transposase or the SB11 transposase (a hyperactive transposase which can be expressed, for example, from a cytomegalovirus promoter). See, for example, Aronovich et al.; Kebriaei et al.; and Grabundzija et al., all of which are incorporated herein by reference.

Use of the SBTS permits efficient integration and expression of a transgene, for example, a nucleic acid encoding a CAR described herein. Provided herein are methods of generating a cell, for example, T cell or NK cell, that stably expresses a CAR described herein, for example, using a transposon system such as SBTS.

In accordance with methods described herein, in some embodiments, one or more nucleic acids, for example, plasmids, containing the SBTS components are delivered to a cell (for example, T or NK cell). For example, the nucleic acid(s) are delivered by standard methods of nucleic acid (for example, plasmid DNA) delivery, for example, methods described herein, for example, electroporation, transfection, or lipofection. In some embodiments, the nucleic acid contains a transposon comprising a transgene, for example, a nucleic acid encoding a CAR described herein. In some embodiments, the nucleic acid contains a transposon comprising a transgene (for example, a nucleic acid encoding a CAR described herein) as well as a nucleic acid sequence encoding a transposase enzyme. In other embodiments, a system with two nucleic acids is provided, for example, a dual-plasmid system, for example, where a first plasmid contains a transposon comprising a transgene, and a second plasmid contains a nucleic acid sequence encoding a transposase enzyme. For example, the first and the second nucleic acids are co-delivered into a host cell.

In some embodiments, cells, for example, T or NK cells, are generated that express a CAR described herein by using a combination of gene insertion using the SBTS and genetic editing using a nuclease (for example, Zinc finger nucleases (ZFNs), Transcription Activator-Like Effector Nucleases (TALENs), the CRISPR/Cas system, or engineered meganuclease re-engineered homing endonucleases).

In some embodiments, use of a non-viral method of delivery permits reprogramming of cells, for example, T or NK cells, and direct infusion of the cells into a subject. Advantages of non-viral vectors include but are not limited to the case and relatively low cost of producing sufficient amounts required to meet a patient population, stability during storage, and lack of immunogenicity.

### Methods of Manufacture/Production

In some embodiments, the methods disclosed herein further include administering a T cell depleting agent after treatment with the cell (for example, an immune effector cell as described herein), thereby reducing (for example, depleting) the CAR-expressing cells (for example, the CD19CAR-expressing cells). Such T cell depleting agents can be used to effectively deplete CAR-expressing cells (for example, CD19CAR-expressing cells) to mitigate toxicity. In some embodiments, the CAR-expressing cells were manufactured according to a method herein, for example, assayed (for example, before or after transfection or transduction) according to a method herein.

In some embodiments, the T cell depleting agent is administered one, two, three, four, or five weeks after administration of the cell, for example, the population of immune effector cells, described herein.

In some embodiments, the T cell depleting agent is an agent that depletes CAR-expressing cells, for example, by inducing antibody dependent cell-mediated cytotoxicity (ADCC) and/or complement-induced cell death. For example, CAR-expressing cells described herein may also express an antigen (for example, a target antigen) that is recognized by molecules capable of inducing cell death, for example, ADCC or complement-induced cell death. For example, CAR expressing cells described herein may also express a target protein (for example, a receptor) capable of being targeted by an antibody or antibody fragment. Examples of such target proteins include, but are not limited to, EpCAM, VEGFR, integrins (for example, integrins αvβ3, α4, αI3/4Bβ3, α4β7, α5β1, αvβ3, αv), members of the TNF receptor superfamily (for example, TRAIL-R1, TRAIL-R2), PDGF Receptor, interferon receptor, folate receptor, GPNMB, ICAM-1, HLA-DR, CEA, CA-125, MUC1, TAG-72, IL-6 receptor, 5T4, GD2, GD3, CD2, CD3, CD4, CD5, CD11, CD11a/LFA-1, CD15, CD18/ITGB2, CD19, CD20, CD22, CD23/1gE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5, CD319/SLAMF7, and EGFR, and truncated versions thereof (for example, versions preserving one or more extracellular epitopes but lacking one or more regions within the cytoplasmic domain).

In some embodiments, the CAR expressing cell co-expresses the CAR and the target protein, for example, naturally expresses the target protein or is engineered to express the target protein. For example, the cell, for example, the population of immune effector cells, can include a nucleic acid (for example, vector) comprising the CAR nucleic acid (for example, a CAR nucleic acid as described herein) and a nucleic acid encoding the target protein.

In some embodiments, the T cell depleting agent is a CD52 inhibitor, for example, an anti-CD52 antibody molecule, for example, alemtuzumab.

In other embodiments, the cell, for example, the population of immune effector cells, expresses a CAR molecule as described herein (for example, CD19CAR) and the target protein recognized by the T cell depleting agent. In some embodiments, the target protein is CD20. In embodiments where the target protein is CD20, the T cell depleting agent is an anti-CD20 antibody, for example, rituximab.

In further embodiments of any of the aforesaid methods, the methods further include transplanting a cell, for example, a hematopoietic stem cell, or a bone marrow, into the mammal.

In some embodiments, the invention features a method of conditioning a mammal prior to cell transplantation. The method includes administering to the mammal an effective amount of the cell comprising a CAR nucleic acid or polypeptide, for example, a CD19 CAR nucleic acid or polypeptide. In some embodiments, the cell transplantation is a stem cell transplantation, for example, a hematopoietic stem cell transplantation, or a bone marrow transplantation. In other embodiments, conditioning a subject prior to cell transplantation includes reducing the number of target-expressing cells in a subject. In some embodiments, prior to administration of the CAR therapy (e.g., a CD19 CAR), the subject receives lymphodepleting therapy. In some embodiments, the subject receives a lympodepleting therapy about two weeks prior to administration of the CAR therapy (e.g., a CD19 CAR). In some embodiments, the lympodepleting therapy comprises fludarabine (e.g., 25 mg/m² IV daily for three doses) and cyclophosphamide (e.g., 250 mg/m² IV daily for three doses).

### Elutriation

In some embodiments, the methods described herein feature an elutriation method that removes unwanted cells, for example, monocytes and blasts, thereby resulting in an improved enrichment of desired immune effector cells suitable for CAR expression. In some embodiments, the elutriation method described herein is optimized for the enrichment of desired immune effector cells suitable for CAR expression from a previously frozen sample, for example, a thawed sample. In some embodiments, the elutriation method described herein provides a preparation of cells with improved purity as compared to a preparation of cells collected from the elutriation protocols known in the art. In some embodiments, the elutriation method described herein includes using an optimized viscosity of the starting sample, for example, cell sample, for example, thawed cell sample, by dilution with certain isotonic solutions (for example, PBS), and using an optimized combination of flow rates and collection volume for each fraction collected by an elutriation device. Exemplary elutriation methods that could be applied in the present invention are described on pages 48-51 of WO 2017/117112, herein incorporated by reference in its entirety.

### Density Gradient Centrifugation

Manufacturing of adoptive cell therapeutic product requires processing the desired cells, for example, immune effector cells, away from a complex mixture of blood cells and blood elements present in peripheral blood apheresis starting materials. Peripheral blood-derived lymphocyte samples have been successfully isolated using density gradient centrifugation through Ficoll solution. However, Ficoll is not a preferred reagent for isolating cells for therapeutic use, as Ficoll is not qualified for clinical use. In addition, Ficoll contains glycol, which has toxic potential to the cells. Furthermore, Ficoll density gradient centrifugation of thawed apheresis products after cryopreservation yields a suboptimal T cell product, for example, as described in the Examples herein. For example, a loss of T cells in the final product, with a relative gain of non-T cells, especially undesirable B cells, blast cells and monocytes was observed in cell preparations isolated by density gradient centrifugation through Ficoll solution.

Without wishing to be bound by theory, it is believed that immune effector cells, for example, T cells, dehydrate during cryopreservation to become denser than fresh cells. Without wishing to be bound by theory, it is also believed that immune effector cells, for example, T cells, remain denser longer than the other blood cells, and thus are more readily lost during Ficoll density gradient separation as compared to other cells. Accordingly, without wishing to be bound by theory, a medium with a density greater than Ficoll is believed to provide improved isolation of desired immune effector cells in comparison to Ficoll or other mediums with the same density as Ficoll, for example, 1.077 g/mL.

In some embodiments, the density gradient centrifugation method described herein includes the use of a density gradient medium comprising iodixanol. In some embodiments, the density gradient medium comprises about 60% iodixanol in water.

In some embodiments, the density gradient centrifugation method described herein includes the use of a density gradient medium having a density greater than Ficoll. In some embodiments, the density gradient centrifugation method described herein includes the use of a density gradient medium having a density greater than 1.077 g/mL, for example, greater than 1.077 g/mL, greater than 1.1 g/mL, greater than 1.15 g/mL, greater than 1.2 g/mL, greater than 1.25 g/mL, greater than 1.3 g/mL, greater than 1.31 g/mL. In some embodiments, the density gradient medium has a density of about 1.32 g/mL.

Additional embodiments of density gradient centrifugation are described on pages 51-53 of WO 2017/117112, herein incorporated by reference in its entirety.

### Enrichment by Selection

Provided herein are methods for selection of specific cells to improve the enrichment of the desired immune effector cells suitable for CAR expression. In some embodiments, the selection comprises a positive selection, for example, selection for the desired immune effector cells. In some embodiments, the selection comprises a negative selection, for example, selection for unwanted cells, for example, removal of unwanted cells. In embodiments, the positive or negative selection methods described herein are performed under flow conditions, for example, by using a flow-through device, for example, a flow-through device described herein. Exemplary positive and negative selections are described on pages 53-57 of WO 2017/117112, herein incorporated by reference in its entirety. Selection methods can be performed under flow conditions, for example, by using a flow-through device, also referred to as a cell processing system, to further enrich a preparation of cells for desired immune effector cells, for example, T cells, suitable for CAR expression. Exemplary flow-through devices are described on pages 57-70 of WO 2017/117112, herein incorporated by reference in its entirety. Exemplary cell separation and debeading methods are described on pages 70-78 of WO 2017/117112, herein incorporated by reference in its entirety.

Selection procedures are not limited to ones described on pages 57-70 of WO 2017/117112. Negative T cell selection via removal of unwanted cells with CD19, CD14 and CD26 Miltenyi beads in combination with column technology (CliniMACS® Plus or CliniMACS® Prodigy®) or positive T cell selection with a combination of CD4 and CD8 Miltenyi beads and column technology (CliniMACS® Plus or CliniMACS® Prodigy®) can be used. Alternatively, column-free technology with releasable CD3 beads (GE Healthcare) can be used.

In addition, bead-free technologies such as ThermoGenesis X-series devices can be utilized as well.

### Clinical Applications

All of the processes herein may be conducted according to clinical good manufacturing practice (cGMP) standards.

The processes may be used for cell purification, enrichment, harvesting, washing, concentration or for cell media exchange, particularly during the collection of raw, starting materials (particularly cells) at the start of the manufacturing process, as well as during the manufacturing process for the selection or expansion of cells for cell therapy.

The cells may include any plurality of cells. The cells may be of the same cell type, or mixed cell types. In addition, the cells may be from one donor, such as an autologous donor or a single allogenic donor for cell therapy. The cells may be obtained from patients e.g., having an autoimmune disease or disorder, e.g., lupus (e.g., systemic lupus erythematosus (SLE), e.g., severe refractory systemic lupus erythematosus (srSLE), or lupus nephritis), systemic sclerosis (e.g., rapidly progressing systemic sclerosis (SSc) with significant lung involvement (e.g. as for autoHSCT)), idiopathic inflammatory myopathies (e.g., polymyositis, dermatomyositis, anti-synthetase syndrome, immune-mediated necrotizing myopathy, inclusion body myositis, overlap myositis, cancer associated myositis, e.g. anti-synthetase syndrome with ILD), vasculitis (e.g., ANCA-associated vasculitis), severe refractory Sjögren's, severe refractory neuroimmune disease (e.g. myasthenia gravis (MG), neuromyelitis optica (NMO), MOG associated disease (MOGAD), multiple sclerosis (MS)), severe refractory rheumatoid arthritis, antibody mediated neuroimmune diseases (e.g., AChR+ and MuSK+ myasthenia gravis (MG), AQP4+ neuromyelitis optica (NMO), MOGAD (anti-MOG associated disease), NMDAR+ encephalitis, or antibody-associated neurological paraneoplastic diseases), Addison's disease, Goodpasture's syndrome, thyrotoxicosis, chronic active hepatitis, relapsing polychondritis, pemphigus vulgaris, or amyotrophic lateral sclerosis, by, for example, leukapheresis or apheresis. The cells may include T cells, for example may include a population that has greater than 50% T cells, greater than 60% T cells, greater than 70% T cells, greater than 80% T cells, or 90% T cells.

Selection processes may be particularly useful in selecting cells prior to culture and expansion. For instance, paramagnetic particles coated with anti-CD3 and/or anti CD28 may be used to select T cells for expansion or for introduction of a nucleic acid encoding a chimeric antigen receptor (CAR) or other protein. Such a process is used to produce CTL019 T cells for treatment of acute lymphoblastic leukemia (ALL).

The debeading processes and modules disclosed herein may be particularly useful in the manufacture of cells for cell therapy, for example in purifying cells prior to, or after, culture and expansion. For instance, paramagnetic particles coated with anti-CD3 and/or anti CD28 antibodies may be used to selectively expand T cells, for example T cells that are, or will be, modified by introduction of a nucleic acid encoding a chimeric antigen receptor (CAR) or other protein, such that the CAR is expressed by the T cells. During the manufacture of such T cells, the debeading processes or modules may be used to separate T cells from the paramagnetic particles. Such a debeading process or module is used to produce, for example, CTL019 T cells for treatment of acute lymphoblastic leukemia (ALL).

In one such process, illustrated here by way of example, cells, for example, T cells, are collected from a donor (for example, a patient having an autoimmune disease or disorder, e.g., lupus (e.g., systemic lupus erythematosus (SLE), e.g., severe refractory systemic lupus erythematosus (srSLE), or lupus nephritis), systemic sclerosis (e.g., rapidly progressing systemic sclerosis (SSc) with significant lung involvement (e.g. as for autoHSCT)), idiopathic inflammatory myopathies (e.g., polymyositis, dermatomyositis, anti-synthetase syndrome, immune-mediated necrotizing myopathy, inclusion body myositis, overlap myositis, cancer associated myositis, e.g. anti-synthetase syndrome with ILD), vasculitis (e.g., ANCA-associated vasculitis), severe refractory Sjögren's, severe refractory neuroimmune disease (e.g. myasthenia gravis (MG), neuromyelitis optica (NMO), MOG associated disease (MOGAD), multiple sclerosis (MS)), severe refractory rheumatoid arthritis, antibody mediated neuroimmune diseases (e.g., AChR+ and MuSK+ myasthenia gravis (MG), AQP4+ neuromyelitis optica (NMO), MOGAD (anti-MOG associated disease), NMDAR+ encephalitis, or antibody-associated neurological paraneoplastic diseases), Addison's disease, Goodpasture's syndrome, thyrotoxicosis, chronic active hepatitis, relapsing polychondritis, pemphigus vulgaris, or amyotrophic lateral sclerosis to be treated with an autologous chimeric antigen receptor T cell product) via apheresis (for example, leukapheresis). Collected cells may then be optionally purified, for example, by an elutriation step, or via positive or negative selection of target cells (for example, T cells). Paramagnetic particles, for example, anti-CD3/anti-CD28-coated paramagnetic particles, may then be added to the cell population, to expand the T cells. The process may also include a transduction step, wherein nucleic acid encoding one or more desired proteins, for example, a CAR, for example a CAR targeting CD19, is introduced into the cell. The nucleic acid may be introduced in a lentiviral vector. The cells, for example, the lentivirally transduced cells, may then be expanded for a period of days, for example, 1, 2, 3, 4, 5, 6, 7, 8, 9, 10, or more days, for example in the presence of a suitable medium. After expansion, the debeading processes/modules disclosed herein may be used to separate the desired T cells from the paramagnetic particles. The process may include one or more debeading steps according to the processes of the present disclosure. The debeaded cells may then be formulated for administration to the patient. Examples of CAR T cells and their manufacture are further described, for example, in WO2012/079000, which is incorporated herein by reference in its entirety. The systems and methods of the present disclosure may be used for any cell separation/purification/debeading processes described in or associated with WO2012/079000. Additional CAR T manufacturing processes are described in, for example, WO2016109410 and WO2017117112, herein incorporated by reference in their entireties.

The systems and methods herein may similarly benefit other cell therapy products by wasting fewer desirable cells, causing less cell trauma, and more reliably removing magnetic and any non-paramagnetic particles from cells with less or no exposure to chemical agents, as compared to conventional systems and methods.

Although only exemplary embodiments of the disclosure are specifically described above, it will be appreciated that modifications and variations of these examples are possible without departing from the spirit and intended scope of the disclosure. For example, the magnetic modules and systems containing them may be arranged and used in a variety of configurations in addition to those described. Besides, non-magnetic modules can be utilized as well. In addition, the systems and methods may include additional components and steps not specifically described herein. For instance, methods may include priming, where a fluid is first introduced into a component to remove bubbles and reduce resistance to cell suspension or buffer movement. Furthermore, embodiments may include only a portion of the systems described herein for use with the methods described herein. For example, embodiments may relate to disposable modules, hoses, etc. usable within non-disposable equipment to form a complete system able to separate or debead cells to produce a cell product.

Additional manufacturing methods and processes that can be combined with the present invention have been described in the art. For examples, pages 86-91 of WO 2017/117112 describe improved wash steps and improved manufacturing process.

### Sources of Immune Effector Cells

This section provides additional methods or steps for obtaining an input sample comprising desired immune effector cells, isolating and processing desired immune effector cells, for example, T cells, and removing unwanted materials, for example, unwanted cells. The additional methods or steps described in this section can be used in combination with any of the elutriation, density gradient centrifugation, selection under flow conditions, or improved wash step described in the preceding sections.

A source of cells, for example, T cells or natural killer (NK) cells, can be obtained from a subject e,g, a subject having an autoimmune disease or disorder, e.g., lupus (e.g., systemic lupus erythematosus (SLE), e.g., severe refractory systemic lupus erythematosus (srSLE), or lupus nephritis), systemic sclerosis (e.g., rapidly progressing systemic sclerosis (SSc) with significant lung involvement (e.g. as for autoHISCT)), idiopathic inflammatory myopathies (e.g., polymyositis, dermatomyositis, anti-synthetase syndrome, immune-mediated necrotizing myopathy, inclusion body myositis, overlap myositis, cancer associated myositis, e.g. anti-synthetase syndrome with ILD), vasculitis (e.g., ANCA-associated vasculitis), severe refractory Sjögren's, severe refractory neuroimmune disease (e.g. myasthenia gravis (MG), neuromyelitis optica (NMO), MOG associated disease (MOGAD), multiple sclerosis (MS)), severe refractory rheumatoid arthritis, antibody mediated neuroimmune diseases (e.g., AChR+ and MuSK+ myasthenia gravis (MG), AQP4+ neuromyelitis optica (NMO), MOGAD (anti-MOG associated disease), NMDAR+ encephalitis, or antibody-associated neurological paraneoplastic diseases), Addison's disease, Goodpasture's syndrome, thyrotoxicosis, chronic active hepatitis, relapsing polychondritis, pemphigus vulgaris, or amyotrophic lateral sclerosis. Examples of subjects include humans, monkeys, chimpanzees, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors.

In some embodiments of the present disclosure, immune effector cells, for example, T cells, can be obtained from a unit of blood collected from a subject using any number of techniques known to the skilled artisan, and any of the methods disclosed herein, in any combination of steps thereof. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and, optionally, to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. In some embodiments, the cells are washed using the improved wash step described herein.

Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate™, or the Haemonetics Cell Saver 5), Haemonetics Cell Saver Elite (GE Healthcare Sepax or Sefia), or a device utilizing the spinning membrane filtration technology (Fresenius Kabi LOVO), according to the manufacturer's instructions. After washing, the cells may be resuspended in a variety of biocompatible buffers, such as, for example, Ca-free, Mg-free PBS, PlasmaLyte A, PBS-EDTA supplemented with human serum albumin (HSA), or other saline solution with or without buffer. Alternatively, the undesirable components of the apheresis sample may be removed and the cells directly resuspended in culture media.

In some embodiments, desired immune effector cells, for example, T cells, are isolated from peripheral blood lymphocytes by lysing the red blood cells and depleting the monocytes, for example, by centrifugation through a PERCOLL™ gradient or by counterflow centrifugal elutriation.

The methods described herein can include, for example, selection of a specific subpopulation of immune effector cells, for example, T cells, that are a T regulatory cell-depleted population, for example, CD25+depleted cells or CD25^{high} depleted cells, using, for example, a negative selection technique, for example, described herein. In some embodiments, the population of T regulatory-depleted cells contains less than 30%, 25%, 20%, 15%, 10%, 5%, 4%, 3%, 2%, 1% of CD25+ cells or CD25^{high} cells.

In some embodiments, T regulatory cells, for example, CD25+ T cells or CD25^{high} T cells, are removed from the population using an anti-CD25 antibody, or fragment thereof, or a CD25-binding ligand, for example IL-2. In some embodiments, the anti-CD25 antibody, or fragment thereof, or CD25-binding ligand is conjugated to a substrate, for example, a bead, or is otherwise coated on a substrate, for example, a bead. In some embodiments, the anti-CD25 antibody, or fragment thereof, is conjugated to a substrate as described herein.

In some embodiments, the T regulatory cells, for example, CD25+ T cells or CD25^{high} T cells, are removed from the population using CD25 depleting reagent from Miltenyi™. In some embodiments, the ratio of cells to CD25 depletion reagent is 1e7 cells to 20 μL, or 1e7 cells to 15 μL, or 1e7 cells to 10 μL, or 1e7 cells to 5 μL, or 1e7 cells to 2.5 μL, or 1e7 cells to 1.25 L. In some embodiments, for example, for T regulatory cells, greater than 500 million cells/ml is used. In some embodiments, a concentration of cells of 600, 700, 800, or 900 million cells/ml is used.

In some embodiments, the population of immune effector cells to be depleted includes about 6×10⁹ CD25+ T cells. In some embodiments, the population of immune effector cells to be depleted include about 1×10⁹ to 1×10¹⁰ CD25+ T cell, and any integer value in between. In some embodiments, the resulting population T regulatory-depleted cells has 2×10⁹ T regulatory cells, for example, CD25+ cells or CD25^{high} cells, or less (for example, 1×10⁹, 5×10⁸, 1×10⁸, 5×10⁷, 1×10⁷, or less T regulatory cells).

In some embodiments, the T regulatory cells, for example, CD25+ cells or CD25^{high} cells, are removed from the population using the CliniMAC system with a depletion tubing set, such as, for example, tubing 162-01. In some embodiments, the CliniMAC system is run on a depletion setting such as, for example, DEPLETION2.1.

Without wishing to be bound by a particular theory, decreasing the level of negative regulators of immune cells (for example, decreasing the number of unwanted immune cells, for example, Treg cells), in a subject prior to apheresis or during manufacturing of a CAR-expressing cell product significantly reduces the risk of subject relapse. For example, methods of depleting Treg cells are known in the art. Methods of decreasing Treg cells include, but are not limited to, cyclophosphamide, anti-GITR antibody (an anti-GITR antibody described herein), CD25-depletion, and combinations thereof.

In some embodiments, the manufacturing methods comprise reducing the number of (for example, depleting) Treg cells prior to manufacturing of the CAR-expressing cell. For example, manufacturing methods comprise contacting the sample, for example, the apheresis sample, with an anti-GITR antibody and/or an anti-CD25 antibody (or fragment thereof, or a CD25-binding ligand), for example, to deplete Treg cells prior to manufacturing of the CAR-expressing cell (for example, T cell, NK cell) product.

Without wishing to be bound by a particular theory, decreasing the level of negative regulators of immune cells (for example, decreasing the number of unwanted immune cells, for example, Treg cells), in a subject prior to apheresis or during manufacturing of a CAR-expressing cell product can reduce the risk of a subject's relapse. In some embodiments, a subject is pre-treated with one or more therapies that reduce Treg cells prior to collection of cells for CAR-expressing cell product manufacturing, thereby reducing the risk of subject relapse to CAR-expressing cell treatment. In some embodiments, methods of decreasing Treg cells include, but are not limited to, administration to the subject of one or more of cyclophosphamide, anti-GITR antibody, CD25-depletion, or a combination thereof. In some embodiments, methods of decreasing Treg cells include, but are not limited to, administration to the subject of one or more of cyclophosphamide, anti-GITR antibody, CD25-depletion, or a combination thereof. Administration of one or more of cyclophosphamide, anti-GITR antibody, CD25-depletion, or a combination thereof, can occur before, during or after an infusion of the CAR-expressing cell product. Administration of one or more of cyclophosphamide, anti-GITR antibody, CD25-depletion, or a combination thereof, can occur before, during or after an infusion of the CAR-expressing cell product.

In some embodiments, the manufacturing methods comprise reducing the number of (for example, depleting) Treg cells prior to manufacturing of the CAR-expressing cell. For example, manufacturing methods comprise contacting the sample, for example, the apheresis sample, with an anti-GITR antibody and/or an anti-CD25 antibody (or fragment thereof, or a CD25-binding ligand), for example, to deplete Treg cells prior to manufacturing of the CAR-expressing cell (for example, T cell, NK cell) product.

In some embodiments, a subject is pre-treated with cyclophosphamide prior to collection of cells for CAR-expressing cell product manufacturing, thereby reducing the risk of subject relapse to CAR-expressing cell treatment (for example, CTL019 treatment). In some embodiments, a subject is pre-treated with an anti-GITR antibody prior to collection of cells for CAR-expressing cell (for example, T cell or NK cell) product manufacturing, thereby reducing the risk of subject relapse to CAR-expressing cell treatment.

In some embodiments, the CAR-expressing cell (for example, T cell, NK cell) manufacturing process is modified to deplete Treg cells prior to manufacturing of the CAR-expressing cell (for example, T cell, NK cell) product (for example, a CTL019 product). In some embodiments, CD25-depletion is used to deplete Treg cells prior to manufacturing of the CAR-expressing cell (for example, T cell, NK cell) product (for example, a CTL019 product).

In some embodiments, the population of cells to be removed are neither the regulatory T cells, but cells that otherwise negatively affect the expansion and/or function of CART cells, for example cells expressing CD14, CD11b, CD33, CD15, or other markers expressed by potentially immune suppressive cells. In some embodiments, such cells are envisioned to be removed concurrently with regulatory T cells, or following said depletion, or in another order.

The methods described herein can include more than one selection step, for example, more than one depletion step. Enrichment of a T cell population by negative selection can be accomplished, for example, with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail can include antibodies to CD14, CD20, CD11b, CD16, HLA-DR, and CD8.

Also provided are methods that include removing cells from the population which express a check point inhibitor, for example, a check point inhibitor described herein, for example, one or more of PD1+ cells, LAG3+ cells, and TIM3+ cells, to thereby provide a population of T regulatory-depleted, for example, CD25+depleted cells, and check point inhibitor depleted cells, for example, PD1+, LAG3+ and/or TIM3+depleted cells. Exemplary check point inhibitors include PD1, PD-L1, PD-L2, CTLA4, TIM3, CEACAM (for example, CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF (for example, TGF beta), for example, as described herein. In some embodiments, check point inhibitor expressing cells are removed simultaneously with the T regulatory, for example, CD25+ cells or CD25^{high} cells. For example, an anti-CD25 antibody, or fragment thereof, and an anti-check point inhibitor antibody, or fragment thereof, can be attached to the same bead which can be used to remove the cells, or an anti-CD25 antibody, or fragment thereof, and the anti-check point inhibitor antibody, or fragment there, can be attached to separate beads, a mixture of which can be used to remove the cells. In other embodiments, the removal of T regulatory cells, for example, CD25+ cells or CD25^{high} cells, and the removal of the check point inhibitor expressing cells is sequential, and can occur, for example, in either order.

Methods described herein can include a positive selection step. For example, T cells can isolated by incubation with anti-CD3/anti-CD28 (for example, 3×28)-conjugated beads, such as Dynabeads® M-450 CD3/CD28 T, for a time period sufficient for positive selection of the desired T cells. In some embodiments, the time period is about 30 minutes. In some embodiments, the time period ranges from 30 minutes to 36 hours or longer and all integer values there between. In some embodiments, the time period is at least 1, 2, 3, 4, 5, or 6 hours. In some embodiments, the time period is 10 to 24 hours, for example, 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points.

In some embodiments, a T cell population can be selected that expresses one or more of IFN-γ, TNFα, IL-17A, IL-2, IL-3, IL-4, GM-CSF, IL-10, IL-13, granzyme B, and perforin, or other appropriate molecules, for example, other cytokines. Methods for screening for cell expression can be determined, for example, by the methods described in PCT Publication No.: WO 2013/126712.

For isolation of a desired population of cells by positive or negative selection, the concentration of cells and surface (for example, particles such as beads) can be varied. In some embodiments, it may be desirable to significantly decrease the volume in which beads and cells are mixed together (for example, increase the concentration of cells), to ensure maximum contact of cells and beads. For example, in some embodiments, a concentration of 10 billion cells/ml, 9 billion/ml, 8 billion/ml, 7 billion/ml, 6 billion/ml, or 5 billion/ml is used. In some embodiments, a concentration of 1 billion cells/ml is used. In some embodiments, a concentration of cells from 75, 80, 85, 90, 95, or 100 million cells/ml is used. In some embodiments, concentrations of 125 or 150 million cells/ml can be used.

Using high concentrations can result in increased cell yield, cell activation, and cell expansion. Further, use of high cell concentrations allows more efficient capture of cells that may weakly express target antigens of interest, such as CD28-negative T cells (for example, leukemic blood, etc.). Such populations of cells may have therapeutic value and would be desirable to obtain. For example, using high concentration of cells allows more efficient selection of CD8+ T cells that normally have weaker CD28 expression.

In some embodiments, it may be desirable to use lower concentrations of cells. By significantly diluting the mixture of T cells and surface (for example, particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In some embodiments, the concentration of cells used is 5×10⁶/ml. In some embodiments, the concentration used can be from about 1×10⁵/ml to 1×10⁶/ml, and any integer value in between.

In some embodiments, the cells may be incubated on a rotator for varying lengths of time at varying speeds at either 2-10° C. or at room temperature.

In some embodiments, a plurality of the immune effector cells of the population do not express diaglycerol kinase (DGK), for example, is DGK-deficient. In some embodiments, a plurality of the immune effector cells of the population do not express Ikaros, for example, is Ikaros-deficient. In some embodiments, a plurality of the immune effector cells of the population do not express DGK and Ikaros, for example, is both DGK and Ikaros-deficient.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In some embodiments, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in immune effector cell therapy for any number of diseases or conditions that would benefit from immune effector cell therapy, such as those described herein. In some embodiments a blood sample or an apheresis is taken from a generally healthy subject. In some embodiments, a blood sample or an apheresis is taken from a generally healthy subject who is at risk of developing a disease, but who has not yet developed a disease (e.g., an autoimmune disease or disorder, e.g., lupus (e.g., systemic lupus erythematosus (SLE), e.g., severe refractory systemic lupus erythematosus (srSLE), or lupus nephritis), systemic sclerosis (e.g., rapidly progressing systemic sclerosis (SSc) with significant lung involvement (e.g. as for autoHSCT)), idiopathic inflammatory myopathies (e.g., polymyositis, dermatomyositis, anti-synthetase syndrome, immune-mediated necrotizing myopathy, inclusion body myositis, overlap myositis, cancer associated myositis, e.g. anti-synthetase syndrome with ILD), vasculitis (e.g., ANCA-associated vasculitis), severe refractory Sjögren's, severe refractory neuroimmune disease (e.g. myasthenia gravis (MG), neuromyelitis optica (NMO), MOG associated disease (MOGAD), multiple sclerosis (MS)), severe refractory rheumatoid arthritis, antibody mediated neuroimmune diseases (e.g., AChR+ and MuSK+myasthenia gravis (MG), AQP4+neuromyelitis optica (NMO), MOGAD (anti-MOG associated disease), NMDAR+ encephalitis, or antibody-associated neurological paraneoplastic diseases), Addison's disease, Goodpasture's syndrome, thyrotoxicosis, chronic active hepatitis, relapsing polychondritis, pemphigus vulgaris, or amyotrophic lateral sclerosis, and the cells of interest are isolated and frozen for later use. In some embodiments, the T cells may be expanded, frozen, and used at a later time. In some embodiments, samples are collected from a patient shortly after diagnosis of a particular disease as described herein but prior to any treatments. In some embodiments, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In some embodiments of the present invention, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in some embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

It is recognized that the methods of the application can utilize culture media conditions comprising 5% or less, for example, 2%, human AB serum, and employ known culture media conditions and compositions, for example those described in Smith et al., “Ex vivo expansion of human T cells for adoptive immunotherapy using the novel Xeno-free CTS™ Immune Cell Serum Replacement” Clinical & Translational Immunology (2015) 4, e31; doi: 10.1038/cti.2014.31.

In some embodiments, the methods of the application can utilize media conditions comprising at least about 0.1%, 0.5%, 1.0%, 1.5%, 2%, 2.5%, 3%, 3.5%, 4%, 4.5%, 5%, 6%, 7%, 8%, 9% or 10% serum. In some embodiments, the media comprises about 0.5%-5%, about 0.5%-4.5%, about 0.5%-4%, about 0.5%-3.5%, about 0.5%-3%, about 0.5%-2.5%, about 0.5%-2%, about 0.5%-1.5%, about 0.5%-1.0%, about 1.0%-5%, about 1.5%-5%, about 2%-5%, about 2.5%-5%, about 3%-5%, about 3.5%-5%, about 4%-5%, or about 4.5%-5% serum. In some embodiments, the media comprises about 0.5% serum. In some embodiments, the media comprises about 0.5% serum. In some embodiments, the media comprises about 1% serum. In some embodiments, the media comprises about 1.5% serum. In some embodiments, the media comprises about 2% serum. In some embodiments, the media comprises about 2.5% serum. In some embodiments, the media comprises about 3% serum. In some embodiments, the media comprises about 3.5% serum. In some embodiments, the media comprises about 4% serum. In some embodiments, the media comprises about 4.5% serum. In some embodiments, the media comprises about 5% serum. In some embodiments, the serum comprises human serum, e.g., human AB serum. In some embodiments, the serum is human serum that has been allowed to naturally coagulate after collection, e.g., off-the-clot (OTC) serum. In some embodiments, the serum is plasma-derived serum human serum. Plasma-derived serum can be produced by defibrinating pooled human plasma collected in the presence of an anticoagulant, e.g., sodium citrate.

In some embodiments, the methods of the application can utilize culture media conditions comprising serum-free medium. In some embodiments, the serum free medium is OpTmizer™ CTS™ (LifeTech), Immunocult™ XF (Stemcell technologies), CellGro™ (CellGenix), TexMacs™ (Miltenyi), Stemline™ (Sigma), Xvivo15TM (Lonza), PrimeXV® (Irvine Scientific), or StemXVivo® (RandD systems). The serum-free medium can be supplemented with a serum substitute such as ICSR (immune cell serum replacement) from LifeTech. The level of serum substitute (for example, ICSR) can be, for example, up to 5%, for example, about 1%, 2%, 3%, 4%, or 5%. In some embodiments, the serum-free medium can be supplemented with serum, e.g., human serum, e.g., human AB serum. In some embodiments, the serum is human serum that has been allowed to naturally coagulate after collection, e.g., off-the-clot (OTC) serum. In some embodiments, the serum is plasma-derived human serum. Plasma-derived serum can be produced by defibrinating pooled human plasma collected in the presence of an anticoagulant, e.g., sodium citrate.

In some embodiments, a T cell population is diaglycerol kinase (DGK)-deficient. DGK-deficient cells include cells that do not express DGK RNA or protein, or have reduced or inhibited DGK activity. DGK-deficient cells can be generated by genetic approaches, for example, administering RNA-interfering agents, for example, siRNA, shRNA, miRNA, to reduce or prevent DGK expression. Alternatively, DGK-deficient cells can be generated by treatment with DGK inhibitors described herein.

In some embodiments, a T cell population is Ikaros-deficient. Ikaros-deficient cells include cells that do not express Ikaros RNA or protein, or have reduced or inhibited Ikaros activity, Ikaros-deficient cells can be generated by genetic approaches, for example, administering RNA-interfering agents, for example, siRNA, shRNA, miRNA, to reduce or prevent Ikaros expression. Alternatively, Ikaros-deficient cells can be generated by treatment with Ikaros inhibitors, for example, lenalidomide.

In embodiments, a T cell population is DGK-deficient and Ikaros-deficient, for example, does not express DGK and Ikaros, or has reduced or inhibited DGK and Ikaros activity. Such DGK and Ikaros-deficient cells can be generated by any of the methods described herein.

In some embodiments, the NK cells are obtained from the subject. In some embodiments, the NK cells are an NK cell line, for example, NK-92 cell line (Conkwest).

### Allogeneic CAR-Expressing Cells

In embodiments described herein, the immune effector cell can be an allogeneic immune effector cell, for example, T cell or NK cell. For example, the cell can be an allogeneic T cell, for example, an allogeneic T cell lacking expression of a functional T cell receptor (TCR) and/or human leukocyte antigen (HLA), for example, HLA class I and/or HLA class II.

A T cell lacking a functional TCR can be, for example, engineered such that it does not express any functional TCR on its surface, engineered such that it does not express one or more subunits that comprise a functional TCR (for example, engineered such that it does not express (or exhibits reduced expression) of TCR alpha, TCR beta, TCR gamma, TCR delta, TCR epsilon, and/or TCR zeta) or engineered such that it produces very little functional TCR on its surface. Alternatively, the T cell can express a substantially impaired TCR, for example, by expression of mutated or truncated forms of one or more of the subunits of the TCR. The term “substantially impaired TCR” means that this TCR will not elicit an adverse immune reaction in a host.

A T cell described herein can be, for example, engineered such that it does not express a functional HLA on its surface. For example, a T cell described herein, can be engineered such that cell surface expression HLA, for example, HLA class 1 and/or HLA class II, is downregulated. In some embodiments, downregulation of HLA may be accomplished by reducing or eliminating expression of beta-2 microglobulin (B2M).

In some embodiments, the T cell can lack a functional TCR and a functional HLA, for example, HLA class I and/or HLA class II.

Modified T cells that lack expression of a functional TCR and/or HLA can be obtained by any suitable means, including a knock out or knock down of one or more subunit of TCR or HLA. For example, the T cell can include a knock down of TCR and/or HLA using siRNA, shRNA, clustered regularly interspaced short palindromic repeats (CRISPR) transcription-activator like effector nuclease (TALEN), or zinc finger endonuclease (ZFN).

In some embodiments, the allogeneic cell can be a cell which does not express or expresses at low levels an inhibitory molecule, for example by any method described herein. For example, the cell can be a cell that does not express or expresses at low levels an inhibitory molecule, for example, that can decrease the ability of a CAR-expressing cell to mount an immune effector response. Examples of inhibitory molecules include PD1, PD-L1, PD-L2, CTLA4, TIM3, CEACAM (for example, CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF (for example, TGF beta). Inhibition of an inhibitory molecule, for example, by inhibition at the DNA, RNA or protein level, can optimize a CAR-expressing cell performance. In embodiments, an inhibitory nucleic acid, for example, an inhibitory nucleic acid, for example, a dsRNA, for example, an siRNA or shRNA, a clustered regularly interspaced short palindromic repeats (CRISPR), a transcription-activator like effector nuclease (TALEN), or a zinc finger endonuclease (ZFN), for example, as described herein, can be used.

siRNA and shRNA to Inhibit TCR or HLA

In some embodiments, TCR expression and/or HLA expression can be inhibited using siRNA or shRNA that targets a nucleic acid encoding a TCR and/or HLA, and/or an inhibitory molecule described herein (for example, PD1, PD-L1, PD-L2, CTLA4, TIM3, CEACAM (for example, CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, for example, T cell.

Expression systems for siRNA and shRNAs, and exemplary shRNAs, are described, for example, in paragraphs 649 and 650 of International Application WO2015/142675, filed Mar. 13, 2015, which is incorporated by reference in its entirety.

### CRISPR to Inhibit TCR or HLA

“CRISPR” or “CRISPR to TCR and/or HLA” or “CRISPR to inhibit TCR and/or HLA” as used herein refers to a set of clustered regularly interspaced short palindromic repeats, or a system comprising such a set of repeats. “Cas”, as used herein, refers to a CRISPR-associated protein. A “CRISPR/Cas” system refers to a system derived from CRISPR and Cas which can be used to silence or mutate a TCR and/or HLA gene, and/or an inhibitory molecule described herein (for example, PD1, PD-L1, PD-L2, CTLA4, TIM3, CEACAM (for example, CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, for example, T cell.

The CRISPR/Cas system, and uses thereof, are described, for example, in paragraphs 651-658 of International Application WO2015/142675, filed Mar. 13, 2015, which is incorporated by reference in its entirety.

TALEN to Inhibit TCR and/or HLA

“TALEN” or “TALEN to HLA and/or TCR” or “TALEN to inhibit HLA and/or TCR” refers to a transcription activator-like effector nuclease, an artificial nuclease which can be used to edit the HLA and/or TCR gene, and/or an inhibitory molecule described herein (for example, PD1, PD-L1, PD-L2, CTLA4, TIM3, CEACAM (for example, CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, for example, T cell.

TALENs, and uses thereof, are described, for example, in paragraphs 659-665 of International Application WO2015/142675, filed Mar. 13, 2015, which is incorporated by reference in its entirety.

Zinc Finger Nuclease to Inhibit HLA and/or TCR

“ZFN” or “Zinc Finger Nuclease” or “ZFN to HLA and/or TCR” or “ZEN to inhibit HLA and/or TCR” refer to a zinc finger nuclease, an artificial nuclease which can be used to edit the HLA and/or TCR gene, and/or an inhibitory molecule described herein (for example, PD1, PD-L1, PD-L2, CTLA4, TIM3, CEACAM (for example, CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, for example, T cell.

ZFNs, and uses thereof, are described, for example, in paragraphs 666-671 of International Application WO2015/142675, filed Mar. 13, 2015, which is incorporated by reference in its entirety.

### Telomerase Expression

Telomeres play a crucial role in somatic cell persistence, and their length is maintained by telomerase (TERT). Telomere length in CLL cells may be very short (Roth et al., “Significantly shorter telomeres in T-cells of patients with ZAP-70+/CD38 chronic lymphocytic leukaemia” British Journal of Haematology, 143, 383-386., Aug. 28 2008), and may be even shorter in manufactured CAR-expressing cells, for example, CART19 cells, limiting their potential to expand after adoptive transfer to a patient. Telomerase expression can rescue CAR-expressing cells from replicative exhaustion.

While not wishing to be bound by any particular theory, in some embodiments, a therapeutic T cell has short term persistence in a patient, due to shortened telomeres in the T cell; accordingly, transfection with a telomerase gene can lengthen the telomeres of the T cell and improve persistence of the T cell in the patient. See Carl June, “Adoptive T cell therapy for cancer in the clinic”, Journal of Clinical Investigation, 117:1466-1476 (2007). Thus, in some embodiments, an immune effector cell, for example, a T cell, ectopically expresses a telomerase subunit, for example, the catalytic subunit of telomerase, for example, TERT, for example, hTERT. In some embodiments, this disclosure provides a method of producing a CAR-expressing cell, comprising contacting a cell with a nucleic acid encoding a telomerase subunit, for example, the catalytic subunit of telomerase, for example, TERT, for example, hTERT. The cell may be contacted with the nucleic acid before, simultaneous with, or after being contacted with a construct encoding a CAR.

Telomerase expression may be stable (for example, the nucleic acid may integrate into the cell's genome) or transient (for example, the nucleic acid does not integrate, and expression declines after a period of time, for example, several days). Stable expression may be accomplished by transfecting or transducing the cell with DNA encoding the telomerase subunit and a selectable marker, and selecting for stable integrants. Alternatively or in combination, stable expression may be accomplished by site-specific recombination, for example, using the Cre/Lox or FLP/FRT system.

Transient expression may involve transfection or transduction with a nucleic acid, for example, DNA or RNA such as mRNA. In some embodiments, transient mRNA transfection avoids the genetic instability sometimes associated with stable transfection with TERT. Transient expression of exogenous telomerase activity is described, for example, in International Application WO2014/130909, which is incorporated by reference herein in its entirety. In embodiments, mRNA-based transfection of a telomerase subunit is performed according to the messenger RNA Therapeutics™ platform commercialized by Moderna Therapeutics. For instance, the method may be a method described in U.S. Pat. Nos. 8,710,200, 8,822,663, 8,680,069, 8,754,062, 8,664,194, or 8680069.

In some embodiments, hTERT has the amino acid sequence of GenBank Protein ID AAC51724.1 (Meyerson et al., “hEST2, the Putative Human Telomerase Catalytic Subunit Gene, Is Up-Regulated in Tumor Cells and during Immortalization” Cell Volume 90, Issue 4, 22 Aug. 1997, Pages 785-795):

(SEQ ID NO: 284)
MPRAPRCRAVRSLLRSHYREVLPLATFVRRLGPQGWRLVQRGDPAAFRAL
VAQCLVCVPWDARPPPAAPSFRQVSCLKELVARVLQRLCERGAKNVLAFG
FALLDGARGGPPEAFTTSVRSYLPNTVTDALRGSGAWGLLLRRVGDDVLV
HLLARCALFVLVAPSCAYQVCGPPLYQLGAATQARPPPHASGPRRRLGCE
RAWNHSVREAGVPLGLPAPGARRRGGSASRSLPLPKRPRRGAAPEPERTP
VGQGSWAHPGRTRGPSDRGFCVVSPARPAEEATSLEGALSGTRHSHPSVG
RQHHAGPPSTSRPPRPWDTPCPPVYAETKHFLYSSGDKEQLRPSFLLSSL
RPSLTGARRLVETIFLGSRPWMPGTPRRLPRLPQRYWQMRPLFLELLGNH
AQCPYGVLLKTHCPLRAAVTPAAGVCAREKPQGSVAAPEEEDTDPRRLVQ
LLRQHSSPWQVYGFVRACLRRLVPPGLWGSRHNERRFLRNTKKFISLGKH
AKLSLQELTWKMSVRGCAWLRRSPGVGCVPAAEHRLREEILAKFLHWLMS
VYVVELLRSFFYVTETTFQKNRLFFYRKSVWSKLQSIGIRQHLKRVQLRE
LSEAEVRQHREARPALLTSRLRFIPKPDGLRPIVNMDYVVGARTFRREKR
AERLTSRVKALFSVLNYERARRPGLLGASVLGLDDIHRAWRTFVLRVRAQ
DPPPELYFVKVDVTGAYDTIPQDRLTEVIASIIKPQNTYCVRRYAVVQKA
AHGHVRKAFKSHVSTLTDLQPYMRQFVAHLQETSPLRDAVVIEQSSSLNE
ASSGLFDVFLRFMCHHAVRIRGKSYVQCQGIPQGSILSTLLCSLCYGDME
NKLFAGIRRDGLLLRLVDDFLLVTPHLTHAKTFLRTLVRGVPEYGCVVNL
RKTVVNFPVEDEALGGTAFVQMPAHGLFPWCGLLLDTRTLEVQSDYSSYA
RTSIRASLTFNRGFKAGRNMRRKLFGVLRLKCHSLFLDLQVNSLQTVCTN
IYKILLLQAYRFHACVLQLPFHQQVWKNPTFFLRVISDTASLCYSILKAK
NAGMSLGAKGAAGPLPSEAVQWLCHQAFLLKLTRHRVTYVPLLGSLRTAQ
TQLSRKLPGTTLTALEAAANPALPSDFKTILD

In some embodiments, the hTERT has a sequence at least 80%, 85%, 90%, 95%, 96%, 97%, 98%, or 99% identical to the sequence of SEQ ID NO: 284. In some embodiments, the hTERT has a sequence of SEQ ID NO: 284. In some embodiments, the hTERT comprises a deletion (for example, of no more than 5, 10, 15, 20, or 30 amino acids) at the N-terminus, the C-terminus, or both. In some embodiments, the hTERT comprises a transgenic amino acid sequence (for example, of no more than 5, 10, 15, 20, or 30 amino acids) at the N-terminus, the C-terminus, or both.

In some embodiments, the hTERT is encoded by the nucleic acid sequence of GenBank Accession No. AF018167 (Meyerson et al., “hEST2, the Putative Human Telomerase Catalytic Subunit Gene, Is Up-Regulated in Tumor Cells and during Immortalization” Cell Volume 90, Issue 4, 22 Aug. 1997, Pages 785−795).

### Activation and Expansion of Immune Effector Cells (for Example, T Cells)

Immune effector cells such as T cells generated or enriched by the methods described herein may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, a population of immune effector cells may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (for example, bryostatin) in conjunction with a calcium ionophore. For costimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besançon, France) can be used as can other methods commonly known in the art (Berg et al., Transplant Proc. 30 (8): 3975−3977, 1998; Haanen et al., J. Exp. Med. 190 (9): 13191328, 1999; Garland et al., J. Immunol Meth. 227 (1-2): 53-63, 1999).

In some embodiments, the primary stimulatory signal and the costimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In some embodiments, the agent providing the costimulatory signal is bound to a cell surface and the agent providing the primary activation signal is in solution or coupled to a surface. In some embodiments, both agents can be in solution. In some embodiments, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In some embodiments, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the costimulatory signal is an anti-CD28 antibody or antigen-binding fragment thereof; and both agents are co-immobilized to the same bead in equivalent molecular amounts. In some embodiments, a 1:1 ratio of each antibody bound to the beads for CD4+ T cell expansion and T cell growth is used. In some embodiments of the present invention, a ratio of anti CD3: CD28 antibodies bound to the beads is used such that an increase in T cell expansion is observed as compared to the expansion observed using a ratio of 1:1. In some embodiments an increase of from about 1 to about 3 fold is observed as compared to the expansion observed using a ratio of 1:1. In some embodiments, the ratio of CD3: CD28 antibody bound to the beads ranges from 100:1 to 1:100 and all integer values there between. In some embodiments, more anti-CD28 antibody is bound to the particles than anti-CD3 antibody, i.e., the ratio of CD3: CD28 is less than one. In some embodiments, the ratio of anti CD28 antibody to anti CD3 antibody bound to the beads is greater than 2:1. In some embodiments, a 1:100 CD3: CD28 ratio of antibody bound to beads is used. In some embodiments, a 1:75 CD3: CD28 ratio of antibody bound to beads is used. In some embodiments, a 1:50 CD3: CD28 ratio of antibody bound to beads is used. In some embodiments, a 1:30 CD3: CD28 ratio of antibody bound to beads is used. In some embodiments, a 1:10 CD3: CD28 ratio of antibody bound to beads is used. In some embodiments, a 1:3 CD3: CD28 ratio of antibody bound to the beads is used. In some embodiments, a 3:1 CD3: CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In some embodiments the ratio of cells to particles ranges from 1:100 to 100:1 and any integer values in-between and in some embodiments the ratio comprises 1:9 to 9:1 and any integer values in between, can also be used to stimulate T cells. The ratio of anti-CD3- and anti-CD28-coupled particles to T cells that result in T cell stimulation can vary as noted above, however certain suitable values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one suitable ratio being at least 1:1 particles per T cell. In some embodiments, a ratio of particles to cells of 1:1 or less is used. In some embodiments, a suitable particle: cell ratio is 1:5. In some embodiments, the ratio of particles to cells can be varied depending on the day of stimulation. For example, in some embodiments, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In some embodiments, the ratio of particles to cells is 1:1 on the first day of stimulation and adjusted to 1:5 on the third and fifth days of stimulation. In some embodiments, particles are added on a daily or every other day basis to a final ratio of 1:1 on the first day, and 1:5 on the third and fifth days of stimulation. In some embodiments, the ratio of particles to cells is 2:1 on the first day of stimulation and adjusted to 1:10 on the third and fifth days of stimulation. In some embodiments, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type. In some embodiments, the most typical ratios for use are in the neighborhood of 1:1, 2:1 and 3:1 on the first day.

In some embodiments, the cells, such as T cells, are combined with agent-coated beads, the beads and the cells are subsequently separated, and then the cells are cultured. In some embodiments, prior to culture, the agent-coated beads and cells are not separated but are cultured together. In some embodiments, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In some embodiments the cells (for example, 10⁴ to 10⁹ T cells) and beads (for example, Dynabeads® M-450 CD3/CD28 T paramagnetic beads at a ratio of 1:1) are combined in a buffer, for example PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In some embodiments, it may be desirable to significantly decrease the volume in which particles and cells are mixed together (i.e., increase the concentration of cells), to ensure maximum contact of cells and particles. For example, in some embodiments, a concentration of about 10 billion cells/ml, 9 billion/ml, 8 billion/ml, 7 billion/ml, 6 billion/ml, 5 billion/ml, or 2 billion cells/ml is used. In some embodiments, greater than 100 million cells/ml is used. In some embodiments, a concentration of cells of 10, 15, 20, 25, 30, 35, 40, 45, or 50 million cells/ml is used. In some embodiments, a concentration of cells from 75, 80, 85, 90, 95, or 100 million cells/ml is used. In some embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in some embodiments. For example, using high concentration of cells allows more efficient selection of CD8+ T cells that normally have weaker CD28 expression.

In some embodiments, cells transduced with a nucleic acid encoding a CAR, for example, a CAR described herein, for example, a CD19 CAR described herein, are expanded, for example, by a method described herein. In some embodiments, the cells are expanded in culture for a period of several hours (for example, about 2, 3, 4, 5, 6, 7, 8, 9, 10, 15, 18, 21 hours) to about 14 days (for example, 1, 2, 3, 4, 5, 6, 7, 8, 9, 10, 11, 12, 13 or 14 days). In some embodiments, the cells are expanded for a period of 4 to 9 days. In some embodiments, the cells are expanded for a period of 8 days or less, for example, 7, 6 or 5 days. In some embodiments, the cells are expanded in culture for 5 days, and the resulting cells are more potent than the same cells expanded in culture for 9 days under the same culture conditions. Potency can be defined, for example, by various T cell functions, for example proliferation, target cell killing, cytokine production, activation, migration, surface CAR expression, CAR quantitative PCR, or combinations thereof. In some embodiments, the cells, for example, a CD19 CAR cell described herein, expanded for 5 days show at least a one, two, three or four-fold increase in cells doublings upon antigen stimulation as compared to the same cells expanded in culture for 9 days under the same culture conditions. In some embodiments, the cells, for example, the cells expressing a CD19 CAR described herein, are expanded in culture for 5 days, and the resulting cells exhibit higher proinflammatory cytokine production, for example, IFN-γ and/or GM-CSF levels, as compared to the same cells expanded in culture for 9 days under the same culture conditions. In some embodiments, the cells, for example, a CD19 CAR cell described herein, expanded for 5 days show at least a one, two, three, four, five, ten-fold or more increase in pg/ml of proinflammatory cytokine production, for example, IFN—Y and/or GM-CSF levels, as compared to the same cells expanded in culture for 9 days under the same culture conditions.

Several cycles of stimulation may also be desired such that culture time of T cells can be 60 days or more. Conditions appropriate for T cell culture include an appropriate media (for example, Minimal Essential Media, α-MEM, RPMI Media 1640, AIM-V, DMEM, F-12, or X-vivo 15 (Lonza), X-Vivo 20, OpTmizer, and IMDM) that may contain factors necessary for proliferation and viability, including serum (for example, fetal bovine or human serum), interleukin-2 (IL-2), insulin, IFNγ, IL-4, IL-7, GM-CSF, IL-10, IL-12, IL-15, TGFβ, and TNFα or any other additives for the growth of cells known to the skilled artisan. Other additives for the growth of cells include, but are not limited to, surfactant, plasmanate, and reducing agents such as N-acetyl-cysteine and 2-mercaptoethanol. Media can include, but is not limited to RPMI 1640, AIM-V, DMEM, MEM, α-MEM, F-12, X-Vivo 15, X-Vivo 20, OpTmizer, and IMDM with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, for example, penicillin and streptomycin, are included only in experimental cultures, not in cultures of cells that are to be infused into a subject. The target cells are maintained under conditions necessary to support growth, for example, an appropriate temperature (for example, 37° C.) and atmosphere (for example, air plus 5% CO₂).

In some embodiments, the cells are expanded in an appropriate media (for example, media described herein) that includes one or more interleukin that result in at least a 200-fold (for example, 200-fold, 250-fold, 300-fold, 350-fold) increase in cells over a 14-day expansion period, for example, as measured by a method described herein such as flow cytometry. In some embodiments, the cells are expanded in the presence IL-15 and/or IL-7 (for example, IL-15 and IL-7).

In embodiments, methods described herein, for example, CAR-expressing cell manufacturing methods, comprise removing T regulatory cells, for example, CD25+ T cells or CD25^{high} T cells, from a cell population, for example, using an anti-CD25 antibody, or fragment thereof, or a CD25-binding ligand, IL-2. Methods of removing T regulatory cells, for example, CD25+ T cells or CD25^{high} T cells, from a cell population are described herein. In embodiments, the methods, for example, manufacturing methods, further comprise contacting a cell population (for example, a cell population in which T regulatory cells, such as CD25+ T cells or CD25^{high} T cells, have been depleted; or a cell population that has previously contacted an anti-CD25 antibody, fragment thereof, or CD25-binding ligand) with IL-15 and/or IL-7. For example, the cell population (for example, that has previously contacted an anti-CD25 antibody, fragment thereof, or CD25-binding ligand) is expanded in the presence of IL-15 and/or IL-7.

In some embodiments a CAR-expressing cell described herein is contacted with a composition comprising a interleukin-15 (IL-15) polypeptide, a interleukin-15 receptor alpha (IL-15Ra) polypeptide, or a combination of both a IL-15 polypeptide and a IL-15Ra polypeptide for example, hetIL-15, during the manufacturing of the CAR-expressing cell, for example, ex vivo. In embodiments, a CAR-expressing cell described herein is contacted with a composition comprising a IL-15 polypeptide during the manufacturing of the CAR-expressing cell, for example, ex vivo. In embodiments, a CAR-expressing cell described herein is contacted with a composition comprising a combination of both a IL-15 polypeptide and a IL-15 Ra polypeptide during the manufacturing of the CAR-expressing cell, for example, ex vivo. In embodiments, a CAR-expressing cell described herein is contacted with a composition comprising hetIL-15 during the manufacturing of the CAR-expressing cell, for example, ex vivo.

In some embodiments the CAR-expressing cell described herein is contacted with a composition comprising hetIL-15 during ex vivo expansion. In some embodiments, the CAR-expressing cell described herein is contacted with a composition comprising an IL-15 polypeptide during ex vivo expansion. In some embodiments, the CAR-expressing cell described herein is contacted with a composition comprising both an IL-15 polypeptide and an IL-15Ra polypeptide during ex vivo expansion. In some embodiments the contacting results in the survival and proliferation of a lymphocyte subpopulation, for example, CD8+ T cells.

T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8+). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Once a CAR described herein is constructed, various assays can be used to evaluate the activity of the molecule, such as but not limited to, the ability to expand T cells following antigen stimulation, sustain T cell expansion in the absence of re-stimulation, and anti-cancer activities in appropriate in vitro and animal models. Assays to evaluate the effects of a CAR of the present invention are described in further detail below

Western blot analysis of CAR expression in primary T cells can be used to detect the presence of monomers and dimers, for example, as described in paragraph 695 of International Application WO2015/142675, filed Mar. 13, 2015, which is herein incorporated by reference in its entirety.

In vitro expansion of CAR+ T cells following antigen stimulation can be measured by flow cytometry. For example, a mixture of CD4+ and CD8+ T cells are stimulated with αCD3/αCD28 aAPCs followed by transduction with lentiviral vectors expressing GFP under the control of the promoters to be analyzed. Exemplary promoters include the CMV IE gene, EF-1a, ubiquitin C, or phosphoglycerokinase (PGK) promoters. GFP fluorescence is evaluated on day 6 of culture in the CD4⁺ and/or CD8⁺ T cell subsets by flow cytometry. See, for example, Milone et al., Molecular Therapy 17 (8): 1453-1464 (2009). Alternatively, a mixture of CD4⁺ and CD8₊ T cells are stimulated with αCD3/αCD28 coated magnetic beads on day 0, and transduced with CAR on day 1 using a bicistronic lentiviral vector expressing CAR along with eGFP using a 2A ribosomal skipping sequence. Cultures are re-stimulated with either a cancer associated antigen as described herein⁺ K562 cells (K562-expressing a cancer associated antigen as described herein), wild-type K562 cells (K562 wild type) or K562 cells expressing hCD32 and 4-1BBL in the presence of antiCD3 and anti-CD28 antibody (K562-BBL-3/28). Exogenous IL-2 is added to the cultures every other day at 100 IU/ml. GFP+ T cells are enumerated by flow cytometry using bead-based counting. See, for example, Milone et al., Molecular Therapy 17 (8): 1453-1464 (2009).

Sustained CAR⁺ T cell expansion in the absence of re-stimulation can also be measured. See, for example, Milone et al., Molecular Therapy 17 (8): 1453-1464 (2009). Briefly, mean T cell volume (fl) is measured on day 8 of culture using a Coulter Multisizer III particle counter or a higher version, a Nexcelom Cellometer Vision, Millipore Scepter or other cell counters, following stimulation with αCD3/αCD28 coated magnetic beads on day 0, and transduction with the indicated CAR on day 1.

Animal models can also be used to measure a CAR-expressing cell activity, for example, as described in paragraph 698 of International Application WO2015/142675, filed Mar. 13, 2015, which is herein incorporated by reference in its entirety.

Dose dependent CAR treatment response can be evaluated, for example, as described in paragraph 699 of International Application WO2015/142675, filed Mar. 13, 2015, which is herein incorporated by reference in its entirety.

Assessment of cell proliferation and cytokine production has been previously described, as described in paragraph 700 of International Application WO2015/142675, filed Mar. 13, 2015, which is herein incorporated by reference in its entirety.

Cytotoxicity can be assessed by a standard 51Cr-release assay, for example, as described in paragraph 701 of International Application WO2015/142675, filed Mar. 13, 2015, which is herein incorporated by reference in its entirety. Alternative non-radioactive methods can be utilized as well.

Cytotoxicity can also be assessed by measuring changes in adherent cell's electrical impedance, for example, using an xCELLigence real time cell analyzer (RTCA). In some embodiments, cytotoxicity is measured at multiple time points.

Imaging technologies can be used to evaluate specific trafficking and proliferation of CARs in tumor-bearing animal models, for example, as described in paragraph 702 of International Application WO2015/142675, filed Mar. 13, 2015, which is herein incorporated by reference in its entirety.

Other assays, including those described in the Example section herein as well as those that are known in the art can also be used to evaluate the CARs described herein.

Alternatively, or in combination to the methods disclosed herein, methods and compositions for one or more of: detection and/or quantification of CAR-expressing cells (for example, in vitro or in vivo (for example, clinical monitoring)); immune cell expansion and/or activation; and/or CAR-specific selection, that involve the use of a CAR ligand, are disclosed. In some embodiments, the CAR ligand is an antibody that binds to the CAR molecule, for example, binds to the extracellular antigen-binding domain of CAR (for example, an antibody that binds to the antigen-binding domain, for example, an anti-idiotypic antibody; or an antibody that binds to a constant region of the extracellular binding domain). In other embodiments, the CAR ligand is a CAR antigen molecule (for example, a CAR antigen molecule as described herein).

In some embodiments, a method for detecting and/or quantifying CAR-expressing cells is disclosed. For example, the CAR ligand can be used to detect and/or quantify CAR-expressing cells in vitro or in vivo (for example, clinical monitoring of CAR-expressing cells in a patient, or dosing a patient). The method includes:
providing the CAR ligand (optionally, a labelled CAR ligand, for example, a CAR ligand that includes a tag, a bead, a radioactive or fluorescent label);
acquiring the CAR-expressing cell (for example, acquiring a sample containing CAR-expressing cells, such as a manufacturing sample or a clinical sample);
contacting the CAR-expressing cell with the CAR ligand under conditions where binding occurs, thereby detecting the level (for example, amount) of the CAR-expressing cells present. Binding of the CAR-expressing cell with the CAR ligand can be detected using standard techniques such as FACS, ELISA and the like.

In some embodiments, a method of expanding and/or activating cells (for example, immune effector cells) is disclosed. The method includes:
providing a CAR-expressing cell (for example, a first CAR-expressing cell or a transiently expressing CAR cell);
contacting said CAR-expressing cell with a CAR ligand, for example, a CAR ligand as described herein), under conditions where immune cell expansion and/or proliferation occurs, thereby producing the activated and/or expanded cell population.

In certain embodiments, the CAR ligand is present on a substrate (for example, is immobilized or attached to a substrate, for example, a non-naturally occurring substrate). In some embodiments, the substrate is a non-cellular substrate. The non-cellular substrate can be a solid support chosen from, for example, a plate (for example, a microtiter plate), a membrane (for example, a nitrocellulose membrane), a matrix, a chip or a bead. In embodiments, the CAR ligand is present in the substrate (for example, on the substrate surface). The CAR ligand can be immobilized, attached, or associated covalently or non-covalently (for example, cross-linked) to the substrate. In some embodiments, the CAR ligand is attached (for example, covalently attached) to a bead. In the aforesaid embodiments, the immune cell population can be expanded in vitro or ex vivo. The method can further include culturing the population of immune cells in the presence of the ligand of the CAR molecule, for example, using any of the methods described herein.

In other embodiments, the method of expanding and/or activating the cells further comprises addition of a second stimulatory molecule, for example, CD28. For example, the CAR ligand and the second stimulatory molecule can be immobilized to a substrate, for example, one or more beads, thereby providing increased cell expansion and/or activation.

In some embodiments, a method for selecting or enriching for a CAR expressing cell is provided. The method includes contacting the CAR expressing cell with a CAR ligand as described herein; and selecting the cell on the basis of binding of the CAR ligand.

In yet other embodiments, a method for depleting, reducing and/or killing a CAR expressing cell is provided. The method includes contacting the CAR expressing cell with a CAR ligand as described herein; and targeting the cell on the basis of binding of the CAR ligand, thereby reducing the number, and/or killing, the CAR-expressing cell. In some embodiments, the CAR ligand is coupled to a toxic agent (for example, a toxin or a cell ablative drug). In some embodiments, the anti-idiotypic antibody can cause effector cell activity, for example, ADCC or ADC activities.

Exemplary anti-CAR antibodies that can be used in the methods disclosed herein are described, for example, in WO 2014/190273 and by Jena et al., “Chimeric Antigen Receptor (CAR)-Specific Monoclonal Antibody to Detect CD19-Specific T cells in Clinical Trials”, PLOS March 2013 8:3 e57838, the contents of which are incorporated by reference.

In some embodiments, the compositions and methods herein are optimized for a specific subset of T cells, for example, as described in US Ser. No. PCT/US2015/043219 filed Jul. 31, 2015, the contents of which are incorporated herein by reference in their entirety. In some embodiments, the optimized subsets of T cells display an enhanced persistence compared to a control T cell, for example, a T cell of a different type (for example, CD8+ or CD4+) expressing the same construct.

In some embodiments, a CD4+ T cell comprises a CAR described herein, which CAR comprises an intracellular signaling domain suitable for (for example, optimized for, for example, leading to enhanced persistence in) a CD4+ T cell, for example, an ICOS domain. In some embodiments, a CD8+ T cell comprises a CAR described herein, which CAR comprises an intracellular signaling domain suitable for (for example, optimized for, for example, leading to enhanced persistence of) a CD8+ T cell, for example, a 4-1BB domain, a CD28 domain, or another costimulatory domain other than an ICOS domain. In some embodiments, the CAR described herein comprises an antigen-binding domain described herein, for example, a CAR comprising an antigen-binding domain.

In some embodiments, described herein is a method of treating a subject, for example, a subject having an autoimmune disease or disorder, e.g., lupus (e.g., systemic lupus erythematosus (SLE), e.g., severe refractory systemic lupus erythematosus (srSLE), or lupus nephritis), systemic sclerosis (e.g., rapidly progressing systemic sclerosis (SSc) with significant lung involvement (e.g. as for autoHSCT)), idiopathic inflammatory myopathies (e.g., polymyositis, dermatomyositis, anti-synthetase syndrome, immune-mediated necrotizing myopathy, inclusion body myositis, overlap myositis, cancer associated myositis, e.g. anti-synthetase syndrome with ILD), vasculitis (e.g., ANCA-associated vasculitis), severe refractory Sjögren's, severe refractory neuroimmune disease (e.g. myasthenia gravis (MG), neuromyelitis optica (NMO), MOG associated disease (MOGAD), multiple sclerosis (MS)), severe refractory rheumatoid arthritis, antibody mediated neuroimmune diseases (e.g., AChR+ and MuSK+myasthenia gravis (MG), AQP4+ neuromyelitis optica (NMO), MOGAD (anti-MOG associated disease), NMDAR+ encephalitis, or antibody-associated neurological paraneoplastic diseases), Addison's disease, Goodpasture's syndrome, thyrotoxicosis, chronic active hepatitis, relapsing polychondritis, pemphigus vulgaris, or amyotrophic lateral sclerosis. The method includes administering to said subject, an effective amount of:
1) a CD4+ T cell comprising a CAR (the CARCD4+) comprising:
an antigen-binding domain, for example, an antigen-binding domain described herein;
a transmembrane domain; and
an intracellular signaling domain, for example, a first costimulatory domain, for example, an ICOS domain; and
2) a CD8+ T cell comprising a CAR (the CARCD8+) comprising:
an antigen-binding domain, for example, an antigen-binding domain described herein;
a transmembrane domain; and
an intracellular signaling domain, for example, a second costimulatory domain, for example, a 4-1BB domain, a CD28 domain, or another costimulatory domain other than an ICOS domain;
wherein the CARCD4+ and the CARCD8+differ from one another.

Optionally, the method further includes administering:
3) a second CD8+ T cell comprising a CAR (the second CARCD8+) comprising:
an antigen-binding domain, for example, an antigen-binding domain described herein;
a transmembrane domain; and
an intracellular signaling domain, wherein the second CARCD8+comprises an intracellular signaling domain, for example, a costimulatory signaling domain, not present on the CARCD8+, and, optionally, does not comprise an ICOS signaling domain.

### Biopolymer Delivery Methods

In some embodiments, one or more CAR-expressing cells as disclosed herein can be administered or delivered to the subject via a biopolymer scaffold, for example, a biopolymer implant. Biopolymer scaffolds can support or enhance the delivery, expansion, and/or dispersion of the CAR-expressing cells described herein. A biopolymer scaffold comprises a biocompatible (for example, does not substantially induce an inflammatory or immune response) and/or a biodegradable polymer that can be naturally occurring or synthetic. Exemplary biopolymers are described, for example, in paragraphs 1004-1006 of International Application WO2015/142675, filed Mar. 13, 2015, which is herein incorporated by reference in its entirety.

### Pharmaceutical Compositions and Treatments

In some embodiments, the disclosure provides a method of treating a patient, e.g., a patient having an autoimmune disease or disorder, e.g., lupus (e.g., systemic lupus erythematosus (SLE), e.g., severe refractory systemic lupus erythematosus (srSLE), or lupus nephritis), systemic sclerosis (e.g., rapidly progressing systemic sclerosis (SSc) with significant lung involvement (e.g. as for autoHSCT)), idiopathic inflammatory myopathies (e.g., polymyositis, dermatomyositis, anti-synthetase syndrome, immune-mediated necrotizing myopathy, inclusion body myositis, overlap myositis, cancer associated myositis, e.g. anti-synthetase syndrome with ILD), vasculitis (e.g., ANCA-associated vasculitis), severe refractory Sjögren's, severe refractory neuroimmune disease (e.g. myasthenia gravis (MG), neuromyelitis optica (NMO), MOG associated disease (MOGAD), multiple sclerosis (MS)), severe refractory rheumatoid arthritis, antibody mediated neuroimmune diseases (e.g., AChR+ and MuSK+ myasthenia gravis (MG), AQP4+ neuromyelitis optica (NMO), MOGAD (anti-MOG associated disease), NMDAR+ encephalitis, or antibody-associated neurological paraneoplastic diseases), Addison's disease, Goodpasture's syndrome, thyrotoxicosis, chronic active hepatitis, relapsing polychondritis, pemphigus vulgaris, or amyotrophic lateral sclerosis, comprising administering CAR-expressing cells produced as described herein, optionally in combination with one or more other therapies. In some embodiments, the disclosure provides a method of treating a patient, comprising administering a reaction mixture comprising CAR-expressing cells as described herein, optionally in combination with one or more other therapies. In some embodiments, the disclosure provides a method of shipping or receiving a reaction mixture comprising CAR-expressing cells as described herein. In some embodiments, the disclosure provides a method of treating a patient, comprising receiving a CAR-expressing cell that was produced as described herein, and further comprising administering the CAR-expressing cell to the patient, optionally in combination with one or more other therapies. In some embodiments, the disclosure provides a method of treating a patient, comprising producing a CAR-expressing cell as described herein, and further comprising administering the CAR-expressing cell to the patient, optionally in combination with one or more other therapies. The other therapy may be, for example, one or more of an antimalarial (e.g., hydroxychloroquine or quinacrine), a glucocorticoid (e.g., prednisone), a calcineurin inhibitor, an immunomodulatory agent (e.g., methotrexate, azathioprine, mycophenolate moefetil, cyclophosphamide, or tacrolimus), a biological agent (e.g., belimumab, rituximab, a disease-modifying antirheumatic drug (DMARD) (e.g., leflunomide).

The methods described herein can further include formulating a CAR-expressing cell in a pharmaceutical composition. Pharmaceutical compositions may comprise a CAR-expressing cell, for example, a plurality of CAR-expressing cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (for example, aluminum hydroxide); and preservatives. Compositions can be formulated, for example, for intravenous administration.

In some embodiments, the pharmaceutical composition is substantially free of, for example, there are no detectable levels of a contaminant, for example, selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In some embodiments, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

When “an immunologically effective amount” or “therapeutic amount” is indicated, the precise amount of the compositions to be administered can be determined by a physician with consideration of individual differences in age, weight, tumor size, extent of infection or metastasis, and condition of the patient (subject). It can generally be stated that a pharmaceutical composition comprising the immune effector cells (for example, T cells, NK cells) described herein may be administered at a dosage of about 0.5×10⁶ to 50×10⁶ viable CAR-expressing cells, in some instances about 5×10⁶ viable CAR-expressing cells, including all integer values within those ranges. T cell compositions may also be administered multiple times at these dosages. The cells can be administered by using infusion techniques that are commonly known in immunotherapy (see, for example, Rosenberg et al., New Eng. J. of Med. 319:1676, 1988).

In some embodiments, it may be desired to administer activated immune effector cells (for example, T cells, NK cells) to a subject and then subsequently redraw blood (or have an apheresis performed), activate immune effector cells (for example, T cells, NK cells) therefrom, and reinfuse the patient with these activated and expanded immune effector cells (for example, T cells, NK cells). This process can be carried out multiple times every few weeks. In some embodiments, immune effector cells (for example, T cells, NK cells) can be activated from blood draws of from 10cc to 400cc. In some embodiments, immune effector cells (for example, T cells, NK cells) are activated from blood draws of 20cc, 30cc, 40cc, 50cc, 60cc, 70cc, 80cc, 90cc, or 100cc.

The administration of the subject compositions may be carried out in any convenient manner. The compositions described herein may be administered to a patient trans arterially, subcutaneously, intradermally, intratumorally, intranodally, intramedullary, intramuscularly, by intravenous (i.v.) injection, or intraperitoneally, for example, by intradermal or subcutaneous injection. The compositions of immune effector cells (for example, T cells, NK cells) may be injected directly into a lymph node or site of disease.

### Dosage Regimen

In some embodiments, a dose of viable CAR-expressing cells (for example, viable CD19 CAR-expressing cells, or any dual CARs thereof) comprises about 0.5×10⁶ viable CAR-expressing cells to about 1.25×10⁹ viable CAR-expressing cells (for example, 0.5×10⁶ viable CAR-expressing cells to 1.25×10⁹ viable CAR-expressing cells). In some embodiments, (for example, viable CD19 CAR-expressing cells) comprises about 1×10⁶, about 2.5×10⁶, about 5×10⁶, about 1.25×10⁷, about 2.5×10⁷, about 5×10⁷, about 5.75×10⁷, or about 8×10⁷ viable CAR-expressing cells. In some embodiments, a dose of viable CAR-expressing cells comprises about 0.5×10⁶ to 50×10⁶ viable CAR-expressing cells. In some embodiments, a dose of viable CAR-expressing cells comprises about 5×10⁶ viable CAR-expressing cells. In some embodiments, a dose of viable CAR-expressing cells comprises about 2.5×10⁶ to 2.5×10⁸ viable CAR-expressing cells. In some embodiments, a dose of viable CAR-expressing cells comprises about 1.25×10⁷ viable CAR-expressing cells. In some embodiments, a dose of viable CAR-expressing cells comprises about 1.25×10⁷ to 1.25×10⁹ viable CAR-expressing cells. In some embodiments, a dose of viable CAR-expressing cells comprises about 1.25×10⁸ viable CAR-expressing cells. In some embodiments, a dose of viable CAR-expressing cells comprises about 2.5×10⁶ to 2.5×10⁸ viable CAR-expressing cells. In some embodiments, a dose of viable CAR-expressing cells comprises about 1×10⁷ or 5×10⁷ viable CAR-expressing cells.

In some embodiments, a dose of viable CAR-positive cells (for example, viable CD19 CAR-positive cells, or any dual CARs thereof) comprises about 0.5×10⁶ viable CAR-positive cells to about 1.25×10⁹ viable CAR-positive cells (for example, 0.5×10⁶ viable CAR-positive cells to 1.25×10⁹ viable CAR-positive cells). In some embodiments, (for example, viable CD19 CAR-positive cells) comprises about 1×10⁶, about 2.5×10⁶, about 5×10⁶, about 1.25×10⁷, about 2.5×10⁷, about 5×10⁷, about 5.75×10⁷, or about 8×10⁷ viable CAR-positive cells. In some embodiments, a dose of viable CAR-positive cells comprises about 0.5×10⁶ to 50×10⁶ viable CAR-positive cells. In some embodiments, a dose of viable CAR-positive cells comprises about 5×10⁶ viable CAR-positive cells. In some embodiments, a dose of viable CAR-positive cells comprises about 2.5×10⁶ to 2.5×10⁸ viable CAR-positive cells. In some embodiments, a dose of viable CAR-positive cells comprises about 1.25×10⁷ viable CAR-positive cells. In some embodiments, a dose of viable CAR-positive cells comprises about 1.25×10⁷ to 1.25×10⁹ viable CAR-positive cells. In some embodiments, a dose of viable CAR-positive cells comprises about 1.25×10⁸ viable CAR-positive cells. In some embodiments, a dose of viable CAR-positive cells comprises about 2.5×10⁶ to 2.5×10⁸ viable CAR-positive cells. In some embodiments, a dose of viable CAR-positive cells comprises about 1×10⁷ or 5×10⁷ viable CAR-positive cells.

In some embodiments, a dose of CAR cells (for example, CD19 CAR cells) comprises about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (for example, CD19 CAR cells) comprises at least about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (for example, CD19 CAR cells) comprises up to about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (for example, CD19 CAR cells) comprises about 1.1×10⁶-1.8×10⁷ cells/kg. In some embodiments, a dose of CAR cells (for example, CD19 CAR cells) comprises about 1×10⁷, 2×10⁷, 5×10⁷, 1×10⁸, 2×10⁸, 5×10⁸, 1×10⁹, 2×10⁹, or 5×10⁹ cells. In some embodiments, a dose of CAR cells (for example, CD19 CAR cells) comprises at least about 1×10⁷, 2×10⁷, 5×10⁷, 1×10⁸, 2×10⁸, 5×10⁸, 1×10⁹, 2×10⁹, or 5×10⁹ cells. In some embodiments, a dose of CAR cells (for example, CD19 CAR cells) comprises up to about 1×10⁷, 2×10⁷, 5×10⁷, 1×10⁸, 2×10⁸, 5×10⁸, 1×10⁹, 2×10⁹, or 5×10⁹ cells.

The level of CAR-positive cells can be determined according to the methods disclosed in Example 8 of WO/2021/173985. Briefly, for CAR T cells manufactured using a continuous Activated Rapid Manufacturing (ARM) process, e.g., ARM-CD19 CAR T cells, a sentinel vial of cryopreserved cells may be thawed and cultured for up to 5 days and the CAR expression analyzed by flow cytometry. The measurement of CAR expression on, e.g., day 2 or day 3 may be used to determine the dose of viable CAR-positive T cells.

In some embodiments, the disclosure provides a method of treating a patient, e.g., a patient having an autoimmune disease or disorder, e.g., lupus (e.g., systemic lupus erythematosus (SLE), e.g., severe refractory systemic lupus erythematosus (srSLE), or lupus nephritis), systemic sclerosis (e.g., rapidly progressing systemic sclerosis (SSc) with significant lung involvement (e.g. as for autoHSCT)), idiopathic inflammatory myopathies (e.g., polymyositis, dermatomyositis, anti-synthetase syndrome, immune-mediated necrotizing myopathy, inclusion body myositis, overlap myositis, cancer associated myositis, e.g. anti-synthetase syndrome with ILD), vasculitis (e.g., ANCA-associated vasculitis), severe refractory Sjögren's, severe refractory neuroimmune disease (e.g. myasthenia gravis (MG), neuromyelitis optica (NMO), MOG associated disease (MOGAD), multiple sclerosis (MS)), severe refractory rheumatoid arthritis, antibody mediated neuroimmune diseases (e.g., AChR+ and MuSK+myasthenia gravis (MG), AQP4+ neuromyelitis optica (NMO), MOGAD (anti-MOG associated disease), NMDAR+ encephalitis, or antibody-associated neurological paraneoplastic diseases), Addison's disease, Goodpasture's syndrome, thyrotoxicosis, chronic active hepatitis, relapsing polychondritis, pemphigus vulgaris, or amyotrophic lateral sclerosis, comprising administering to said patient CAR-expressing cells produced as described herein, at a dose of viable CAR-expressing or CAR-positive cells (for example, viable CD19 CAR-expressing cells, viable CD19 CAR-positive cells, or any dual CARs thereof) from about 0.5×10⁶ viable CAR-expressing or CAR-positive cells to about 50×10⁶ viable CAR-expressing or CAR-positive cells (for example, from about 0.5×10⁶ viable CD19 CAR-expressing or CAR-positive cells to about 50×10⁶ viable CD19 CAR-expressing or CAR-positive cells), e.g. at a dose of viable CAR-expressing or CAR-positive cells (for example, viable CD19 CAR-expressing cells or viable CD19 CAR-positive cells) from about 2×10⁶ viable CAR-expressing or CAR-positive cells to about 40×10⁶ viable CAR-expressing or CAR-positive cells.

In some embodiments, the disclosure provides a method of treating a patient, e.g., a patient having an autoimmune disease or disorder, e.g., lupus (e.g., systemic lupus erythematosus (SLE), e.g., severe refractory systemic lupus erythematosus (srSLE), or lupus nephritis), comprising administering to said patient CAR-expressing or CAR-positive cells produced as described herein, at a dose of viable CAR-expressing or CAR-positive cells (for example, viable CD19 CAR-expressing cells, viable CD19 CAR-positive cells, or any dual CARs thereof) from about 0.5×10⁶ viable CAR-expressing or CAR-positive cells to about 50×10⁶ viable CAR-expressing or CAR-positive cells (for example, from about 0.5×10⁶ viable CD19 CAR-expressing or CAR-positive cells to about 50×10⁶ viable CD19 CAR-expressing or CAR-positive cells).

In some embodiments, the disclosure provides a method of treating a patient, e.g., a patient having severe refractory systemic lupus erythematosus (srSLE), or lupus nephritis, comprising administering to said patient CAR-expressing cells produced as described herein, at a dose of viable CAR-expressing or CAR-positive cells (for example, viable CD19 CAR-expressing cells, viable CD19 CAR-positive cells, or any dual CARs thereof) from about 0.5×10⁶ viable CAR-expressing or CAR-positive cells to about 50×10⁶ viable CAR-expressing or CAR-positive cells (for example, from about 0.5×10⁶ viable CD19 CAR-expressing cells or CAR-positive to about 50×10⁶ viable CD19 CAR-expressing or CAR-positive cells).

In some embodiments, the disclosure provides a method of treating a patient, e.g., a patient having severe refractory systemic lupus erythematosus (srSLE), or lupus nephritis, comprising administering to said patient CAR-expressing or CAR-positive cells produced as described herein, at a dose of from about 2.5×10⁶ viable CD19 CAR-expressing or CAR-positive cells to about 40×10⁶ viable CD19 CAR-expressing or CAR-positive cells.

In some embodiments, the disclosure provides a method of treating a patient, e.g., a patient having severe refractory systemic lupus erythematosus (srSLE), or lupus nephritis, comprising administering to said patient CAR-expressing or CAR-positive cells produced as described herein, at a dose of from about 9×10⁶ viable CD19 CAR-expressing cells or CAR-positive to about 40×10⁶ viable CD19 CAR-expressing or CAR-positive cells.

In some embodiments, the disclosure provides a method of treating a patient, e.g., a patient having severe refractory systemic lupus erythematosus (srSLE), or lupus nephritis, comprising administering to said patient CAR-expressing or CAR-positive cells produced as described herein, at a dose of from about 1×10⁶ viable CD19 CAR-expressing or CAR-positive cells to about 2.5×10⁶ viable CD19 CAR-expressing or CAR-positive cells.

In some embodiments, the disclosure provides a method of treating a patient, e.g., a patient having severe refractory systemic lupus erythematosus (srSLE), or lupus nephritis, comprising administering to said patient CAR-expressing or CAR-positive cells produced as described herein, at a dose of from about 5×10⁶ viable CD19 CAR-expressing or CAR-positive cells to about 12.5×10⁶ viable CD19 CAR-expressing or CAR-positive cells.

In some embodiments, the disclosure provides a method of treating a patient, e.g., a patient having severe refractory systemic lupus erythematosus (srSLE), or lupus nephritis, comprising administering to said patient CAR-expressing or CAR-positive cells produced as described herein, at a dose of from about 25×10⁶ viable CD19 CAR-expressing or CAR-positive cells to about 40×10⁶ viable CD19 CAR-expressing or CAR-positive cells.

In some embodiments, the disclosure provides a method of treating a patient, e.g., a patient having severe refractory systemic lupus erythematosus (srSLE), or lupus nephritis, comprising administering to said patient CAR-expressing or CAR-positive cells produced as described herein, at a dose of about 1×10⁶, 2×10⁶, 2.5×10⁶, 3×10⁶, 4×10⁶, 5×10⁶, 6×10⁶, 7×10⁶, 8×10⁶, 9×10⁶, 10×10⁶, 11×10⁶, 12×10⁶, or about 12.5×10⁶ of viable CD19 CAR-expressing or CAR-positive cells.

In some embodiments, the disclosure provides a method of treating a patient, e.g., a patient having severe refractory systemic lupus erythematosus (srSLE), or lupus nephritis, comprising administering to said patient CAR-expressing or CAR-positive cells produced as described herein, at a dose of about 2.5×10⁶ viable CD19 CAR-expressing or CAR-positive cells. In some embodiments, the disclosure provides a method of treating a patient, e.g., a patient having severe refractory systemic lupus erythematosus (srSLE), or lupus nephritis, comprising administering to said patient CAR-expressing or CAR-positive cells produced as described herein, at a dose of about 5×10⁶ viable CD19 CAR-expressing or CAR-positive cells. In some embodiments, the disclosure provides a method of treating a patient, e.g., a patient having severe refractory systemic lupus erythematosus (srSLE), or lupus nephritis, comprising administering to said patient CAR-expressing or CAR-positive cells produced as described herein, at a dose of about 9×10⁶ viable CD19 CAR-expressing or CAR-positive cells. In some embodiments, the disclosure provides a method of treating a patient, e.g., a patient having severe refractory systemic lupus erythematosus (srSLE), or lupus nephritis, comprising administering to said patient CAR-expressing or CAR-positive cells produced as described herein, at a dose of about 12.5×10⁶ viable CD19 CAR-expressing or CAR-positive cells.

### Patient Selection

In some embodiments of any of the methods of treating a subject, or composition for use disclosed herein, the subject has an autoimmune disease or disorder, e.g., lupus (e.g., systemic lupus erythematosus (SLE), e.g., severe refractory systemic lupus erythematosus (srSLE), or lupus nephritis), systemic sclerosis (e.g., rapidly progressing systemic sclerosis (SSc) with significant lung involvement (e.g. as for autoHSCT)), idiopathic inflammatory myopathies (e.g., polymyositis, dermatomyositis, anti-synthetase syndrome, immune-mediated necrotizing myopathy, inclusion body myositis, overlap myositis, cancer associated myositis, e.g. anti-synthetase syndrome with ILD), vasculitis (e.g., ANCA-associated vasculitis), severe refractory Sjögren's, severe refractory neuroimmune disease (e.g. myasthenia gravis (MG), neuromyelitis optica (NMO), MOG associated disease (MOGAD), multiple sclerosis (MS)), severe refractory rheumatoid arthritis, antibody mediated neuroimmune diseases (e.g., AChR+ and MuSK+myasthenia gravis (MG), AQP4+ neuromyelitis optica (NMO), MOGAD (anti-MOG associated disease), NMDAR+ encephalitis, or antibody-associated neurological paraneoplastic diseases), Addison's disease, Goodpasture's syndrome, thyrotoxicosis, chronic active hepatitis, relapsing polychondritis, pemphigus vulgaris, or amyotrophic lateral sclerosis.

In some embodiments, the subject having srSLE has previously been administered one or more of an antimalarial (e.g., hydroxychloroquine or quinacrine), a glucocorticoid (e.g., prednisone), a calcineurin inhibitor, an immunomodulatory agent (e.g., methotrexate, azathioprine, mycophenolate moefetil, cyclophosphamide, or tacrolimus), a biological agent (e.g., belimumab, rituximab, a disease-modifying antirheumatic drug (DMARD) (e.g., leflunomide). In some embodiments, the subject has been identified as not responding to treatment comprising two or more immunosuppressive therapies (e.g., mycophenolate or cyclophosphamide) in combination with a glucocorticoid) and one biological agent. In some embodiments, the subject has not previously received a therapy comprising a CD19 CAR, an adoptive T cell therapy, or a gene therapy product.

In some embodiments, prior to administration of the CAR therapy (e.g., a CD19 CAR), the subject receives lymphodepleting therapy. In some embodiments, the subject receives a lympodepleting therapy about two weeks prior to administration of the CAR therapy (e.g., a CD19 CAR). In some embodiments, the lympodepleting therapy comprises fludarabine (e.g., 25 mg/m² IV daily for three doses) and cyclophosphamide (e.g., 250 mg/m² IV daily for three doses).

In some embodiments, the subject is an adult, for example, at least 18 years of age.

### Evaluating CAR Safety

In some embodiments of any of the therapeutic methods disclosed herein, the method further involves evaluating the safety of the CAR-expressing cell therapy in a subject. In some embodiments, safety of the CAR-expressing cell therapy is evaluated by measuring or recording one or more of a subject's vital signs, adverse events experienced by the subject, various laboratory parementers, and/or an electrocardiogram of the subject.

In some embodiments, the subject does not experience an adverse event of grade 3 or higher. In some embodiments, the subject does not experience cytokine release syndrome (CRS). In some embodiments, the subject does not experience CRS of grade 3 or higher. In some embodiments, the subject does not experience immune effector cell-associated neurotoxicity syndrome (ICANS).

### Biomarkers for Evaluating CAR-Effectiveness

In some embodiments, disclosed herein is a method of evaluating or monitoring the effectiveness of a CAR-expressing cell therapy (for example, a CD19 CAR therapy), in a subject (for example, a subject having an autoimmune disease or disorder, e.g., lupus (e.g., systemic lupus erythematosus (SLE), e.g., severe refractory systemic lupus erythematosus (srSLE), or lupus nephritis), systemic sclerosis (e.g., rapidly progressing systemic sclerosis (SSc) with significant lung involvement (e.g. as for autoHSCT)), idiopathic inflammatory myopathies (e.g., polymyositis, dermatomyositis, anti-synthetase syndrome, immune-mediated necrotizing myopathy, inclusion body myositis, overlap myositis, cancer associated myositis, e.g. anti-synthetase syndrome with ILD), vasculitis (e.g., ANCA-associated vasculitis), severe refractory Sjögren's, severe refractory neuroimmune disease (e.g. myasthenia gravis (MG), neuromyelitis optica (NMO), MOG associated disease (MOGAD), multiple sclerosis (MS)), severe refractory rheumatoid arthritis, antibody mediated neuroimmune diseases (e.g., AChR+ and MuSK+ myasthenia gravis (MG), AQP4+ neuromyelitis optica (NMO), MOGAD (anti-MOG associated disease), NMDAR+ encephalitis, or antibody-associated neurological paraneoplastic diseases), Addison's disease, Goodpasture's syndrome, thyrotoxicosis, chronic active hepatitis, relapsing polychondritis, pemphigus vulgaris, or amyotrophic lateral sclerosis). The method includes acquiring a value of effectiveness to the CAR therapy, wherein said value is indicative of the effectiveness or suitability of the CAR-expressing cell therapy.

In embodiments, the value of effectiveness to the CAR therapy in a subject having an autoimmune disease or disorder, e.g., lupus (e.g., systemic lupus erythematosus (SLE), e.g., severe refractory systemic lupus erythematosus (srSLE), or lupus nephritis), systemic sclerosis (e.g., rapidly progressing systemic sclerosis (SSc) with significant lung involvement (e.g. as for autoHSCT)), idiopathic inflammatory myopathies (e.g., polymyositis, dermatomyositis, anti-synthetase syndrome, immune-mediated necrotizing myopathy, inclusion body myositis, overlap myositis, cancer associated myositis, e.g. anti-synthetase syndrome with ILD), vasculitis (e.g., ANCA-associated vasculitis), severe refractory Sjögren's, severe refractory neuroimmune disease (e.g. myasthenia gravis (MG), neuromyelitis optica (NMO), MOG associated disease (MOGAD), multiple sclerosis (MS)), severe refractory rheumatoid arthritis, antibody mediated neuroimmune diseases (e.g., AChR+ and MuSK+myasthenia gravis (MG), AQP4+ neuromyelitis optica (NMO), MOGAD (anti-MOG associated disease), NMDAR+encephalitis, or antibody-associated neurological paraneoplastic diseases), Addison's disease, Goodpasture's syndrome, thyrotoxicosis, chronic active hepatitis, relapsing polychondritis, pemphigus vulgaris, or amyotrophic lateral sclerosis, comprises a measure of one, two, three, or more parameters described herein.

In other embodiments, the value of effectiveness to the CAR therapy, further comprises a measure of one, two, three, four, five, six or more (all) of the following parameters:
(i) the level or activity of one, two, three, or more (for example, all) of resting T_{EFF} cells, resting T_{REG} cells, younger T cells (for example, naïve T cells (for example, naïve CD4 or CD8 T cells, naïve gamma/delta T cells), or stem memory T cells (for example, stem memory CD4 or CD8 T cells, or stem memory gamma/delta T cells), or early memory T cells, or a combination thereof, in a sample (for example, an apheresis sample or a manufactured CAR-expressing cell product sample);
(ii) the level or activity of one, two, three, or more (for example, all) of activated T_{EFF} cells, activated T_{REG} cells, older T cells (for example, older CD4 or CD8 cells), or late memory T cells, or a combination thereof, in a sample (for example, an apheresis sample or a manufactured CAR-expressing cell product sample);
(iii) the level or activity of an immune cell exhaustion marker, for example, one, two or more immune checkpoint inhibitors (for example, PD-1, PD-L1, TIM-3, TIGIT and/or LAG-3) in a sample (for example, an apheresis sample or a manufactured CAR-expressing cell product sample). In some embodiments, an immune cell has an exhausted phenotype, for example, co-expresses at least two exhaustion markers, for example, co-expresses PD-1 and TIM-3. In other embodiments, an immune cell has an exhausted phenotype, for example, co-expresses at least two exhaustion markers, for example, co-expresses PD-1 and LAG-3;
(iv) the level or activity of CD27 and/or CD45RO-(for example, CD27+CD45RO−) immune effector cells, for example, in a CD4+ or a CD8+ T cell population, in a sample (for example, an apheresis sample or a manufactured CAR-expressing cell product sample);
(v) the level or activity of one, two, three, four, five, six, seven, eight, nine, ten, eleven or all of the biomarkers chosen from CCL20, IL-17a, IL-6, PD-1, PD-L1, LAG-3, TIM-3, CD57, CD27, CD122, CD62L, KLRG1;
(vi) a cytokine level or activity (for example, quality of cytokine reportoire) in a CAR-expressing cell product sample, for example, CLL-1-expressing cell product sample; or
(vii) a transduction efficiency of a CAR-expressing cell in a manufactured CAR-expressing cell product sample.

In some embodiments of any of the methods disclosed herein, the CAR-expressing cell therapy comprises a plurality (for example, a population) of CAR-expressing immune effector cells, for example, a plurality (for example, a population) of T cells or NK cells, or a combination thereof. In some embodiments, the CAR-expressing cell therapy is a CD19 CAR therapy. In some embodiments of any of the methods disclosed herein, the measure of one or more of the parameters disclosed herein is obtained from an apheresis sample acquired from the subject. The apheresis sample can be evaluated prior to infusion or re-infusion.

In some embodiments of any of the methods disclosed herein, the measure of one or more of the parameters disclosed herein is obtained from a sample acquired from the subject.

In some embodiments of any of the methods disclosed herein, the measure of one or more of the parameters disclosed herein is obtained from a manufactured CAR-expressing cell product sample, for example, CD19 CAR-expressing cell product sample. The manufactured CAR-expressing cell product can be evaluated prior to infusion or re-infusion.

In some embodiments of any of the methods disclosed herein, the subject is evaluated prior to receiving, during, or after receiving, the CAR-expressing cell therapy.

In some embodiments of any of the methods disclosed herein, the measure of one or more of the parameters disclosed herein evaluates a profile for one or more of gene expression, flow cytometry or protein expression.

In some embodiments of any of the methods disclosed herein, the method further comprises identifying the subject as a responder or a non-responder, and/or one who has achieved remission, based on a measure of one or more of the parameters disclosed herein.

In embodiments, a subject who is a responder or a non-responder, identified by the methods herein can be further evaluated according to clinical criteria. For example, a complete responder has, or is identified as, a subject having a disease, for example, an autoimmune disease or disorder, e.g., lupus (e.g., systemic lupus erythematosus (SLE), e.g., severe refractory systemic lupus erythematosus (srSLE) or lupus nephritis), who exhibits a complete response, for example, to a treatment. Efficacy may be evaluated, for example, using the lupus low disease activity state (LLDAS) (as described in Franklyn et al, Ann Rheum Dis. 2016), e.g., as informed by SLEDAI-2K (described in Gladman et al, J Rheumatol. 2000), and physician's global assessment; SLE responder index (SRI-4) (as described in Furie et al, Arthritis Rheumatol. 2017); British Isles Lupus Assessment Group-based Composite Lupus Assessment (BICLA) (as described in Wallace et al. Arthritis Rheum. 2011); British Isles Lupus Activity Group score (BILAG) (as described in Isenberg et al Ann Rheum Dis 2005); urinary protein creatinine ratio (UPCR) at various time points; and/or complete renal response (CRR) at various time points. In some embodiments, remission may be evaluated using the DORIS definition (as described in van Vollenhoven et al, Lupus Sci Med. 2021).

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1: Description of the Activated Rapid Manufacturing (ARM) Process

In some embodiments, CART cells are manufactured using a continuous Activated Rapid Manufacturing (ARM) process, over approximately 2 days, which will potentially allow for a greater number of less differentiated T cells (T naïve and TSCM (stem central memory T) cells) to be returned to a patient for in vivo cellular expansion. The short manufacturing time period allows the early differentiated T cells profile to proliferate in the body for their desired terminal differentiated state rather that in an ex vivo culture vessel.

In some embodiments, CART cells are manufactured using cryopreserved leukapheresis source material, for example, non-mobilized autologous peripheral blood leukapheresis (LKPK) material. Cryopreserved source material undergoes processing steps for T cell enrichment on the first day of production (Day 0) by means of anti-CD4/anti-CD8 immunomagnetic system. Positive fraction is then seeded in G-rex culture vessel, activated with an anti-CD3/CD28 system (TransACT) and on the same day transduced with a lentiviral vector (LV) encoding a CAR. On the following day, after 20-28 hours of transduction, the T cells are harvested, washed four times, formulated in freezing medium, and then frozen by a Controlled Rate Freezer (CRF). From the start of the process on Day 0 to the initiation of harvest on the following day, cells are cultured for 20-28 hours with a target of 24 hours after Day 0 seeding.

Media for Day 0 were prepared according to Table 21.

TABLE 21
Media type and point of use during CART manufacturing

| Media/Buffer Type | Composition | Point of Use |
|---|---|---|
| Rapid Buffer (RB) | CliniMACS ® Buffer | Day 0 Processing on |
| | (+0.5% human serum | Cell Wash/Separator |
| | albumin (HSA)) | |
| Rapid Media (RM) | OpTmizer ™ Media, CTS ™, | Day 0 for Processing |
| | IL-2, Glutamax | on Cell Wash/Separator |
| | and ICSR | and Cell Seeding |
| Harvest Buffer (HB) | PBS no EDTA and | Harvest Wash |
| (also called Harvest | 2% HSA | Buffer (Day 1) |
| Buffer Solution) | | |
| Cryomedia | Cryostor10 (CS10) | Harvest Formulation |

The cryopreserved leukapheresis material is thawed. The thawed cells are diluted with the Rapid Buffer (Table 21) and washed on the CliniMACS® Prodigy® device. The T cells are selected by CliniMACS® CD4 and CD8 microbeads. Once the program is finished for T cell selection (approximately 3 h 40 min to 4 h 40 min), the reapplication bag containing the cells suspended in Rapid Media (Table 21) are transferred in a transfer pack. A sample is taken for viability and cell count. The cell count and viability data from the positive fraction bag is used to determine the cell concentration when seeding the culture vessel for activation and vector transduction.

Following positive selection of T cells via the CliniMACS® microbeads (CD4 and CD8), the cells are seeded in the culture vessel, G-Rex. Once the cells are seeded, the activation reagent (TransACT) is then added to the culture vessel. The cells are then transduced with a lentiviral vector encoding a CAR at a target MOI of 1.0 (0.8-1.2). Following the vector addition, the culture vessel is transported to an incubator where it is incubated for a target of 24 hours (operating range 20-28 hours) at a nominal temperature of 37° C. (operating range 36-38° C.) with nominal 5% CO₂ (operating range 4.5−5.5%). Following the incubation, the cells are washed with Harvest Wash Solution (Table 21) four times to remove any non-integrated vector and residual viral particles, as well as any other process related impurities. Then, the cells are eluted and a sample for cell count and viability is taken for testing and the results are used to determine the volume required to re-suspend the cells for final formulation with CryoStor® CS10. The cells are then centrifugated to remove the Harvest Wash Solution and proceed with cryopreservation.

In some embodiments, the CAR expressed in CART cells binds to CD19. In some embodiments, IL-2 used in the Rapid Media (RM) (Table 21) can be replaced with IL-15, hetIL-15 (IL-15/sIL-15Ra), IL-6, or IL-6/sIL-6Ra.

### Example 2: Generation and In Vitro Characterization Assessment of CD19-Targeting CAR-T Cells Using T Cells from Systemic Lupus Erythematosus Patients

Fresh blood from five SLE patients with moderate SLE disease activity (SLEDAI score of 8) and 5 age/gender-matched (±5 years) healthy donors (HD) was collected. T cell generation and ex vivo expansion were performed according to the methods disclosed herein.

Cell characterization, including cell viability, CAR-T cells final product recovery rate post-harvesting (or post-wash), and ex vivo fold expansion was analyzed in both SLE patients and HDs.

Results: Highly pure and viable T cells were obtained from both SLE patients and HDs. The manufacturing process used allowed preservation of CD4 and CD8 naive/T scm cells in the final product. Ex vivo expanded CAR-T cells from SLE patients in this study were fully functional in vitro.

These results support the feasibility of generating cells via the methodologies described herein, using T cells from SLE patients. Overall, SLE patient-derived CAR-T cells displayed comparable transduction rates, T cell stemness feature and functional properties (cytolytic activity and cytokine production) to CAR-T cells generated from age/gender-matched HDs.

### Example 3: Phase 1/2 Study, Open-Label, Multi-Center, to Assess Safety, Efficacy and Cellular Kinetics of ARM-CD19 CAR T Cells in Participants with Severe, Refractory Autoimmune Disorders

This study evaluates the safety, efficacy, and in vivo cellular kinetics (pharmacokinetics, PK) of ARM-CD19 CAR T treatment in participants with severe refractory systemic lupus erythematosus (srSLE) and other severe forms of autoimmune diseases.

ARM-CD19 CAR T is an autologous CD19-directed CAR-T cell therapy that is comprised of CD4+/CD8+ T cells that have undergone ex vivo T cell activation and gene modification. ARM-CD19 CAR T utilizes the FMC63 scFv domain for CD19 recognition and the same lentiviral vector as tisagenlecleucel (Kymriah, CTL019) and is manufactured the activated rapid manufacturing (ARM) process. The ARM process reduces the turnaround time compared to traditional manufacturing processes and preserves T cell stemness, the ability to self-renew and mature, resulting in a product with greater proliferative potential and fewer exhausted T cells compared to traditionally manufactured CAR-T cells. With ARM, CAR-T cell expansion occurs primarily within a patient's body (in vivo), eliminating the need for an extended culture time outside of the body (ex vivo). These unique characteristics may lead to better and more durable responses, improved long-term outcomes and a reduced risk of severe adverse events compared to CAR-T cell products manufactured via traditional manufacturing methods.

Non-clinical studies show that ARM-CD19 CAR T is a product with potentially superior antitumor efficacy, a similar safety profile, and delayed expansion compared to other CD19-directed CAR-T cell therapy relying on a traditional manufacturing process (e.g. tisagenlecleucel). Both ARM-CD19 CAR T and tisagenlecleucel induced tumor-regression in a dose-dependent manner in animal models. IL-6 production was similar between ARM-CD19 CAR T and traditionally manufactured CAR-T cells. In a non-GLP compliant toxicology study, NSG mice engrafted with ARM-CD19 CAR T did not cause unexpected findings in comparison to traditionally manufactured CAR-T cells and untransduced cells undergoing the ARM manufacturing process.

### Disease

Systemic lupus erythematosus (SLE) is a chronic, molecularly, pathologically and clinically heterogeneous autoimmune disease characterized by a wide range of organ damage manifestations (Fanouriakis et al 2021). Current standard of care includes conventional immunomodulatory and anti-inflammatory agents such as antimalarials, glucocorticoids and immunosuppressives (e.g. methotrexate, azathioprine, mycophenolate and cyclophosphamide) and biologics (such as, belimumab and very recently, anifrolumab as well as rituximab commonly used in this severe stage of this disease). Approximately 70% of the patients follow a relapsing-remitting disease course, the remaining divided equally between a prolonged remission and a persistently active disease despite therapy. For organ-threatening or life-threatening SLE, treatment usually includes an initial period of high-intensity immunosuppressive therapy to control disease activity, followed by a longer period of less intensive therapy to consolidate response and prevent relapses (Fanouriakis et al 2021).

Severe, refractory SLE (srSLE) patients are usually excluded from randomized clinical trials. srSLE patients, with or without renal involvement, after having failed immunosuppressive and biological therapies, have very limited treatment options. Autologous stem cell transplantation (ASCT) may be performed, however, it remains experimental and is associated with significant toxicities including mortality (Jayne et al 2004). In the present study, srSLE patients with or without lupus nephritis are included. If the initial data from srSLE patients are supportive, patients with other severe autoimmune diseases may be enrolled into a new study part (e.g. Part B, described in more detail below) after a substantial amendment.

### Overall Study Design

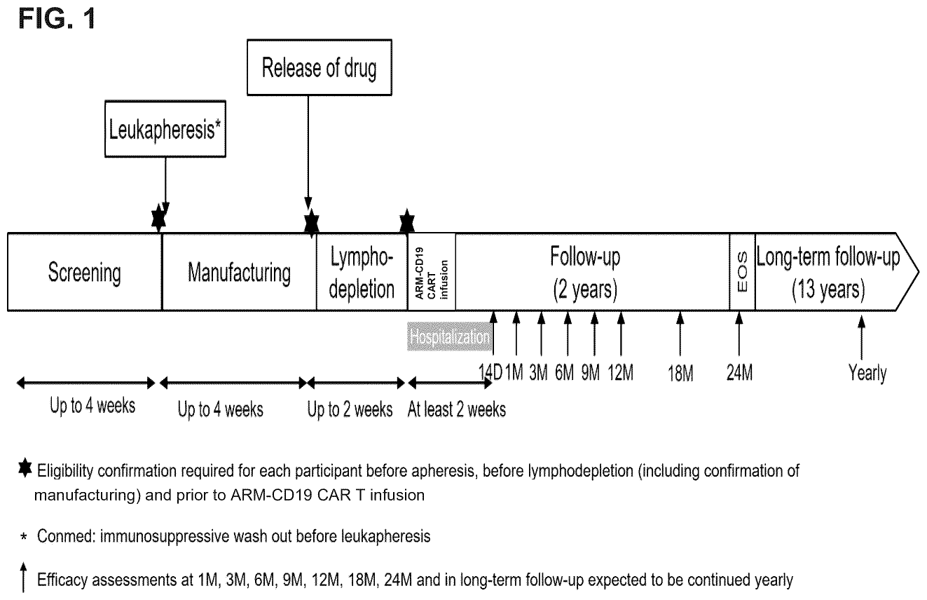

The study starts with Part A in participants diagnosed with srSLE. Inclusion of up to 3 additional parts (Part B, C, and D) may be initiated in parallel to Part A once first clinical data is available from this study and can include one or more of the following changes: (1) to assess the effect of lymphodepletion on safety/efficacy, (2) to assess a different dose level, (3) to broaden the study population by adding other indications such as other subtypes of SLE (including other organ involvements such cutaneous or articular), or other severe, B-cell driven, autoimmune diseases, e.g. systemic sclerosis or anti-synthetase syndrome where the benefit/risk assessment of ARM-CD19 CAR T is positive. The study design/participant journey is illustrated in FIG. 1. The total study duration for a participant is up to 27 months, after which a long-term follow-up is initiated until 15 years after ARM-CD19 CAR T administration. Participants are first evaluated for clinical eligibility (weeks −10 to −6) as detailed below. If clinically eligible, leukapheresis is scheduled (weeks −6 to −2). Once the leukapheresis product has been confirmed to be suitable for ARM-CD19 CAR T manufacturing, the manufacturing process is commenced. Before the planned apheresis and CAR-T cell injection, immunosuppressive treatments are stopped except for low-dose prednisolone (up to 10 mg/day) and antimalarials, which are allowed to continue. After the final product has been confirmed to be available, participants receive lymphodepleting therapy (up to two weeks prior to Day 1) as detailed below. Following pre-injection check on Day 1 and premedication, ARM-CD19 CAR T is administered as a single injection on Day 1.

Each participant is hospitalized until at least Day 14 following ARM-CD19 CAR T injection. Participants are closely monitored for any safety events for the first 2 months (twice a week visits for the first 5 weeks followed by weekly visits up to end of Month 2 (Day 60)). After that, the visit frequency is reduced to quarterly visits at Months 3, 6, 9, 12, and then twice a year (Months 18 and 24). End of study visit for a participant in this study is completed at Month 24, but participants will continue to be followed in the long-term follow-up.

Part A of this study will evaluate whether ARM-CD19 CAR T, following lymphodepletion, reduces disease activity to a low disease state for severe refractory SLE patients with at least one organ involvement. The estimand for Part A is described by the following attributes:
1. Population: srSLE patients with at least one organ involvement.
2. Endpoint: Achievement of LLDAS at Month 6.
3. Treatment of interest: Participants receiving lymphodepletion and a successful injection of ARM-CD19 CAR T within specified dose range, and not receiving any prohibited concomitant medication as per the protocol.
4. Measure: Percentage of participants achieving LLDAS at Month 6.

### Participant Inclusion and Exclusion Criteria

In Part A of this Phase 1/2 study, approximately 12 participants diagnosed with srSLE are treated with ARM-CD19 CAR T. Of these 12 participants, at least 8 (2/3rds) have renal involvement, as defined below. Each subject is first evaluated for clinical eligibility during screening. Participants eligible for inclusion in this study must meet the requirements for adequate renal, hepatic, cardiac, hematological and pulmonary function as follows: (1) renal function defined as serum creatinine of ≤1.5×ULN OR eGFR≥45 ml/min/1.73 m², (2) hepatic function defined as ALT and AST≤4×ULN and total bilirubin≤1.5×ULN with the exception of participants with Gilbert syndrome who may be included if their total bilirubin is ≤3.0×ULN and direct bilirubin≤1.5×ULN, and cardiac function defined as LVEF≥50% as determined by ECHO or MRA or MUGA at screening, unless cardiac impairment is clearly attributable to SLE, (3) hematologic function (regardless of transfusion) defined as absolute neutrophil count (ANC)>600/μL (only for participants with non-historical leukapheresis), platelets ≥50,000/μL, white blood cells count (WBC) ≥1,000 cells/uL, and absolute lymphocyte count ≥200/μL, and (4) pulmonary function defined as no or mild dyspnea (≤Grade 1) and oxygen saturation measured by pulse oximetry >90% on room air. The study includes men and women with SLE, aged ≥18 years and ≤65 years at screening, fulfilling the 2019 American College of Rheumatology (ACR) classification criteria for SLE (see Table X1, Aringer et al. (2019) Arthritis Rheumatol, p. 1400-1412, Aringer et al. (2019) Ann Rheum Dis, p. 1151-1159) at least 12 months prior to and at screening. SLE can be classified by criteria set out in Aringer et al. (2019) Ann Rheum Dis, p. 1151-1159.

TABLE X1
ACR 2019 definitions of SLE classification criteria

| Criteria | Definition |
|---|---|
| Antinuclear | Antinuclear antibodies (ANA) at a titer of ≥1:80 on HEp-2 cells or |
| antibodies (ANA) | an equivalent positive test at least once. Testing by |
| | immunofluorescence on HEp-2 cells or a solid phase ANA |
| | screening immunoassay with at least equivalent performance is |
| | highly recommended. |
| Fever | Temperature >38.3° Celsius. |
| Leukopenia | White blood cell count <4,000/mm³. |
| Thrombocytopenia | Platelet count <100,000/mm³. |
| Autoimmune | Evidence of hemolysis, such as reticulocytosis, low haptoglobin, |
| hemolysis | elevated indirect bilirubin, elevated LDH AND positive Coomb's |
| | (direct antiglobulin) test. |
| Delirium | Characterized by (1) change in consciousness or level of arousal |
| | with reduced ability to focus, and (2) symptom development over |
| | hours to <2 days, and (3) symptom fluctuation throughout the day, |
| | and (4) either (4a) acute/subacute change in cognition (e.g. memory |
| | deficit or disorientation), or (4b) change in behavior, mood, or |
| | affect (e.g. restlessness, reversal of sleep/wake cycle). |
| Psychosis | Characterized by (1) delusions and/or hallucinations without |
| | insight and (2) absence of delirium. |
| Seizure | Primary generalized seizure or partial/focal seizure. |
| Non-scarring alopecia | Non-scarring alopecia observed by a clinician*. |
| Oral ulcers | Oral ulcers observed by a clinician*. |
| Subacute cutaneous or discoid | Subacute cutaneous lupus erythematosus observed by a clinician*: |
| lupus | Annular or papulosquamous (psoriasiform) cutaneous eruption, |
| | usually photodistributed. Discoid lupus erythematosus observed by |
| | a clinician*: Erythematous-violaceous cutaneous lesions with |
| | secondary changes of atrophic scarring, dyspigmentation, often |
| | follicular hyperkeratosis/plugging (scalp), leading to scarring |
| | alopecia on the scalp. If skin biopsy is performed, typical changes |
| | must be present. Subacute cutaneous lupus: interface vacuolar |
| | dermatitis consisting of a perivascular lymphohistiocytic infiltrate, |
| | often with dermal mucin noted. Discoid lupus: interface vacuolar |
| | dermatitis consisting of a perivascular and/or periappendageal |
| | lymphohistiocytic infiltrate. In the scalp, follicular keratin plugs |
| | may be seen. In longstanding lesions, mucin deposition and |
| | basement membrane thickening may be noted. |
| Acute cutaneous | Malar rash or generalized maculopapular rash observed by a |
| lupus | clinician*. If skin biopsy is performed, typical changes must be |
| | present (Acute cutaneous lupus: interface vacuolar dermatitis |
| | consisting of a perivascular lymphohistiocytic infiltrate, often with |
| | dermal mucin noted. Perivascular neutrophilic infiltrate may be |
| | present early in the course. |
| Pleural or | Imaging evidence (such as ultrasound, x-ray, CT scan, MRI) of |
| pericardial effusion | pleural or pericardial effusion, or both. |
| Acute pericarditis | ≥2 of (1) pericardial chest pain (typically sharp, worse with |
| | inspiration, improved by leaning forward), (2) pericardial rub, (3) |
| | EKG with new widespread ST-elevation or PR depression, (4) new |
| | or worsened pericardial effusion on imaging (such as ultrasound, x- |
| | ray, CT scan, MRI). |
| Joint involvement | EITHER (1) synovitis involving 2 or more joints characterized by |
| | swelling or effusion OR (2) tenderness in 2 or more joints and at |
| | least 30 minutes of morning stiffness. |
| Proteinuria >0.5 g/24 h | Proteinuria >0.5 g/24 h by 24 hour urine or equivalent spot urine |
| | protein-to-creatinine ratio. |
| Class II or V lupus nephritis on | Class II: Mesangial proliferative lupus nephritis: Purely mesangial |
| renal biopsy according to | hypercellularity of any degree or mesangial matrix expansion by |
| ISN/RPS 2003 classification. | light microscopy, with mesangial immune deposit. A few isolated |
| | subepithelial or subendothelial deposits may be visible by immune- |
| | fluorescence or electron microscopy, but not by light microscopy. |
| | Class V: Membranous lupus nephritis: Global or segmental |
| | subepithelial immune deposits or their morphologic sequelae by |
| | light microscopy and by immunofluorescence or electron |
| | microscopy, with or without mesangial alterations. |
| Class III or IV lupus nephritis | Class III: Focal lupus nephritis: Active or inactive focal, segmental |
| on renal biopsy according to | or global endo- or extracapillary glomerulonephritis involving <50% |
| ISN/RPS 2003. | of all glomeruli, typically with focal subendothelial immune |
| | deposits, with or without mesangial alterations. Class IV: Diffuse |
| | lupus nephritis: Active or inactive diffuse, segmental or global |
| | endo- or extracapillary glomerulonephritis involving ≥50% of all |
| | glomeruli, typically with diffuse subendothelial immune deposits, |
| | with or without mesangial alterations. This class includes cases |
| | with diffuse wire loop deposits but with little or no glomerular |
| | proliferation. |
| Positive anti-phospholipid | Anti-Cardiolipin antibodies (IgA, IgG, or IgM) at medium or high |
| antibodies | titer (>40 APL, GPL or MPL, or >the 99th percentile) or positive |
| | anti-β2GP1 antibodies (IgA, IgG, or IgM) or positive lupus |
| | anticoagulant. |
| Low C3 OR low C4 | C3 OR C4 below the lower limit of normal. |
| Low C3 AND low C4 | Both C3 AND C4 below their lower limits of normal. |
| Anti-dsDNA antibodies OR | Anti-dsDNA antibodies in an immunoassay with demonstrated ≥90% |
| Anti-Smith (Sm) antibodies. | specificity for SLE against relevant disease controls OR Anti- |
| | Smith (Sm) antibodies. |

*This may include physical examination or review of a photograph. Source: Aringer et al. (2019) Ann Rheum Dis, p. 1151-1159.

Participants are positive for at least one of the following autoantibodies at screening: antinuclear antibodies (ANA) at a titer of ≥1:80 (on HEp-2 cells or an equivalent positive test), or anti dsDNA (above the ULN); or anti-Sm (above the ULN). Participants also have active (severe) disease as defined by SLEDAI-2K ≥8 (not including the SLEDAI-2K domains of lupus headache, cerebrovascular accident, organic brain syndrome) and at least one of the following significant SLE related organ involvements: (1) renal: histological diagnosis of proliferative lupus nephritis World Health Organization (WHO) ISN/RPS (Weening et al 2004) Class III or IV within 2 years of screening, AND at least one of the following at screening: first morning void UPCR: 0.7 to 4 mg/mg or urine sediment consistent with active proliferative lupus nephritis such as presence of cellular (granular or red blood cell) casts or hematuria (>5 red blood cells per high power field) if other causes such as menstrual bleeding are excluded; (2) endo/peri/myocarditis; (3) pleuritis or other lung involvement; or (4) vasculitis. At least 2/3rds of participants have lupus nephritis as defined by the above.

Patients also have failed to respond (i.e., having high disease activity as defined above despite the following therapy) to two or more standard immunosuppressive therapies (including one of mycophenolate or cyclophosphamide), unless contraindicated or having experienced documented adverse events or intolerance related to such immunosuppressive drugs not allowing their further use, in combination with glucocorticoids and failure to respond to at least one biological agent (unless contraindicated or the patient is deemed ineligible).

Exclusion criteria include: (1) prior treatment with anti-CD19 therapy, adoptive T cell therapy or any prior gene therapy product (e.g. CAR-T cell therapy), (2) any acute, severe lupus related flare during screening that needs immediate treatment and/or makes the immunosuppressive washout impossible and thus makes the patient ineligible for CD19 CAR-T therapy, such as acute CNS lupus (e.g. psychosis, epilepsy) or catastrophic antiphospholipid syndrome, and (3) any significant, likely irreversible organ damage related to SLE, e.g. end stage renal disease, where CD19 CAR-T cell therapy is deemed to be unlikely to benefit the patient.

### Study Treatment

Eligible participants undergo the following sequence of events prior to ARM-CD19 CAR T administration: (1) leukapheresis, (2) pre-lymphodepletion evaluation, (3) lymphodepletion, (4) premedication, and (5) pre-ARM-CD19 CAR T injection check.

Lymphodepleting therapy starts within one week before ARM-CD19 CAR T injection, which means that ARM-CD19 CAR T is injected 2 to 6 days after lymphodepleting therapy is completed. Lymphodepleting therapy may be repeated in the case ARM-CD19 CAR T has been delayed by more than 2 weeks. The lymphodepleting therapy regime is as follows: (1) fludarabine administered 25 mg/m² intravenously [i.v.] daily for 3 doses (for participants with renal impairment, the dose may be reduced as per local approved labels of fludarabine), and (2) cyclophosphamide administered 250 mg/m² i.v. daily for 3 doses starting with the first dose of fludarabine.

All participants are pre-medicated with acetaminophen (paracetamol, 650 mg, orally) and diphenhydramine (25−50 mg, i.v. or orally) or another H1 antihistamine approximately 30 to 60 minutes prior to injection. These medications can be repeated every 6 hours as needed. Non-steroidal anti-inflammatory medication may be prescribed if the participant continues to have fever not relieved with acetaminophen (paracetamol).

In Part A of this Phase 1/2 study, ARM-CD19 CAR T treatment consists of a single intravenous (i.v.) injection of a target dose of 12.5×10⁶ CAR-positive viable T cells (range 5 ×10⁶-12.5×10⁶ CAR-positive viable T cells) in cell suspension, without weight-based dose adjustment. The 12.5×10⁶ starting dose is selected based on data from the DLBCL arm of the Phase I oncology study, CYTB323A12101. In CYTB323A12101 DLBCL, high rates of complete tumor response by elimination of CD19 expressing cells were shown from doses of 12.5×10⁶ (63.2% CR at month 3), indicating that depletion of CD19 expressing B cells, the intended pharmacodynamic effect in srSLE, can be expected in srSLE from this dose. A dose of 2.5×10⁶ in CYTB323A12101 DLBCL resulted in lower CR rates at Month 3 (25%). A study of treatment of four patients with srSLE with a traditionally manufactured CD19 targeting CAR-T (Mougiakakos et al 2021, Schett et al 2022) reported a dose of 46×10⁶ CAR-T cells bein used.

Doses within the range of 2.5−40×10⁶ CAR-positive viable T cells can also be used, and toxicity expected to be lower than observed for participants with DLBCL in CYTB323A12101. In participants with DLBCL in CYTB323A12101, manageable safety was shown at target doses of 2.5−40×10⁶, with only 3 of 45 patients experiencing a ≥grade 3 CRS or ICANS. There was no evidence of a dose-safety relationship for DLBCL.

Prior to leukapheresis, immunosuppressive/immunomodulatory treatments are discontinued as they may interfere with the expansion or function of the CAR-T cells and, together with the profound B cell depletion, may cause excessive immunosuppression. The washout periods for the immunosuppressive agents are relatively short to minimize the risk of disease worsening; furthermore, antimalarials and corticosteroid treatment are allowed to continue, for the latter at a maximum dose of 10 mg per day (prednisone or equivalent) during leukapheresis and after ARM-CD19 CAR T injection. Corticosteroids are tapered to max 10 mg prednisone or equivalent at least one week before leukapheresis based on baseline dose, disease activity and clinical symptoms. After ARM-CD19 CAR T injection any remaining steroids are tapered any time post injection per the following: (a) if SLEDAI-2K reduced by 4 (or more) for at least 2 weeks: taper to 7.5 mg/day or less; (b) if SLEDAI-2K below 4: taper to 5 or 0 mg/day; or (3) if in remission steroids should be discontinued completely. In the first 24 weeks after the ARM-CD19 CAR T injection, the use of all immunosuppressive/immunomodulatory treatments are prohibited with the exception of corticosteroids (max. 10 mg prednisone or equivalent per day) and/or antimalarials. It is recommended to schedule leukapheresis prior to any planned corticosteroids as an absolute T cell count (absolute lymphocyte count multiplied by the percentage of CD3 positive lymphocytes)≤300/mm³ may result in a poor T cell collection and manufacturing failure. If the patient shows signs of worsening after leukapheresis but still deemed eligible for CAR-T therapy, an additional 5 mg prednisone or equivalent (so max 15 mg prednisone per day) as bridging therapy may be administered, which should be discontinued prior to ARM-CD19 CAR T administration. Antimalarials will be allowed to be continued during the full study as needed. After 24 weeks, SLE standard of care therapy may be given. Intravenous Immunoglobulin replacement therapy can be administered using the guideline by Hill et al, 2019.

### Safety, Pharmacokinetic (PK) and Efficacy Assessments

Objectives and related endpoints are summarized in Table X2. Safety of ARM-CD19 CAR T in participants with srSLE and other severe forms of autoimmune diseases is evaluated using parameters including vital signs, adverse events, laboratory parameters and electrocardiogram (ECG) evaluation.

TABLE X2
Objectives and related endpoints

| Objective(s) | Endpoint(s) |
|---|---|
| Primary objective(s) | Endpoint(s) for primary objective(s) |
| To assess safety of ARM-CD19 CAR T in | Safety parameters include vital signs, |
| participants with srSLE and other severe forms | adverse events, laboratory parameters and |
| of autoimmune diseases | ECG evaluation |
| Secondary objective(s) | Endpoint(s) for secondary objective(s) |
| To characterize the in vivo cellular kinetics | ARM-CD19 CAR transgene concentrations |
| (pharmacokinetics, PK) of ARM-CD19 CAR T | by qPCR over time in peripheral blood; |
| in peripheral blood by quantitative polymerase | cellular kinetics parameters (Cmax, AUC, |
| chain reaction (qPCR) | Tmax, T1/2, Clast, Tlast) |
| To characterize the incidence and prevalence of | Pre-existing and treatment induced |
| pre-existing and treatment induced | immunogenicity (cellular, humoral, |
| immunogenicity (cellular and humoral) of | neutralizing antibodies) of ARM-CD19 |
| ARM-CD19 CAR T | CAR T |
| To evaluate feasibility of the manufacturing | Manufacture success (defined as meeting |
| process in autoimmune disorders | release specifications and at or above the |
| | planned target dose) |
| Part A: To assess the effect of ARM-CD19 | At various timepoints: |
| CAR T on the following SLE disease activity | SLEDAI-2K |
| scores | BILAG-2004 |
| | Physician's global assessment |
| | LLDAS |
| | Remission (DORIS) |
| | SRI-4 |
| | BICLA |
| Part A: To evaluate effect of ARM-CD19 | UPCR at various timepoints |
| CAR T for srSLE participants who also have | Complete Renal Response (CRR) at |
| active lupus nephritis | various timepoints |

For pharmacokinetic (PK) analysis, serial blood samples are collected at different time points to measure ARM-CD19 CAR transgene concentrations in peripheral blood by quantitative polymerase chain reaction (qPCR). Levels of ARM-CD19 CAR transduced cells will be measured by flow cytometry of CD3-positive, ARM-CD19 CAR-positive cells. The absolute number of CD19+ B cells in the peripheral blood is measured by flow cytometry and used as the pharmacodynamics (PD) marker. The flow cytometry analysis can be performed using a validated panel that also includes the analysis of T cells and NK cells (TBNK).

Pre-existing and treatment-induced immunogenicity (cellular, humoral, neutralizing antibodies) of ARM-CD19 CAR T is assessed by one or more of the following: (1) humoral immunogenicity (anti-drug antibodies, ADA) measured by flow cytometry analysis of ADA binding to ARM-CD19 CAR-expressing cells, (2) presence of neutralizing antibodies measured by a reporter gene assay, (3) cellular immunogenicity measured by flow cytometry analysis of T cell interferon-gamma expression. Analytical methods for PK and immunogenicity assessments are listed in Table X3.

TABLE X3
Analytical methods associated with the PK and immunogenicity assessments

| | | | Lower limit of | Types of |
|---|---|---|---|---|
| | Analytical | | quantification | biological |
| Analyte | method | Units | (LLOQ) | sample |
| ARM-CD19 CAR | qPCR | copies/ug | 10 copies per | Peripheral |
| transgene | | | reaction | blood |
| | | | equivalent to | |
| | | | approx. 50 | |
| | | | copies/ug of | |
| | | | genomic DNA | |
| Surface expression | Flow cytometry | % of CD3+ | 1% of CD3+ T | Peripheral |
| of CAR-positive T | | viable | cells | blood |
| cells | | T cells | | |
| Anti-mCAR | Flow cytometry | MFI (mean | N/A - response | Serum |
| antibodies | | fluorescence | compared to CP | |
| (Humoral | | intensity) | defined during | |
| immunogenicity) | | | validation | |
| Neutralizing anti- | Cell-based | RLU (relative | N/A - response | Serum |
| mCAR antibodies | reporter | light | compared to CP | |
| | gene assay | units) | defined during | |
| | | | validation | |
| Antigen specific T | Intracellular | % IFN-g+ | % IFN-g+ CD4 | PBMC |
| cell lymphocytes | Interferon gamma | CD4/CD8 T cells | T cells for SEB | |
| (Cellular | staining and flow | (for both pool 1 | treated PBMC: | |
| immunogenicity) | cytometry | and pool 2) | 0.083%; | |
| | | | % IFN-g+ CD8 | |
| | | | T cells for SEB | |
| | | | treated PBMC: | |
| | | | 0.062%; | |
| | | | % IFN-g+ for | |
| | | | CEF treated | |
| | | | PBMC: 0.059% | |

To assess the effects of ARM-CD19 CAR T on disease activity in patients with srSLE, the following assessments are performed at various time points: LLDAS, Remission (DORIS), SLEDAI-2K, BILAG-2004, Physician's global assessment, SRI-4, and/or BICLA. Furthermore, for srSLE patients who have active lupus nephritis, in addition to the beforementioned assessments, UPCR and Complete Renal Response (CRR) at various timepoints are analyzed. These assessments are described below.

Lupus Low Disease Activity State (LLDAS) is defined by the following criteria: (1) SLEDAI-2K≤4, with no activity in major organ systems (renal, CNS, cardiopulmonary, vasculitis, fever) and no hemolytic anemia or gastrointestinal activity; (2) no new features of lupus disease activity compared with the previous assessment (SLEDAI-2K); (3) physician global assessment (PhGA, scale 0-3)≤1; (4) current corticosteroid (prednisolone or equivalent) dose≤7.5 mg/day; (5) well tolerated standard maintenance doses of immunosuppressive drugs and approved biological agents (Franklyn et al 2016). LLDAS is derived at assessment visits when SLEDAI-2K and PhGA are assessed.

For the remission evaluations, the DORIS definition (van Vollenhoven et al 2021) is: (1) clinical SLEDAI-2K=0; (2) Physician Global Assessment <0.5 (0-3). The participant may be on antimalarials, low-dose glucocorticoids (prednisolone≤5 mg/day), and/or stable immunosuppressives including biologics.

The Systemic Lupus Erythematosus Disease Activity Index (SLEDAI) is a validated model of experienced clinicians' global assessments of disease activity in systemic lupus erythematosus based on the consensus of a group of experts in the field of lupus research (Bombardier et al 1992). SLEDAI is calculated as a total score derived from a weighted index of 9 organ systems for disease activity in SLE, as follows: 8 for central nervous system and vascular descriptors, 4 for renal and musculoskeletal descriptors, 2 for serosal, dermal, and immunologic descriptors, and 1 for constitutional and hematologic descriptors. Systemic Lupus Erythematosus Disease Activity Index 2000 (SLEDAI-2K) is a modified version of SLEDAI to reflect persistent, active disease in those descriptors that had previously only considered new or recurrent occurrences (Gladman et al 2002). The range of SLEDAI-2K score is 0 to 105; a higher score indicating more severe disease.

British Isles Lupus Activity Group score (BILAG) records disease activity occurring over the past 4 weeks. The BILAG-2004 index covers 97 items and the assessment based on the principle of the doctor's intent to treat, which requires an assessment as 0=not present, 1=improving, 2=same, 3=worse, or 4=new over the last month (Isenberg et al 2005, Isenberg et al 2011, Yee et al 2006, Yee et al 2010). There are nine general headings: Constitutional, Mucocutaneous, Neuropsychiatric, Musculoskeletal, Cardiorespiratory, Gastrointestinal,

Ophthalmic, Renal, Haematological. Within each organ system, multiple manifestations and laboratory tests are combined into a single score for that organ. The resulting scores for each organ can be A through E, where A is very active disease, B is moderate activity, C is mild stable disease, D is resolved activity, and E indicates the organ was never involved. The BILAG-2004 system provides a disease activity measure that scores longitudinally and is clinically meaningful and easier to analyze in comparison with multiple categorical variables. This system has expected associations with change in therapy.

The Physician's global assessment (PhGA) of disease activity is performed using 100 mm visual analog scale (VAS) ranging from “0-no disease activity” to “3-severe disease activity”, after the question on how well the participant is doing with the disease considering all aspects affected by the disease. The distance in mm from the left edge of the scale is then measured and recorded.

The SLE responder index (SRI-4) utilizes the SLEDAI-2K score to determine global improvement, BILAG-2004 domain scores to ensure no significant worsening in heretofore unaffected organ systems, and physician's global assessment (PhGA) to ensure that improvements in disease activity are not achieved at the expense of the patient's overall condition (Furie et al 2017). The SRI is calculated any time the SLE disease activity scores are measured in individual participants. A responder is defined as having a ≥4-point reduction from baseline in SLEDAI-2K score AND no new BILAG-2004 A organ domain scores or ≥2 new BILAG-2004 B organ domain scores compared with baseline AND no worsening in PhGA (<0.3-point increase from baseline). If all 3 criteria are met, the participant will be considered a responder at that particular point in time; otherwise, the participant will be considered a non-responder.

BICLA (British Isles Lupus Assessment Group-based Composite Lupus Assessment, Wallace et al 2011) is a validated composite global measure of SLE disease activity that is derived from existing outcome assessments. Participants are considered as responders if they meet following criteria: (1) reduction of all baseline BILAG-2004 A to B/C/D and baseline B to C/D and no worsening in other organ systems defined as ≥1 new A or ≥2 new B items compared to baseline; (2) no worsening from baseline in SLEDAI-2K, defined as an increase from baseline of >0 points; (3) no worsening in PhGA, defined as an increase of ≥0.3 from baseline on a 0 to 3 visual analog scale.

Complete Renal Response (CRR) is defined as a decrease in UPCR (urinary protein creatinine ratio) to ≤0.5 mg/mg in 2 consecutive, first morning void urine specimens, plus an eGFR >60 ml/min per 1.73 m² or no decrease of ≥20% of screening eGFR on 2 consecutive occasions (Rovin et al 2019).

Patient reported outcomes (PRO) may also be completed at the scheduled visit before any clinical assessments are conducted. The Short Form Health Survey (SF-36 v2) is a widely used and extensively studied instrument to measure health-related quality of life among healthy participants and participants with acute and chronic conditions. It consists of eight subscales that can be scored individually: Physical Functioning, Role-Physical, Bodily Pain, General Health, Vitality, Social Functioning, Role-Emotional, and Mental Health. Two overall summary scores, the Physical Component Summary (PCS) and the Mental Component Summary (MCS) also can be computed. The SF-36 has proven useful in monitoring general and specific populations, comparing the relative burden of different diseases, differentiating the health benefits produced by different treatments, and in screening individual participants. The patient's global assessment of disease activity is performed using a Visual Analogue Scale (VAS) of 100 mm ranging from “no disease activity” to “severe disease activity”, after the question on how well the participant is doing with the disease considering all aspects affected by the disease. The distance in mm from the left edge of the scale and the value is measured.

### Results

Three patients received ARM-CD19 CAR T cells. Safety was assessed by evaluating vital signs, adverse events, laboratory parameters, and an electrocardiogram. No death or serious adverse events were reported. Adverse events: multiple grade 3 and 4 cytopenia, including a grade 4 neutropenia (recovered within 28 days) was reported; all these events were suspected to be related to lymphodepletion by the investigator. No ICANS and one case of CMV infection (recovered) has been reported. Preliminary efficacy suggests marked decreases in SLEDAI (the first subject has SLEDAI of zero (is in remission) by Day 90) and PhGA, in line with improvements in relevant disease biomarkers such as dsDNA, complement levels, and proteinuria. No unexpected safety signal was observed.

### EQUIVALENTS

The disclosures of each and every patent, patent application, and publication cited herein are hereby incorporated herein by reference in their entirety. While this invention has been disclosed with reference to certain embodiments, it is apparent that further embodiments and variations of this invention may be devised by others skilled in the art without departing from the true spirit and scope of the invention. The appended claims are intended to be construed to include all such embodiments and equivalent variations.

What is claimed is:

1. A method of treating systemic lupus erythematosus (SLE) in a human subject, the method comprising administering to the subject a population of T cells that express, or comprise a nucleic acid configured to express, a CD19 chimeric antigen receptor (CAR) at a dose of 0.5-50×10⁶ viable CAR+ T cells, wherein the CAR comprises a CD19 binding domain, a transmembrane domain and an intracellular signaling domain.

2. The method of claim 1, wherein the subject responds to the treatment as defined as having:
a) a ≥4-point reduction from baseline in SLEDAI-2K score;
b) no new BILAG-2004 A organ domain scores or ≥2 new BILAG-2004 B organ domain scores compared with baseline; or
c) no worsening in PhGA (<0.3-point increase from baseline).

3. The method of claim 1, wherein the subject experiences a decrease in SLEDAI score and a decrease in PhGA.

4. The method of claim 1, wherein the cells are administered at a dose of 5−12.5×10⁶ viable CAR+ T cells.

5. The method of claim 1, wherein the CD19 binding domain comprises:
(a) a heavy chain complementarity determining region 1 (HC CDR1) comprising the amino acid sequence of SEQ ID NO: 295, an HC CDR2 comprising the amino acid sequence of SEQ ID NO: 296, an HC CDR3 comprising the amino acid sequence of SEQ ID NO: 297, a light chain complementarity determining region 1 (LC CDR1) comprising the amino acid sequence of SEQ ID NO: 298, an LC CDR2 comprising the amino acid sequence of SEQ ID NO: 299, and an LC CDR3 comprising the amino acid sequence of SEQ ID NO: 300; or
(b) an scFv comprising the amino acid sequence of SEQ ID NO: 303.

6. The method of claim 1, wherein:
(a) the intracellular signaling domain comprises a primary signaling domain comprising a functional signaling domain derived from CD3 zeta;
(b) the intracellular signaling domain comprises a costimulatory signaling domain comprising a functional signaling domain derived from 4-1BB; or
(c) the intracellular signaling domain comprises a primary signaling domain comprising the amino acid sequence of SEQ ID NO: 9 or 10, or a costimulatory signaling domain comprising the amino acid sequence of SEQ ID NO: 7.

7. The method of claim 1, wherein the transmembrane domain comprises a transmembrane domain of protein chosen from the alpha, beta, or zeta chain of T-cell receptor, CD28, CD3 epsilon, CD45, CD4, CD5, CD8, CD9, CD16, CD22, CD33, CD37, CD64, CD80, CD86, CD134, CD137 and CD154.

8. The method of claim 1, wherein the transmembrane domain is attached to the CD19 binding domain via an IgG4 hinge or a CD8a hinge.

9. The method of claim 1, wherein the systemic lupus erythematosus is severe refractory systemic lupus erythematosus.

10. The method of claim 1, wherein the subject has been previously treated with, or is concurrently treated with, one or more of an antimalarial, a glucocorticoid, a calcineurin inhibitor, an immunomodulatory agent, a biological agent, rituximab, or a disease-modifying antirheumatic drug (DMARD).

11. The method of claim 1, wherein the population of cells was made by a method comprising:
(i) contacting a population of T cells with an agent that stimulates a CD3/TCR complex or an agent that stimulates a costimulatory molecule on the surface of the cells;
(ii) contacting the population of cells with a nucleic acid molecule encoding the CAR, thereby providing a population of T cells comprising the nucleic acid molecule; and
(iii) harvesting the population of T cells for storage or administration, wherein:
(a) step (ii) is performed together with step (i) or no later than 20 hours after the beginning of step (i), and step (iii) is performed no later than 30 hours after the beginning of step (i);
(b) step (ii) is performed together with step (i) or no later than 20 hours after the beginning of step (i), and step (iii) is performed no later than 30 hours after the beginning of step (ii); or
(c) the population of cells from step (iii) are not expanded, or expanded by no more than 5, 10, 15, 20, 25, 30, 35, or 40%, as assessed by the number of living cells, compared to the population of cells at the beginning of step (i).

12. The method of claim 11, wherein the agent that stimulates a CD3/TCR complex comprises an anti-CD3 antibody and the agent that stimulates a costimulatory molecule comprises an anti-CD28 antibody.

13. The method of claim 11, wherein the percentage of naïve T cells in the population of cells from step (iii) differs by no more than 10% from the percentage of naïve T cells in the population of cells at the beginning of step (i).

14. The method of claim 13, wherein the naïve T cells comprise CD45RA+CD45RO− CCR7+ T cells.

15. The method of claim 11, wherein the population of cells from step (iii) shows a higher percentage of naïve T cells compared with cells made by an otherwise similar method in which step (iii) is performed more than 5 days after the beginning of step (i).

16. The method of claim 15, wherein the naïve T cells comprise CD45RA+CD45RO− CCR7+ T cells.

17. The method of claim 11, wherein the population comprises:
(a) about the same percentage of naïve T cells as compared to the percentage of naïve T cells in the same population of cells prior to being engineered to express the CAR;
(b) a change within about 5% to about 10% of naïve T cells as compared to the percentage of naïve T cells in the same population of cells prior to being engineered to express the CAR;
(c) an increased percentage of naïve T cells as compared to the percentage of naïve T cells in the same population of cells prior to being engineered to express the CAR;
(d) about the same percentage of central memory T cells as compared to the percentage of central memory T cells in the same population of cells prior to being engineered to express the CAR;
(e) a change within about 5% to about 10% of central memory T cells as compared to the percentage of central memory T cells in the same population of cells prior to being engineered to express the CAR;
(f) a decreased percentage of central memory T cells as compared to the percentage of central memory T cells in the same population of cells prior to being engineered to express the CAR;
(g) about the same percentage of stem memory T cells as compared to the percentage of stem memory T cells in the same population of cells prior to being engineered to express the CAR;
(h) a change within about 5% to about 10% of stem memory T cells as compared to the percentage of stem memory T cells in the same population of cells prior to being engineered to express the CAR; or
(i) an increased percentage of stem memory T cells as compared to the percentage of stem memory T cells in the same population of cells prior to being engineered to express the CAR.

18. The method of claim 17, wherein:
(a) the percentage of naïve T cells is increased by at least 1.2, 1.4, 1.6, 1.8, 2.0, 2.2, 2.4, 2.6, 2.8, or 3-fold as compared to the percentage of naïve T cells in the same population of cells prior to being engineered to express the CAR;
(b) the percentage of central memory T cells is decreased by at least 20, 25, 30, 35, 40, 45, or 50%, as compared to the percentage of central memory T cells in the same population of cells prior to being engineered to express the CAR;
(c) the naïve T cells comprise CD45RO− CCR7+ T cells;
(d) the central memory T cells comprise CCR7+CD45RO+ T cells; or
(e) the stem memory T cells comprise CD45RA+CD95+IL-2 receptor β+CCR7+CD62L+ T cells.

19. A method of treating a subject having an autoimmune disease or disorder, the method comprising:
(I) administering to the subject a population of cells that express, or comprise a nucleic acid configured to express, a CD19 CAR, wherein the population of cells was made by a method comprising:
(i) contacting a population of cells with an agent that stimulates a CD3/TCR complex or an agent that stimulates a costimulatory molecule on the surface of the cells;
(ii) contacting the population of cells with a nucleic acid molecule encoding the CAR, thereby providing a population of cells comprising the nucleic acid molecule; and
(iii) harvesting the population of cells for storage or administration,
wherein step (ii) is performed together with step (i) or no later than 20 hours after the beginning of step (i), and step (iii) is performed no later than 30 hours after the beginning of step (i); or
(II) administering to the subject rapcabtagene autoleucel.

20. A method of making a population of cells that express a CAR, the method comprising:
(i) contacting a population of cells with an agent that stimulates a CD3/TCR complex or an agent that stimulates a costimulatory molecule on the surface of the cells, wherein the population of cells is from a subject having an autoimmune disease or disorder;
(ii) contacting the population of cells with a nucleic acid molecule encoding the CAR, thereby providing a population of cells comprising the nucleic acid molecule; and
(iii) harvesting the population of cells for storage or administration,
wherein step (ii) is performed together with step (i) or no later than 20 hours after the beginning of step (i), and step (iii) is performed no later than 30 hours after the beginning of step (i).
